(19) United States
(12) Patent Application Publication
(10) Pub. No.: US 2024/0033358 A1
(43) Pub. Date: Feb. 1, 2024

(54) COMBINATION THERAPIES WITH CHIMERIC ANTIGEN RECEPTOR (CAR)-EXPRESSING CELLS

(71) Applicant: Novartis AG, Basel (CH)

(72) Inventors: Ana Maria Chadbourne, Weston, MA (US); Sergei I. Agoulnik, Myrtle Beach, SC (US)

(73) Assignee: Novartis AG, Basel (CH)

(21) Appl. No.: 18/036,749

(22) PCT Filed: Nov. 12, 2021
(86) PCT No.: PCT/US2021/059143

§ 371 Date: May 12, 2023

Related U.S. Application Data
(60) Provisional application No. 63/113,749, filed on Nov. 13, 2020.

(51) Int. Cl. A61K 39/00; A61K 31/635; A61K 45/06; A61P 35/00

(52) U.S. Cl. CPC A61K 39/464412; A61K 39/4631; A61K 31/635; A61K 45/06; A61P 35/00; A61K 39/4611

(57) ABSTRACT

This disclosure provides methods for treating a B-cell lymphoma, by administering a CD 19 CAR therapy as described herein, in combination with a BCL2 inhibitor as described herein.

### RELATED APPLICATIONS

This application claims priority to U.S. Provisional Application 63/113,749 filed on Nov. 13, 2020, the entire contents of which are hereby incorporated by reference.

### SEQUENCE LISTING

The instant application contains a Sequence Listing which has been submitted electronically in ASCII format and is hereby incorporated by reference in its entirety. Said ASCII copy, created on Nov. 9, 2021, is named N2067-7171WO_SL.txt and is 490,552 bytes in size.

### FIELD OF THE INVENTION

The present invention relates generally to the use of immune effector cells that express a Chimeric Antigen Receptor (CAR) in combination with a BCL2 inhibitor, to treat a cancer, e.g., a lymphoma, e.g., a B-cell lymphoma.

### BACKGROUND OF THE INVENTION

Adoptive transfer (ACT) therapy with autologous T-cells, especially T-cells transduced with Chimeric Antigen Receptors (CAR) has shown promise in the treatment of relapsed or refractory hematological cancers. There is a medical need for CAR T cell therapies and combination therapies with improved efficacy for the treatment of, e.g., relapsed or refractory B-cell lymphomas.

### SUMMARY OF THE INVENTION

The present disclosure pertains, at least in part, to a method of treating a hematological cancer, e.g., a lymphoma, e.g., a B-cell lymphoma, comprising administering immune effector cells that express a chimeric antigen receptor (CAR) that binds a B-cell antigen, e.g., a B-cell antigen described herein, in combination with one or more of: an apoptosis inhibitor (e.g., a BCL2 inhibitor, a BCL6 inhibitor, or a combination thereof), or a MYC inhibitor. In some embodiments, the CAR-expressing cells bind CD19, e.g., a CD19 CAR-expressing cell described herein. In some embodiments, the B-cell lymphoma is a high-grade B-cell lymphoma (e.g., a double and/or triple hit lymphoma or a non-specified (NOS) high-grade lymphoma), DLBCL (e.g., relapsed and/or refractory DLBCL), a multiple myeloma, or a follicular lymphoma. Also described herein are compositions comprising the aforesaid combinations and additional methods of administrating said combinations to selected subjects.

In one aspect, the disclosure provides a method for treating a subject having, or identified as having, a lymphoma, e.g., B-cell lymphoma, e.g., wherein said lymphoma has an increased level and/or activity of a MYC gene or gene product and/or an anti-apoptotic gene or gene product. The method comprises:
administering to the subject a therapy comprising a population of immune effector cells that expresses a chimeric antigen receptor (CAR) that binds to a B cell antigen, in combination with one or more of a BCL-2 inhibitor, a BCL-6 inhibitor, or a MYC inhibitor, thereby treating the lymphoma in the subject.

In another aspect, the disclosure provides a method for treating a subject having a lymphoma, e.g., a B-cell lymphoma having an increased level and/or activity of a MYC gene or gene product and/or an anti-apoptotic gene or gene product (e.g., a high grade B-cell lymphoma). The method comprises:
administering to the subject one or more of: a BCL-2 inhibitor, a BCL-6 inhibitor, or a MYC inhibitor, wherein the subject is, or is identified as being, a non-responder, a partial responder, or a relapser to a CAR therapy that binds to a B-cell antigen.

In yet another aspect, the present disclosure provides a method for treating or preventing a relapse to a population of immune effector cells that expresses a Chimeric Antigen Receptor (CAR) that binds a B cell antigen in a subject with a lymphoma, e.g., a B-cell lymphoma, having increased level and/or activity of a MYC gene or gene product and/or an anti-apoptotic gene or gene product. The method comprises:
administering a BCL-2 inhibitor, a BCL-6 inhibitor, or a MYC inhibitor, or a combination thereof, to a subject who has undergone, is undergoing, or will receive, the CAR therapy,
thereby treating or preventing the relapse to the CAR therapy.

In some embodiments of any of the methods provided herein, the CAR binds to the B-cell antigen chosen from CD19, CD22, CD20, CD34, CD123, BCMA, FLT-3, ROR1, CD79b, CD179b, and/or CD79a. In some embodiments, the CAR binds to CD19 (“CD19 CAR therapy”). In some embodiments, the CAR19 therapy is a therapy comprising immune effector cells expressing an anti-CD19 binding domain, a transmembrane domain, and an intracellular signaling domain comprising a stimulatory domain.

In some embodiments, the BCL2 inhibitor is venetoclax.

In some embodiments of any of the methods provided herein, the lymphoma is a B-cell lymphoma. In some embodiments, the B-cell lymphoma is chosen from high grade B-cell lymphoma (e.g., a double and/or triple hit lymphoma or a non-specified NOS high-grade lymphoma), diffuse large B-cell lymphoma (DLBCL) (e.g., relapsed and/or refractory DLBCL), or follicular lymphoma (FL). In some embodiments, the B-cell lymphoma is a high-grade B-cell lymphoma, e.g., a double and/or triple hit (DH/TH) lymphoma or a non-specified NOS high-grade lymphoma. In some embodiments, the DH/TH lymphoma is a relapsed or refractory DH/TH lymphoma. In some embodiments, the high-grade B-cell lymphoma is a double hit (DH) lymphoma. In some embodiments, the high-grade B-cell lymphoma is a triple hit (TH) lymphoma. In some embodiments, the lymphoma is DLBCL, e.g., relapsed and/or refractory DLBCL. In some embodiments, the lymphoma is FL, e.g., relapsed and/or refractory FL. In some embodiments, the lymphoma is a multiple myeloma.

Those skilled in the art will recognize or be able to ascertain using no more than routine experimentation, many equivalents to the specific embodiments of the invention described herein. Such equivalents are intended to be encompassed by the following enumerated embodiments.

### ENUMERATED EMBODIMENTS

1. A method for treating a subject having, or identified as having, a lymphoma, e.g., B cell lymphoma, e.g., wherein said lymphoma has an increased level and/or activity of a MYC gene or gene product and/or an anti-apoptotic gene or gene product, wherein the method comprises:
   administering to the subject a therapy comprising a population of immune effector cells that expresses a Chimeric Antigen Receptor (CAR) that binds to a B cell antigen, in combination with one or more of a BCL-2 inhibitor, a BCL-6 inhibitor, or a MYC inhibitor, thereby treating the lymphoma in the subject.
2. A method of treating a subject having a lymphoma having an increased level and/or activity of a MYC gene or gene product and/or an anti-apoptotic gene or gene product, said method comprising:
   administering to the subject one or more of a BCL-2 inhibitor, a BCL-6 inhibitor, or a MYC inhibitor, wherein the subject is, or is identified as being, a non-responder, a partial responder, or a relapser to the CAR therapy.
3. The method of embodiment 2, wherein the subject has undergone, is undergoing, or will receive, the CAR therapy, e.g., a CD19 CAR therapy.
4. A method of treating or preventing a relapse to a population of immune effector cells that expresses a Chimeric Antigen Receptor (CAR) that binds to a B cell antigen, in a subject with a lymphoma having increased level and/or activity of a MYC gene or gene product and/or an anti-apoptotic gene or gene product, comprising:
   administering one or more of a BCL-2 inhibitor, a BCL-6 inhibitor, or a MYC inhibitor to a subject who has undergone, is undergoing, or will receive, the CAR therapy,
   thereby treating or preventing the relapse to the CAR therapy.
5. The method of any of embodiments 1 to 4, wherein the CAR binds a B-cell antigen chosen from CD19, CD20 CD22, CD20, CD34, CD123, BCMA, FLT-3, ROR1, CD79b, CD179b, and/or CD79a.
6. The method of any of embodiments 1 to 5, wherein, the CAR binds to CD19 (“CD19 CAR therapy”).
7. The method of any of embodiments 1 to 6, wherein the subject has, or is identified as having, an alteration in a MYC gene or gene product, or an alteration in an anti-apoptotic gene or gene product, or a combination thereof.
8. The method of embodiment 7, wherein the alteration results in increased level, e.g., expression, and/or activity of the MYC gene or gene product, and/or the anti-apoptotic gene or gene product.
9. The method of embodiment 7 or 8, wherein the alteration in the anti-apoptotic gene comprises an alteration in a BCL2 gene or a BCL6 gene, or a combination thereof.
10. The method of any of embodiments 7 to 9, wherein the alteration of the MYC gene or the anti-apoptotic gene induces high expression of the gene or gene product (e.g., protein), e.g., compared to a MYC or an anti-apoptotic gene not having the alteration.
11. The method of any of embodiments 7 to 10, wherein the alteration of the MYC gene or the anti-apoptotic gene is a rearrangement, e.g., translocation.
12. The method of any of embodiments 1 to 11, wherein the subject has, or is identified as having, a rearrangement, e.g., translocation, in the MYC gene and a rearrangement, e.g., translocation, in one or both of the BCL2 gene or the BCL6 gene.
13. The method of any of embodiments 1 to 12, wherein the subject has, or is identified as having, an increased level of a BCL2 or BCL6 gene or gene product, compared to a reference, e.g., a healthy subject or a subject not having a high-grade lymphoma.
14. The method of any of embodiments 1 to 13, wherein the subject has, or is identified as having an increased level of, e.g., increased number of cells positive for, a MYC gene or MYC gene product, e.g., as identified by detecting a rearrangement, e.g., translocation, using a FISH assay or an immunohistochemistry assay.
15. The method of embodiment 14, wherein the subject is identified as being MYC positive, e.g., by detecting greater than 40% of cells in a sample, e.g., a tumor biopsy or blood sample, from the subject as being positive for expression of a MYC gene product, e.g., by an immunohistochemistry assay.
16. The method of embodiment 15, wherein the MYC-positive subject, is further identified as having an increased level of a BCL2 gene or gene product and/or a BCL6 gene or gene product e.g., as identified by detecting a rearrangement, e.g., translocation, in a sample, e.g., a tumor biopsy or a blood sample, using a FISH assay or an immunohistochemistry assay.
17. The method of either of embodiments 15-16, wherein the MYC-positive subject having an increased level of the BCL2 gene or gene product or an increased level of the BCL6 gene or gene product is identified as having, a double hit (DH) lymphoma, e.g., a MYC and BCL2 or BCL6-positive lymphoma.
18. The method of any of embodiments 15 to 17, wherein the MYC-positive subject having an increased level of a BCL2 gene or gene product and a BCL6 gene or gene product is identified as having, a triple hit (TH) lymphoma, e.g., a MYC, BCL2, and BCL6-positive lymphoma.
19. The method of any of the preceding embodiments, wherein the lymphoma is chosen from a high grade B-cell lymphoma (e.g., a double or triple hit lymphoma, or a non-specified NOS high-grade lymphoma), a diffuse large B-cell lymphoma (DLBCL), or follicular lymphoma.
20. The method of embodiment 19, wherein the lymphoma is a high grade B-cell lymphoma.
21. The method of embodiment 20, wherein the high grade B-cell lymphoma is a double hit lymphoma.
22. The method of embodiment 20, wherein the high grade B-cell lymphoma is a triple hit lymphoma.
23. The method of embodiment 19, wherein lymphoma is DLBCL, e.g., a relapsed or refractory DLBCL.
24. The method of embodiment 19 or 16, wherein the DLBCL arises from a cell population comprising a Germinal Center B-Cell (GCB cell), an activated B-Cell (ABC cell), or an unclassified cell.
25. The method of embodiment 24, wherein the DLBCL arises from a cell population comprising a Germinal Center B-Cell (GCB cell).
26. The method of any of embodiments 19 or 23-25, wherein the DLBCL is relapsed or refractory DLBCL.
27. The method of embodiment 19, wherein the lymphoma is a follicular lymphoma.
28. The method of embodiment 19 or 27, wherein the follicular lymphoma is a relapsed or refractory FL.
29. The method of embodiment 19, wherein the lymphoma is a multiple myeloma.
30. The method of embodiment 19 or 29, wherein the multiple myeloma is a relapsed or refractory multiple myeloma.
31. The method of any of the preceding embodiments, wherein the subject has or has been identified as having low levels of tumor infiltrating CD3+ T cells, e.g., less than or equal to at least about 0%-3%, 0.5%-2.5%, 1%-2%, 1.5%-2.5%, 2%-3%, 3%, 2.5%, 2%, 1.5%, 1%, 0.5%, or 0% CD3+ T cells, e.g., as identified in a sample, e.g., a tumor biopsy sample or a blood sample, e.g., by using a fluorescent immunohistochemistry assay.
32. The method of any of the preceding embodiments, wherein the subject has or has been identified as having increased numbers of LAG3+CD3+ T cells, e.g., greater than or equal to at least about 5%-30%, 5%-20%, 10%-25%, 10%-20%, 15%-20%, 15%-25%, 15%-30%, 5%-15%, 5%, 10%, 15%, 20%, 25%, or 30% LAG3+CD3+ T cells, e.g., as identified in a sample, e.g., a tumor biopsy sample or a blood sample, e.g., by using a fluorescent immunohistochemistry assay.
33. The method of any of embodiments 1 to 2 or 4 to 32, wherein the subject has undergone, is undergoing the CAR therapy, e.g., the CD19 CAR therapy or will receive the CAR therapy, e.g., the CD19 CAR therapy.
34. The method of any of the preceding embodiments, wherein the subject has relapsed, or is identified as having relapsed, after treatment with the CAR therapy, e.g., the CD19 CAR therapy.
35. The method of any of the preceding embodiments, wherein the subject has relapsed or is identified as having relapsed based on one or more of:
   (1) a reappearance of a bone marrow involvement, e.g., a lesion, after a complete response;
   (2) a reappearance of a malignant effusion, after a complete response;
   (3) a reappearance of a nodal lesion greater than 1.5 cm (e.g., a previously normal lymph node becoming greater than 1.5 cm) by CT scan or MRI, after a complete response;
   (4) a reappearance of a discrete extranodal lesion (including liver or spleen) by CT scan or MRI after a complete response; or
   (5) a ≥50% increase in the size of a residual lymph node or mass, e.g., the long axis from baseline of the lymph node or mass.
36. The method of any of the preceding embodiments, wherein the CAR therapy, e.g., the CD19 CAR therapy and the BCL2 inhibitor, BCL6 inhibitor, MYC inhibitor or combination thereof are administered concurrently or sequentially.
37. The method of any of the preceding embodiments, wherein the subject is treated with one or more of the BCL2 inhibitor, the BCL6 inhibitor, or the MYC inhibitor before, concurrently and/or after the CD19 CAR therapy.
38. The method of any of embodiments 1 to 37, wherein the BCL2 inhibitor, the BCL6 inhibitor, or the MYC inhibitor, or combination thereof is administered prior to the CAR therapy, e.g., the CD19 CAR therapy.
39. The method of any of embodiments 1 to 37, wherein the CAR therapy, e.g., the CD19 CAR therapy is administered prior to the administration of the BCL2 inhibitor, the BCL6 inhibitor, or the MYC inhibitor, or combination thereof.
40. The method of any of embodiments 38 or 39, further comprising administering one or more, e.g., 1, 2, 3, 4, 5, 10, 20, 30 or more, subsequent doses of the BCL2 inhibitor, the BCL6 inhibitor, or the MYC inhibitor, or combination thereof.
41. The method of any of embodiments 1-38, wherein the CAR therapy, e.g., the CD19 CAR therapy, is administered at least 1 day, 2 days, 3 days, 4 days, 5 days, 6 days, 7 days, 10 days, 14 days, 15 days, 21 days, or 28 days after the BCL2 inhibitor, the BCL6 inhibitor, or the MYC inhibitor, or combination thereof, is administered.
42. The method of any of embodiments 2-41, wherein responsive to a determination of the subject as having relapsed to the CAR therapy, e.g., the CD19 CAR therapy, administering the BCL2 inhibitor. the BCL6 inhibitor, the MYC inhibitor, or the combination thereof.
43. The method of embodiment 42, further comprising administering a second therapy, e.g., a B-cell inhibitor.
44. The method of embodiment 43, wherein the second therapy comprises a second CAR therapy that binds to a B cell antigen, e.g., a CD19, CD22, CD20, CD34, CD123, BCMA, FLT-3, ROR1, CD79b, CD179b, or CD79a antigen.
45. The method of any of the preceding embodiments, wherein the subject is evaluated prior to, during, or after receiving the CAR therapy, e.g., the CD19 CAR therapy or one or more of the BCL2 inhibitor, BCL6 inhibitor, or the MYC inhibitor, for the presence or absence of the alteration in the MYC gene or gene product, or the alteration in the anti-apoptotic gene or gene product, or a combination thereof.
46. The method of embodiment 45, wherein the subject is evaluated prior to receiving the CAR therapy, e.g., the CD19 CAR therapy.
47. The method of embodiment 45, wherein the subject is evaluated prior to receiving the BCL2 inhibitor, the BCL6 inhibitor, the MYC inhibitor or the combination thereof.
48. The method of embodiment 45, wherein the subject is evaluated prior to receiving either or both of the CAR therapy, e.g., the CD19 CAR therapy, or the BCL2 inhibitor, the BCL6 inhibitor, the MYC inhibitor, or the combination thereof.
49. The method of embodiment 45, wherein the subject is evaluated after receiving the CAR therapy, e.g., the CD19 CAR therapy, but prior to the initiation of the administration of the BCL2 inhibitor, the BCL6 inhibitor, the MYC inhibitor or the combination thereof.
50. The method of any of embodiments 1 to 44, further comprising evaluating the subject prior to, during, or after receiving the CAR therapy, e.g., the CD19 CAR therapy, or one or more the BCL2 inhibitor, the BCL6 inhibitor, or the MYC inhibitor, for the presence or absence of the alteration in the MYC gene or gene product, or the alteration in the anti-apoptotic gene or gene product, or a combination thereof.
51. The method of embodiment 50, wherein the subject is evaluated prior to receiving either or both of the CAR therapy, e.g., the CD19 CAR therapy, or one or more of the BCL2 inhibitor, the BCL6 inhibitor, or the MYC inhibitor.
52. The method of embodiment 51, wherein the subject is evaluated after receiving the CD19 CAR therapy but prior to the initiation of the administration of one or more of the BCL2 inhibitor, BCL6 inhibitor, MYC inhibitor, or combination thereof.
53. The method of any of embodiments 50 to 52, wherein the evaluation occurs at least two time points before, after and/or during the CAR therapy, e.g., the CD19 CAR therapy.
54. The method of any one of the preceding embodiments, wherein the CAR therapy is a CD19 CAR therapy, wherein the CD19 CAR therapy comprises a CD19 CAR comprising an anti-CD19 binding domain, a transmembrane domain, and an intracellular signaling domain comprising a stimulatory domain.
55. The method of embodiment 54, wherein the anti-CD19 binding domain of the CD19 CAR comprises one or more of light chain complementary determining region 1 (LC CDR1), light chain complementary determining region 2 (LC CDR2), and light chain complementary determining region 3 (LC CDR3) of any anti-CD19 light chain binding domain amino acid sequence listed in Tables 2 or 3, and one or more of heavy chain complementary determining region 1 (HC CDR1), heavy chain complementary determining region 2 (HC CDR2), and heavy chain complementary determining region 3 (HC CDR3) of any anti-CD19 heavy chain binding domain amino acid sequence listed in Tables 2 or 3.
56. The method of embodiments 54 or 55, wherein the anti-CD19 binding domain of the CD19 CAR comprises an amino acid sequence of SEQ ID NO: 1-12 or SEQ ID NO: 59, or a sequence at least 95% identical thereto.
57. The method of embodiments 54 or 55, wherein the anti-CD19 binding domain comprises a sequence of SEQ ID NO: 2, or SEQ ID NO:59, or a sequence at least 95% identical thereto.
58. The method of any of the preceding embodiments, wherein the CD19 CAR comprises an amino acid sequence of any of SEQ ID NO: 31-42, SEQ ID NO: 5008, or SEQ ID NO: 58, or a sequence at least 95% identical thereto.
59. The method of any of the preceding embodiments, wherein the CD19 CAR comprises an amino acid sequence of any of SEQ ID NO: 31-42 or SEQ ID NO: 58, wherein the CAR comprises or does not comprise a leader sequence comprising the amino acid sequence of SEQ ID NO: 13.
60. The method of any of the preceding embodiments, wherein the CD19 CAR comprises a polypeptide having the amino acid sequence of SEQ ID NO:32, or SEQ ID NO: 58, or a sequence at least 95% identical thereto.
61. The method of any of the preceding embodiments, wherein the CD19 CAR therapy is a therapy comprising CTL-019 or CTL-119 or both.
62. The method of any of the preceding embodiments, wherein the CAR is a CD19 CAR, e.g., a CAR comprising an scFv amino acid sequence of SEQ ID NO: 5002, SEQ ID NO: 5005, SEQ ID NO: 5013, or SEQ ID NO: 5018, or a CAR comprising the amino acid sequence of SEQ ID NO: 5001, SEQ ID NO: 5004, SEQ ID NO: 5011, or SEQ ID NO: 5016.
63. The method of embodiment 62, wherein the anti-CD19 binding domain comprises the amino acid sequence of SEQ ID NO: 5002, SEQ ID NO: 5005, SEQ ID NO: 5013, or SEQ ID NO: 5018.
64. The method of embodiment 62 or 63, wherein the CD19 CAR comprises a polypeptide having the amino acid sequence of SEQ ID NO: 5001, SEQ ID NO: 5004, SEQ ID NO: 5011, or SEQ ID NO: 5016.
65. The method of any of the preceding embodiments, wherein the CAR, e.g., the CD19 CAR, comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154.
66. The method of any of the preceding embodiments the antigen binding domain is connected to the transmembrane domain by a hinge region, wherein, optionally, the hinge region comprises SEQ ID NO:14, or an amino acid sequence with at least 95% identity thereto.
67. The method of any of the preceding embodiments, wherein the intracellular signaling domain:
   a. comprises a costimulatory domain and/or a primary signaling domain;
   b. comprises a costimulatory domain comprising a functional signaling domain obtained from a protein selected from the group consisting of OX40, CD2, CD27, CD28, CDS, ICAM-1, LFA-1 (CD11a/CD18), ICOS (CD278), and 4-1BB (CD137),
   c. comprises a costimulatory domain comprising the amino acid sequence of SEQ ID NO:16 or SEQ ID NO:51;
   d. comprises a functional signaling domain of 4-1BB and/or a functional signaling domain of CD3 zeta; or
   e. comprises the amino acid sequence of SEQ ID NO: 16 and/or the amino acid sequence of SEQ ID NO:17 or SEQ ID NO:43.
68. The method of any of the preceding embodiments wherein the CAR further comprises a leader sequence, wherein, optionally, the leader sequence comprises SEQ ID NO: 13 or SEQ ID NO: 5020.
69. The method of embodiment 68, wherein the leader sequence comprises SEQ ID NO: 13.
70. The method of embodiment 68, wherein the leader sequence comprises SEQ ID NO: 5020.
71. The method of any of the preceding embodiments, wherein the CAR therapy, e.g., the CD19 CAR therapy, is administered intravenously.
72. The method of any of the preceding embodiments, wherein the CAR therapy, e.g., the CD19 CAR therapy, is administered intravenously over a period of about 15 minutes to about 45 minutes.
73. The method of any of the preceding embodiments, wherein the CAR therapy, e.g. the CD19 CAR therapy, is administered at a concentration of at least about 1-3×10⁶ to 1-3×10⁹ cells.
74. The method of any of the preceding embodiment, wherein the BCL2 inhibitor is chosen from venetoclax (ABT-199), navitoclax (ABT-263), ABT-737, BP1002, SPC2996, APG-1252, obatoclax mesylate (GX15-070MS), PNT2258, or oblimersen (G3139), or a combination.
75. The method of any of the preceding embodiments, wherein the BCL2 inhibitor is venetoclax.
76. The method of any of the preceding embodiments, wherein the BCL2 inhibitor is administered at fixed dose.
77. The method of any of embodiments 1 to 75, wherein the BCL2 inhibitor is administered at multiple doses, e.g., a ramp-up cycle.
78. The method of embodiment 77, wherein the BCL2 inhibitor is administered in a ramp-up cycle for e.g., about 5 weeks, followed by a fixed dose for, e.g., about 24 months.
79. The method of any of the preceding embodiments, wherein the BCL2 inhibitor is administered at a dose of about 10 mg to about 400 mg, e.g., about 10 mg to about 30 mg, about 40 mg to about 60 mg, about 80 mg to about 120 mg, about 150 mg to about 250 mg, or about 350 mg to about 450 mg.
80. The method of embodiment 79, wherein the BCL2 inhibitor is administered at a dose of about 20 mg, 100 mg, about 200 mg, or about 400 mg.
81. The method of any of embodiments 77 to 80, wherein the BCL2 inhibitor is administered (a) at a dose of about 20 mg once a day for e.g., about 1 week, (b) at a dose of about 50 mg once a day for e.g., about 1 week, (c) at a dose of about 100 mg once a day for, e.g., about 1 week, (d) at a dose of about 200 mg once a day for, e.g., about 1 week, (e) at a dose of about 400 mg once a day for, e.g., about 1 week, and (f) at a fixed dose of about 400 mg once a day for, e.g., about 24 months.
82. The method of any of the preceding embodiments, wherein the BCL2 inhibitor is administered daily.
83. The method of any of the preceding embodiments, wherein the BCL2 inhibitor is administered once a day.
84. The method of any of the preceding embodiments, wherein the BCL2 inhibitor is administered for at least 5-10 consecutive days.
85. The method of any of the preceding embodiments, wherein the BCL2 inhibitor is administered orally.
86. The method of any of the preceding embodiments, wherein the BCL6 inhibitor comprises BI-3812, compound 79-6, or FX1.
87. The method of any of the preceding embodiments, wherein the MYC inhibitor comprises MLN0128, 9-ING-41, CUDC-907, or Oncomyc.
88. The method of any of the preceding embodiments, further comprising administering a standard of care for a B-cell lymphoma, e.g., a high-grade B-cell lymphoma or a DLBCL, e.g., a CD20 inhibitor, a chemotherapeutic agent, and/or a corticosteroid.
89. The method of embodiment 88, wherein the CD20 inhibitor is an anti-CD20 antibody.
90. The method of embodiment 89, wherein the anti-CD20 antibody is rituximab or obinutuzumab.
91. The method of any of embodiments 88-55, wherein the chemotherapeutic agent is cyclophosphamide, vincristine, and/or doxorubicin.
92. The method of any of embodiments 88-56, wherein the corticosteroid is prednisone.
93. The method of any of the preceding embodiments, wherein the subject is a mammal, e.g., a human.
94. The method of any of the preceding embodiments, which results in prevention, delay, or reduction of progression of the high grade lymphoma.
95. The method of any of the preceding embodiments, which results in an improved response in the subject.
96. The method of any of the preceding embodiments, which results in an increase in disease free survival compared to treating with the CAR therapy, e.g., CD19 CAR therapy, alone.
97. The method of any of the preceding embodiments, which results in an increase in progression free survival compared to treating with CAR therapy, e.g., CD19 CAR therapy alone.
98. A combination comprising a CAR that binds a B-cell antigen and one or more of a BCL2 inhibitor, a BCL6 inhibitor, or a MYC inhibitor, e.g., for use according to a method of any of the preceding embodiments.
99. A CAR that binds a B-cell antigen for use in combination with one or more of a BCL2 inhibitor, a BCL6 inhibitor, or a MYC inhibitor in a method of any of embodiments 1-97.
100. One or more of a BCL2 inhibitor, a BCL6 inhibitor, or a MYC inhibitor for use in combination with a CAR that binds a B-cell antigen in a method of any of embodiments 1-97.
101. A combination comprising a CD19 CAR therapy and a BCL2 inhibitor, e.g., for use according to a method of any of embodiments 1-97.
102. A CD19 CAR therapy for use in combination with a BCL2 inhibitor in a method of any of embodiments 1-97.
103. A BCL2 inhibitor for use in combination with a CD19 CAR in a method of any of embodiments 1-97.
104. A combination comprising the CAR therapy, e.g., a CD19 CAR therapy and one or more of the BCL2 inhibitor, the BCL6 inhibitor, or the MYC inhibitor for use in a method of treating the lymphoma, e.g., a B-cell lymphoma, according to any one of the preceding embodiments.
105. A combination comprising a CD123 CAR therapy and a BCL2 inhibitor, e.g., for use according to a method of any of embodiments 1-97.
106. A CD123 CAR therapy for use in combination with a BCL2 inhibitor in a method of any of embodiments 1-97.
107. A BCL2 inhibitor for use in combination with a CD123 CAR in a method of any of embodiments 1-97.
108. A method for treating a subject having, or identified as having, a leukemia, e.g., B-cell leukemia, wherein the method comprises:
   administering to the subject a therapy comprising a population of immune effector cells that expresses a Chimeric Antigen Receptor (CAR) that binds to a B cell antigen, e.g., a CD123 CAR, in combination with one or more of a BCL-2 inhibitor, a BCL-6 inhibitor, or a MYC inhibitor, thereby treating the leukemia in the subject.
109. The method of embodiment 108, wherein the B-cell antigen is CD123.
110. A combination comprising a CAR therapy, e.g., a CD123 CAR therapy and one or more of the BCL2 inhibitor, the BCL6 inhibitor, or the MYC inhibitor for use in a method of treating a leukemia, e.g., a B-cell leukemia.
111. The combination for use of embodiment 110, wherein the CAR therapy is a CD123 CAR therapy.
112. The method of embodiment 108-109 or the combination for use of claim 114-111, wherein the leukemia is an acute myeloid leukemia (AML).
113. The method of embodiment 108-109 or 112 or the combination for use of claim 110-111, wherein the BCL-2 inhibitor comprises venetoclax.

### BRIEF DESCRIPTION OF THE DRAWINGS

FIGS. 1A-1B shows response to CART19 therapy in patients with relapsed or refractory diffuse large B-cell lymphoma (DLBCL) classified by the presence or absence of MYC expression, as well as MYC, BCL2, and/or BCL6 gene rearrangements to further identify double/triple hit (DH/TH) lymphoma. FIG. 1A shows the PFS, and FIG. 1B shows OS following CART19 therapy in patients stratified as MYC(+) DH/TH, MYC(+) Non-DH/TH, or MYC(−).

FIGS. 2A-2B shows response to CART19 therapy in patients with relapsed or refractory diffuse large B-cell lymphoma (DLBCL) classified by the presence or absence of MYC expression, as well as MYC, BCL2, and/or BCL6 gene rearrangements to further identify double/triple hit (DH/TH) lymphoma. FIG. 2A shows the PFS, and FIG. 2B shows the OS following CART19 therapy in patients stratified as MYC(+) DH/TH, or MYC(−) Non-DH/TH.

FIGS. 3A-3B show duration of response (DOR) to CART19 therapy in patients with relapsed or refractory diffuse large B-cell lymphoma (DLBCL) classified by the presence or absence of MYC expression, and MYC, BCL2, and/or BCL6 gene rearrangements to further identify double/triple hit (DH/TH) lymphoma. FIG. 3A shows the DOR in patients stratified as MYC(+) DH/TH, MYC(+) Non-DH/TH, or MYC(−). FIG. 3B shows the DOR in patients stratified as MYC(+) DH/TH, or MYC(−) Non-DH/TH.

FIGS. 4A-4C shows duration of response (DOR), progression free survival (PFS), and overall survival (OS) following treatment with CART19 therapy in patients whose baseline tumor biopsies were tested for baseline MYC expression. FIG. 4A shows duration of response in the months post remission in MYC(+) compared to MYC(−) patients. FIG. 4B shows PFS in MYC(+) compared to MYC(−) patients. FIG. 4C shows OS in MYC(+) compared to MYC(−) patients.

FIG. 5 shows the best overall response (BOR) to CART19 therapy in patients with relapsed or refractory DLBCL and additional B-cell lymphoma subsets, including those patients positive for MYC, patients negative for MYC, and/or patients positive for DH/TH lymphomas at 1 month post-treatment.

FIG. 6 shows the response to CART19 therapy in patients with relapsed or refractory DLBCL and additional B-cell lymphoma subsets, including those patients positive for MYC, patients negative for MYC, and/or patients positive for DH/TH lymphomas at 6 months post-treatment.

FIG. 7 shows the in vitro activity of CART19 cells on SuDHL6 double-hit lymphoma cells. The CART19 cells resulted in less than 50% killing of the SuDHL6 cells, indicating that these cells appear to be refractory to CART19 activity.

FIG. 8 shows the in vitro activity of CART19 cells in combination with a BCL2 inhibitor on SuDHL6 double-hit lymphoma cells. The BCL2 inhibitor improved responses to CART19 cells in the SuDHL6, double-hit lymphoma cells in vitro and increased tumor cell killing.

FIG. 9 shows the tumor volume over the days post-implant in mice implanted with SuDHL6 double hit lymphoma cell, which indicated that SuDHL6 cells implanted in mice can be used as an in vivo double-hit lymphoma model for investigating responses to CART19 combination therapies, e.g., a CART19 combination therapy described herein.

FIGS. 10A-10C shows the in vivo activity of CART19 cells in combination with a BCL2 inhibitor in double-hit lymphoma model. FIG. 10A shows the tumor volume in mice over the days post-treatment with a PBS vehicle control (left) or a BCL2 inhibitor (right). FIG. 10B shows the tumor volume in mice over the days post-treatment with untransduced CART control cells (UTD) (left) or untransduced CART control cells in combination with a BCL2 inhibitor (right). FIG. 10C shows the tumor volume in mice over the days post-treatment with CART19 cells (UTD) (left) or CART19 cells in combination with a BCL2 inhibitor (right).

FIGS. 11A-11C shows the effects of BCL2 inhibitors on T cell proliferation and kinetics. The number of CD3+ T cells in 20 μL of blood was quantified weekly post-treatment. FIG. 11A shows the number of T cells following treatment with untransduced CART control cells (UTD) (left) or a BCL2 inhibitor (venetoclax) (right). FIG. 11B shows the number of T cells following treatment with CART19 cells alone (left) or CART19 cells in combination with a BCL2 inhibitor (venetoclax) (right). FIG. 11C shows, summarizes the data presented in FIGS. 11A-11B, depicting the average number of T cells quantified per 20 μL of blood each week in the indicated treatment groups.

FIGS. 13A-13C shows duration of response (DOR), progression free survival (PFS), and overall survival (OS) following treatment with CART19 therapy in patients stratified by the percentage (%) CD3 positive cells (TIM3/LAG3 assay) measured in a baseline tumor biopsy. FIG. 13A shows duration of response in the months post remission in patients with ≤3% CD3+ cells compared to patients with >3% CD3+ cells patients. FIG. 13B shows PFS in patients with ≤3% CD3+ cells compared to patients with >3% CD3+ cells patients. FIG. 13C shows OS in patients with ≤3% CD3+ cells compared to patients with >3% CD3+ cells patients.

FIG. 14 shows the response to CART19 therapy in patients with relapsed or refractory DLBCL and additional B-cell lymphoma subsets, including those patients positive for MYC, patients negative for MYC, and/or patients positive for DH/TH lymphomas at 3 months post-treatment.

FIGS. 15A-15C shows duration of response (DOR), progression free survival (PFS), and overall survival (OS) following treatment with CART19 therapy in patients stratified by the percentage (%) LAG3 positive, CD3 positive cells (TIM3/LAG3 assay) measured in a baseline tumor biopsy. FIG. 15A shows duration of response in the months post remission in patients with ≤20% LAG3+CD3+ cells compared to patients with ≥20% LAG3+CD3+ cells patients. FIG. 15B shows PFS in patients with ≤20% LAG3+CD3+ cells compared to patients with >20% LAG3+CD3+ cells patients. FIG. 15C shows OS in patients with ≤20% LAG3+CD3+ cells compared to patients with ≥20% LAG3+CD3+ cells patients.

FIG. 16 shows the probability of progression free survival (%) in patients of the JULIET trial following autologous anti-CD19 CAR-T cell infusion.

FIGS. 17A-17B shows the percentage of mycloid derived suppressor cells (MDSCs) in a baseline biopsy at month 3 (FIG. 17A) and month 9 (FIG. 17B). The percentage of CD11b+HLADR(−) cells (MDSCs) of all cells is shown on the X-axis and the percentage of CD11b+ cells (myeloid lineage) of all cells is shown on the Y-axis.

FIG. 18 depicts a survival tree analysis of MYC status and normal pre-infusion LDH levels. The probability of progression free survival (%) over the months following infusion is shown for MYC(−) and normal pre-infusion levels of LDH (left), MYC(+) and normal pre-infusion levels of LDH (center), and 1-2×ULN pre-infusion LDH levels (right).

FIG. 19 shows the event-free probability (relapse-free) (%) over the time from the onset of a response to the CD19 CAR-T infusion.

FIG. 20 shows the probability of survival (%) in patients over time since CD19 CAR-T cell infusion.

FIG. 21 shows CD19+ B cells per μL over the day post infusion in patients by M3 response. The left graph shows subjects with CR/PR and the right graph shows progressive disease (PD)/stable disease (SD), or unknown response.

FIG. 22 shows the percentage of CD3+ T cells by month 3 response in CR/PR patients (left), and non-responders (right).

FIG. 23 shows the percentage of LAG3+CD3+ T cells by month 3 response in CR/PR patients (left), and non-responders (right).

FIG. 24 shows the correlation between genetic subtypes and M3 response in patients experiencing CR, PR, PD, or unknown at month 3. The left graph is the Chapuy DLBCL subset and the right graph is the Schmitz DLBCL subset. BN2 refers to BCL6 fusions and NOTCH2 mutations, EZB refers to EZH2 mutations and BCL2 translocations, N1 refers to NOTCH1 mutations, and UNK refers to unknown.

### DETAILED DESCRIPTION

Described herein, inter alia, is a method for treating a subject having, or identified as having, a lymphoma, e.g., a B-cell lymphoma, e.g., wherein said lymphoma has an increased level and/or activity of a MYC gene or gene product and/or an anti-apoptotic gene or gene product (e.g., high grade B-cell lymphoma, DLBCL, multiple myeloma, or FL), comprising administering to the subject a therapy comprising a population of immune effector cells that expresses a chimeric antigen receptor (CAR) that binds to a B cell antigen (e.g., a CD19 CAR), in combination with one or more of a BCL-2 inhibitor, a BCL-6 inhibitor, or a MYC inhibitor, thereby treating the lymphoma in the subject. In some embodiments, the BCL2 inhibitor is venetoclax.

Without wishing to be bound by theory, it is believed that a subject having or identified as having a lymphoma comprising an increased level and/or activity of a MYC gene or gene product and/or an anti-apoptotic gene or gene product (e.g., a high grade lymphoma, e.g., a double/triple hit lymphoma), are likely to have a decreased response to and/or increased relapse in response to CAR19 therapy. Bcl-2 has been shown to inhibit apoptosis of factor-deprived cells but does not prevent apoptosis of immune cell mediated killing, indicating different mechanisms of apoptosis induction (Vaux et al. Int Immunol. 1992; 4(7): 821-824). Without wishing to be bound by theory, it is believed that in some embodiments, inhibition of Bcl-2, which promotes direct cell apoptosis in combination with a CAR therapy targeting a B cell antigen, e.g., a CAR19 therapy, can improve the efficacy of CAR therapy responses and the durability of response in a subject having, e.g., a high grade lymphoma (e.g., double/triple hit lymphoma).

Also disclosed herein, is a method for treating a subject having, or identified as having, a lymphoma, e.g., B-cell lymphoma, e.g., wherein said lymphoma has an increased level and/or activity of a MYC gene or gene product and/or an anti-apoptotic gene or gene product, comprising administering to the subject one or more of a BCL-2 inhibitor, a BCL-6 inhibitor, or a MYC inhibitor, wherein the subject is, or is identified as being, a non-responder, a partial responder, or a relapser to a CAR therapy that binds to a B-cell antigen. Also disclosed herein is a method for treating or preventing a relapse to a population of immune effector cells that expresses a Chimeric Antigen Receptor (CAR) that binds a B cell antigen in a subject with a lymphoma, e.g., a B-cell lymphoma, having increased level and/or activity of a MYC gene or gene product and/or an anti-apoptotic gene or gene product, comprising administering a BCL-2 inhibitor, a BCL-6 inhibitor, or a MYC inhibitor, or a combination thereof, to a subject who has undergone, is undergoing, or will receive, the CAR therapy, thereby treating or preventing the relapse to the CAR therapy. The combinations described herein can be used according to a dosage regimen described herein. Compositions comprising the aforesaid combinations and additional methods of administrating said combinations to selected subjects, as described herein, are also provided.

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances±10%, or in some instances±5%, or in some instances±1%, or in some instances±0.1% from the specified value, as such variations are appropriate to perform the disclosed methods.

The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain (e.g., comprise a chimeric fusion protein). In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one embodiment, the stimulatory molecule of the CAR is the zeta chain associated with the T cell receptor complex. In one aspect, the cytoplasmic signaling domain comprises a primary signaling domain (e.g., a primary signaling domain of CD3-zeta). In one embodiment, the cytoplasmic signaling domain further comprises one or more functional signaling domains of at least one costimulatory molecule as defined below. In one embodiment, the costimulatory molecule is a costimulatory molecule described herein, e.g., 4-1BB (i.e., CD137), CD27, ICOS, and/or CD28. In one embodiment, the CAR comprises a chimeric fusion protein comprising an extracellular antigen binding domain, a transmembrane domain and an intracellular signaling domain comprising a functional signaling domain of a stimulatory molecule. In one embodiment, the CAR comprises a chimeric fusion protein comprising an extracellular antigen binding domain, a transmembrane domain and an intracellular signaling domain comprising a functional signaling domain of a co-stimulatory molecule and a functional signaling domain of a stimulatory molecule. In one embodiment, the CAR comprises a chimeric fusion protein comprising an extracellular antigen binding domain, a transmembrane domain and an intracellular signaling domain comprising two functional signaling domains of one or more co-stimulatory molecule(s) and a functional signaling domain of a stimulatory molecule. In one embodiment, the CAR comprises a chimeric fusion protein comprising an extracellular antigen binding domain, a transmembrane domain and an intracellular signaling domain comprising at least two functional signaling domains of one or more co-stimulatory molecule(s) and a functional signaling domain of a stimulatory molecule. In one embodiment, the CAR comprises an optional leader sequence at the amino-terminus (N-terminus) of the CAR fusion protein. In one embodiment, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

A CAR that comprises an antigen binding domain (e.g., a scFv, or TCR) that binds to a specific tumor antigen X, such as those described herein, is also referred to as XCAR or CARX. For example, a CAR that comprises an antigen binding domain that binds to CD19 is referred to as CD19 CAR or CAR19.

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CHli domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The portion of a CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In one embodiment, the antigen binding domain of a CAR comprises an antibody fragment. In a further embodiment, the CAR comprises an antibody fragment that comprises a scFv.

As used herein, the term “antigen binding domain” or “antibody molecule” refers to a protein, e.g., an immunoglobulin chain or fragment thereof, comprising at least one immunoglobulin variable domain sequence. The term “antigen binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The portion of the CAR of the invention comprising an antigen binding domain, e.g., an antibody or antibody fragment thereof, may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises a scFv.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically

The term “xenogeneic” refers to any material derived from an animal of a different species.

“Derived from” as that term is used herein, indicates a relationship between a first and a second molecule. It generally refers to structural similarity between the first molecule and a second molecule and does not connote or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connote or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR described herein can be replaced with other amino acid residues from the same side chain family and the altered CAR can be tested using the functional assays described herein.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing cytoplasmic signaling sequence that is of particular use in the invention includes, but is not limited to, those derived from CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In a specific CAR of the invention, the intracellular signaling domain in any one or more CARS of the invention comprises an intracellular signaling sequence, e.g., a primary signaling sequence of CD3-zeta. In a specific CAR of the invention, the primary signaling sequence of CD3-zeta is the sequence provided as SEQ ID NO:9 (mutant CD3 zeta), or the equivalent residues from a non-human species, e.g., mouse, rodent, monkey, ape and the like. In a specific CAR of the invention, the primary signaling sequence of CD3-zeta is the sequence as provided in SEQ ID NO:10 (wild-type human CD3 zeta), or the equivalent residues from a non-human species, e.g., mouse, rodent, monkey, ape and the like.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. The intracellular signaling domain can generate a signal that promotes an immune effector function of the CAR containing cell, e.g., a CART cell. Examples of immune effector function, e.g., in a CART cell, include cytolytic activity and helper activity, including the secretion of cytokines. In embodiments, the intracellular signaling domain is the portion of a protein which transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (“ICOS”), FcεRJ, and CD66d, CD32, DAP10, and DAP12.

The term “zeta” or alternatively “zeta chain”, “CD3-zeta” or “TCR-zeta” is defined as the protein provided as GenBank Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof. In one aspect, the “zeta stimulatory domain” or a “CD3-zeta stimulatory domain” is the sequence provided as SEQ ID NO:9. In one aspect, the “zeta stimulatory domain” or a “CD3-zeta stimulatory domain” is the sequence provided as SEQ ID NO:10.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to MHC class I molecule, TNF receptor proteins, Immunoglobulin-like proteins, cytokine receptors, integrins, signalling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to an intracellular portion of a costimulatory molecule. The intracellular signaling domain can comprise the entire intracellular portion, or the entire native intracellular signaling domain, of the molecule from which it is derived, or a functional fragment thereof.

The intracellular signaling domain can comprise the entire intracellular portion, or the entire native intracellular signaling domain, of the molecule from which it is derived, or a functional fragment thereof.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like. In one aspect, the “4-1BB costimulatory domain” is the sequence provided as SEQ ID NO:7 or the equivalent residues from a non-human species, e.g., mouse, rodent, monkey, ape and the like.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “depletion” or “depleting”, as used interchangeably herein, refers to the decrease or reduction of the level or amount of a cell, a protein, or macromolecule in a sample after a process, e.g., a selection step, e.g., a negative selection, is performed. The depletion can be a complete or partial depletion of the cell, protein, or macromolecule. In an embodiment, the depletion is at least a 1%, 2%, 5%, 10%, 15%, 20%, 25%, 30%, 35%, 40%, 45%, 50%, 55%, 60%, 65%, 70%, 75%, 80%, 85%, 90%, 95%, or 99% decrease or reduction of the level or amount of a cell, a protein, or macromolecule, as compared to the level or amount of the cell, protein or macromolecule in the sample before the process was performed.

The term “enriched” or “enrichment”, as used interchangeably herein, refers to the increase of the level or amount of a cell, a protein, or macromolecule in a sample after a process, e.g., a selection step, e.g., a positive selection, is performed. The enrichment can be a complete or partial enrichment of the cell, protein, or macromolecule. In an embodiment, the enrichment is at least 1%, e.g., at least 1-200%, e.g., at least 1-10, 10-20, 20-30, 30-50, 50-70, 70-90, 90-110, 110-130, 130-150, 150-170, or 170-200% increase of the level or amount of a cell, a protein, or macromolecule, as compared to the level or amount of the cell, protein or macromolecule in a reference sample. In some embodiments, the enrichment is at least 5%, e.g., at least 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 99% increase of the level or amount of a cell, a protein, or macromolecule, as compared to the level or amount of the cell, protein or macromolecule in a reference sample. In some embodiments, the enrichment is at least 1.1 fold, e.g., 1.1-200 fold, e.g., 1.1-10, 10-20, 20-30, 30-50, 50-70, 70-90, or 90-100 fold increase of the level or amount of a cell, a protein, or macromolecule, as compared to the level or amount of the cell, protein or macromolecule in a reference sample. In some embodiments, the reference sample can be a same sample, e.g., the sample before the process was performed. In some embodiments, the same sample refers to the sample on which the enrichment is subsequently performed, e.g., a pre-enrichment population, e.g., a starting population. In some embodiments, the reference sample can be a different sample, e.g., a sample on which the process is not performed.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acid (DNA) or ribonucleic acid (RNA), or a combination of a DNA or RNA thereof, and polymers thereof in either single- or double-stranded form. The term “nucleic acid” includes a gene, cDNA or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ (SEQ ID NO: 5023), where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄ Ser)₄ (SEQ ID NO:106) or (Gly₄ Ser)₃ (SEQ ID NO:28). In another embodiment, the linkers include multiple repeats of (Gly₂Ser), (GlySer) or (Gly₃Ser) (SEQ ID NO:29). Also included within the scope of the invention are linkers described in WO2012/138475, incorporated herein by reference).

As used herein, a 5′ cap (also termed an RNA cap, an RNA 7-methylguanosine cap or an RNA m⁷G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, e.g., mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In some embodiments of a construct for transient expression, the polyA is between 50 and 5000 (SEQ ID NO: 30), e.g., greater than 64, e.g., greater than 100, e.g., greater than 300 or 400 poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

Apheresis is the process in which whole blood is removed from an individual, separated into select components, and the remainder returned to circulation. Generally, there are two methods for the separation of blood components, centrifugal and non-centrifugal. Leukapheresis results in the active selection and removal of the patient's white blood cells.

As used herein, the terms “treat”, “treatment” and “treating” refer to the reduction or amelioration of the progression, severity and/or duration of a disease or disorder (e.g., a proliferative disorder), or the amelioration of one or more symptoms (preferably, one or more discernible symptoms) of a disease or disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of disease or disorder, such as growth of a tumor, not necessarily discernible by the patient. In other embodiments the terms “treat”, “treatment” and “treating”-refer to the inhibition of the progression of a disease or disorder (e.g., a proliferative disorder), either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

As used herein, unless otherwise specified, the terms “prevent,” “preventing” and “prevention” refer to an action that occurs before the subject begins to suffer from the condition, or relapse of the condition. Prevention need not result in a complete prevention of the condition; partial prevention or reduction of the condition or a symptom of the condition, or reduction of the risk of developing the condition, is encompassed by this term.

Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. In one embodiment, the CAR-expressing cell is administered at a dose and/or dosing schedule described herein, and the BCL2 inhibitor, or agent that enhances the activity of the CD19 CAR-expressing cell is administered at a dose and/or dosing schedule described herein.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human). In one embodiment, the subject is a patient.

A subject “responds” to treatment if a parameter of a cancer (e.g., a hematological cancer, e.g., cancer cell growth, proliferation and/or survival) in the subject is retarded or reduced by a detectable amount, e.g., about 5%, 10%, 20%, 30%, 40%, 50%, 60%, 70%, 80%, 90% or more as determined by any appropriate measure, e.g., by mass, cell count or volume. In one example, a subject responds to treatment if the subject experiences a life expectancy extended by about 5%, 10%, 20%, 30%, 40%, 50% or more beyond the life expectancy predicted if no treatment is administered. In another example, a subject responds to treatment, if the subject has an increased disease-free survival, overall survival or increased time to progression (e.g., progression free survival). Several methods can be used to determine if a patient responds to a treatment including, for example, criteria provided by NCCN Clinical Practice Guidelines in Oncology (NCCN Guidelines®). For example, in the context of DLBCL and FL, a complete response or complete responder may involve one or more of the criteria in Table 8 for complete metabolic response and complete radiological response. A partial responder may involve one or more of the criteria in Table 8 for partial metabolic response and partial radiological response. A non-responder can show disease progression, e.g., one or more of the critea in Table 8 for progressive metabolic disease or progressive disease.

The term “relapse” as used herein refers to reappearance of a cancer after an initial period of responsiveness (e.g., complete response or partial response). The initial period of responsiveness may involve the level of cancer cells falling below a certain threshold, e.g., below 20%, 1%, 10%, 5%, 4%, 3%, 2%, or 1%. The reappearance may involve the level of cancer cells rising above a certain threshold, e.g., above 20%, 1%, 10%, 5%, 4%, 3%, 2%, or 1%. For example, e.g., in the context of a B-cell lymphoma, e.g., a DLBCL or FL, the reappearance may comprise, e.g., a reappearance of a bone marrow involvement, e.g., a lesion, a reappearance of a malignant effusion, a reappearance of a nodal lesion greater than 1.5 cm as measured in any axis (e.g., a previously normal lymph node becoming greater than 1.5 cm in any axis) on CT scan or MRI after baseline, reappearance of a discrete extranodal lesion (including liver or spleen) on CT scan or MRI after baseline, or a ≥50% increase in the measurement of any residual lymph node or mass, e.g., the long axis from baseline. More generally, in an embodiment, a response (e.g., complete response or partial response) can involve the absence of detectable MRD (minimal residual disease). In an embodiment, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

In some embodiments, a therapy that includes a CD19 inhibitor, e.g., a CD19 CAR therapy, may relapse or be refractory to treatment. The relapse or resistance can be caused by CD19 loss (e.g., an antigen loss mutation) or other CD19 alteration that reduces the level of CD19 (e.g., caused by clonal selection of CD19-negative clones). A cancer that harbors such CD19 loss or alteration is referred to herein as a “CD19-negative cancer” or a “CD19-negative relapsed cancer”). It shall be understood that a CD19-negative cancer need not have 100% loss of CD19, but a sufficient reduction to reduce the effectiveness of a CD19 therapy such that the cancer relapses or becomes refractory. In some embodiments, a CD19-negative cancer results from a CD19 CAR therapy.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19.

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

In the context of the present invention, “tumor antigen” or “hyperproliferative disorder antigen” or “antigen associated with a hyperproliferative disorder” refers to antigens that are common to specific hyperproliferative disorders. In certain embodiments, the tumor antigen is derived from a cancer including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner protein present in a sample, but which antibody or ligand does not substantially recognize or bind other molecules in the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### CAR Therapies

The CAR-expressing cells described herein, may comprise one or more of the compositions described herein, e.g., a transmembrane domain, intracellular signaling domain, costimulatory domain, leader sequence, or hinge.

In one aspect, the present invention encompasses a recombinant nucleic acid construct comprising a transgene encoding a CAR. In some embodiments, the nucleic acid molecule comprises a nucleic acid sequence encoding an anti-CD19 binding domain selected from one or more of SEQ ID NOS:61-72, wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, e.g., CD3-zeta, CD28, 4-1BB, and the like. In some instances, the CAR can comprise any combination of CD3-zeta, CD28, 4-1BB, and the like.

In one aspect, the present invention contemplates modifications of the starting antibody or fragment (e.g., scFv) amino acid sequence that generate functionally equivalent molecules. For example, the VH or VL of an antigen binding domain, e.g., scFv, comprised in the CAR can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting VH or VL framework region of the antigen binding domain, e.g., scFv. The present invention contemplates modifications of the entire CAR construct, e.g., modifications in one or more amino acid sequences of the various domains of the CAR construct in order to generate functionally equivalent molecules. The CAR construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting CAR construct. The present invention also contemplates modifications of CDRs, e.g., modifications in one or more amino acid sequences of one or more CDRs of a CAR construct in order to generate functionally equivalent molecules. For instance, the CDR may have, e.g., up to and including 1, 2, 3, 4, 5, or 6 alterations (e.g., substitutions) relative to a CDR sequence provided herein.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the nucleic acid of interest can be produced synthetically, rather than cloned.

The present invention includes, among other things, retroviral and lentiviral vector constructs expressing a CAR that can be directly transduced into a cell.

The present invention also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length (SEQ ID NO:118). RNA so produced can efficiently transfect different kinds of cells. In one embodiment, the template includes sequences for the CAR. In an embodiment, an RNA CAR vector is transduced into a T cell by electroporation.

Antigen Binding Domain

In one aspect, the CAR of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus examples of cell surface markers that may act as ligands for the antigen binding domain in a CAR of the invention include those associated with viral, bacterial and parasitic infections, autoimmune disease and cancer cells. The antigen-binding domain can bind a B-cell antigen, e.g., CD19, CD22, CD20, CD123, BCMA, CD34, FLT-3, ROR1, CD179b, and/or CD79a antigen.

In one aspect, the CAR-mediated T-cell response can be directed to an antigen of interest by way of engineering an antigen binding domain that specifically binds a desired antigen into the CAR.

The antigen binding domain (e.g., an antigen-binding domain that binds a B-cell antigen, e.g., CD19, CD22, CD20, CD123, BCMA, CD34, FLT-3, ROR1, CD179b, and/or CD79a antigen) can be any domain that binds to the antigen including but not limited to a monoclonal antibody, a polyclonal antibody, a recombinant antibody, a murine antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, and the like.

In some instances, it is beneficial for the antigen binding domain to be derived from the same species in which the CAR will ultimately be used in. For example, for use in humans, it may be beneficial for the antigen binding domain (e.g., a B-cell antigen, e.g., CD19, CD22, CD20, CD123, BCMA, CD34, FLT-3, ROR1, CD179b, and/or CD79a antigen) of the CAR to comprise human or humanized residues for the antigen binding domain of an antibody or antibody fragment.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a VH4_4-59 germline sequence. In one embodiment, the framework region can comprise, one, two, three, four or five modifications, e.g., substitutions, e.g., from the amino acid at the corresponding murine sequence (e.g., of SEQ ID NO:59). In one embodiment, the framework region, e.g., all four framework regions of the light chain variable region are derived from a VK31.25 germline sequence. In one embodiment, the framework region can comprise, one, two, three, four or five modifications, e.g., substitutions, e.g., from the amino acid at the corresponding murine sequence (e.g., of SEQ ID NO:59).

In some aspects, the portion of a CAR composition of the invention that comprises an antibody fragment is humanized with retention of high affinity for the target antigen and other favorable biological properties. According to one aspect of the invention, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., the ability to bind human B-cell antigen, e.g., CD19, CD22, CD20, CD123, BCMA, CD34, FLT-3, ROR1, CD179b, and/or CD79a antigen. In some embodiments, a humanized antibody or antibody fragment may have improved affinity and/or specificity of binding to human B-cell antigen, e.g., CD19, CD22, CD20, CD123, BCMA, CD34, FLT-3, ROR1, CD179b, and/or CD79a antigen.

In one aspect, the binding domain (e.g., an antigen-binding domain that binds B-cell antigen, e.g., CD19, CD22, CD20, CD123, BCMA, CD34, FLT-3, ROR1, CD179b, and/or CD79a antigen) is a fragment, e.g., a single chain variable fragment (scFv). In one aspect, the binding domain is a Fv, a Fab, a (Fab′)2, or a bi-functional (e.g. bi-specific) hybrid antibody (e.g., Lanzavecchia et al., Eur. J. Immunol. 17, 105 (1987)). In one aspect, the antibodies and fragments thereof of the invention binds a B-cell antigen/protein, e.g., a CD19, CD22, CD20, CD123, BCMA, CD34, FLT-3, ROR1, CD179b, or CD79a protein with wild-type or enhanced affinity. In one aspect, the antibodies and fragments thereof of the invention binds a B-cell protein, e.g., CD19, CD22, CD20, CD123, BCMA, CD34, FLT-3, ROR1, CD179b, and/or CD79a, protein with wild-type or enhanced affinity.

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO:18). In one embodiment, the linker can be (Gly₄Ser)₄ (SEQ ID NO:106) or (Gly₄Ser)₃(SEQ ID NO:107). Variation in the linker length may retain or enhance activity, giving rise to superior efficacy in activity studies.

In some embodiments, the amino acid sequence of the antigen binding domain (e.g., an antigen-binding domain that binds B-cell antigen, e.g., CD19, CD22, CD20, CD123, BCMA, CD34, FLT-3, ROR1, CD179b, and/or CD79a antigen) or other portions or the entire CAR) can be modified, e.g., an amino acid sequence described herein can be modified, e.g., by a conservative substitution. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

In one aspect, the present invention contemplates modifications of the starting antibody or fragment (e.g., scFv) amino acid sequence that generate functionally equivalent molecules. For example, the VH or VL of a binding domain (e.g., an antigen-binding domain that binds B-cell antigen, e.g., CD19, CD22, CD20, CD123, BCMA, CD34, FLT-3, ROR1, CD179b, and/or CD79a antigen), e.g., scFv, comprised in the CAR can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting VH or VL framework region of the binding domain, e.g., scFv. In an aspect, the VH or VL of a CD19 antigen binding domain, e.g., scFv, comprised in the CAR can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting VH or VL framework region of the anti-CD19 antigen binding domain, e.g., scFv. The present invention contemplates modifications of the entire CAR construct, e.g., modifications in one or more amino acid sequences of the various domains of the CAR construct in order to generate functionally equivalent molecules. The CAR construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting CAR construct.

### CD19 CARs and Binding Domains

Provided herein are compositions and methods of use for the treatment of a disease such as cancer using CD19 chimeric antigen receptors (CAR). The methods include, inter alia, administering a CD19 CAR described herein in combination with another agent such as a BCL2 inhibitor, a BCL6 inhibitor, a MYC inhibitor, or a combination therof. In some embodiments, the CD19 CAR, e.g., a CD19 CAR as described herein is administered in combination with a BCL2 inhibitor, e.g., a BCL2 inhibitor as described herein. The methods also include, e.g., administering a CD19 CAR described herein to treat a lymphoma such as a B-cell lymphoma, e.g., high-grade B-cell lymphoma, DLBCL, or FL).

In one aspect, the invention provides a number of chimeric antigen receptors (CAR) comprising an antibody or antibody fragment engineered for specific binding to a CD19 protein. In one aspect, the invention provides a cell (e.g., T cell) engineered to express a CAR, wherein the CAR T cell (“CART”) exhibits an anticancer property. In one aspect a cell is transformed with the CAR and the CAR is expressed on the cell surface. In some embodiments, the cell (e.g., T cell) is transduced with a viral vector encoding a CAR. In some embodiments, the viral vector is a retroviral vector. In some embodiments, the viral vector is a lentiviral vector. In some such embodiments, the cell may stably express the CAR. In another embodiment, the cell (e.g., T cell) is transfected with a nucleic acid, e.g., mRNA, cDNA, DNA, encoding a CAR. In some such embodiments, the cell may transiently express the CAR.

In one aspect, the anti-CD19 protein binding portion of the CAR is a scFv antibody fragment. In one aspect such antibody fragments are functional in that they retain the equivalent binding affinity, e.g., they bind the same antigen with comparable affinity, as the IgG antibody from which it is derived. In one aspect such antibody fragments are functional in that they provide a biological response that can include, but is not limited to, activation of an immune response, inhibition of signal-transduction origination from its target antigen, inhibition of kinase activity, and the like, as will be understood by a skilled artisan. In one aspect, the anti-CD19 antigen binding domain of the CAR is a scFv antibody fragment that is humanized compared to the murine sequence of the scFv from which it is derived. In an embodiment, the humanized anti-CD19 binding domain comprises the amino acid sequence of SEQ ID NO:2, or an amino acid sequence at least 95%, 96%, 97%, 09%, or 99% identical thereto. In one aspect, the parental murine scFv sequence is the CAR19 construct provided in PCT publication WO2012/079000 and provided herein as SEQ ID NO:59. In one embodiment, the anti-CD19 binding domain is a scFv described in WO2012/079000 and provided in SEQ ID NO:59, or a sequence at least 95%, e.g., 95-99%, identical thereto. In an embodiment, the anti-CD19 binding domain is part of a CAR construct provided in PCT publication WO2012/079000 and provided herein as SEQ ID NO:58, or a sequence at least 95%, e.g., 95%-99%, identical thereto. In an embodiment, the anti-CD19 binding domain comprises at least one (e.g., 2, 3, 4, 5, or 6) CDRs selected from Table 4 and/or Table 5.

In some aspects, the antibodies of the invention are incorporated into a chimeric antigen receptor (CAR). In one aspect, the CAR comprises the polypeptide sequence provided as SEQ ID NO: 12 in PCT publication WO2012/079000, and provided herein as SEQ ID NO: 58, wherein the scFv domain is substituted by one or more sequences selected from SEQ ID NOS: 1-12. In one aspect, the scFv domains of SEQ ID NOS:1-12 are humanized variants of the scFv domain of SEQ ID NO:59, which is an scFv fragment of murine origin that specifically binds to human CD19. Humanization of this mouse scFv may be desired for the clinical setting, where the mouse-specific residues may induce a human-anti-mouse antigen (HAMA) response in patients who receive CART19 treatment, e.g., treatment with T cells transduced with the CAR19 construct.

In one aspect, the anti-CD19 binding domain, e.g., humanized scFv, portion of a CAR of the invention is encoded by a transgene whose sequence has been codon optimized for expression in a mammalian cell. In one aspect, entire CAR construct of the invention is encoded by a transgene whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

In one aspect, the humanized CAR19 comprises the scFv portion provided in SEQ ID NO:1. In one aspect, the humanized CAR19 comprises the scFv portion provided in SEQ ID NO:2. In one aspect, the humanized CAR19 comprises the scFv portion provided in SEQ ID NO:3. In one aspect, the humanized CAR19 comprises the scFv portion provided in SEQ ID NO:4. In one aspect, the humanized CAR19 comprises the scFv portion provided in SEQ ID NO:5. In one aspect, the humanized CAR19 comprises the scFv portion provided in SEQ ID NO:6. In one aspect, the humanized CAR19 comprises the scFv portion provided in SEQ ID NO:7. In one aspect, the humanized CAR19 comprises the scFv portion provided in SEQ ID NO:8. In one aspect, the humanized CAR19 comprises the scFv portion provided in SEQ ID NO:9. In one aspect, the humanized CAR19 comprises the scFv portion provided in SEQ ID NO:10. In one aspect, the humanized CAR19 comprises the scFv portion provided in SEQ ID NO:11. In one aspect, the humanized CAR19 comprises the scFv portion provided in SEQ ID NO:12.

In one aspect the CAR19 comprises the scFv portion provided in SEQ ID NO: 5002. In one aspect the CAR19 comprises the scFv portion provided in SEQ ID NO: 5005. In one aspect the CAR19 comprises the scFv portion provided in SEQ ID NO: 5013. In one aspect the CAR19 comprises the scFv portion provided in SEQ ID NO: 5018.

In one aspect, the CARs of the invention combine an antigen binding domain of a specific antibody with an intracellular signaling molecule. For example, in some aspects, the intracellular signaling molecule includes, but is not limited to, CD3-zeta chain, 4-1BB and CD28 signaling modules and combinations thereof. In one aspect, the CD19 CAR comprises a CAR selected from the sequence provided in one or more of SEQ ID NOS: 31-42, 5001, 5004, 5008, 5011, or 5016. In one aspect, the CD19 CAR comprises the sequence provided in SEQ ID NO:31. In one aspect, the CD19 CAR comprises the sequence provided in SEQ ID NO:32. In one aspect, the CD19 CAR comprises the sequence provided in SEQ ID NO:33. In one aspect, the CD19 CAR comprises the sequence provided in SEQ ID NO:34. In one aspect, the CD19 CAR comprises the sequence provided in SEQ ID NO:35. In one aspect, the CD19 CAR comprises the sequence provided in SEQ ID NO:36. In one aspect, the CD19 CAR comprises the sequence provided in SEQ ID NO:37. In one aspect, the CD19 CAR comprises the sequence provided in SEQ ID NO:38. In one aspect, the CD19 CAR comprises the sequence provided in SEQ ID NO:39. In one aspect, the CD19 CAR comprises the sequence provided in SEQ ID NO:40. In one aspect, the CD19 CAR comprises the sequence provided in SEQ ID NO:41. In one aspect, the CD19 CAR comprises the sequence provided in SEQ ID NO:42. In one aspect, the CD19 CAR comprises the sequence provided in SEQ ID NO: 5001. In one aspect, the CD19 CAR comprises the sequence provided in SEQ ID NO: 5004. In one aspect, the CD19 CAR comprises the sequence provided in SEQ ID NO: 5008. In one aspect, the CD19 CAR comprises the sequence provided in SEQ ID NO: 5011. In one aspect, the CD19 CAR comprises the sequence provided in SEQ ID NO: 5016.

In one aspect, the CD19 CAR comprises a CAR selected from the sequence provided in one or more of SEQ ID NOS: 31-42, wherein the CAR does not comprise a leader sequence comprising the amino acid sequence of SEQ ID NO: 13.

In one aspect, the CD19 CAR comprises a CAR selected from the sequence provided in one or more of SEQ ID NOS: 5001 or SEQ ID NO: 5004, wherein the CAR does not comprise a leader sequence comprising the amino acid sequence of SEQ ID NO: 5020.

Thus, in one aspect, the antigen binding domain comprises a humanized antibody or an antibody fragment. In one embodiment, the humanized anti-CD19 binding domain comprises one or more (e.g., all three) light chain complementarity determining region 1 (LC CDR1), light chain complementarity determining region 2 (LC CDR2), and light chain complementarity determining region 3 (LC CDR3) of a murine or humanized anti-CD19 binding domain described herein, and/or one or more (e.g., all three) heavy chain complementarity determining region 1 (HC CDR1), heavy chain complementarity determining region 2 (HC CDR2), and heavy chain complementarity determining region 3 (HC CDR3) of a murine or humanized anti-CD19 binding domain described herein, e.g., a humanized anti-CD19 binding domain comprising one or more, e.g., all three, LC CDRs and one or more, e.g., all three, HC CDRs. In one embodiment, the humanized anti-CD19 binding domain comprises one or more (e.g., all three) heavy chain complementarity determining region 1 (HC CDR1), heavy chain complementarity determining region 2 (HC CDR2), and heavy chain complementarity determining region 3 (HC CDR3) of a murine or humanized anti-CD19 binding domain described herein, e.g., the humanized anti-CD19 binding domain has two variable heavy chain regions, each comprising a HC CDR1, a HC CDR2 and a HC CDR3 described herein. In one embodiment, the humanized anti-CD19 binding domain comprises a humanized light chain variable region described herein (e.g., in Table 2) and/or a humanized heavy chain variable region described herein (e.g., in Table 2). In one embodiment, the humanized anti-CD19 binding domain comprises a humanized heavy chain variable region described herein (e.g., in Table 2), e.g., at least two humanized heavy chain variable regions described herein (e.g., in Table 2). In one embodiment, the anti-CD19 binding domain is a scFv comprising a light chain and a heavy chain of an amino acid sequence of Table 2. In an embodiment, the anti-CD19 binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 2, or a sequence with 95-99% identity with an amino acid sequence of Table 2; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 2, or a sequence with 95-99% identity to an amino acid sequence of Table 2. In one embodiment, the humanized anti-CD19 binding domain comprises a sequence selected from a group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, or a sequence with 95-99% identity thereof. In one embodiment, the nucleic acid sequence encoding the humanized anti-CD19 binding domain comprises a sequence selected from a group consisting of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:70, SEQ ID NO:71 and SEQ ID NO:72, or a sequence with 95-99% identity thereof. In one embodiment, the humanized anti-CD19 binding domain is a scFv, and a light chain variable region comprising an amino acid sequence described herein, e.g., in Table 2, is attached to a heavy chain variable region comprising an amino acid sequence described herein, e.g., in Table 2, via a linker, e.g., a linker described herein. In one embodiment, the humanized anti-CD19 binding domain includes a (Gly₄-Ser)n linker, wherein n is 1, 2, 3, 4, 5, or 6, e.g., 3 or 4 (SEQ ID NO:53). The light chain variable region and heavy chain variable region of a scFv can be, e.g., in any of the following orientations: light chain variable region-linker-heavy chain variable region or heavy chain variable region-linker-light chain variable region.

In one aspect, the antigen binding domain portion comprises one or more sequence selected from SEQ ID NOS:1-12. In one aspect the humanized CAR is selected from one or more sequence selected from SEQ ID NOS: 31-42. In some aspects, a non-human antibody is humanized, where specific sequences or regions of the antibody are modified to increase similarity to an antibody naturally produced in a human or fragment thereof.

In one embodiment, the CAR molecule comprises an anti-CD19 binding domain comprising one or more (e.g., all three) light chain complementarity determining region 1 (LC CDR1), light chain complementarity determining region 2 (LC CDR2), and light chain complementarity determining region 3 (LC CDR3) of an anti-CD19 binding domain described herein, and one or more (e.g., all three) heavy chain complementarity determining region 1 (HC CDR1), heavy chain complementarity determining region 2 (HC CDR2), and heavy chain complementarity determining region 3 (HC CDR3) of an anti-CD19 binding domain described herein, e.g., an anti-CD19 binding domain comprising one or more, e.g., all three, LC CDRs and one or more, e.g., all three, HC CDRs. In one embodiment, the anti-CD19 binding domain comprises one or more (e.g., all three) heavy chain complementarity determining region 1 (HC CDR1), heavy chain complementarity determining region 2 (HC CDR2), and heavy chain complementarity determining region 3 (HC CDR3) of an anti-CD19 binding domain described herein, e.g., the anti-CD19 binding domain has two variable heavy chain regions, each comprising a HC CDR1, a HC CDR2 and a HC CDR3 described herein.

In one aspect, the anti-CD19 binding domain is characterized by particular functional features or properties of an antibody or antibody fragment. For example, in one aspect, the portion of a CAR composition of the invention that comprises an antigen binding domain specifically binds human CD19. In one aspect, the invention relates to an antigen binding domain comprising an antibody or antibody fragment, wherein the antibody binding domain specifically binds to a CD19 protein or fragment thereof, wherein the antibody or antibody fragment comprises a variable light chain and/or a variable heavy chain that includes an amino acid sequence of SEQ ID NO: 1-12 or SEQ ID NO:59. In one aspect, the antigen binding domain comprises an amino acid sequence of an scFv selected from SEQ ID NOs: 1-12 or SEQ ID NO:59. In certain aspects, the scFv is contiguous with and in the same reading frame as a leader sequence. In one aspect the leader sequence is the polypeptide sequence provided as SEQ ID NO:13. In some aspects, the scFv does not comprises a leader sequence, e.g., a leader sequence comprising the amino acid sequence of SEQ ID NO: 13. In some aspects, the scFv does not comprises a leader sequence, e.g., a leader sequence comprising the amino acid sequence of SEQ ID NO: 5020.

In one aspect, the portion of the CAR comprising the antigen binding domain comprises an antigen binding domain that targets CD19. In one aspect, the antigen binding domain targets human CD19. In one aspect, the antigen binding domain of the CAR has the same or a similar binding specificity as, or includes, the FMC63 scFv fragment described in Nicholson et al. Mol. Immun. 34 (16-17): 1157-1165 (1997). In one aspect, the portion of the CAR comprising the antigen binding domain comprises an antigen binding domain that targets a B-cell antigen, e.g., a human B-cell antigen. A CD19 antibody molecule can be, e.g., an antibody molecule (e.g., a humanized anti-CD19 antibody molecule) described in WO2014/153270, which is incorporated herein by reference in its entirety. WO2014/153270 also describes methods of assaying the binding and efficacy of various CART constructs.

In one embodiment, the anti-CD19 binding domain comprises a murine light chain variable region described herein (e.g., in Table 3) and/or a murine heavy chain variable region described herein (e.g., in Table 3). In one embodiment, the anti-CD19 binding domain is a scFv comprising a murine light chain and a murine heavy chain of an amino acid sequence of Table 3. In an embodiment, the anti-CD19 binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 3, or a sequence with 95-99% identity with an amino acid sequence of Table 3; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 3, or a sequence with 95-99% identity to an amino acid sequence of Table 3. In one embodiment, the anti-CD19 binding domain comprises a sequence of SEQ ID NO:59, or a sequence with 95-99% identity thereof. In one embodiment, the anti-CD19 binding domain is a scFv, and a light chain variable region comprising an amino acid sequence described herein, e.g., in Table 3, is attached to a heavy chain variable region comprising an amino acid sequence described herein, e.g., in Table 3, via a linker, e.g., a linker described herein. In one embodiment, the antigen binding domain includes a (Gly₄-Ser)n linker, wherein n is 1, 2, 3, 4, 5, or 6, e.g., 3 or 4 (SEQ ID NO: 53). The light chain variable region and heavy chain variable region of a scFv can be, e.g., in any of the following orientations: light chain variable region-linker-heavy chain variable region or heavy chain variable region-linker-light chain variable region.

Furthermore, the present invention provides (among other things) CD19 CAR compositions, optionally in combination with a BCL2 inhibitor, a BCL6 inhibitor, a MYC inhibitor, or a combination thereof, and their use in medicaments or methods for treating, among other diseases, cancer or any malignancy involving cells or tissues which express CD19.

In one aspect, the CAR of the invention can be used to eradicate CD19-expressing normal cells, thereby applicable for use as a cellular conditioning therapy prior to cell transplantation. In one aspect, the CD19-expressing normal cell is a CD19-expressing normal stem cell and the cell transplantation is a stem cell transplantation.

In one aspect, the invention provides a cell (e.g., T cell) engineered to express a chimeric antigen receptor (CAR), wherein the CAR-expressing cell, e.g., CAR T cell (“CART”) exhibits an anticancer property. A suitable antigen is CD19. In one aspect, the antigen binding domain of the CAR comprises a partially humanized anti-CD19 antibody fragment. In one aspect, the antigen binding domain of the CAR comprises a partially humanized anti-CD19 antibody fragment comprising an scFv. Accordingly, the invention provides (among other things) a CD19-CAR that comprises a humanized anti-CD19 binding domain and is engineered into an immune effector cell, e.g., a T cell or an NK cell, and methods of their use for adoptive therapy.

In one aspect, the CAR, e.g., CD19-CAR comprises at least one intracellular domain selected from the group of a CD137 (4-1BB) signaling domain, a CD28 signaling domain, a CD3zeta signal domain, and any combination thereof. In one aspect, the CAR, e.g., CD19-CAR comprises at least one intracellular signaling domain is from one or more co-stimulatory molecule(s) other than a CD137 (4-1BB) or CD28.

The present invention encompasses, but is not limited to, a recombinant DNA construct comprising sequences encoding a CAR, wherein the CAR comprises an antibody or antibody fragment that binds specifically to CD19, wherein the sequence of the antibody fragment is contiguous with and in the same reading frame as a nucleic acid sequence encoding an intracellular signaling domain. The intracellular signaling domain can comprise a costimulatory signaling domain and/or a primary signaling domain, e.g., a zeta chain. The costimulatory signaling domain refers to a portion of the CAR comprising at least a portion of the intracellular domain of a costimulatory molecule. In one embodiment, the antigen binding domain is a murine antibody or antibody fragment described herein. In one embodiment, the antigen binding domain is a humanized antibody or antibody fragment.

In specific aspects, a CAR construct of the invention comprises a scFv domain selected from the group consisting of SEQ ID NOS:1-12 or an scFV domain of SEQ ID NO:59, wherein the scFv may be preceded by an optional leader sequence such as provided in SEQ ID NO: 13, and followed by an optional hinge sequence such as provided in SEQ ID NO:14 or SEQ ID NO:45 or SEQ ID NO:47 or SEQ ID NO:49, a transmembrane region such as provided in SEQ ID NO:15, an intracellular signalling domain that includes SEQ ID NO:16 or SEQ ID NO:51 and a CD3 zeta sequence that includes SEQ ID NO:17 or SEQ ID NO:43, wherein the domains are contiguous with and in the same reading frame to form a single fusion protein.

Also included in the invention (among other things) is a nucleotide sequence that encodes the polypeptide of each of the scFv fragments selected from the group consisting of SEQ IS NO:1, SEQ ID NO:2, SEQ ID NO:3, SEQ ID NO:4, SEQ ID NO:5, SEQ IS NO:6, SEQ ID NO:7, SEQ ID NO:8, SEQ ID NO:9, SEQ ID NO:10, SEQ ID NO:11, SEQ ID NO:12 and SEQ ID NO:59. Also included in the invention (among other things) is a nucleotide sequence that encodes the polypeptide of each of the scFv fragments selected from the group consisting of SEQ IS NO:1, SEQ ID NO:2, SEQ ID NO:3, SEQ ID NO:4, SEQ ID NO:5, SEQ IS NO:6, SEQ ID NO:7, SEQ ID NO:8, SEQ ID NO:9, SEQ ID NO:10, SEQ ID NO:11, SEQ ID NO:12 and SEQ ID NO:59, and each of the domains of SEQ ID NOS: 13-17, plus an encoded CD19 CAR fusion protein of the invention.

In one aspect an exemplary CD19 CAR constructs comprise an optional leader sequence, an extracellular antigen binding domain, a hinge, a transmembrane domain, and an intracellular stimulatory domain.

In one aspect an exemplary CD19 CAR construct comprises an optional leader sequence, an extracellular antigen binding domain, a hinge, a transmembrane domain, an intracellular costimulatory domain and an intracellular stimulatory domain. In some embodiments, specific CD19 CAR constructs containing humanized scFv domains of the invention are provided as SEQ ID NOS: 31-42, or a murine scFv domain as provided as SEQ ID NO:59.

Full-length CAR sequences are also provided herein as SEQ ID NOS: 31-42 and 58, as shown in Table 2 and Table 3.

An exemplary leader sequence is provided as SEQ ID NO: 13. An exemplary hinge/spacer sequence is provided as SEQ ID NO: 14 or SEQ ID NO:45 or SEQ ID NO:47 or SEQ ID NO:49. An exemplary transmembrane domain sequence is provided as SEQ ID NO:15. An exemplary sequence of the intracellular signaling domain of the 4-1BB protein is provided as SEQ ID NO: 16. An exemplary sequence of the intracellular signaling domain of CD27 is provided as SEQ ID NO:51. An exemplary CD3zeta domain sequence is provided as SEQ ID NO: 17 or SEQ ID NO:43. These sequences may be used, e.g., in combination with an scFv that recognizes CD19.

Exemplary sequences of various scFv fragments and other CAR components are provided herein. It is noted that these CAR components (e.g., of SEQ ID NO: 121, or a sequence of Table 2 or 3) without a leader sequence (e.g., without the amino acid sequence of SEQ ID NO: 13 or a nucleotide sequence of SEQ ID NO: 54), are also provided herein.

In embodiments, the CAR sequences described herein contain a Q/K residue change in the signal domain of the co-stimulatory domain derived from CD3zeta chain.

In one aspect, the present invention encompasses a recombinant nucleic acid construct comprising a nucleic acid molecule encoding a CAR, wherein the nucleic acid molecule comprises the nucleic acid sequence encoding an anti-CD19 binding domain, e.g., described herein, that is contiguous with and in the same reading frame as a nucleic acid sequence encoding an intracellular signaling domain. In one aspect, the anti-CD19 binding domain is selected from one or more of SEQ ID NOS:1-12 and 58. In one aspect, the anti-CD19 binding domain is encoded by a nucleotide residues 64 to 813 of the sequence provided in one or more of SEQ ID NOS:61-72 and 97. In one aspect, the anti-CD19 binding domain is encoded by a nucleotide residues 64 to 813 of SEQ ID NO:61. In one aspect, the anti-CD19 binding domain is encoded by a nucleotide residues 64 to 813 of SEQ ID NO:62. In one aspect, the anti-CD19 binding domain is encoded by a nucleotide residues 64 to 813 of SEQ ID NO:63. In one aspect, the anti-CD19 binding domain is encoded by a nucleotide residues 64 to 813 of SEQ ID NO:64. In one aspect, the anti-CD19 binding domain is encoded by a nucleotide residues 64 to 813 of SEQ ID NO:65. In one aspect, the anti-CD19 binding domain is encoded by a nucleotide residues 64 to 813 of SEQ ID NO:66. In one aspect, the anti-CD19 binding domain is encoded by a nucleotide residues 64 to 813 of SEQ ID NO:67. In one aspect, the anti-CD19 binding domain is encoded by a nucleotide residues 64 to 813 of SEQ ID NO:68. In one aspect, the anti-CD19 binding domain is encoded by a nucleotide residues 64 to 813 of SEQ ID NO:69. In one aspect, the anti-CD19 binding domain is encoded by a nucleotide residues 64 to 813 of SEQ ID NO:70. In one aspect, the anti-CD19 binding domain is encoded by a nucleotide residues 64 to 813 of SEQ ID NO:71. In one aspect, the anti-CD19 binding domain is encoded by a nucleotide residues 64 to 813 of SEQ ID NO:72.

Provided herein are CD19 inhibitors and combination therapies. In some embodiments, the CD19 inhibitor (e.g., a cell therapy or an antibody) is administered in combination with another B cell inhibitor, e.g., one or more inhibitors of CD19, CD20, CD22, CD34, CD123, BCMA, CD179b, CD79b, CD79a, FLT-3, or ROR1. A CD19 inhibitor includes but is not limited to a CD19 CAR-expressing cell, e.g., a CD19 CART cell, or an anti-CD19 antibody (e.g., an anti-CD19 mono- or bispecific antibody) or a fragment or conjugate thereof. In an embodiment, the CD19 inhibitor is administered in combination with a B-cell inhibitor, e.g., a CAR-expressing cell described herein.

In some embodiments, the CD19 inhibitor, e.g., a CD19 CAR-expressing cell described herein, is administered in combination with a BCL2 inhibitor, a BCL6 inhibitor, a MYC inhibitor or a combination thereof. In some embodiments, the CD19 inhibitor, e.g., a CD19 CAR-expressing cell described herein, is administered in combination with a BCL2 inhibitor, e.g., a BCL2 inhibitor described herein. In some embodiments, the BCL2 inhibitor is venetoclax. In some embodiments, the CD19 CAR-expressing cell is administered in combination with venetoclax.

Numerous CD19 CAR-expressing cells are described in this disclosure. For instance, in some embodiments, a CD19 inhibitor includes an anti-CD19 CAR-expressing cell, e.g., CART, e.g., a cell expressing an anti-CD19 CAR construct described in Table 2 or encoded by a CD19 binding CAR comprising a scFv, CDRs, or VH and VL chains described in Tables 2, 4, or 5. For example, an anti-CD19 CAR-expressing cell, e.g., CART, is a generated by engineering a CD19-CAR (that comprises a CD19 binding domain) into a cell (e.g., a T cell or NK cell), e.g., for administration in combination with a CAR-expressing cell described herein. Also provided herein are methods of use of the CAR-expressing cells described herein for adoptive therapy.

In one embodiment, an antigen binding domain comprises one, two three (e.g., all three) heavy chain CDRs, HC CDR1, HC CDR2 and HC CDR3, from an antibody listed herein, e.g., in Table 2, 4, or 5 and/or one, two, three (e.g., all three) light chain CDRs, LC CDR1, LC CDR2 and LC CDR3, from an antibody listed herein, e.g., in Table 2, 4, or 5. In one embodiment, the antigen binding domain comprises a heavy chain variable region and/or a variable light chain region of an antibody listed or described above.

In an embodiment, the CD19 binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 2, or a sequence with 95-99% identity with an amino acid sequence of Table 2; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 2, or a sequence with 95-99% identity to an amino acid sequence of Table 2. In embodiments, the CD19 binding domain comprises one or more CDRs (e.g., one each of a HC CDR1, HC CDR2, HC CDR3, LC CDR1, LC CDR2, and LC CDR3) of Table 4 or Table 5, or CDRs having one, two, three, four, five, or six modifications (e.g., substitutions) of one or more of the CDRs.

Exemplary anti-CD19 antibodies or fragments or conjugates thereof include but are not limited to blinatumomab, SAR3419 (Sanofi), MEDI-551 (MedImmune LLC), Combotox, DT2219ARL (Masonic Cancer Center), MOR-208 (also called XmAb-5574; MorphoSys), XmAb-5871 (Xencor), MDX-1342 (Bristol-Myers Squibb), SGN-CD19A (Seattle Genetics), and AFM11 (Affimed Therapeutics). See, e.g., Hammer. MAbs. 4.5(2012): 571-77. Blinatomomab is a bispecific antibody comprised of two scFvs-one that binds to CD19 and one that binds to CD3. Blinatomomab directs T cells to attack cancer cells. See, e.g., Hammer et al.; Clinical Trial Identifier No. NCT00274742 and NCT01209286. MEDI-551 is a humanized anti-CD19 antibody with a Fe engineered to have enhanced antibody-dependent cell-mediated cytotoxicity (ADCC). See, e.g., Hammer et al.; and Clinical Trial Identifier No. NCT01957579. Combotox is a mixture of immunotoxins that bind to CD19 and CD22. The immunotoxins are made up of scFv antibody fragments fused to a deglycosylated ricin A chain. See, e.g., Hammer et al.; and Herrera et al. J. Pediatr. Hematol. Oncol. 31.12(2009):936-41; Schindler et al. Br. J. Haematol. 154.4(2011):471-6. DT2219ARL is a bispecific immunotoxin targeting CD19 and CD22, comprising two scFvs and a truncated diphtheria toxin. See, e.g., Hammer et al.; and Clinical Trial Identifier No. NCT00889408. SGN-CD19A is an antibody-drug conjugate (ADC) comprised of an anti-CD19 humanized monoclonal antibody linked to a synthetic cytotoxic cell-killing agent, monomethyl auristatin F (MMAF). See, e.g., Hammer et al.; and Clinical Trial Identifier Nos. NCT01786096 and NCT01786135. SAR3419 is an anti-CD19 antibody-drug conjugate (ADC) comprising an anti-CD19 humanized monoclonal antibody conjugated to a maytansine derivative via a cleavable linker. See, e.g., Younes et al. J. Clin. Oncol. 30.2(2012): 2776-82; Hammer et al.: Clinical Trial Identifier No. NCT00549185; and Blanc et al. Clin Cancer Res. 2011; 17:6448-58. XmAb-5871 is an Fc-engineered, humanized anti-CD19 antibody. See, e.g., Hammer et al. MDX-1342 is a human Fc-engineered anti-CD19 antibody with enhanced ADCC. See, e.g., Hammer et al. In embodiments, the antibody molecule is a bispecific anti-CD19 and anti-CD3 molecule. For instance, AFM11 is a bispecific antibody that targets CD19 and CD3. See, e.g., Hammer et al.; and Clinical Trial Identifier No. NCT02106091. In some embodiments, an anti-CD19 antibody described herein is conjugated or otherwise bound to a therapeutic agent, e.g., a chemotherapeutic agent, peptide vaccine (such as that described in Izumoto et al. 2008 J Neurosurg 108:963-971), immunosuppressive agent, or immunoablative agent, e.g., cyclosporin, azathioprine, methotrexate, mycophenolate, FK506, CAMPATH, anti-CD3 antibody, cytoxin, fludarabine, rapamycin, mycophenolic acid, steroid, FR901228, or cytokine.

Exemplary anti-CD19 antibody molecules (including antibodies or fragments or conjugates thereof) can include a scFv, CDRs, or VH and VL chains described in Tables 2, 4, or 5. In an embodiment, the CD19-binding antibody molecule comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 2, or a sequence with 95-99% identity with an amino acid sequence of Table 2; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 2, or a sequence with 95-99% identity to an amino acid sequence of Table 2. In embodiments, the CD19-binding antibody molecule comprises one or more CDRs (e.g., one each of a HC CDR1, HC CDR2, HC CDR3, LC CDR1, LC CDR2, and LC CDR3) of Table 4 or Table 5, or CDRs having one, two, three, four, five, or six modifications (e.g., substitutions) of one or more of the CDRs. The antibody molecule may be, e.g., an isolated antibody molecule.

In one embodiment, an antigen binding domain against CD19 is an antigen binding portion, e.g., CDRs, of an antigen binding domain described in a Table herein. In one embodiment, a CD19 antigen binding domain can be from any CD19 CAR, e.g., LG-740; U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10, each of which is herein incorporated by reference in its entirety.

In one embodiment, the CAR T cell that specifically binds to CD19 has the INN designation Tisagenlecleucel. CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In one embodiment, the CAR T cell that specifically binds to CD19 has the INN designation Axicabtagene ciloleucel. In one embodiment, the CAR T cell that specifically binds to CD19 has the USAN designation brexucabtagene autoleucel. In some embodiments, Axicabtagene ciloleucel is also known as YESCARTA®, Axi-cel, or KTE-C19. In some embodiments, brexucabtagene autoleucel is also known as KTE-X19 or TECARTUS®.

In one embodiment, the CAR T cell that specifically binds to CD19 has the INN designation Lisocabtagene maraleucel. In some embodiments, Lisocabtagene maraleucel is also known as JCAR017.

In one aspect the nucleic acid sequence of a CAR construct of the invention is selected from one or more of SEQ ID NOS:85-96, 5000, 5003, 5007, 5010, or 5015. In one aspect the nucleic acid sequence of a CAR construct is SEQ ID NO:85. In one aspect the nucleic acid sequence of a CAR construct is SEQ ID NO:86. In one aspect, the nucleic acid sequence of a CAR construct is SEQ ID NO: 5007. In one aspect the nucleic acid sequence of a CAR construct is SEQ ID NO:87. In one aspect the nucleic acid sequence of a CAR construct is SEQ ID NO:88. In one aspect the nucleic acid sequence of a CAR construct is SEQ ID NO:89. In one aspect the nucleic acid sequence of a CAR construct is SEQ ID NO:90. In one aspect the nucleic acid sequence of a CAR construct is SEQ ID NO:91. In one aspect the nucleic acid sequence of a CAR construct is SEQ ID NO:92. In one aspect the nucleic acid sequence of a CAR construct is SEQ ID NO:93. In one aspect the nucleic acid sequence of a CAR construct is SEQ ID NO:94. In one aspect the nucleic acid sequence of a CAR construct is SEQ ID NO:95. In one aspect the nucleic acid sequence of a CAR construct is SEQ ID NO:96. In one aspect, the nucleic acid sequence of a CAR construct is SEQ ID NO: 5000. In one aspect, the nucleic acid sequence of a CAR construct is SEQ ID NO: 5010. In one aspect, the nucleic acid sequence of a CAR construct is SEQ ID NO: 5003. In one aspect, the nucleic acid sequence of a CAR construct is SEQ ID NO: 5015. In one aspect the nucleic acid sequence of a CAR construct is SEQ ID NO:97. In one aspect the nucleic acid sequence of a CAR construct is SEQ ID NO:98. In one aspect the nucleic acid sequence of a CAR construct is SEQ ID NO:99.

### Humanization of Murine Anti-CD19 Antibody

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159), for instance Tables 3, 4, and 5 (p. 125-147).

CAR constructs, e.g., CD19 CAR Constructs

Of the CD19 CAR constructs described in International Application WO2014/153270, certain sequences are reproduced herein. It is understood that the sequences in this section can also be used in the context of other CARs, e.g., CARs binding a B-cell antigen.

The sequences of the humanized scFv fragments (SEQ ID NOS: 1-12) are provided below in Table 2. Additional scFv fragments (SEQ ID NOs: 5002, 5005, 5013, or 5018) are provided below in Table 2. Full CAR constructs were generated using SEQ ID NOs: 1-12, 5002, 5005, 5013, or 5018, with additional sequences, SEQ ID NOs: 13-17, and/or 5020 shown below, to generate full CAR constructs with SEQ ID NOs: 31-42, 5001, 5004, 5008, 5011, or 5016.

leader (amino acid sequence)
(SEQ ID NO: 13)
MALPVTALLLPLALLLHAARP
leader (nucleic acid sequence)
(SEQ ID NO: 54)
ATGGCCCTGCCTGTGACAGCCCTGCTGCTGCCTCTGGCTC
TGCTGCTGCATGCCGCTAGACCC
leader (nucleic acid sequence)
(SEQ ID NO: 5019)
ATGGCCCTCCCTGTCACCGCCCTGCTGCTTCCGCTGGCTC
TTCTGCTCCACGCCGCTCGGCCC
leader (amino acid sequence)
(SEQ ID NO: 5020)
MLLLVTSLLLCELPHPAFLLIP
leader (nucleic acid sequence)
(SEQ ID NO: 5021)
ATGCTTCTCCTGGTGACAAGCCTTCTGCTCTGTGAGTTAC
CACACCCAGCATTCCTCCTGATCCCA
leader (nucleic acid sequence)
(SEQ ID NO: 5022)
ATGCTGCTGCTGGTGACCAGCCTGCTGCTGTGCGAGCTGC
CCCACCCCGCCTTTCTGCTGATCCCC
CD8 hinge (amino acid sequence)
(SEQ ID NO: 14)
TTTPAPRPPTPAPTIASQPLSLRPEACRPAAGGAVHTRGL
DFACD
CD8 hinge (nucleic acid sequence)
(SEQ ID NO: 55)
ACCACGACGCCAGCGCCGCGACCACCAACACCGGCGCCCA
CCATCGCGTCGCAGCCCCTGTCCCTGCGCCCAGAGGCGTG
CCGGCCAGCGGCGGGGGGCGCAGTGCACACGAGGGGGCTG
GACTTCGCCTGTGAT
CD8 transmembrane (amino acid sequence)
(SEQ ID NO: 15)
IYIWAPLAGTCGVLLLSLVITLYC
transmembrane (nucleic acid sequence)
(SEQ ID NO: 56)
ATCTACATCTGGGCGCCCTTGGCCGGGACTTGTGGGGTCC
TTCTCCTGTCACTGGTTATCACCCTTTACTGC
4-1BB Intracellular domain
(amino acid sequence)
(SEQ ID NO: 16)
KRGRKKLLYIFKQPFMRPVQTTQEEDGCSCRFPEEEEGGC
EL
4-1BB Intracellular domain
(nucleic acid sequence)
(SEQ ID NO: 60)
AAACGGGGCAGAAAGAAACTCCTGTATATATTCAAACAAC
CATTTATGAGACCAGTACAAACTACTCAAGAGGAAGATGG
CTGTAGCTGCCGATTTCCAGAAGAAGAAGAAGGAGGATGT
GAACTG
CD3 zeta domain (amino acid sequence)
(SEQ ID NO: 17)
RVKFSRSADAPAYKQGQNQLYNELNLGRREEYDVLDKRRG
RDPEMGGKPRRKNPQEGLYNELQKDKMAEAYSEIGMKGER
RRGKGHDGLYQGLSTATKDTYDALHMQALPPR
CD3 zeta (nucleic acid sequence)
(SEQ ID NO: 101)
AGAGTGAAGTTCAGCAGGAGCGCAGACGCCCCCGCGTACA
AGCAGGGCCAGAACCAGCTCTATAACGAGCTCAATCTAGG
ACGAAGAGAGGAGTACGATGTTTTGGACAAGAGACGTGGC
CGGGACCCTGAGATGGGGGGAAAGCCGAGAAGGAAGAACC
CTCAGGAAGGCCTGTACAATGAACTGCAGAAAGATAAGAT
GGCGGAGGCCTACAGTGAGATTGGGATGAAAGGCGAGCGC
CGGAGGGGCAAGGGGCACGATGGCCTTTACCAGGGTCTCA
GTACAGCCACCAAGGACACCTACGACGCCCTTCACATGCA
GGCCCTGCCCCCTCGC
CD3 zeta domain (amino acid sequence;
NCBI Reference Sequence NM_000734.3)
(SEQ ID NO: 43)
RVKFSRSADAPAYQQGQNQLYNELNLGRREEYDVLDKRRG
RDPEMGGKPRRKNPQEGLYNELQKDKMAEAYSEIGMKGER
RRGKGHDGLYQGLSTATKDTYDALHMQALPPR
CD3 zeta (nucleic acid sequence; NCBI
Reference Sequence NM_000734.3);
(SEQ ID NO: 44)
AGAGTGAAGTTCAGCAGGAGCGCAGACGCCCCCGCGTACC
AGCAGGGCCAGAACCAGCTCTATAACGAGCTCAATCTAGG
ACGAAGAGAGGAGTACGATGTTTTGGACAAGAGACGTGGC
CGGGACCCTGAGATGGGGGGAAAGCCGAGAAGGAAGAACC
CTCAGGAAGGCCTGTACAATGAACTGCAGAAAGATAAGAT
GGCGGAGGCCTACAGTGAGATTGGGATGAAAGGCGAGCGC
CGGAGGGGCAAGGGGCACGATGGCCTTTACCAGGGTCTCA
GTACAGCCACCAAGGACACCTACGACGCCCTTCACATGCA
GGCCCTGCCCCCTCGC
CD28 domain
(amino acid sequence, SEQ ID NO: 1317)
RSKRSRLLHSDYMNMTPRRPGPTRKHYQPYAPPRDFAAYRS
CD28 domain
(nucleotide sequence, SEQ ID NO: 1318)
AGGAGTAAGAGGAGCAGGCTCCTGCACAGTGACTACATGA
ACATGACTCCCCGCCGCCCCGGGCCCACCCGCAAGCATTA
CCAGCCCTATGCCCCACCACGCGACTTCGCAGCCTATCGC
TCC
Wild-type ICOS domain
(amino acid sequence, SEQ ID NO: 1319)
TKKKYSSSVHDPNGEYMFMRAVNTAKKSRLTDVTL
Wild-type ICOS domain
(nucleotide sequence, SEQ ID NO: 1320)
ACAAAAAAGAAGTATTCATCCAGTGTGCACGACCCTAACG
GTGAATACATGTTCATGAGAGCAGTGAACACAGCCAAAAA
ATCCAGACTCACAGATGTGACCCTA
Y to F mutant ICOS domain
(amino acid sequence, SEQ ID NO: 1321)
TKKKYSSSVHDPNGEFMFMRAVNTAKKSRLTDVTL
IgG4 Hinge (amino acid sequence)
(SEQ ID NO: 102)
ESKYGPPCPPCPAPEFLGGPSVFLFPPKPKDTLMISRTPE
VTCVVVDVSQEDPEVQFNWYVDGVEVHNAKTKPREEQFNS
TYRVVSVLTVLHQDWLNGKEYKCKVSNKGLPSSIEKTISK
AKGQPREPQVYTLPPSQEEMTKNQVSLTCLVKGFYPSDIA
VEWESNGQPENNYKTTPPVLDSDGSFFLYSRLTVDKSRW
QEGNVFSCSVMHEALHNHYTQKSLSLSLGKM
IgG4 Hinge (nucleotide sequence)
(SEQ ID NO: 103)
GAGAGCAAGTACGGCCCTCCCTGCCCCCCTTGCCCTGCCC
CCGAGTTCCTGGGCGGACCCAGCGTGTTCCTGTTCCCCCC
CAAGCCCAAGGACACCCTGATGATCAGCCGGACCCCCGAG
GTGACCTGTGTGGTGGTGGACGTGTCCCAGGAGGACCCCG
AGGTCCAGTTCAACTGGTACGTGGACGGCGTGGAGGTGCA
CAACGCCAAGACCAAGCCCCGGGAGGAGCAGTTCAATAGC
ACCTACCGGGTGGTGTCCGTGCTGACCGTGCTGCACCAGG
ACTGGCTGAACGGCAAGGAATACAAGTGTAAGGTGTCCAA
CAAGGGCCTGCCCAGCAGCATCGAGAAAACCATCAGCAAG
GCCAAGGGCCAGCCTCGGGAGCCCCAGGTGTACACCCTGC
CCCCTAGCCAAGAGGAGATGACCAAGAACCAGGTGTCCCT
GACCTGCCTGGTGAAGGGCTTCTACCCCAGCGACATCGCC
GTGGAGTGGGAGAGCAACGGCCAGCCCGAGAACAACTACA
AGACCACCCCCCCTGTGCTGGACAGCGACGGCAGCTTCTT
CCTGTACAGCCGGCTGACCGTGGACAAGAGCCGGTGGCAG
GAGGGCAACGTCTTTAGCTGCTCCGTGATGCACGAGGCCC
TGCACAACCACTACACCCAGAAGAGCCTGAGCCTGTCCCT
GGGCAAGATG

These clones all contained a Q/K residue change in the signal domain of the co-stimulatory domain derived from 4-1BB.

TABLE 2
CD19 CAR Constructs

| | | |
|---|---|---|
| | SEQ | |
| Name | ID | Sequence |

CAR 1

| | | |
|---|---|---|
| CAR1 scFv | 1 | EIVMTQSPATLSLSPGERATLSCRASQDISKYLNWYQQKPGQAPRLL |
| domain | | IYHTSRLHSGIPARFSGSGSGTDYTLTISSLQPEDFAVYFCQQGNTL |
| | | PYTFGQGTKLEIKGGGGSGGGGSGGGGSQVQLQESGPGLVKPSETLS |
| | | LTCTVSGVSLPDYGVSWIRQPPGKGLEWIGVIWGSETTYYSSSLKSR |
| | | VTISKDNSKNQVSLKLSSVTAADTAVYYCAKHYYYGGSYAMDYWGQG |
| | | TSVTVSS |
| 103101 | 61 | atggccctccctgtcaccgccctgctgcttccgctggctcttctgctccacg |
| CAR1 | | ccgctcggcccgaaattgtgatgacccagtcacccgccactcttagcctttc |
| Soluble scFv-nt | | acccggtgagcgcgcaaccctgtcttgcagagcctcccaagacatctcaaaa |
| | | taccttaattggtatcaacagaagcccggacaggctcctcgccttctgatct |
| | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagccaggtccaactccaagaaagcggaccgggtcttgtgaag |
| | | ccatcagaaactctttcactgacttgtactgtgagcggagtgtctctccccg |
| | | attacggggtgtcttggatcagacagccaccggggaagggtctggaatggat |
| | | tggagtgatttggggctctgagactacttactactcttcatccctcaagtca |
| | | cgcgtcaccatctcaaaggacaactctaagaatcaggtgtcactgaaactgt |
| | | catctgtgaccgcagccgacaccgccgtgtactattgcgctaagcattacta |
| | | ttatggcgggagctacgcaatggattactggggacagggtactctggtcacc |
| | | gtgtccagccaccaccatcatcaccatcaccat |
| 103101 | 73 | MALPVTALLLPLALLLHAARPeivmtqspatlslspgeratlscrasqdisk |
| CAR1 | | y-nwyqqkpgqaprlliyhtsrlhsgiparfsgsgsgtdytltisslqpedf |
| Soluble scFv-aa | | avyfcqqgntlpytfgqgtkleikggggsggggsggggsqvqlqesgpglvk |
| | | psetlsltctvsgvslpdygvswirqppgkglewigviwgsettyyssslks |
| | | rvtiskdnsknqvslklssvtaadtavyycakhyyyggsyamdywgqgtlvt |
| | | vsshhhhhhhh |
| 104875 | 85 | atggccctccctgtcaccgccctgctgcttccgctggctcttctgctccacg |
| CAR 1-Full-nt | | ccgctcggcccgaaattgtgatgacccagtcacccgccactcttagcctttc |
| | | acccggtgagcgcgcaaccctgtcttgcagagcctcccaagacatctcaaaa |
| | | taccttaattggtatcaacagaagcccggacaggctcctcgccttctgatct |
| | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagccaggtccaactccaagaaagcggaccgggtcttgtgaag |
| | | ccatcagaaactctttcactgacttgtactgtgagcggagtgtctctccccg |
| | | attacggggtgtcttggatcagacagccaccggggaagggtctggaatggat |
| | | tggagtgatttggggctctgagactacttactactcttcatccctcaagtca |
| | | cgcgtcaccatctcaaaggacaactctaagaatcaggtgtcactgaaactgt |
| | | catctgtgaccgcagccgacaccgccgtgtactattgcgctaagcattacta |
| | | ttatggcgggagctacgcaatggattactggggacagggtactctggtcacc |
| | | gtgtccagcaccactaccccagcaccgaggccacccaccccggctcctacca |
| | | tcgcctcccagcctctgtccctgcgtccggaggcatgtagacccgcagctgg |
| | | tggggccgtgcatacccggggtcttgacttcgcctgcgatatctacatttgg |
| | | gcccctctggctggtacttgcggggtcctgctgctttcactcgtgatcactc |
| | | tttactgtaagcgcggtcggaagaagctgctgtacatctttaagcaaccctt |
| | | catgaggcctgtgcagactactcaagaggaggacggctgttcatgccggttc |
| | | ccagaggaggaggaaggcggctgcgaactgcgcgtgaaattcagccgcagcg |
| | | cagatgctccagcctacaagcaggggcagaaccagctctacaacgaactcaa |
| | | tcttggtcggagagaggagtacgacgtgctggacaagcggagaggacgggac |
| | | ccagaaatgggcgggaagccgcgcagaaagaatccccaagagggcctgtaca |
| | | acgagctccaaaaggataagatggcagaagcctatagcgagattggtatgaa |
| | | aggggaacgcagaagaggcaaaggccacgacggactgtaccagggactcagc |
| | | accgccaccaaggacacctatgacgctcttcacatgcaggccctgccgcctc |
| | | gg |
| 104875 | 31 | MALPVTALLLPLALLLHAARPeivmtqspatlslspgeratlscrasqdisk |
| CAR 1-Full-aa | | ylnwyqqkpgqaprlliyhtsrlhsgiparfsgsgsgtdytltisslqpedf |
| | | avyfcqqgntlpytfgqgtkleikgggggsgggsggggsqvqlqesgpglvk |
| | | psetlsltctvsgvslpdygvswirqppgkglewigviwgsettyyssslks |
| | | rvtiskdnsknqvslklssvtaadtavyycakhyyyggsyamdywgqgtlvt |
| | | vsstttpaprpptpaptiasqplslrpeacrpaaggavhtrgldfacdiyiw |
| | | aplagtcgvlllslvitiyckrgrkkllyifkqpfmrpvqttqeedgcscrf |
| | | peeeeggcelrvkfsrsadapaykqgqnqlynelnlgrreeydvldkrrgrd |
| | | pemggkprrknpqeglynelqkdkmaeayseigmkgerrgkghdglyqgls |
| | | tatkdtydalhmqalppr |

CAR 2

| | | |
|---|---|---|
| CAR2 scFv | 2 | eivmtqspatlslspgeratlscrasqdiskylnwyqqkpgqaprlliyhts |
| domain | | rlhsgiparfsgsgsgtdytltisslqpedfavyfcqqgntlpytfgqgtkl |
| | | eikggggsggggsggggsqvqlqesgpglvkpsetlsltctvsgvslpdygv |
| | | swirqppgkglewigviwgsettyyqsslksrvtiskdnsknqvslklssvt |
| | | aadtavyycakhyyyggsyamdywgqgtlvtvss |
| CAR2 scFv | 5006 | gaaattgtgatgacccagtcacccgccactcttagcctttcacccggtgagc |
| domain-nt | | gcgcaaccctgtcttgcagagcctcccaagacatctcaaaataccttaattg |
| | | gtatcaacagaagcccggacaggctcctcgccttctgatctaccacaccagc |
| | | cggctccattctggaatccctgccaggttcagcggtagcggatctgggaccg |
| | | actacaccctcactatcagctcactgcagccagaggacttcgctgtctattt |
| | | ctgtcagcaagggaacaccctgccctacacctttggacagggcaccaagctc |
| | | gagattaaaggtggaggtggcagcggaggaggtgggtccggcggtggaggaa |
| | | gccaggtccaactccaagaaagcggaccgggtcttgtgaagccatcagaaac |
| | | tctttcactgacttgtactgtgagcggagtgtctctccccgattacggggtg |
| | | tcttggatcagacagccaccggggaagggtctggaatggattggagtgattt |
| | | ggggctctgagactacttactaccaatcatccctcaagtcacgcgtcaccat |
| | | ctcaaaggacaactctaagaatcaggtgtcactgaaactgtcatctgtgacc |
| | | gcagccgacaccgccgtgtactattgcgctaagcattactattatggcggga |
| | | gctacgcaatggattactggggacagggtactctggtcaccgtgtccagc |
| 103102 | 62 | atggccctccctgtcaccgccctgctgcttccgctggctcttctgctccacg |
| CAR2-Soluble | | ccgctcggcccgaaattgtgatgacccagtcacccgccactcttagcctttc |
| scFv-nt | | acccggtgagcgcgcaaccctgtcttgcagagcctcccaagacatctcaaaa |
| | | taccttaattggtatcaacagaagcccggacaggctcctcgccttctgatct |
| | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagccaggtccaactccaagaaagcggaccgggtcttgtgaag |
| | | ccatcagaaactctttcactgacttgtactgtgagcggagtgtctctccccg |
| | | attacggggtgtcttggatcagacagccaccggggaagggtctggaatggat |
| | | tggagtgatttggggctctgagactacttactaccaatcatccctcaagtca |
| | | cgcgtcaccatctcaaaggacaactctaagaatcaggtgtcactgaaactgt |
| | | catctgtgaccgcagccgacaccgccgtgtactattgcgctaagcattacta |
| | | ttatggcgggagctacgcaatggattactggggacagggtactctggtcacc |
| | | gtgtccagccaccaccatcatcaccatcaccat |
| 103102 | 74 | MALPVTALLLPLALLLHAARPeivmtqspatlslspgeratlscrasqdisk |
| CAR2-Soluble | | ylnwyqqkpgqaprlliyhtsrlhsgiparfsgsgsgtdytltisslqpedf |
| scFv-aa | | avyfcqqgntlpytfgqgtkleikggggsggggsggggsqvqlqesgpglvk |
| | | psetlsltctvsgvslpdygvswirqppgkglewigviwgsettyyqsslks |
| | | rvtiskdnsknqvslklssvtaadtavyycakhyyyggsyamdywgqgtlvt |
| | | vsshhhhhhhh |
| 104876 | 86 | atggccctccctgtcaccgccctgctgcttccgctggctcttctgctccacg |
| CAR 2-Full-nt | | ccgctcggcccgaaattgtgatgacccagtcacccgccactcttagcctttc |
| | | acccggtgagcgcgcaaccctgtcttgcagagcctcccaagacatctcaaaa |
| | | taccttaattggtatcaacagaagcccggacaggctcctcgccttctgatct |
| | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagccaggtccaactccaagaaagcggaccgggtcttgtgaag |
| | | ccatcagaaactctttcactgacttgtactgtgagcggagtgtctctccccg |
| | | attacggggtgtcttggatcagacagccaccggggaagggtctggaatggat |
| | | tggagtgatttggggctctgagactacttactaccaatcatccctcaagtca |
| | | cgcgtcaccatctcaaaggacaactctaagaatcaggtgtcactgaaactgt |
| | | catctgtgaccgcagccgacaccgccgtgtactattgcgctaagcattacta |
| | | ttatggcgggagctacgcaatggattactggggacagggtactctggtcacc |
| | | gtgtccagcaccactaccccagcaccgaggccacccaccccggctcctacca |
| | | tcgcctcccagcctctgtccctgcgtccggaggcatgtagacccgcagctgg |
| | | tggggccgtgcatacccggggtcttgacttcgcctgcgatatctacatttgg |
| | | gcccctctggctggtacttgcggggtcctgctgctttcactcgtgatcactc |
| | | tttactgtaagcgcggtcggaagaagctgctgtacatctttaagcaaccctt |
| | | catgaggcctgtgcagactactcaagaggaggacggctgttcatgccggttc |
| | | ccagaggaggaggaaggCggctgcgaactgcgcgtgaaattcagccgcagcg |
| | | cagatgctccagcctacaagcaggggcagaaccagctctacaacgaactcaa |
| | | tcttggtcggagagaggagtacgacgtgctggacaagcggagaggacgggac |
| | | ccagaaatgggcgggaagccgcgcagaaagaatccccaagagggcctgtaca |
| | | acgagctccaaaaggataagatggcagaagcctatagcgagattggtatgaa |
| | | aggggaacgcagaagaggcaaaggccacgacggactgtaccagggactcagc |
| | | accgccaccaaggacacctatgacgctcttcacatgcaggccctgccgcctc |
| | | gg |
| 104876 | 32 | MALPVTALLLPLALLLHAARPeivmtqspatlslspgeratlscrasqdisk |
| CAR 2-Full-aa | | ylnwyqqkpgqaprlliyhtsrlhsgiparfsgsgsgtdytltisslqpedf |
| | | avyfcqqgntlpytfgqgtkleikggggsggggsggggsqvqlqesgpglvk |
| | | psetlsltctvsgvslpdygvswirqppgkglewigviwgsettyygsslks |
| | | rvtiskdnsknqvslklssvtaadtavyycakhyyyggsyamdywgqgtlvt |
| | | vsstttpaprpptpaptiasqplslrpeacrpaaggavhtrgldfacdiyiw |
| | | aplagtcgvlllslvitiyckrgrkkllyifkqpfmrpvqttqeedgescrf |
| | | peeeeggcelrvkfsrsadapaykqgqnqlynelnlgrreeydvldkrrgrd |
| | | pemggkprrknpqeglynelqkdkmaeayseigmkgerrrgkghdglyqgls |
| | | tatkdtydalhmqalppr |
| CAR 2-Full-nt | 5007 | gaaattgtgatgacccagtcacccgccactcttagcctttcacccggtgagc |
| | | gcgcaaccctgtcttgcagagcctcccaagacatctcaaaataccttaattg |
| | | gtatcaacagaagcccggacaggctcctcgccttctgatctaccacaccagc |
| | | cggctccattctggaatccctgccaggttcagcggtagcggatctgggaccg |
| | | actacaccctcactatcagctcactgcagccagaggacttcgctgtctattt |
| | | ctgtcagcaagggaacaccctgccctacacctttggacagggcaccaagctc |
| | | gagattaaaggtggaggtggcagcggaggaggtgggtccggcggtggaggaa |
| | | gccaggtccaactccaagaaagcggaccgggtcttgtgaagccatcagaaac |
| | | tctttcactgacttgtactgtgagcggagtgtctctccccgattacggggtg |
| | | tcttggatcagacagccaccggggaagggtctggaatggattggagtgattt |
| | | ggggctctgagactacttactaccaatcatccctcaagtcacgcgtcaccat |
| | | ctcaaaggacaactctaagaatcaggtgtcactgaaactgtcatctgtgacc |
| | | gcagccgacaccgccgtgtactattgcgctaagcattactattatggcggga |
| | | gctacgcaatggattactggggacagggtactctggtcaccgtgtccagcac |
| | | cactaccccagcaccgaggccacccaccccggctcctaccatcgcctcccag |
| | | cctctgtccctgcgtccggaggcatgtagacccgcagctggtggggccgtgc |
| | | atacccggggtcttgacttcgcctgcgatatctacatttgggcccctctggc |
| | | tggtacttgcggggtcctgctgctttcactcgtgatcactctttactgtaag |
| | | cgcggtcggaagaagctgctgtacatctttaagcaacccttcatgaggcctg |
| | | tgcagactactcaagaggaggacggctgttcatgccggttcccagaggagga |
| | | ggaaggcggctgcgaactgcgcgtgaaattcagccgcagcgcagatgctcca |
| | | gcctacaagcaggggcagaaccagctctacaacgaactcaatcttggtcgga |
| | | gagaggagtacgacgtgctggacaagcggagaggacgggacccagaaatggg |
| | | cgggaagccgcgcagaaagaatccccaagagggcctgtacaacgagctccaa |
| | | aaggataagatggcagaagcctatagcgagattggtatgaaaggggaacgca |
| | | gaagaggcaaaggccacgacggactgtaccagggactcagcaccgccaccaa |
| | | ggacacctatgacgctcttcacatgcaggccctgccgcctcgg |
| CAR 2-Full-aa | 5008 | eivmtqspatlslspgeratlscrasqdiskylnwyqqkpgqaprlliyhts |
| | | rlhsgiparfsgsgsgtdytltisslqpedfavyfcqqgntlpytfgqgtkl |
| | | eikggggsggggsggggsqvqlqesgpglvkpsetlsltctvsgvslpdygv |
| | | swirqppgkglewigviwgsettyyqsslksrvtiskdnsknqvslklssvt |
| | | aadtavyycakhyyyggsyamdywgqgtlvtvsstttpaprpptpaptiasq |
| | | pislrpeacrpaaggavhtrgldfacdiyiwaplagtcgvlllslvitlyck |
| | | rgrkkllyifkqpfmrpvqttqeedgcscrfpeeeeggcelrvkfsrsadap |
| | | aykqgqnqlynelnlgrreeydvldkrrgrdpemggkprrknpqeglynelq |
| | | kdkmaeayseigmkgerrrgkghdglyqglstatkdtydalhmqalppr |

CAR 3

| | | |
|---|---|---|
| CAR3 scFv | 3 | qvqlqesgpglvkpsetlsltctvsgvslpdygvswirqppgkglewigviw |
| domain | | gsettyyssslksrvtiskdnsknqvslklssvtaadtavyycakhyyyggs |
| | | yamdywgqgtlvtvssggggsggggsggggseivmtqspatlslspgeratl |
| | | scrasqdiskylnwyqqkpgqaprlliyhtsrlhsgiparfsgsgsgtdytl |
| | | tisslqpedfavyfcqqgntlpytfgqgtkleik |
| 103104 | 63 | atggctctgcccgtgaccgcactcctcctgccactggctctgctgcttcacg |
| CAR 3-Soluble | | ccgctcgcccacaagtccagcttcaagaatcagggcctggtctggtgaagcc |
| scFv-nt | | atctgagactctgtccctcacttgcaccgtgagcggagtgtccctcccagac |
| | | tacggagtgagctggattagacagcctcccggaaagggactggagtggatcg |
| | | gagtgatttggggtagcgaaaccacttactattcatcttccctgaagtcacg |
| | | ggtcaccatttcaaaggataactcaaagaatcaagtgagcctcaagctctca |
| | | tcagtcaccgccgctgacaccgccgtgtattactgtgccaagcattactact |
| | | atggagggtcctacgccatggactactggggccagggaactctggtcactgt |
| | | gtcatctggtggaggaggtagcggaggaggcgggagcggtggaggtggctcc |
| | | gaaatcgtgatgacccagagccctgcaaccctgtccctttctcccggggaac |
| | | gggctaccctttcttgtcgggcatcacaagatatctcaaaatacctcaattg |
| | | gtatcaacagaagccgggacaggcccctaggcttcttatctaccacacctct |
| | | cgcctgcatagcgggattcccgcacgctttagcgggtctggaagcgggaccg |
| | | actacactctgaccatctcatctctccagcccgaggacttcgccgtctactt |
| | | ctgccagcagggtaacaccctgccgtacaccttcggccagggcaccaagctt |
| | | gagatcaaacatcaccaccatcatcaccatcac |
| 103104 | 75 | MALPVTALLLPLALLLHAARPqvqlqesgpglvkpsetlsltctvsgvslpd |
| CAR 3-Soluble | | ygvswirqppgkglewigviwgsettyyssslksrvtiskdnsknqvslkls |
| scFv-aa | | svtaadtavyycakhyyyggsyamdywgqgtlvtvssggggsggggsggggs |
| | | eivmtqspatlslspgeratlscrasqdiskylnwyqqkpgqaprlliyhts |
| | | rlhsgiparfsgsgsgtdytltisslqpedfavyfcqqgntlpytfgqgtk1 |
| | | eikhhhhhhhh |
| 104877 | 87 | atggctctgcccgtgaccgcactcctcctgccactggctctgctgcttcacg |
| CAR 3-Full-nt | | ccgctcgcccacaagtccagcttcaagaatcagggcctggtctggtgaagcc |
| | | atctgagactctgtccctcacttgcaccgtgagcggagtgtccctcccagac |
| | | tacggagtgagctggattagacagcctcccggaaagggactggagtggatcg |
| | | gagtgatttggggtagcgaaaccacttactattcatcttccctgaagtcacg |
| | | ggtcaccatttcaaaggataactcaaagaatcaagtgagcctcaagctctca |
| | | tcagtcaccgccgctgacaccgccgtgtattactgtgccaagcattactact |
| | | atggagggtcctacgccatggactactggggccagggaactctggtcactgt |
| | | gtcatctggtggaggaggtagcggaggaggcgggagcggtggaggtggctcc |
| | | gaaatcgtgatgacccagagccctgcaaccctgtccctttctcccggggaac |
| | | gggctaccctttcttgtcgggcatcacaagatatctcaaaatacctcaattg |
| | | gtatcaacagaagccgggacaggcccctaggcttcttatctaccacacctct |
| | | cgcctgcatagcgggattcccgcacgctttagcgggtctggaagcgggaccg |
| | | actacactctgaccatctcatctctccagcccgaggacttcgccgtctactt |
| | | ctgccagcagggtaacaccctgccgtacaccttcggccagggcaccaagctt |
| | | gagatcaaaaccactactcccgctccaaggccacccacccctgccccgacca |
| | | tcgcctctcagccgctttccctgcgtccggaggcatgtagacccgcagctgg |
| | | tggggccgtgcatacccggggtcttgacttcgcctgcgatatctacatttgg |
| | | gcccctctggctggtacttgcggggtcctgctgctttcactcgtgatcactc |
| | | tttactgtaagcgcggtcggaagaagctgctgtacatctttaagcaaccctt |
| | | catgaggcctgtgcagactactcaagaggaggacggctgttcatgccggttc |
| | | ccagaggaggaggaaggcggctgcgaactgcgcgtgaaattcagccgcagcg |
| | | cagatgctccagcctacaagcaggggcagaaccagctctacaacgaactcaa |
| | | tcttggtcggagagaggagtacgacgtgctggacaagcggagaggacgggac |
| | | ccagaaatgggcgggaagccgcgcagaaagaatccccaagagggcctgtaca |
| | | acgagctccaaaaggataagatggcagaagcctatagcgagattggtatgaa |
| | | aggggaacgcagaagaggcaaaggccacgacggactgtaccagggactcagc |
| | | accgccaccaaggacacctatgacgctcttcacatgcaggccctgccgcctc |
| | | gg |
| 104877 | 33 | MALPVTALLLPLALLLHAARPqvqlqesgpglvkpsetlsltctvsgvslpd |
| CAR 3-Full-aa | | ygvswirqppgkglewigviwgsettyyssslksrvtiskdnsknqvslkls |
| | | svtaadtavyycakhyyyggsyamdywgqgtlvtvssggggsggggsggggs |
| | | eivmtqspatlslspgeratlscrasqdiskylnwyqqkpgqaprlliyhts |
| | | rlhsgiparfsgsgsgtdytltisslqpedfavyfcqqgntlpytfgqgtkl |
| | | eiktttpaprpptpaptiasqplslrpeacrpaaggavhtrgldfacdiyiw |
| | | aplagtcgvlllslvitiyckrgrkkllyifkqpfmrpvqttqeedgcscrf |
| | | peeeeggcelrvkfsrsadapaykqgqnqlynelnlgrreeydvldkrrgrd |
| | | pemggkprrknpqeglynelqkdkmaeayseigmkgerrrgkghdglyqgls |
| | | tatkdtydalhmqalppr |

CAR 4

| | | |
|---|---|---|
| CAR4 scFv | 4 | qvqlqesgpglvkpsetisltctvsgvslpdygvswirqppgkglewigviw |
| domain | | gsettyyqsslksrvtiskdnsknqvslklssvtaadtavyycakhyyyggs |
| | | yamdywgqgtlvtvssggggsggggsggggseivmtqspatlslspgeratl |
| | | scrasqdiskylnwyqqkpgqaprlliyhtsrlhsgiparfsgsgsgtdytl |
| | | tisslqpedfavyfcqqgntlpytfgqgtkleik |
| 103106 CAR4- | 64 | atggctctgcccgtgaccgcactcctcctgccactggctctgctgcttcacg |
| Soluble scFv-nt | | ccgctcgcccacaagtccagcttcaagaatcagggcctggtctggtgaagcc |
| | | atctgagactctgtccctcacttgcaccgtgagcggagtgtccctcccagac |
| | | tacggagtgagctggattagacagcctcccggaaagggactggagtggatcg |
| | | gagtgatttggggtagcgaaaccacttactatcaatcttccctgaagtcacg |
| | | ggtcaccatttcaaaggataactcaaagaatcaagtgagcctcaagctctca |
| | | tcagtcaccgccgctgacaccgccgtgtattactgtgccaagcattactact |
| | | atggagggtcctacgccatggactactggggccagggaactctggtcactgt |
| | | gtcatctggtggaggaggtagcggaggaggcgggagcggtggaggtggctcc |
| | | gaaatcgtgatgacccagagccctgcaaccctgtccctttctcccggggaac |
| | | gggctaccctttcttgtcgggcatcacaagatatctcaaaatacctcaattg |
| | | gtatcaacagaagccgggacaggcccctaggcttcttatctaccacacctct |
| | | cgcctgcatagcgggattcccgcacgctttagcgggtctggaagcgggaccg |
| | | actacactctgaccatctcatctctccagcccgaggacttcgccgtctactt |
| | | ctgccagcagggtaacaccctgccgtacaccttcggccagggcaccaagctt |
| | | gagatcaaacatcaccaccatcatcaccatcac |
| 103106 CAR4- | 76 | MALPVTALLLPLALLLHAARPqvqlqesgpglvkpsetlsltctvsgvslpd |
| Soluble scFv-aa | | ygvswirqppgkglewigviwgsettyyqsslksrvtiskdnsknqvslkls |
| | | svtaadtavyycakhyyyggsyamdywgqgtlvtvssggggsggggsggggs |
| | | eivmtqspatlslspgeratlscrasqdiskylnwyqqkpgqaprlliyhts |
| | | rlhsgiparfsgsgsgtdytltisslqpedfavyfcqqgntlpytfgqgtkl |
| | | eikhhhhhhhh |
| 104878 | 88 | atggctctgcccgtgaccgcactcctcctgccactggctctgctgcttcacg |
| CAR 4-Full-nt | | ccgctcgcccacaagtccagcttcaagaatcagggcctggtctggtgaagcc |
| | | atctgagactctgtccctcacttgcaccgtgagcggagtgtccctcccagac |
| | | tacggagtgagctggattagacagcctcccggaaagggactggagtggatcg |
| | | gagtgatttggggtagcgaaaccacttactatcaatcttccctgaagtcacg |
| | | ggtcaccatttcaaaggataactcaaagaatcaagtgagcctcaagctctca |
| | | tcagtcaccgccgctgacaccgccgtgtattactgtgccaagcattactact |
| | | atggagggtcctacgccatggactactggggccagggaactctggtcactgt |
| | | gtcatctggtggaggaggtagcggaggaggcgggagcggtggaggtggctcc |
| | | gaaatcgtgatgacccagagccctgcaaccctgtccctttctcccggggaac |
| | | gggctaccctttcttgtcgggcatcacaagatatctcaaaatacctcaattg |
| | | gtatcaacagaagccgggacaggcccctaggcttcttatctaccacacctct |
| | | cgcctgcatagcgggattcccgcacgctttagcgggtctggaagcgggaccg |
| | | actacactctgaccatctcatctctccagcccgaggacttcgccgtctactt |
| | | ctgccagcagggtaacaccctgccgtacaccttcggccagggcaccaagctt |
| | | gagatcaaaaccactactcccgctccaaggccacccacccctgccccgacca |
| | | tcgcctctcagccgctttccctgcgtccggaggcatgtagacccgcagctgg |
| | | tggggccgtgcatacccggggtcttgacttcgcctgcgatatctacatttgg |
| | | gcccctctggctggtacttgcggggtcctgctgctttcactcgtgatcactc |
| | | tttactgtaagcgcggtcggaagaagctgctgtacatctttaagcaaccctt |
| | | catgaggcctgtgcagactactcaagaggaggacggctgttcatgccggttc |
| | | ccagaggaggaggaaggcggctgcgaactgcgcgtgaaattcagccgcagcg |
| | | cagatgctccagcctacaagcaggggcagaaccagctctacaacgaactcaa |
| | | tcttggtcggagagaggagtacgacgtgctggacaagcggagaggacgggac |
| | | ccagaaatgggcgggaagccgcgcagaaagaatccccaagagggcctgtaca |
| | | acgagctccaaaaggataagatggcagaagcctatagcgagattggtatgaa |
| | | aggggaacgcagaagaggcaaaggccacgacggactgtaccagggactcagc |
| | | accgccaccaaggacacctatgacgctcttcacatgcaggccctgccgcctc |
| | | gg |
| 104878 | 34 | MALPVTALLLPLALLLHAARPqvqlqesgpglvkpsetlsltctvsgvslpd |
| CAR 4-Full-aa | | ygvswirqppgkglewigviwgsettyyqsslksrvtiskdnsknqvslkls |
| | | svtaadtavyycakhyyyggsyamdywgqgtlvtvssggggsggggsggggs |
| | | eivmtqspatlslspgeratlscrasqdiskylnwyqqkpgqaprlliyhts |
| | | rlhsgiparfsgsgsgtdytltisslqpedfavyfcqqgntlpytfgqgtkl |
| | | eiktttpaprpptpaptiasqplslrpeacrpaaggavhtrgldfacdiyiw |
| | | aplagtcgvlllslvitIyckrgrkkllyifkqpfmrpvqttqeedgescrf |
| | | peeeeggcelrvkfsrsadapaykqgqnqlynelnlgrreeydvldkrrgrd |
| | | pemggkprrknpqeglynelqkdkmaeayseigmkgerrrgkghdglyqgls |
| | | tatkdtydalhmqalppr |

CAR 5

| | | |
|---|---|---|
| CAR5 scFv | 5 | eivmtqspatlslspgeratlscrasqdiskylnwyqqkpgqaprlliyhts |
| domain | | rlhsgiparfsgsgsgtdytltisslqpedfavyfcqqgntlpytfgqgtkl |
| | | eikggggsggggsggggsggggsqvqlqesgpglvkpsetlsltctvsgvsl |
| | | pdygvswirqppgkglewigviwgsettyyssslksrvtiskdnsknqvslk |
| | | lssvtaadtavyycakhyyyggsyamdywgqgtlvtvss |
| 99789 | 65 | atggccctcccagtgaccgctctgctgctgcctctcgcacttcttctccatg |
| CAR5-Soluble | | ccgctcggcctgagatcgtcatgacccaaagccccgctaccctgtccctgtc |
| scFv-nt | | acccggcgagagggcaaccctttcatgcagggccagccaggacatttctaag |
| | | tacctcaactggtatcagcagaagccagggcaggctcctcgcctgctgatct |
| | | accacaccagccgcctccacagcggtatccccgccagattttccgggagcgg |
| | | gtctggaaccgactacaccctcaccatctcttctctgcagcccgaggatttc |
| | | gccgtctatttctgccagcaggggaatactctgccgtacaccttcggtcaag |
| | | gtaccaagctggaaatcaagggaggcggaggatcaggcggtggcggaagcgg |
| | | aggaggtggctccggaggaggaggttcccaagtgcagcttcaagaatcagga |
| | | cccggacttgtgaagccatcagaaaccctctccctgacttgtaccgtgtccg |
| | | gtgtgagcctccccgactacggagtctcttggattcgccagcctccggggaa |
| | | gggtcttgaatggattggggtgatttggggatcagagactacttactactct |
| | | tcatcacttaagtcacgggtcaccatcagcaaagataatagcaagaaccaag |
| | | tgtcacttaagctgtcatctgtgaccgccgctgacaccgccgtgtactattg |
| | | tgccaaacattactattacggagggtcttatgctatggactactggggacag |
| | | gggaccctggtgactgtctctagccatcaccatcaccaccatcatcac |
| 99789 | 77 | MALPVTALLLPLALLLHAARPeivmtqspatlslspgeratlscrasqdisk |
| CAR5-Soluble | | ylnwyqqkpgqaprlliyhtsrlhsgiparfsgsgsgtdytltisslqpedf |
| scFv-aa | | avyfcqqgntlpytfgqgtkleikggggsggggsggggsggggsqvqlqesg |
| | | pglvkpsetlsltctvsgvslpdygvswirqppgkglewigviwgsettyys |
| | | sslksrvtiskdnsknqvslklssvtaadtavyycakhyyyggsyamdywgq |
| | | gtlvtvsshhhhhhhh |
| 104879 | 89 | atggccctccctgtcaccgccctgctgcttccgctggctcttctgctccacg |
| CAR 5-Full-nt | | ccgctcggcccgaaattgtgatgacccagtcacccgccactcttagcctttc |
| | | acccggtgagcgcgcaaccctgtcttgcagagcctcccaagacatctcaaaa |
| | | taccttaattggtatcaacagaagcccggacaggctcctcgccttctgatct |
| | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagcggcggaggcgggagccaggtccaactccaagaaagcgga |
| | | ccgggtcttgtgaagccatcagaaactctttcactgacttgtactgtgagcg |
| | | gagtgtctctccccgattacggggtgtcttggatcagacagccaccggggaa |
| | | gggtctggaatggattggagtgatttggggctctgagactacttactactct |
| | | tcatccctcaagtcacgcgtcaccatctcaaaggacaactctaagaatcagg |
| | | tgtcactgaaactgtcatctgtgaccgcagccgacaccgccgtgtactattg |
| | | cgctaagcattactattatggcgggagctacgcaatggattactggggacag |
| | | ggtactctggtcaccgtgtccagcaccactaccccagcaccgaggccaccca |
| | | ccccggctcctaccatcgcctcccagcctctgtccctgcgtccggaggcatg |
| | | tagacccgcagctggtggggccgtgcatacccggggtcttgacttcgcctgc |
| | | gatatctacatttgggcccctctggctggtacttgcggggtcctgctgcttt |
| | | cactcgtgatcactctttactgtaagcgcggtcggaagaagctgctgtacat |
| | | ctttaagcaacccttcatgaggcctgtgcagactactcaagaggaggacggc |
| | | tgttcatgccggttcccagaggaggaggaaggcggctgcgaactgcgcgtga |
| | | aattcagccgcagcgcagatgctccagcctacaagcaggggcagaaccagct |
| | | ctacaacgaactcaatcttggtcggagagaggagtacgacgtgctggacaag |
| | | cggagaggacgggacccagaaatgggcgggaagccgcgcagaaagaatcccc |
| | | aagagggcctgtacaacgagctccaaaaggataagatggcagaagcctatag |
| | | cgagattggtatgaaaggggaacgcagaagaggcaaaggccacgacggactg |
| | | taccagggactcagcaccgccaccaaggacacctatgacgctcttcacatgc |
| | | aggccctgccgcctcgg |
| 104879 | 35 | MALPVTALLLPLALLLHAARPeivmtqspatlslspgeratlscrasqdisk |
| CAR 5-Full-aa | | ylnwyqqkpgqaprlliyhtsrlhsgiparfsgsgsgtdytltisslqpedf |
| | | avyfcqqgntlpytfgqgtkleikggggsggggsggggsggggsqvqlqesg |
| | | pglvkpsetlsltctvsgvslpdygvswirqppgkglewigviwgsettyys |
| | | sslksrvtiskdnsknqvslklssvtaadtavyycakhyyyggsyamdywgq |
| | | gtlvtvsstttpaprpptpaptiasqplslrpeacrpaaggavhtrgldfac |
| | | diyiwaplagtcgvlllslvitlyckrgrkkllyifkqpfmrpvqttqeedg |
| | | cscrfpeeeeggcelrvkfsrsadapaykqgqnqlynelnlgrreeydvldk |
| | | rrgrdpemggkprrknpqeglynelqkdkmaeayseigmkgerrrgkghdgl |
| | | yqglstatkdtydalhmqalppr |

CAR 6

| | | |
|---|---|---|
| CAR6 | 6 | eivmtqspatlslspgeratlscrasqdiskylnwyqqkpgqaprlliyhts |
| scFv domain | | rlhsgiparfsgsgsgtdytltisslqpedfavyfcqqgntlpytfgqgtkl |
| | | eikggggsggggsggggsggggsqvqlqesgpglvkpsetlsltctvsgvsl |
| | | pdygvswirqppgkglewigviwgsettyyqsslksrvtiskdnsknqvslk |
| | | lssvtaadtavyycakhyyyggsyamdywgqgtlvtvss |
| 99790 | 66 | atggccctcccagtgaccgctctgctgctgcctctcgcacttcttctccatg |
| CAR6-Soluble | | ccgctcggcctgagatcgtcatgacccaaagccccgctaccctgtccctgtc |
| scFv-nt | | acccggcgagagggcaaccctttcatgcagggccagccaggacatttctaag |
| | | tacctcaactggtatcagcagaagccagggcaggctcctcgcctgctgatct |
| | | accacaccagccgcctccacagcggtatccccgccagattttccgggagcgg |
| | | gtctggaaccgactacaccctcaccatctcttctctgcagcccgaggatttc |
| | | gccgtctatttctgccagcaggggaatactctgccgtacaccttcggtcaag |
| | | gtaccaagctggaaatcaagggaggcggaggatcaggcggtggcggaagcgg |
| | | aggaggtggctccggaggaggaggttcccaagtgcagcttcaagaatcagga |
| | | cccggacttgtgaagccatcagaaaccctctccctgacttgtaccgtgtccg |
| | | gtgtgagcctccccgactacggagtctcttggattcgccagcctccggggaa |
| | | gggtcttgaatggattggggtgatttggggatcagagactacttactaccag |
| | | tcatcacttaagtcacgggtcaccatcagcaaagataatagcaagaaccaag |
| | | tgtcacttaagctgtcatctgtgaccgccgctgacaccgccgtgtactattg |
| | | tgccaaacattactattacggagggtcttatgctatggactactggggacag |
| | | gggaccctggtgactgtctctagccatcaccatcaccaccatcatcac |
| 99790 | 78 | MALPVTALLLPLALLLHAARPeivmtqspatlslspgeratlscrasqdisk |
| CAR6-Soluble | | ylnwyqqkpgqaprlliyhtsrlhsgiparfsgsgsgtdytltisslqpedf |
| scFv-aa | | avyfcqqgntlpytfgqgtkleikggggsggggsggggsggggsqvqlqesg |
| | | pglvkpsetlsltctvsgvslpdygvswirqppgkglewigviwgsettyyq |
| | | sslksrvtiskdnsknqvslklssvtaadtavyycakhyyyggsyamdywgq |
| | | gtlvtvsshhhhhhhh |
| 104880 | 90 | atggccctccctgtcaccgccctgctgcttccgctggctcttctgctccacg |
| CAR6- | | ccgctcggcccgaaattgtgatgacccagtcacccgccactcttagcctttc |
| Full-nt | | acccggtgagcgcgcaaccctgtcttgcagagcctcccaagacatctcaaaa |
| | | taccttaattggtatcaacagaagcccggacaggctcctcgccttctgatct |
| | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagcggaggcggagggagccaggtccaactccaagaaagcgga |
| | | ccgggtcttgtgaagccatcagaaactctttcactgacttgtactgtgagcg |
| | | gagtgtctctccccgattacggggtgtcttggatcagacagccaccggggaa |
| | | gggtctggaatggattggagtgatttggggctctgagactacttactaccaa |
| | | tcatccctcaagtcacgcgtcaccatctcaaaggacaactctaagaatcagg |
| | | tgtcactgaaactgtcatctgtgaccgcagccgacaccgccgtgtactattg |
| | | cgctaagcattactattatggcgggagctacgcaatggattactggggacag |
| | | ggtactctggtcaccgtgtccagcaccactaccccagcaccgaggccaccca |
| | | ccccggctcctaccatcgcctcccagcctctgtccctgcgtccggaggcatg |
| | | tagacccgcagctggtggggccgtgcatacccggggtcttgacttcgcctgc |
| | | gatatctacatttgggcccctctggctggtacttgcggggtcctgctgcttt |
| | | cactcgtgatcactctttactgtaagcgcggtcggaagaagctgctgtacat |
| | | ctttaagcaacccttcatgaggcctgtgcagactactcaagaggaggacggc |
| | | tgttcatgccggttcccagaggaggaggaaggcggctgcgaactgcgcgtga |
| | | aattcagccgcagcgcagatgctccagcctacaagcaggggcagaaccagct |
| | | ctacaacgaactcaatcttggtcggagagaggagtacgacgtgctggacaag |
| | | cggagaggacgggacccagaaatgggcgggaagccgcgcagaaagaatcccc |
| | | aagagggcctgtacaacgagctccaaaaggataagatggcagaagcctatag |
| | | cgagattggtatgaaaggggaacgcagaagaggcaaaggccacgacggactg |
| | | taccagggactcagcaccgccaccaaggacacctatgacgctcttcacatgc |
| | | aggccctgccgcctcgg |
| 104880 | 36 | MALPVTALLLPLALLLHAARPeivmtqspatlslspgeratlscrasqdisk |
| CAR6- | | ylnwyqqkpgqaprlliyhtsrlhsgiparfsgsgsgtdytltisslqpedf |
| Full-aa | | avyfcqqgntlpytfgqgtkleikggggsggggsggggsggggsqvqlqesg |
| | | pglvkpsetlsltctvsgvslpdygvswirqppgkglewigviwgsettyyq |
| | | sslksrvtiskdnsknqvslklssvtaadtavyycakhyyyggsyamdywgq |
| | | gtlvtvsstttpaprpptpaptiasqplslrpeacrpaaggavhtrgldfac |
| | | diyiwaplagtcgvlllslvitlyckrgrkkllyifkqpfmrpvqttqeedg |
| | | cscrfpeeeeggcelrvkfsrsadapaykqgqnqlynelnlgrreeydvldk |
| | | rrgrdpemggkprrknpqeglynelqkdkmaeayseigmkgerrrgkghdgl |
| | | yqglstatkdtydalhmqalppr |

CAR 7

| | | |
|---|---|---|
| CAR7 scFv | 7 | qvqlqesgpglvkpsetlsltctvsgvslpdygvswirqppgkglewigviw |
| domain | | gsettyyssslksrvtiskdnsknqvslklssvtaadtavyycakhyyyggs |
| | | yamdywgqgtlvtvssggggsggggsggggsggggseivmtqspatlslspg |
| | | eratlscrasqdiskylnwyqqkpgqaprlliyhtsrlhsgiparfsgsgsg |
| | | tdytltisslqpedfavyfcqqgntlpytfgqgtkleik |
| 100796 | 67 | atggcactgcctgtcactgccctcctgctgcctctggccctccttctgcatg |
| CAR7-Soluble | | ccgccaggccccaagtccagctgcaagagtcaggacccggactggtgaagcc |
| scFv-nt | | gtctgagactctctcactgacttgtaccgtcagcggcgtgtccctccccgac |
| | | tacggagtgtcatggatccgccaacctcccgggaaagggcttgaatggattg |
| | | gtgtcatctggggttctgaaaccacctactactcatcttccctgaagtccag |
| | | ggtgaccatcagcaaggataattccaagaaccaggtcagccttaagctgtca |
| | | tctgtgaccgctgctgacaccgccgtgtattactgcgccaagcactactatt |
| | | acggaggaagctacgctatggactattggggacagggcactctcgtgactgt |
| | | gagcagcggcggtggagggtctggaggtggaggatccggtggtggtgggtca |
| | | ggcggaggagggagcgagattgtgatgactcagtcaccagccaccctttctc |
| | | tttcacccggcgagagagcaaccctgagctgtagagccagccaggacatttc |
| | | taagtacctcaactggtatcagcaaaaaccggggcaggcccctcgcctcctg |
| | | atctaccatacctcacgccttcactctggtatccccgctcggtttagcggat |
| | | caggatctggtaccgactacactctgaccatttccagcctgcagccagaaga |
| | | tttcgcagtgtatttctgccagcagggcaatacccttccttacaccttcggt |
| | | cagggaaccaagctcgaaatcaagcaccatcaccatcatcaccaccat |
| 100796 | 79 | MALPVTALLLPLALLLHAARPqvqlqesgpglvkpsetlsltctvsgvslpd |
| CAR7-Soluble | | ygvswirqppgkglewigviwgsettyyssslksrvtiskdnsknqvslkls |
| scFv-aa | | svtaadtavyycakhyyyggsyamdywgqgtlvtvssggggsggggsggggs |
| | | ggggseivmtqspatlslspgeratlscrasqdiskylnwyqqkpgqaprll |
| | | iyhtsrlhsgiparfsgsgsgtdytltisslqpedfavyfcqqgntlpytfg |
| | | qgtkleikhhhhhhhh |
| 104881 | 91 | atggctctgcccgtgaccgcactcctcctgccactggctctgctgcttcacg |
| CAR 7 | | ccgctcgcccacaagtccagcttcaagaatcagggcctggtctggtgaagcc |
| Full-nt | | atctgagactctgtccctcacttgcaccgtgagcggagtgtccctcccagac |
| | | tacggagtgagctggattagacagcctcccggaaagggactggagtggatcg |
| | | gagtgatttggggtagcgaaaccacttactattcatcttccctgaagtcacg |
| | | ggtcaccatttcaaaggataactcaaagaatcaagtgagcctcaagctctca |
| | | tcagtcaccgccgctgacaccgccgtgtattactgtgccaagcattactact |
| | | atggagggtcctacgccatggactactggggccagggaactctggtcactgt |
| | | gtcatctggtggaggaggtagcggaggaggcgggagcggtggaggtggctcc |
| | | ggaggtggcggaagcgaaatcgtgatgacccagagccctgcaaccctgtccc |
| | | tttctcccggggaacgggctaccctttcttgtcgggcatcacaagatatctc |
| | | aaaatacctcaattggtatcaacagaagccgggacaggcccctaggcttctt |
| | | atctaccacacctctcgcctgcatagcgggattcccgcacgctttagcgggt |
| | | ctggaagcgggaccgactacactctgaccatctcatctctccagcccgagga |
| | | cttcgccgtctacttctgccagcagggtaacaccctgccgtacaccttcggc |
| | | cagggcaccaagcttgagatcaaaaccactactcccgctccaaggccaccca |
| | | cccctgccccgaccatcgcctctcagccgctttccctgcgtccggaggcatg |
| | | tagacccgcagctggtggggccgtgcatacccggggtcttgacttcgcctgc |
| | | gatatctacatttgggcccctctggctggtacttgcggggtcctgctgcttt |
| | | cactcgtgatcactctttactgtaagcgcggtcggaagaagctgctgtacat |
| | | ctttaagcaacccttcatgaggcctgtgcagactactcaagaggaggacggc |
| | | tgttcatgccggttcccagaggaggaggaaggcggctgcgaactgcgcgtga |
| | | aattcagccgcagcgcagatgctccagcctacaagcaggggcagaaccagct |
| | | ctacaacgaactcaatcttggtcggagagaggagtacgacgtgctggacaag |
| | | cggagaggacgggacccagaaatgggcgggaagccgcgcagaaagaatcccc |
| | | aagagggcctgtacaacgagctccaaaaggataagatggcagaagcctatag |
| | | cgagattggtatgaaaggggaacgcagaagaggcaaaggccacgacggactg |
| | | taccagggactcagcaccgccaccaaggacacctatgacgctcttcacatgc |
| | | aggccctgccgcctcgg |
| 104881 | 37 | MALPVTALLLPLALLLHAARPqvqlqesgpglvkpsetlsltctvsgvslpd |
| CAR 7 | | ygvswirqppgkglewigviwgsettyyssslksrvtiskdnsknqvslkls |
| Full-aa | | svtaadtavyycakhyyyggsyamdywgqgtlvtvssggggsggggsggggs |
| | | ggggseivmtqspatlslspgeratlscrasqdiskylnwyqqkpgqaprll |
| | | iyhtsrlhsgiparfsgsgsgtdytltisslqpedfavyfcqqgntlpytfg |
| | | qgtkleiktttpaprpptpaptiasqplslrpeacrpaaggavhtrgldfac |
| | | diyiwaplagtcgvlllslvitlyckrgrkkllyifkqpfmrpvqttqeedg |
| | | cscrfpeeeeggcelrvkfsrsadapaykqgqnqlynelnlgrreeydvldk |
| | | rrgrdpemggkprrknpqeglynelqkdkmaeayseigmkgerrrgkghdgl |
| | | yqglstatkdtydalhmqalppr |

CAR 8

| | | |
|---|---|---|
| CAR8 scFv | 8 | qvqlqesgpglvkpsetlsltctvsgvslpdygvswirqppgkglewigviw |
| domain | | gsettyyqsslksrvtiskdnsknqvslklssvtaadtavyycakhyyyggs |
| | | yamdywgqgtlvtvssggggsggggsggggsggggseivmtqspatlslspg |
| | | eratlscrasqdiskylnwyqqkpgqaprlliyhtsrlhsgiparfsgsgsg |
| | | tdytltisslqpedfavyfcqqgntlpytfgqgtkleik |
| 100798 | 68 | atggcactgcctgtcactgccctcctgctgcctctggccctccttctgcatg |
| CAR8-Soluble | | ccgccaggccccaagtccagctgcaagagtcaggacccggactggtgaagcc |
| scFv-nt | | gtctgagactctctcactgacttgtaccgtcagcggcgtgtccctccccgac |
| | | tacggagtgtcatggatccgccaacctcccgggaaagggcttgaatggattg |
| | | gtgtcatctggggttctgaaaccacctactaccagtcttccctgaagtccag |
| | | ggtgaccatcagcaaggataattccaagaaccaggtcagccttaagctgtca |
| | | tctgtgaccgctgctgacaccgccgtgtattactgcgccaagcactactatt |
| | | acggaggaagctacgctatggactattggggacagggcactctcgtgactgt |
| | | gagcagcggcggtggagggtctggaggtggaggatccggtggtggtgggtca |
| | | ggcggaggagggagcgagattgtgatgactcagtcaccagccaccctttctc |
| | | tttcacccggcgagagagcaaccctgagctgtagagccagccaggacatttc |
| | | taagtacctcaactggtatcagcaaaaaccggggcaggcccctcgcctcctg |
| | | atctaccatacctcacgccttcactctggtatccccgctcggtttagcggat |
| | | caggatctggtaccgactacactctgaccatttccagcctgcagccagaaga |
| | | tttcgcagtgtatttctgccagcagggcaatacccttccttacaccttcggt |
| | | cagggaaccaagctcgaaatcaagcaccatcaccatcatcatcaccac |
| 100798 | 80 | MALPVTALLLPLALLLHAARPqvqlqesgpglvkpsetlsltctvsgvslpd |
| CAR8-Soluble | | ygvswirqppgkglewigviwgsettyyqsslksrvtiskdnsknqvslkls |
| scFv-aa | | svtaadtavyycakhyyyggsyamdywgqgtlvtvssggggsggggsggggs |
| | | ggggseivmtqspatlslspgeratlscrasqdiskylnwyqqkpgqaprll |
| | | iyhtsrlhsgiparfsgsgsgtdytltisslqpedfavyfcqqgntlpytfg |
| | | qgtkleikhhhhhhhh |
| 104882 CAR 8- | 92 | atggctctgcccgtgaccgcactcctcctgccactggctctgctgcttcacg |
| Full-nt | | ccgctcgcccacaagtccagcttcaagaatcagggcctggtctggtgaagcc |
| | | atctgagactctgtccctcacttgcaccgtgagcggagtgtccctcccagac |
| | | tacggagtgagctggattagacagcctcccggaaagggactggagtggatcg |
| | | gagtgatttggggtagcgaaaccacttactatcaatcttccctgaagtcacg |
| | | ggtcaccatttcaaaggataactcaaagaatcaagtgagcctcaagctctca |
| | | tcagtcaccgccgctgacaccgccgtgtattactgtgccaagcattactact |
| | | atggagggtcctacgccatggactactggggccagggaactctggtcactgt |
| | | gtcatctggtggaggaggtagcggaggaggcgggagcggtggaggtggctcc |
| | | ggaggcggtgggtcagaaatcgtgatgacccagagccctgcaaccctgtccc |
| | | tttctcccggggaacgggctaccctttcttgtcgggcatcacaagatatctc |
| | | aaaatacctcaattggtatcaacagaagccgggacaggcccctaggcttctt |
| | | atctaccacacctctcgcctgcatagcgggattcccgcacgctttagcgggt |
| | | ctggaagcgggaccgactacactctgaccatctcatctctccagcccgagga |
| | | cttcgccgtctacttctgccagcagggtaacaccctgccgtacaccttcggc |
| | | cagggcaccaagcttgagatcaaaaccactactcccgctccaaggccaccca |
| | | cccctgccccgaccatcgcctctcagccgctttccctgcgtccggaggcatg |
| | | tagacccgcagctggtggggccgtgcatacccggggtcttgacttcgcctgc |
| | | gatatctacatttgggcccctctggctggtacttgcggggtcctgctgcttt |
| | | cactcgtgatcactctttactgtaagcgcggtcggaagaagctgctgtacat |
| | | ctttaagcaacccttcatgaggcctgtgcagactactcaagaggaggacggc |
| | | tgttcatgccggttcccagaggaggaggaaggcggctgcgaactgcgcgtga |
| | | aattcagccgcagcgcagatgctccagcctacaagcaggggcagaaccagct |
| | | ctacaacgaactcaatcttggtcggagagaggagtacgacgtgctggacaag |
| | | cggagaggacgggacccagaaatgggcgggaagccgcgcagaaagaatcccc |
| | | aagagggcctgtacaacgagctccaaaaggataagatggcagaagcctatag |
| | | cgagattggtatgaaaggggaacgcagaagaggcaaaggccacgacggactg |
| | | taccagggactcagcaccgccaccaaggacacctatgacgctcttcacatgc |
| | | aggccctgccgcctcgg |
| 104882 | 38 | MALPVTALLLPLALLLHAARPqvqlqesgpglvkpsetlsltctvsgvslpd |
| CAR 8-Full-aa | | ygvswirqppgkglewigviwgsettyyqsslksrvtiskdnsknqvslkls |
| | | svtaadtavyycakhyyyggsyamdywgqgtlvtvssggggsggggsggggs |
| | | ggggseivmtqspatlslspgeratlscrasqdiskylnwyqqkpgqaprll |
| | | iyhtsrlhsgiparfsgsgsgtdytltisslqpedfavyfcqqgntlpytfg |
| | | qgtkleiktttpaprpptpaptiasqplslrpeacrpaaggavhtrgldfac |
| | | diyiwaplagtcgvlllslvitlyckrgrkkllyifkqpfmrpvqttqeedg |
| | | cscrfpeeeeggcelrvkfsrsadapaykqgqnqlynelnlgrreeydvldk |
| | | rrgrdpemggkprrknpqeglynelqkdkmaeayseigmkgerrrgkghdgl |
| | | yqglstatkdtydalhmqalppr |

CAR 9

| | | |
|---|---|---|
| CAR9 scFv | 9 | eivmtqspatlslspgeratlscrasqdiskylnwyqqkpgqaprlliyhts |
| domain | | rlhsgiparfsgsgsgtdytltisslqpedfavyfcqqgntlpytfgqgtkl |
| | | eikggggsggggsggggsggggsqvqlqesgpglvkpsetlsltctvsgvsl |
| | | pdygvswirqppgkglewigviwgsettyynsslksrvtiskdnsknqvslk |
| | | lssvtaadtavyycakhyyyggsyamdywgqgtlvtvss |
| 99789 | 69 | atggccctcccagtgaccgctctgctgctgcctctcgcacttcttctccatg |
| CAR9-Soluble | | ccgctcggcctgagatcgtcatgacccaaagccccgctaccctgtccctgtc |
| scFv-nt | | acccggcgagagggcaaccctttcatgcagggccagccaggacatttctaag |
| | | tacctcaactggtatcagcagaagccagggcaggctcctcgcctgctgatct |
| | | accacaccagccgcctccacagcggtatccccgccagattttccgggagcgg |
| | | gtctggaaccgactacaccctcaccatctcttctctgcagcccgaggatttc |
| | | gccgtctatttctgccagcaggggaatactctgccgtacaccttcggtcaag |
| | | gtaccaagctggaaatcaagggaggcggaggatcaggcggtggcggaagcgg |
| | | aggaggtggctccggaggaggaggttcccaagtgcagcttcaagaatcagga |
| | | cccggacttgtgaagccatcagaaaccctctccctgacttgtaccgtgtccg |
| | | gtgtgagcctccccgactacggagtctcttggattcgccagcctccggggaa |
| | | gggtcttgaatggattggggtgatttggggatcagagactacttactacaat |
| | | tcatcacttaagtcacgggtcaccatcagcaaagataatagcaagaaccaag |
| | | tgtcacttaagctgtcatctgtgaccgccgctgacaccgccgtgtactattg |
| | | tgccaaacattactattacggagggtcttatgctatggactactggggacag |
| | | gggaccctggtgactgtctctagccatcaccatcaccaccatcatcac |
| 99789 | 81 | MALPVTALLLPLALLLHAARPeivmtqspatlslspgeratlscrasqdisk |
| CAR9-Soluble | | ylnwyqqkpgqaprlliyhtsrlhsgiparfsgsgsgtdytltisslqpedf |
| scFv-aa | | avyfcqqgntlpytfgqgtkleikggggsggggsggggsggggsqvqlqesg |
| | | pglvkpsetlsltctvsgvslpdygvswirqppgkglewigviwgsettyyn |
| | | sslksrvtiskdnsknqvslklssvtaadtavyycakhyyyggsyamdywgq |
| | | gtlvtvsshhhhhhhh |
| 105974 | 93 | atggccctccctgtcaccgccctgctgcttccgctggctcttctgctccacg |
| CAR 9-Full-nt | | ccgctcggcccgaaattgtgatgacccagtcacccgccactcttagcctttc |
| | | acccggtgagcgcgcaaccctgtcttgcagagcctcccaagacatctcaaaa |
| | | taccttaattggtatcaacagaagcccggacaggctcctcgccttctgatct |
| | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagcggaggcggtgggagccaggtccaactccaagaaagcgga |
| | | ccgggtcttgtgaagccatcagaaactctttcactgacttgtactgtgagcg |
| | | gagtgtctctccccgattacggggtgtcttggatcagacagccaccggggaa |
| | | gggtctggaatggattggagtgatttggggctctgagactacttactacaac |
| | | tcatccctcaagtcacgcgtcaccatctcaaaggacaactctaagaatcagg |
| | | tgtcactgaaactgtcatctgtgaccgcagccgacaccgccgtgtactattg |
| | | cgctaagcattactattatggcgggagctacgcaatggattactggggacag |
| | | ggtactctggtcaccgtgtccagcaccactaccccagcaccgaggccaccca |
| | | ccccggctcctaccatcgcctcccagcctctgtccctgcgtccggaggcatg |
| | | tagacccgcagctggtggggccgtgcatacccggggtcttgacttcgcctgc |
| | | gatatctacatttgggcccctctggctggtacttgcggggtcctgctgcttt |
| | | cactcgtgatcactctttactgtaagcgcggtcggaagaagctgctgtacat |
| | | ctttaagcaacccttcatgaggcctgtgcagactactcaagaggaggacggc |
| | | tgttcatgccggttcccagaggaggaggaaggcggctgcgaactgcgcgtga |
| | | aattcagccgcagcgcagatgctccagcctacaagcaggggcagaaccagct |
| | | ctacaacgaactcaatcttggtcggagagaggagtacgacgtgctggacaag |
| | | cggagaggacgggacccagaaatgggcgggaagccgcgcagaaagaatcccc |
| | | aagagggcctgtacaacgagctccaaaaggataagatggcagaagcctatag |
| | | cgagattggtatgaaaggggaacgcagaagaggcaaaggccacgacggactg |
| | | taccagggactcagcaccgccaccaaggacacctatgacgctcttcacatgc |
| | | aggccctgccgcctcgg |
| 105974 | 39 | MALPVTALLLPLALLLHAARPeivmtqspatlslspgeratlscrasqdisk |
| CAR 9-Full-aa | | ylnwyqqkpgqaprlliyhtsrlhsgiparfsgsgsgtdytltisslqpedf |
| | | avyfcqqgntlpytfgqgtkleikggggsggggsggggsggggsqvqlqesg |
| | | pglvkpsetlsltctvsgvslpdygvswirqppgkglewigviwgsettyyn |
| | | sslksrvtiskdnsknqvslklssvtaadtavyycakhyyyggsyamdywgq |
| | | gtlvtvsstttpaprpptpaptiasqplslrpeacrpaaggavhtrgldfac |
| | | diyiwaplagtcgvlllslvitlyckrgrkkllyifkqpfmrpvqttqeedg |
| | | cscrfpeeeeggcelrvkfsrsadapaykqgqnqlynelnlgrreeydvldk |
| | | rrgrdpemggkprrknpqeglynelqkdkmaeayseigmkgerrrgkghdgl |
| | | yqglstatkdtydalhmqalppr |

CAR10

| | | |
|---|---|---|
| CAR10 scFv | 10 | qvqlqesgpglvkpsetlsltctvsgvslpdygvswirqppgkglewigviw |
| domain | | gsettyynsslksrvtiskdnsknqvslklssvtaadtavyycakhyyyggs |
| | | yamdywgqgtlvtvssggggsggggsggggsggggseivmtqspatlslspg |
| | | eratlscrasqdiskylnwyqqkpgqaprlliyhtsrlhsgiparfsgsgsg |
| | | tdytltisslqpedfavyfcqqgntlpytfgqgtkleik |
| 100796 CAR10- | 70 | atggcactgcctgtcactgccctcctgctgcctctggccctccttctgcatg |
| Soluble scFv-nt | | ccgccaggccccaagtccagctgcaagagtcaggacccggactggtgaagcc |
| | | gtctgagactctctcactgacttgtaccgtcagcggcgtgtccctccccgac |
| | | tacggagtgtcatggatccgccaacctcccgggaaagggcttgaatggattg |
| | | gtgtcatctggggttctgaaaccacctactacaactcttccctgaagtccag |
| | | ggtgaccatcagcaaggataattccaagaaccaggtcagccttaagctgtca |
| | | tctgtgaccgctgctgacaccgccgtgtattactgcgccaagcactactatt |
| | | acggaggaagctacgctatggactattggggacagggcactctcgtgactgt |
| | | gagcagcggcggtggagggtctggaggtggaggatccggtggtggtgggtca |
| | | ggcggaggagggagcgagattgtgatgactcagtcaccagccaccctttctc |
| | | tttcacccggcgagagagcaaccctgagctgtagagccagccaggacatttc |
| | | taagtacctcaactggtatcagcaaaaaccggggcaggcccctcgcctcctg |
| | | atctaccatacctcacgccttcactctggtatccccgctcggtttagcggat |
| | | caggatctggtaccgactacactctgaccatttccagcctgcagccagaaga |
| | | tttcgcagtgtatttctgccagcagggcaatacccttccttacaccttcggt |
| | | cagggaaccaagctcgaaatcaagcaccatcaccatcatcaccaccat |
| 100796 | 82 | MALPVTALLLPLALLLHAARPqvqlqesgpglvkpsetlsltctvsgvslpd |
| CAR10-Soluble | | ygvswirqppgkglewigviwgsettyynsslksrvtiskdnsknqvslkls |
| scFv-aa | | svtaadtavyycakhyyyggsyamdywgqgtlvtvssggggsggggsggggs |
| | | ggggseivmtqspatlslspgeratlscrasqdiskylnwyqqkpgqaprll |
| | | iyhtsrlhsgiparfsgsgsgtdytltisslqpedfavyfcqqgntlpytfg |
| | | qgtkleikhhhhhhhh |
| 105975 CAR 10 | 94 | atggccctccctgtcaccgccctgctgcttccgctggctcttctgctccacg |
| Full-nt | | ccgctcggcccgaaattgtgatgacccagtcacccgccactcttagcctttc |
| | | acccggtgagcgcgcaaccctgtcttgcagagcctcccaagacatctcaaaa |
| | | taccttaattggtatcaacagaagcccggacaggctcctcgccttctgatct |
| | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagcggaggcggtgggagccaggtccaactccaagaaagcgga |
| | | ccgggtcttgtgaagccatcagaaactctttcactgacttgtactgtgagcg |
| | | gagtgtctctccccgattacggggtgtcttggatcagacagccaccggggaa |
| | | gggtctggaatggattggagtgatttggggctctgagactacttactacaac |
| | | tcatccctcaagtcacgcgtcaccatctcaaaggacaactctaagaatcagg |
| | | tgtcactgaaactgtcatctgtgaccgcagccgacaccgccgtgtactattg |
| | | cgctaagcattactattatggcgggagctacgcaatggattactggggacag |
| | | ggtactctggtcaccgtgtccagcaccactaccccagcaccgaggccaccca |
| | | ccccggctcctaccatcgcctcccagcctctgtccctgcgtccggaggcatg |
| | | tagacccgcagctggtggggccgtgcatacccggggtcttgacttcgcctgc |
| | | gatatctacatttgggcccctctggctggtacttgcggggtcctgctgcttt |
| | | cactcgtgatcactctttactgtaagcgcggtcggaagaagctgctgtacat |
| | | ctttaagcaacccttcatgaggcctgtgcagactactcaagaggaggacggc |
| | | tgttcatgccggttcccagaggaggaggaaggcggctgcgaactgcgcgtga |
| | | aattcagccgcagcgcagatgctccagcctacaagcaggggcagaaccagct |
| | | ctacaacgaactcaatcttggtcggagagaggagtacgacgtgctggacaag |
| | | cggagaggacgggacccagaaatgggcgggaagccgcgcagaaagaatcccc |
| | | aagagggcctgtacaacgagctccaaaaggataagatggcagaagcctatag |
| | | cgagattggtatgaaaggggaacgcagaagaggcaaaggccacgacggactg |
| | | taccagggactcagcaccgccaccaaggacacctatgacgctcttcacatgc |
| | | aggccctgccgcctcgg |
| 105975 | 40 | MALPVTAlLLPLALLLHAARPEIVMTQSPATLSLSPGERATLSCRASQDISK |
| CAR 10 Full-aa | | YLNWYQQKPGQAPRLLIYHTSRLHSGIPARFSGSGSGTDYTLTISSLQPEDF |
| | | AVYFCQQGNTLPYTFGQGTKLEIKGGGGSGGGGSGGGGSGGGGSQVQLQESG |
| | | PGLVKPSETLSLTCTVSGVSLPDYGVSWIRQPPGKGLEWIGVIWGSETTYYN |
| | | SSLKSRVTISKDNSKNQVSLKLSSVTAADTAVYYCAKHYYYGGSYAMDYWGQ |
| | | GTLVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQEEDG |
| | | CSCRFPEEEEGGCELRVKFSRSADAPAYKQGQNQLYNELNLGRREEYDVLDK |
| | | RRGRDPEMGGKPRRKNPQEGLYNELQKDKMAEAYSEIGMKGERRRGKGHDGL |
| | | YQGLSTATKDTYDALHMQALPPR |

CAR11

| | | |
|---|---|---|
| CAR11 scFv | 11 | eivmtqspatlslspgeratlscrasqdiskylnwyqqkpgqaprlliyhts |
| domain | | rlhsgiparfsgsgsgtdytltisslqpedfavyfcqqgntlpytfgqgtkl |
| | | eikggggsggggsggggsqvqlqesgpglvkpsetlsltctvsgvslpdygv |
| | | swirqppgkglewigviwgsettyynsslksrvtiskdnsknqvslklssvt |
| | | aadtavyycakhyyyggsyamdywgqgtlvtvss |
| 103101 | 71 | atggccctccctgtcaccgccctgctgcttccgctggctcttctgctccacg |
| CAR11-Soluble | | ccgctcggcccgaaattgtgatgacccagtcacccgccactcttagcctttc |
| scFv-nt | | acccggtgagcgcgcaaccctgtcttgcagagcctcccaagacatctcaaaa |
| | | taccttaattggtatcaacagaagcccggacaggctcctcgccttctgatct |
| | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagccaggtccaactccaagaaagcggaccgggtcttgtgaag |
| | | ccatcagaaactctttcactgacttgtactgtgagcggagtgtctctccccg |
| | | attacggggtgtcttggatcagacagccaccggggaagggtctggaatggat |
| | | tggagtgatttggggctctgagactacttactacaattcatccctcaagtca |
| | | cgcgtcaccatctcaaaggacaactctaagaatcaggtgtcactgaaactgt |
| | | catctgtgaccgcagccgacaccgccgtgtactattgcgctaagcattacta |
| | | ttatggcgggagctacgcaatggattactggggacagggtactctggtcacc |
| | | gtgtccagccaccaccatcatcaccatcaccat |
| 103101 CAR11- | 83 | MALPVTALLLPLALLLHAARPeivmtqspatlslspgeratlscrasqdisk |
| Soluble scFv-aa | | y-nwyqqkpgqaprlliyhtsrlhsgiparfsgsgsgtdytltisslqpedf |
| | | avyfcqqgntlpytfgqgtkleikggggsggggsggggsqvqlqesgpglvk |
| | | psetlsltctvsgvslpdygvswirqppgkglewigviwgsettyynsslks |
| | | rvtiskdnsknqvslklssvtaadtavyycakhyyyggsyamdywgqgtlvt |
| | | vsshhhhhhhh |
| 105976 CAR 11 | 95 | atggctctgcccgtgaccgcactcctcctgccactggctctgctgcttcacg |
| Full-nt | | ccgctcgcccacaagtccagcttcaagaatcagggcctggtctggtgaagcc |
| | | atctgagactctgtccctcacttgcaccgtgagcggagtgtccctcccagac |
| | | tacggagtgagctggattagacagcctcccggaaagggactggagtggatcg |
| | | gagtgatttggggtagcgaaaccacttactataactcttccctgaagtcacg |
| | | ggtcaccatttcaaaggataactcaaagaatcaagtgagcctcaagctctca |
| | | tcagtcaccgccgctgacaccgccgtgtattactgtgccaagcattactact |
| | | atggagggtcctacgccatggactactggggccagggaactctggtcactgt |
| | | gtcatctggtggaggaggtagcggaggaggcgggagcggtggaggtggctcc |
| | | ggaggtggcggaagcgaaatcgtgatgacccagagccctgcaaccctgtccc |
| | | tttctcccggggaacgggctaccctttcttgtcgggcatcacaagatatctc |
| | | aaaatacctcaattggtatcaacagaagccgggacaggcccctaggcttctt |
| | | atctaccacacctctcgcctgcatagcgggattcccgcacgctttagcgggt |
| | | ctggaagcgggaccgactacactctgaccatctcatctctccagcccgagga |
| | | cttcgccgtctacttctgccagcagggtaacaccctgccgtacaccttcggc |
| | | cagggcaccaagcttgagatcaaaaccactactcccgctccaaggccaccca |
| | | cccctgccccgaccatcgcctctcagccgctttccctgcgtccggaggcatg |
| | | tagacccgcagctggtggggccgtgcatacccggggtcttgacttcgcctgc |
| | | gatatctacatttgggcccctctggctggtacttgcggggtcctgctgcttt |
| | | cactcgtgatcactctttactgtaagcgcggtcggaagaagctgctgtacat |
| | | ctttaagcaacccttcatgaggcctgtgcagactactcaagaggaggacggc |
| | | tgttcatgccggttcccagaggaggaggaaggcggctgcgaactgcgcgtga |
| | | aattcagccgcagcgcagatgctccagcctacaagcaggggcagaaccagct |
| | | ctacaacgaactcaatcttggtcggagagaggagtacgacgtgctggacaag |
| | | cggagaggacgggacccagaaatgggcgggaagccgcgcagaaagaatcccc |
| | | aagagggcctgtacaacgagctccaaaaggataagatggcagaagcctatag |
| | | cgagattggtatgaaaggggaacgcagaagaggcaaaggccacgacggactg |
| | | taccagggactcagcaccgccaccaaggacacctatgacgctcttcacatgc |
| | | aggccctgccgcctcgg |
| 105976 | 41 | MALPVTALLLPLALLLHAARPQVQLQESGPGLVKPSETLSLTCTVSGVSLPD |
| CAR 11 Full-aa | | YGVSWIRQPPGKGLEWIGVIWGSETTYYNSSLKSRVTISKDNSKNQVSLKLS |
| | | SVTAADTAVYYCAKHYYYGGSYAMDYWGQGTLVTVSSGGGGSGGGGSGGGGS |
| | | GGGGSEIVMTQSPATLSLSPGERATLSCRASQDISKYLNWYQQKPGQAPRLL |
| | | IYHTSRLHSGIPARFSGSGSGTDYTLTISSLQPEDFAVYFCQQGNTLPYTFG |
| | | QGTKLEIKITTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQEEDG |
| | | CSCRFPEEEEGGCELRVKFSRSADAPAYKQGQNQLYNELNLGRREEYDVLDK |
| | | RRGRDPEMGGKPRRKNPQEGLYNELQKDKMAEAYSEIGMKGERRRGKGHDGL |
| | | YQGLSTATKDTYDALHMQALPPR |

CAR12

| | | |
|---|---|---|
| CAR12 | 12 | qvqlqesgpglvkpsetlsltctvsgvslpdygvswirqppgkglewigviw |
| scFv domain | | gsettyynsslksrvtiskdnsknqvslklssvtaadtavyycakhyyyggs |
| | | yamdywgqgtlvtvssggggsggggsggggseivmtqspatlslspgeratl |
| | | scrasqdiskylnwyqqkpgqaprlliyhtsrlhsgiparfsgsgsgtdytl |
| | | tisslqpedfavyfcqqgntlpytfgqgtkleik |
| 103104 | 72 | atggctctgcccgtgaccgcactcctcctgccactggctctgctgcttcacg |
| CAR12-Soluble | | ccgctcgcccacaagtccagcttcaagaatcagggcctggtctggtgaagcc |
| scFv-nt | | atctgagactctgtccctcacttgcaccgtgagcggagtgtccctcccagac |
| | | tacggagtgagctggattagacagcctcccggaaagggactggagtggatcg |
| | | gagtgatttggggtagcgaaaccacttactataactcttccctgaagtcacg |
| | | ggtcaccatttcaaaggataactcaaagaatcaagtgagcctcaagctctca |
| | | tcagtcaccgccgctgacaccgccgtgtattactgtgccaagcattactact |
| | | atggagggtcctacgccatggactactggggccagggaactctggtcactgt |
| | | gtcatctggtggaggaggtagcggaggaggcgggagcggtggaggtggctcc |
| | | gaaatcgtgatgacccagagccctgcaaccctgtccctttctcccggggaac |
| | | gggctaccctttcttgtcgggcatcacaagatatctcaaaatacctcaattg |
| | | gtatcaacagaagccgggacaggcccctaggcttcttatctaccacacctct |
| | | cgcctgcatagcgggattcccgcacgctttagcgggtctggaagcgggaccg |
| | | actacactctgaccatctcatctctccagcccgaggacttcgccgtctactt |
| | | ctgccagcagggtaacaccctgccgtacaccttcggccagggcaccaagctt |
| | | gagatcaaacatcaccaccatcatcaccatcac |
| 103104 | 84 | MALPVTALLLPLALLLHAARPqvqlqesgpglvkpsetlsltctvsgvslpd |
| CAR12-Soluble | | ygvswirqppgkglewigviwgsettyynsslksrvtiskdnsknqvslkls |
| scFv-aa | | svtaadtavyycakhyyyggsyamdywgqgtlvtvssggggsggggsggggs |
| | | eivmtqspatlslspgeratlscrasqdiskylnwyqqkpgqaprlliyhts |
| | | rlhsgiparfsgsgsgtdytltisslqpedfavyfcqqgntlpytfgqgtkl |
| | | eikhhhhhhhh |
| 105977 | 96 | atggccctccctgtcaccgccctgctgcttccgctggctcttctgctccacg |
| CAR 12-Full- | | ccgctcggcccgaaattgtgatgacccagtcacccgccactcttagcctttc |
| nt | | acccggtgagcgcgcaaccctgtcttgcagagcctcccaagacatctcaaaa |
| | | taccttaattggtatcaacagaagcccggacaggctcctcgccttctgatct |
| | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagccaggtccaactccaagaaagcggaccgggtcttgtgaag |
| | | ccatcagaaactctttcactgacttgtactgtgagcggagtgtctctccccg |
| | | attacggggtgtcttggatcagacagccaccggggaagggtctggaatggat |
| | | tggagtgatttggggctctgagactacttactacaactcatccctcaagtca |
| | | cgcgtcaccatctcaaaggacaactctaagaatcaggtgtcactgaaactgt |
| | | catctgtgaccgcagccgacaccgccgtgtactattgcgctaagcattacta |
| | | ttatggcgggagctacgcaatggattactggggacagggtactctggtcacc |
| | | gtgtccagcaccactaccccagcaccgaggccacccaccccggctcctacca |
| | | tcgcctcccagcctctgtccctgcgtccggaggcatgtagacccgcagctgg |
| | | tggggccgtgcatacccggggtcttgacttcgcctgcgatatctacatttgg |
| | | gcccctctggctggtacttgcggggtcctgctgctttcactcgtgatcactc |
| | | tttactgtaagcgcggtcggaagaagctgctgtacatctttaagcaaccctt |
| | | catgaggcctgtgcagactactcaagaggaggacggctgttcatgccggttc |
| | | ccagaggaggaggaaggcggctgcgaactgcgcgtgaaattcagccgcagcg |
| | | cagatgctccagcctacaagcaggggcagaaccagctctacaacgaactcaa |
| | | tcttggtcggagagaggagtacgacgtgctggacaagcggagaggacgggac |
| | | ccagaaatgggcgggaagccgcgcagaaagaatccccaagagggcctgtaca |
| | | acgagctccaaaaggataagatggcagaagcctatagcgagattggtatgaa |
| | | aggggaacgcagaagaggcaaaggccacgacggactgtaccagggactcagc |
| | | accgccaccaaggacacctatgacgctcttcacatgcaggccctgccgcctc |
| | | gg |
| 105977 | 42 | MALPVTALLLPLALLLHAARPEIVMTQSPATLSLSPGERATLSCRASQDISK |
| CAR 12-Full- | | YLNWYQQKPGQAPRLLIVHTSRLHSGIPARFSGSGSGTDYTLTISSLQPEDF |
| aa | | AVYFCQQGNTLPYTFGQGTKLEIKGGGGSGGGGSGGGGSQVQLQESGPGLVK |
| | | PSETLSLTCTVSGVSLPDYGVSWIRQPPGKGLEWIGVIWGSETTYYNSSLKS |
| | | RVTISKDNSKNQVSLKLSSVTAADTAVYYCAKHYYYGGSYAMDYWGQGTLVT |
| | | VSSTTTPAPRPPTPAPTIASQPLSLRPEACRPAAGGAVHTRGLDFACDIYIW |
| | | APLAGTCGVLLLSLVITLYCKRGRKKLLYIFKQPFMRPVQTTQEEDGCSCRF |
| | | PEEEEGGCELRVKFSRSADAPAYKQGQNQLYNELNLGRREEYDVLDKRRGRD |
| | | PEMGGKPRRKNPQEGLYNELQKDKMAEAYSEIGMKGERRRGKGHDGLYQGLS |
| | | TATKDTYDALHMQALPPR |

CAR2A

| | | |
|---|---|---|
| CAR 2A- | 5028 | MALPVTALLLPLALLLHAARPEIVMTQSPATLSLSPGERATLSCRASQDISK |
| Full amino acid | | YLNWYQQKPGQAPRLLIYHTSRLHSGIPARFSGSGSGTDYTLTISSLQPEDF |
| sequence; signal | | AVYFCQQGNTLPYTFGQGTKLEIKGGGGSGGGGSGGGGSQVQLQESGPGLVK |
| peptide | | PSETLSLTCTVSGVSLPDYGVSWIRQPPGKGLEWIGVIWGSETTYYQSSLKS |
| underlined | | RVTISKDNSKNQVSLKLSSVTAADTAVYYCAKHYYYGGSYAMDYWGQGTLVT |
| | | VSSTTTPAPRPPTPAPTIASQPLSLRPEACRPAAGGAVHTRGLDFACDIYIW |
| | | APLAGTCGVLLLSLVITLYCKRGRKKLLYIFKQPFMRPVQTTQEEDGCSCRF |
| | | PEEEEGGCELRVKFSRSADAPAYQQGQNQLYNELNLGRREEYDVLDKRRGRD |
| | | PEMGGKPRRKNPQEGLYNELQKDKMAEAYSEIGMKGERRRGKGHDGLYQGLS |
| | | TATKDTYDALHMQALPPR |
| CAR 2A- | 5029 | EIVMTQSPATLSLSPGERATLSCRASQDISKYLNWYQQKPGQAPRLLIYHTS |
| amino acid | | RLHSGIPARFSGSGSGTDYTLTISSLQPEDFAVYFCQQGNTLPYTFGQGTKL |
| sequence; no | | EIKGGGGSGGGGSGGGGSQVQLQESGPGLVKPSETLSLTCTVSGVSLPDYGV |
| signal peptide | | SWIRQPPGKGLEWIGVIWGSETTYYQSSLKSRVTISKDNSKNQVSLKLSSVT |
| | | AADTAVYYCAKHYYYGGSYAMDYWGQGTLVTVSSTTTPAPRPPTPAPTIASQ |
| | | PLSLRPEACRPAAGGAVHTRGLDFACDIYIWAPLAGTCGVLLLSLVITLYCK |
| | | RGRKKLLYIFKQPFMRPVQTTQEEDGCSCRFPEEEEGGCELRVKFSRSADAP |
| | | AYQQGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| CAR 2A full | 5030 | atggccctccctgtcaccgccctgctgcttccgctggctcttctgctccacg |
| nucleic acid | | ccgctcggcccgaaattgtgatgacccagtcacccgccactcttagcctttc |
| sequence; signal | | acccggtgagcgcgcaaccctgtcttgcagagcctcccaagacatctcaaaa |
| peptide and stop | | taccttaattggtatcaacagaagcccggacaggctcctcgccttctgatct |
| codon underlined | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagccaggtccaactccaagaaagcggaccgggtcttgtgaag |
| | | ccatcagaaactctttcactgacttgtactgtgagcggagtgtctctccccg |
| | | attacggggtgtcttggatcagacagccaccggggaagggtctggaatggat |
| | | tggagtgatttggggctctgagactacttactaccaatcatccctcaagtca |
| | | cgcgtcaccatctcaaaggacaactctaagaatcaggtgtcactgaaactgt |
| | | catctgtgaccgcagccgacaccgccgtgtactattgcgctaagcattacta |
| | | ttatggcgggagctacgcaatggattactggggacagggtactctggtcacc |
| | | gtgtccagcaccactaccccagcaccgaggccacccaccccggctcctacca |
| | | tcgcctcccagcctctgtccctgcgtccggaggcatgtagacccgcagctgg |
| | | tggggccgtgcatacccggggtcttgacttcgcctgcgatatctacatttgg |
| | | gcccctctggctggtacttgcggggtcctgctgctttcactcgtgatcactc |
| | | tttactgtaagcgcggtcggaagaagctgctgtacatctttaagcaaccctt |
| | | catgaggcctgtgcagactactcaagaggaggacggctgttcatgccggttc |
| | | ccagaggaggaggaaggcggctgcgaactgcgcgtgaaattcagccgcagcg |
| | | cagatgctccagcctaccagcaggggcagaaccagctctacaacgaactcaa |
| | | tcttggtcggagagaggagtacgacgtgctggacaagcggagaggacgggac |
| | | ccagaaatgggcgggaagccgcgcagaaagaatccccaagagggcctgtaca |
| | | acgagctccaaaaggataagatggcagaagcctatagcgagattggtatgaa |
| | | aggggaacgcagaagaggcaaaggccacgacggactgtaccagggactcagc |
| | | accgccaccaaggacacctatgacgctcttcacatgcaggccctgccgcctc |
| | | ggtaa |
| CAR 2A nucleic | 5031 | atggccctccctgtcaccgccctgctgcttccgctggctcttctgctccacg |
| acid sequence; | | ccgctcggcccgaaattgtgatgacccagtcacccgccactcttagcctttc |
| signal peptide | | acccggtgagcgcgcaaccctgtcttgcagagcctcccaagacatctcaaaa |
| underlined; no | | taccttaattggtatcaacagaagcccggacaggctcctcgccttctgatct |
| stop codon | | accacaccagccggctccattctggaatccctgccaggttcagcggtagcgg |
| | | atctgggaccgactacaccctcactatcagctcactgcagccagaggacttc |
| | | gctgtctatttctgtcagcaagggaacaccctgccctacacctttggacagg |
| | | gcaccaagctcgagattaaaggtggaggtggcagcggaggaggtgggtccgg |
| | | cggtggaggaagccaggtccaactccaagaaagcggaccgggtcttgtgaag |
| | | ccatcagaaactctttcactgacttgtactgtgagcggagtgtctctccccg |
| | | attacggggtgtcttggatcagacagccaccggggaagggtctggaatggat |
| | | tggagtgatttggggctctgagactacttactaccaatcatccctcaagtca |
| | | cgcgtcaccatctcaaaggacaactctaagaatcaggtgtcactgaaactgt |
| | | catctgtgaccgcagccgacaccgccgtgtactattgcgctaagcattacta |
| | | ttatggcgggagctacgcaatggattactggggacagggtactctggtcacc |
| | | gtgtccagcaccactaccccagcaccgaggccacccaccccggctcctacca |
| | | tcgcctcccagcctctgtccctgcgtccggaggcatgtagacccgcagctgg |
| | | tggggccgtgcatacccggggtcttgacttcgcctgcgatatctacatttgg |
| | | gcccctctggctggtacttgcggggtcctgctgctttcactcgtgatcactc |
| | | tttactgtaagcgcggtcggaagaagctgctgtacatctttaagcaaccctt |
| | | catgaggcctgtgcagactactcaagaggaggacggctgttcatgccggttc |
| | | ccagaggaggaggaaggcggctgcgaactgcgcgtgaaattcagccgcagcg |
| | | cagatgctccagcctaccagcaggggcagaaccagctctacaacgaactcaa |
| | | tcttggtcggagagaggagtacgacgtgctggacaagcggagaggacgggac |
| | | ccagaaatgggcgggaagccgcgcagaaagaatccccaagagggcctgtaca |
| | | acgagctccaaaaggataagatggcagaagcctatagcgagattggtatgaa |
| | | aggggaacgcagaagaggcaaaggccacgacggactgtaccagggactcagc |
| | | accgccaccaaggacacctatgacgctcttcacatgcaggccctgccgcctc |
| | | gg |
| CAR 2A nucleic | 5032 | gaaattgtgatgacccagtcacccgccactcttagcctttcacccggtgagc |
| acid sequence; no | | gcgcaaccctgtcttgcagagcctcccaagacatctcaaaataccttaattg |
| signal peptide; | | gtatcaacagaagcccggacaggctcctcgccttctgatctaccacaccagc |
| stop codon | | cggctccattctggaatccctgccaggttcagcggtagcggatctgggaccg |
| underlined | | actacaccctcactatcagctcactgcagccagaggacttcgctgtctattt |
| | | ctgtcagcaagggaacaccctgccctacacctttggacagggcaccaagctc |
| | | gagattaaaggtggaggtggcagcggaggaggtgggtccggcggtggaggaa |
| | | gccaggtccaactccaagaaagcggaccgggtcttgtgaagccatcagaaac |
| | | tctttcactgacttgtactgtgagcggagtgtctctccccgattacggggtg |
| | | tcttggatcagacagccaccggggaagggtctggaatggattggagtgattt |
| | | ggggctctgagactacttactaccaatcatccctcaagtcacgcgtcaccat |
| | | ctcaaaggacaactctaagaatcaggtgtcactgaaactgtcatctgtgacc |
| | | gcagccgacaccgccgtgtactattgcgctaagcattactattatggcggga |
| | | gctacgcaatggattactggggacagggtactctggtcaccgtgtccagcac |
| | | cactaccccagcaccgaggccacccaccccggctcctaccatcgcctcccag |
| | | cctctgtccctgcgtccggaggcatgtagacccgcagctggtggggccgtgc |
| | | atacccggggtcttgacttcgcctgcgatatctacatttgggcccctctggc |
| | | tggtacttgcggggtcctgctgctttcactcgtgatcactctttactgtaag |
| | | cgcggtcggaagaagctgctgtacatctttaagcaacccttcatgaggcctg |
| | | tgcagactactcaagaggaggacggctgttcatgccggttcccagaggagga |
| | | ggaaggcggctgcgaactgcgcgtgaaattcagccgcagcgcagatgctcca |
| | | gcctaccagcaggggcagaaccagctctacaacgaactcaatcttggtcgga |
| | | gagaggagtacgacgtgctggacaagcggagaggacgggacccagaaatggg |
| | | cgggaagccgcgcagaaagaatccccaagagggcctgtacaacgagctccaa |
| | | aaggataagatggcagaagcctatagcgagattggtatgaaaggggaacgca |
| | | gaagaggcaaaggccacgacggactgtaccagggactcagcaccgccaccaa |
| | | ggacacctatgacgctcttcacatgcaggecctgccgcctcggtaa |
| CAR 2A nucleic | 5033 | gaaattgtgatgacccagtcacccgccactcttagcctttcacccgg |
| acid sequence; no | | tgagcgcgcaaccctgtcttgcagagcctcccaagacatctcaaaat |
| signal peptide; no | | accttaattggtatcaacagaagcccggacaggctcctcgccttctg |
| stop codon | | atctaccacaccagccggctccattctggaatccctgccaggttcag |
| | | cggtagcggatctgggaccgactacaccctcactatcagctcactgc |
| | | agccagaggacttcgctgtctatttctgtcagcaagggaacaccctg |
| | | ccctacacctttggacagggcaccaagctcgagattaaaggtggagg |
| | | tggcagcggaggaggtgggtccggcggtggaggaagccaggtccaac |
| | | tccaagaaagcggaccgggtcttgtgaagccatcagaaactctttca |
| | | ctgacttgtactgtgagcggagtgtctctccccgattacggggtgtc |
| | | ttggatcagacagccaccggggaagggtctggaatggattggagtga |
| | | tttggggctctgagactacttactaccaatcatccctcaagtcacgc |
| | | gtcaccatctcaaaggacaactctaagaatcaggtgtcactgaaact |
| | | gtcatctgtgaccgcagccgacaccgccgtgtactattgcgctaagc |
| | | attactattatggcgggagctacgcaatggattactggggacagggt |
| | | actctggtcaccgtgtccagcaccactaccccagcaccgaggccacc |
| | | caccccggctcctaccatcgcctcccagcctctgtccctgcgtccgg |
| | | aggcatgtagacccgcagctggtggggccgtgcatacccggggtctt |
| | | gacttcgcctgcgatatctacatttgggcccctctggctggtacttg |
| | | cggggtcctgctgctttcactcgtgatcactctttactgtaagcgcg |
| | | gtcggaagaagctgctgtacatctttaagcaacccttcatgaggcct |
| | | gtgcagactactcaagaggaggacggctgttcatgccggttcccaga |
| | | ggaggaggaaggcggctgcgaactgcgcgtgaaattcagccgcagcg |
| | | cagatgctccagcctaccagcaggggcagaaccagctctacaacgaa |
| | | ctcaatcttggtcggagagaggagtacgacgtgctggacaagcggag |
| | | aggacgggacccagaaatgggcgggaagccgcgcagaaagaatcccc |
| | | aagagggcctgtacaacgagctccaaaaggataagatggcagaagcc |
| | | tatagcgagattggtatgaaaggggaacgcagaagaggcaaaggcca |
| | | cgacggactgtaccagggactcagcaccgccaccaaggacacctatg |
| | | acgctcttcacatgcaggccctgccgcctcgg |

CAR A

| | | |
|---|---|---|
| CAR A-Full | 5000 | atgcttctcctggtgacaagccttctgctctgtgagttaccacacccagcat |
| nucleotide | | tcctcctgatcccagacatccagatgacacagactacatcctccctgtctgc |
| sequence | | ctctctgggagacagagtcaccatcagttgcagggcaagtcaggacattagt |
| | | aaatatttaaattggtatcagcagaaaccagatggaactgttaaactcctga |
| | | tctaccatacatcaagattacactcaggagtcccatcaaggttcagtggcag |
| | | tgggtctggaacagattattctctcaccattagcaacctggagcaagaagat |
| | | attgccacttacttttgccaacagggtaatacgcttccgtacacgttcggag |
| | | gggggactaagttggaaataacaggctccacctctggatccggcaagcccgg |
| | | atctggcgagggatccaccaagggcgaggtgaaactgcaggagtcaggacct |
| | | ggcctggtggcgccctcacagagcctgtccgtcacatgcactgtctcagggg |
| | | tctcattacccgactatggtgtaagctggattcgccagcctccacgaaaggg |
| | | tctggagtggctgggagtaatatggggtagtgaaaccacatactataattca |
| | | gctctcaaatccagactgaccatcatcaaggacaactccaagagccaagttt |
| | | tcttaaaaatgaacagtctgcaaactgatgacacagccatttactactgtgc |
| | | caaacattattactacggtggtagctatgctatggactactggggtcaagga |
| | | acctcagtcaccgtctcctcagcggccgcaattgaagttatgtatcctcctc |
| | | cttacctagacaatgagaagagcaatggaaccattatccatgtgaaagggaa |
| | | acacctttgtccaagtcccctatttcccggaccttctaagcccttttgggtg |
| | | ctggtggtggttgggggagtcctggcttgctatagcttgctagtaacagtgg |
| | | cctttattattttctgggtgaggagtaagaggagcaggctcctgcacagtga |
| | | ctacatgaacatgactccccgccgccccgggcccacccgcaagcattaccag |
| | | ccctatgccccaccacgcgacttcgcagcctatcgctccagagtgaagttca |
| | | gcaggagcgcagacgcccccgcgtaccagcagggccagaaccagctctataa |
| | | cgagctcaatctaggacgaagagaggagtacgatgttttggacaagagacgt |
| | | ggccgggaccctgagatggggggaaagccgagaaggaagaaccctcaggaag |
| | | gcctgtacaatgaactgcagaaagataagatggcggaggcctacagtgagat |
| | | tgggatgaaaggcgagcgccggaggggcaaggggcacgatggcctttaccag |
| | | ggtctcagtacagccaccaaggacacctacgacgcccttcacatgcaggccc |
| | | tgccccctcgc |
| CAR A-Full AA | 5001 | MLLLVTSLLLCELPHPAFLLIPDIQMTQTTSSLSASLGDRVTISCRASQDIS |
| transgene | | KYLNWYQQKPDGTVKLLIYHTSRLHSGVPSRFSGSGSGTDYSLTISNLEQED |
| sequence | | IATYFCQQGNTLPYTFGGGTKLEITGSTSGSGKPGSGEGSTKGEVKLQESGP |
| | | GLVAPSQSLSVTCTVSGVSLPDYGVSWIRQPPRKGLEWLGVIWGSETTYYNS |
| | | ALKSRLTIIKDNSKSQVFLKMNSLQTDDTAIYYCAKHYYYGGSYAMDYWGQG |
| | | TSVTVSSAAAIEVMYPPPYLDNEKSNGTIIHVKGKHLCPSPLFPGPSKPFWV |
| | | LVVVGGVLACYSLLVTVAFIIFWVRSKRSRLLHSDYMNMTPRRPGPTRKHYQ |
| | | PYAPPRDFAAYRSRVKFSRSADAPAYQQGQNQLYNELNLGRREEYDVLDKRR |
| | | GRDPEMGGKPRRKNPQEGLYNELQKDKMAEAYSEIGMKGERRRGKGHDGLYQ |
| | | GLSTATKDTYDALHMQALPPR |
| CAR A-CD19 | 5009 | atgcttctcctggtgacaagccttctgctctgtgagttaccacacccagcat |
| scFv NT | | tcctcctgatcccagacatccagatgacacagactacatcctccctgtctgc |
| sequence | | ctctctgggagacagagtcaccatcagttgcagggcaagtcaggacattagt |
| | | aaatatttaaattggtatcagcagaaaccagatggaactgttaaactcctga |
| | | tctaccatacatcaagattacactcaggagtcccatcaaggttcagtggcag |
| | | tgggtctggaacagattattctctcaccattagcaacctggagcaagaagat |
| | | attgccacttacttttgccaacagggtaatacgcttccgtacacgttcggag |
| | | gggggactaagttggaaataacaggctccacctctggatccggcaagcccgg |
| | | atctggcgagggatccaccaagggcgaggtgaaactgcaggagtcaggacct |
| | | ggcctggtggcgccctcacagagcctgtccgtcacatgcactgtctcagggg |
| | | tctcattacccgactatggtgtaagctggattcgccagcctccacgaaaggg |
| | | tctggagtggctgggagtaatatggggtagtgaaaccacatactataattca |
| | | gctctcaaatccagactgaccatcatcaaggacaactccaagagccaagttt |
| | | tcttaaaaatgaacagtctgcaaactgatgacacagccatttactactgtgc |
| | | caaacattattactacggtggtagctatgctatggactactggggtcaagga |
| | | acctcagtcaccgtctcctca |
| CAR A-CD19 | 5002 | MLLLVTSLLLCELPHPAFLLIPDIQMTQTTSSLSASLGDRVTISCRASQDIS |
| scFv AA | | KYLNWYQQKPDGTVKLLIYHTSRLHSGVPSRFSGSGSGTDYSLTISNLEQED |
| sequence | | IATYFCQQGNTLPYTFGGGTKLEITGSTSGSGKPGSGEGSTKGEVKLQESGP |
| | | GLVAPSQSLSVTCTVSGVSLPDYGVSWIRQPPRKGLEWLGVIWGSETTYYNS |
| | | ALKSRITIIKDNSKSQVFLKMNSLQTDDTAIYYCAKHYYYGGSYAMDYWGQG |
| | | TSVTVS |
| CAR A-Full | 5010 | gacatccagatgacacagactacatcctccctgtctgcctctctgggagaca |
| nucleotide | | gagtcaccatcagttgcagggcaagtcaggacattagtaaatatttaaattg |
| sequence | | gtatcagcagaaaccagatggaactgttaaactcctgatctaccatacatca |
| | | agattacactcaggagtcccatcaaggttcagtggcagtgggtctggaacag |
| | | attattctctcaccattagcaacctggagcaagaagatattgccacttactt |
| | | ttgccaacagggtaatacgcttccgtacacgttcggaggggggactaagttg |
| | | gaaataacaggctccacctctggatccggcaagcccggatctggcgagggat |
| | | ccaccaagggcgaggtgaaactgcaggagtcaggacctggcctggtggcgcc |
| | | ctcacagagcctgtccgtcacatgcactgtctcaggggtctcattacccgac |
| | | tatggtgtaagctggattcgccagcctccacgaaagggtctggagtggctgg |
| | | gagtaatatggggtagtgaaaccacatactataattcagctctcaaatccag |
| | | actgaccatcatcaaggacaactccaagagccaagttttcttaaaaatgaac |
| | | agtctgcaaactgatgacacagccatttactactgtgccaaacattattact |
| | | acggtggtagctatgctatggactactggggtcaaggaacctcagtcaccgt |
| | | ctcctcagcggccgcaattgaagttatgtatcctcctccttacctagacaat |
| | | gagaagagcaatggaaccattatccatgtgaaagggaaacacctttgtccaa |
| | | gtcccctatttcccggaccttctaagcccttttgggtgctggtggtggttgg |
| | | gggagtcctggcttgctatagcttgctagtaacagtggcctttattattttc |
| | | tgggtgaggagtaagaggagcaggctcctgcacagtgactacatgaacatga |
| | | ctccccgccgccccgggcccacccgcaagcattaccagccctatgccccacc |
| | | acgcgacttcgcagcctatcgctccagagtgaagttcagcaggagcgcagac |
| | | gcccccgcgtaccagcagggccagaaccagctctataacgagctcaatctag |
| | | gacgaagagaggagtacgatgttttggacaagagacgtggccgggaccctga |
| | | gatggggggaaagccgagaaggaagaaccctcaggaaggcctgtacaatgaa |
| | | ctgcagaaagataagatggcggaggcctacagtgagattgggatgaaaggcg |
| | | agcgccggaggggcaaggggcacgatggcctttaccagggtctcagtacagc |
| | | caccaaggacacctacgacgcccttcacatgcaggccctgccccctcgc |
| CAR A-Full AA | 5011 | DIQMTQTTSSLSASLGDRVTISCRASQDISKYLNWYQQKPDGTVKLLIYHTS |
| transgene | | RLHSGVPSRFSGSGSGTDYSLTISNLEQEDIATYFCQQGNTLPYTFGGGTKL |
| sequence | | EZTGSTSGSGKPGSGEGSTKGEVKLQESGPGLVAPSQSLSVTCTVSGVSLPD |
| | | YGVSWIRQPPRKGLEWLGVIWGSETTYYNSALKSRLTIIKDNSKSQVFLKMN |
| | | SLQTDDTAIYYCAKHYYYGGSYAMDYWGQGTSVTVSSAAAIEVMYPPPYLDN |
| | | EKSNGTIIHVKGKHLCPSPLFPGPSKPFWVLVVVGGVLACYSLLVTVAFIIF |
| | | WVRSKRSRLLHSDYMNMZPRRPGPTRKHYQPYAPPRDFAAYRSRVKFSRSAD |
| | | APAYQQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALPPR |
| CAR A-CD19 | 5012 | gacatccagatgacacagactacatcctccctgtctgcctctctgggagaca |
| scFv NT | | gagtcaccatcagttgcagggcaagtcaggacattagtaaatatttaaattg |
| sequence | | gtatcagcagaaaccagatggaactgttaaactcctgatctaccatacatca |
| | | agattacactcaggagtcccatcaaggttcagtggcagtgggtctggaacag |
| | | attattctctcaccattagcaacctggagcaagaagatattgccacttactt |
| | | ttgccaacagggtaatacgcttccgtacacgttcggaggggggactaagttg |
| | | gaaataacaggctccacctctggatccggcaagcccggatctggcgagggat |
| | | ccaccaagggcgaggtgaaactgcaggagtcaggacctggcctggtggcgcc |
| | | ctcacagagcctgtccgtcacatgcactgtctcaggggtctcattacccgac |
| | | tatggtgtaagctggattcgccagcctccacgaaagggtctggagtggctgg |
| | | gagtaatatggggtagtgaaaccacatactataattcagctctcaaatccag |
| | | actgaccatcatcaaggacaactccaagagccaagttttcttaaaaatgaac |
| | | agtctgcaaactgatgacacagccatttactactgtgccaaacattattact |
| | | acggtggtagctatgctatggactactggggtcaaggaacctcagtcaccgt |
| | | ctcctca |
| CAR A-CD19 | 5013 | DIQMTQTTSSLSASLGDRVTISCRASQDISKYLNWYQQKPDGTVKLLIYHTS |
| scFv AA | | RLHSGVPSRFSGSGSGTDYSLTISNLEQEDIATYFCQQGNTLPYTFGGGTKL |
| sequence | | EZTGSTSGSGKPGSGEGSTKGEVKLQESGPGLVAPSQSLSVTCTVSGVSLPD |
| | | YGVSWIRQPPRKGLEWLGVIWGSETTYYNSALKSRLTIIKDNSKSQVFLKMN |
| | | SLQTDDTAIYYCAKHYYYGGSYAMDYWGQGTSVTVSS |

CAR B

| | | |
|---|---|---|
| CAR B full NT | 5003 | ATGCTGCTGCTGGTGACCAGCCTGCTGCTGTGCGAGCTGCCCCACCCCGCCT |
| sequence | | TTCTGCTGATCCCCGACATCCAGATGACCCAGACCACCTCCAGCCTGAGCGC |
| | | CAGCCTGGGCGACCGGGTGACCATCAGCTGCCGGGCCAGCCAGGACATCAGC |
| | | AAGTACCTGAACTGGTATCAGCAGAAGCCCGACGGCACCGTCAAGCTGCTGA |
| | | TCTACCACACCAGCCGGCTGCACAGCGGCGTGCCCAGCCGGTTTAGCGGCAG |
| | | CGGCTCCGGCACCGACTACAGCCTGACCATCTCCAACCTGGAACAGGAAGAT |
| | | ATCGCCACCTACTTTTGCCAGCAGGGCAACACACTGCCCTACACCTTTGGCG |
| | | GCGGAACAAAGCTGGAAATCACCGGCAGCACCTCCGGCAGCGGCAAGCCTGG |
| | | CAGCGGCGAGGGCAGCACCAAGGGCGAGGTGAAGCTGCAGGAAAGCGGCCCT |
| | | GGCCTGGTGGCCCCCAGCCAGAGCCTGAGCGTGACCTGCACCGTGAGCGGCG |
| | | TGAGCCTGCCCGACTACGGCGTGAGCTGGATCCGGCAGCCCCCCAGGAAGGG |
| | | CCTGGAATGGCTGGGCGTGATCTGGGGCAGCGAGACCACCTACTACAACAGC |
| | | GCCCTGAAGAGCCGGCTGACCATCATCAAGGACAACAGCAAGAGCCAGGTGT |
| | | TCCTGAAGATGAACAGCCTGCAGACCGACGACACCGCCATCTACTACTGCGC |
| | | CAAGCACTACTACTACGGCGGCAGCTACGCCATGGACTACTGGGGCCAGGGC |
| | | ACCAGCGTGACCGTGAGCAGCGAATCTAAGTACGGACCGCCCTGCCCCCCTT |
| | | GCCCTATGTTCTGGGTGCTGGTGGTGGTCGGAGGCGTGCTGGCCTGCTACAG |
| | | CCTGCTGGTCACCGTGGCCTTCATCATCTTTTGGGTGAAACGGGGCAGAAAG |
| | | AAACTCCTGTATATATTCAAACAACCATTTATGAGACCAGTACAAACTACTC |
| | | AAGAGGAAGATGGCTGTAGCTGCCGATTTCCAGAAGAAGAAGAAGGAGGATG |
| | | TGAACTGCGGGTGAAGTTCAGCAGAAGCGCCGACGCCCCTGCCTACCAGCAG |
| | | GGCCAGAATCAGCTGTACAACGAGCTGAACCTGGGCAGAAGGGAAGAGTACG |
| | | ACGTCCTGGATAAGCGGAGAGGCCGGGACCCTGAGATGGGCGGCAAGCCTCG |
| | | GCGGAAGAACCCCCAGGAAGGCCTGTATAACGAACTGCAGAAAGACAAGATG |
| | | GCCGAGGCCTACAGCGAGATCGGCATGAAGGGCGAGCGGAGGCGGGGCAAGG |
| | | GCCACGACGGCCTGTATCAGGGCCTGTCCACCGCCACCAAGGATACCTACGA |
| | | CGCCCTGCACATGCAGGCCCTGCCCCCAAGG |
| CAR B full AA | 5004 | MLLLVTSLLLCELPHPAFLLIPDIQMTQTTSSLSASLGDRVTISCRASQDIS |
| transgene | | KYLNWYQQKPDGTVKLLIYHTSRLHSGVPSRFSGSGSGTDYSLTISNLEQED |
| sequence | | IATYFCQQGNTLPYTFGGGTKLEITGSTSGSGKPGSGEGSTKGEVKLQESGP |
| | | GLVAPSQSLSVTCTVSGVSLPDYGVSWIRQPPRKGLEWLGVIWGSETTYYNS |
| | | ALKSRLTIIKDNSKSQVFLKMNSLQTDDTAIYYCAKHYYYGGSYAMDYWGQG |
| | | TSVTVSSESKYGPPCPPCPMFWVLVVVGGVLACYSLLVTVAFIIFWVKRGRK |
| | | KLLYIFKQPFMRPVQTTQEEDGCSCRFPEEEEGGCELRVKFSRSADAPAYQQ |
| | | GQNQLYNELNLGRREEYDVLDKRRGRDPEMGGKPRRKNPQEGLYNELQKDKM |
| | | AEAYSEIGMKGERRRGKGHDGLYQGLSTATKDTYDALHMQALPPR |
| CAR B CD19 | 5014 | ATGCTGCTGCTGGTGACCAGCCTGCTGCTGTGCGAGCTGCCCCACCCCGCCT |
| scFv NT | | TTCTGCTGATCCCCGACATCCAGATGACCCAGACCACCTCCAGCCTGAGCGC |
| sequence | | CAGCCTGGGCGACCGGGTGACCATCAGCTGCCGGGCCAGCCAGGACATCAGC |
| | | AAGTACCTGAACTGGTASCAGCAGAAGCCCGACGGCACCGTCAAGCTGCTGA |
| | | TCTACCACACCAGCCGGCTGCACAGCGGCGTGCCCAGCCGGTTTAGCGGCAG |
| | | CGGCTCCGGCACCGACTACAGCCTGACCATCTCCAACCTGGAACAGGAAGAT |
| | | ATCGCCACCTACTTTTGCCAGCAGGGCAACACACTGCCCTACACCTTTGGCG |
| | | GCGGAACAAAGCTGGAAATCACCGGCAGCACCTCCGGCAGCGGCAAGCCTGG |
| | | CAGCGGCGAGGGCAGCACCAAGGGCGAGGTGAAGCTGCAGGAAAGCGGCCCT |
| | | GGCCTGGTGGCCCCCAGCCAGAGCCTGAGCGTGACCTGCACCGTGAGCGGCG |
| | | TGAGCCTGCCCGACTACGGCGTGAGCTGGATCCGGCAGCCCCCCAGGAAGGG |
| | | CCTGGAATGGCTGGGCGTGATCTGGGGCAGCGAGACCACCTACTACAACAGC |
| | | GCCCTGAAGAGCCGGCTGACCATCATCAAGGACAACAGCAAGAGCCAGGTGT |
| | | TCCTGAAGATGAACAGCCTGCAGACCGACGACACCGCCATCTACTACTGCGC |
| | | CAAGCACTACTACTACGGCGGCAGCTACGCCATGGACTACTGGGGCCAGGGC |
| | | ACCAGCGTGACCGTGAGCAGC |
| CAR B CD19 | 5005 | MLLLVTSLLLCELPHPAFLLIPDIQMTQTTSSLSASLGDRVTISCRASQDIS |
| scFv AA | | KYLNWYQQKPDGTVKLLIYHTSRLHSGVPSRFSGSGSGTDYSLTISNLEQED |
| sequence | | IATYFCQQGNTLPYTFGGGTKLEITGSTSGSGKPGSGEGSTKGEVKLQESGP |
| | | GLVAPSQSLSVTCTVSGVSLPDYGVSWIRQPPRKGLEWLGVIWGSETTYYNS |
| | | ALKSRLTIIKDNSKSQVFLKMNSLQTDDTAIYYCAKHYYYGGSYAMDYWGQG |
| | | TSVTVSS |
| CAR B full NT | 5015 | GACATCCAGATGACCCAGACCACCTCCAGCCTGAGCGCCAGCCTGGGCGACC |
| sequence | | GGGTGACCATCAGCTGCCGGGCCAGCCAGGACATCAGCAAGTACCTGAACTG |
| | | GTATCAGCAGAAGCCCGACGGCACCGTCAAGCTGCTGATCTACCACACCAGC |
| | | CGGCTGCACAGCGGCGTGCCCAGCCGGTTTAGCGGCAGCGGCTCCGGCACCG |
| | | ACTACAGCCTGACCATCTCCAACCTGGAACAGGAAGATATCGCCACCTACTT |
| | | TTGCCAGCAGGGCAACACACTGCCCTACACCTTTGGCGGCGGAACAAAGCTG |
| | | GAAATCACCGGCAGCACCTCCGGCAGCGGCAAGCCTGGCAGCGGCGAGGGCA |
| | | GCACCAAGGGCGAGGTGAAGCTGCAGGAAAGCGGCCCTGGCCTGGTGGCCCC |
| | | CAGCCAGAGCCTGAGCGTGACCTGCACCGTGAGCGGCGTGAGCCTGCCCGAC |
| | | TACGGCGTGAGCTGGATCCGGCAGCCCCCCAGGAAGGGCCTGGAATGGCTGG |
| | | GCGTGATCTGGGGCAGCGAGACCACCTACTACAACAGCGCCCTGAAGAGCCG |
| | | GCTGACCATCATCAAGGACAACAGCAAGAGCCAGGTGTTCCTGAAGATGAAC |
| | | AGCCTGCAGACCGACGACACCGCCATCTACTACTGCGCCAAGCACTACTACT |
| | | ACGGCGGCAGCTACGCCATGGACTACTGGGGCCAGGGCACCAGCGTGACCGT |
| | | GAGCAGCGAATCTAAGTACGGACCGCCCTGCCCCCCTTGCCCTATGTTCTGG |
| | | GTGCTGGTGGTGGTCGGAGGCGTGCTGGCCTGCTACAGCCTGCTGGTCACCG |
| | | TGGCCTTCATCATCTTTTGGGTGAAACGGGGCAGAAAGAAACTCCTGTATAT |
| | | ATTCAAACAACCATTTASGAGACCAGTACAAACTACTCAAGAGGAAGATGGC |
| | | TGTAGCTGCCGATTTCCAGAAGAAGAAGAAGGAGGATGTGAACTGCGGGTGA |
| | | AGTTCAGCAGAAGCGCCGACGCCCCTGCCTACCAGCAGGGCCAGAATCAGCT |
| | | GTACAACGAGCTGAACCTGGGCAGAAGGGAAGAGTACGACGTCCTGGATAAG |
| | | CGGAGAGGCCGGGACCCTGAGATGGGCGGCAAGCCTCGGCGGAAGAACCCCC |
| | | AGGAAGGCCTGTATAACGAACTGCAGAAAGACAAGATGGCCGAGGCCTACAG |
| | | CGAGATCGGCATGAAGGGCGAGCGGAGGCGGGGCAAGGGCCACGACGGCCTG |
| | | TATCAGGGCCTGTCCACCGCCACCAAGGATACCTACGACGCCCTGCACATGC |
| | | AGGCCCTGCCCCCAAGG |
| CAR B full AA | 5016 | DIQMTQTTSSLSASLGDRVTISCRASQDISKYLNWYQQKPDGTVKLLIYHTS |
| transgene | | RLHSGVPSRFSGSGSGTDYSLTISNLEQEDIATYFCQQGNTLPYTFGGGTKL |
| sequence | | EZTGSTSGSGKPGSGEGSTKGEVKLQESGPGLVAPSQSLSVTCTVSGVSLPD |
| | | YGVSWIRQPPRKGLEWLGVIWGSETTYYNSALKSRLTIIKDNSKSQVFLKMN |
| | | SLQTDDTAIYYCAKHYYYGGSYAMDYWGQGTSVTVSSESKYGPPCPPCPMFW |
| | | VLVVVGGVLACYSLLVTVAFIIFWVKRGRKKLLYIFKQPFMRPVQTTQEEDG |
| | | CSCRFPEEEEGGCELRVKFSRSADAPAYQQGQNQLYNELNLGRREEYDVLDK |
| | | RRGRDPEMGGKPRRKNPQEGLYNELQKDKMAEAYSEIGMKGERRRGKGHDGL |
| | | YQGLSTATKDTYDALHMQALPPR |
| CAR B CD19 | 5017 | GACATCCAGATGACCCAGACCACCTCCAGCCTGAGCGCCAGCCTGGGCGACC |
| scFv NT | | GGGTGACCATCAGCTGCCGGGCCAGCCAGGACATCAGCAAGTACCTGAACTG |
| sequence | | GTATCAGCAGAAGCCCGACGGCACCGTCAAGCTGCTGATCTACCACACCAGC |
| | | CGGCTGCACAGCGGCGTGCCCAGCCGGTTTAGCGGCAGCGGCTCCGGCACCG |
| | | ACTACAGCCTGACCATCTCCAACCTGGAACAGGAAGATATCGCCACCTACTT |
| | | TTGCCAGCAGGGCAACACACTGCCCTACACCTTTGGCGGCGGAACAAAGCTG |
| | | GAAATCACCGGCAGCACCTCCGGCAGCGGCAAGCCTGGCAGCGGCGAGGGCA |
| | | GCACCAAGGGCGAGGTGAAGCTGCAGGAAAGCGGCCCTGGCCTGGTGGCCCC |
| | | CAGCCAGAGCCTGAGCGTGACCTGCACCGTGAGCGGCGTGAGCCTGCCCGAC |
| | | TACGGCGTGAGCTGGATCCGGCAGCCCCCCAGGAAGGGCCTGGAATGGCTGG |
| | | GCGTGATCTGGGGCAGCGAGACCACCTACTACAACAGCGCCCTGAAGAGCCG |
| | | GCTGACCATCATCAAGGACAACAGCAAGAGCCAGGTGTTCCTGAAGATGAAC |
| | | AGCCTGCAGACCGACGACACCGCCATCTACTACTGCGCCAAGCACTACTACT |
| | | ACGGCGGCAGCTACGCCATGGACTACTGGGGCCAGGGCACCAGCGTGACCGT |
| | | GAGCAGC |
| CAR B CD19 | 5018 | DIQMTQTTSSLSASLGDRVTISCRASQDISKYLNWYQQKPDGTVKLLIYHTS |
| scFv sequence | | RLHSGVPSRFSGSGSGTDYSLTISNLEQEDIATYFCQQGNTLPYTFGGGTKL |
| | | EITGSTSGSGKPGSGEGSTKGEVKLQESGPGLVAPSQSLSVTCTVSGVSLPD |
| | | YGVSWIRQPPRKGLEWLGVIWGSETTYYNSALKSRLTIIKDNSKSQVFLKMN |
| | | SLQTDDTAIYYCAKHYYYGGSYAMDYWGQGTSVTVSS |

TABLE 3
Murine CD19 CAR Constructs
CTL019

| | | |
|---|---|---|
| CTL019- | 97 | Atggccctgcccgtcaccgctctgctgctgccccttgctctgcttcttcatgcagc |
| Soluble | | aaggccggacatccagatgacccaaaccacctcatccctctctgcctctcttggag |
| scFv- | | acagggtgaccatttcttgtcgcgccagccaggacatcagcaagtatctgaactgg |
| Histag | | tatcagcagaagccggacggaaccgtgaagctcctgatctaccatacctctcgcct |
| -nt | | gcatagcggcgtgccctcacgcttctctggaagcggatcaggaaccgattattctc |
| | | tcactatttcaaatcttgagcaggaagatattgccacctatttctgccagcagggt |
| | | aataccctgccctacaccttcggaggagggaccaagctcgaaatcaccggtggagg |
| | | aggcagcggcggtggagggtctggtggaggtggttctgaggtgaagctgcaagaat |
| | | caggccctggacttgtggccccttcacagtccctgagcgtgacttgcaccgtgtcc |
| | | ggagtctccctgcccgactacggagtgtcatggatcagacaacctccacggaaagg |
| | | actggaatggctcggtgtcatctggggtagcgaaactacttactacaattcagccc |
| | | tcaaaagcaggctgactattatcaaggacaacagcaagtcccaagtctttcttaag |
| | | atgaactcactccagactgacgacaccgcaatctactattgtgctaagcactacta |
| | | ctacggaggatcctacgctatggattactggggacaaggtacttccgtcactgtct |
| | | cttcacaccatcatcaccatcaccatcac |
| CTL019- | 98 | MALPVTALLLPLALLLHAARPdiqmtqttsslsaslgdrvtiscrasqdiskylnw |
| Soluble | | yqqkpdgtvklliyhtsrlhsgvpsrfsgsgsgtdysltisnlegediatyfcqqg |
| scFv- | | ntlpytfgggtkleitggggsggggsggggsevklqesgpglvapsqslsvtctvs |
| Histag | | gvslpdygvswirqpprkglewlgviwgsettyynsalksrltiikdnsksqvilk |
| -aa | | mnslqtddtaiyycakhyyyggsyamdywgqgtsvtvsshhhhhhhh |
| CTL019 | 99 | atggccttaccagtgaccgccttgctcctgccgctggccttgctgctccacgccgc |
| Full-nt | | caggccggacatccagatgacacagactacatcctccctgtctgcctctctgggag |
| | | acagagtcaccatcagttgcagggcaagtcaggacattagtaaatatttaaattgg |
| | | tatcagcagaaaccagatggaactgttaaactcctgatctaccatacatcaagatt |
| | | acactcaggagtcccatcaaggttcagtggcagtgggtctggaacagattattctc |
| | | tcaccattagcaacctggagcaagaagatattgccacttacttttgccaacagggt |
| | | aatacgcttccgtacacgttcggaggggggaccaagctggagatcacaggtggcgg |
| | | tggctcgggcggtggtgggtcgggtggcggcggatctgaggtgaaactgcaggagt |
| | | caggacctggcctggtggcgccctcacagagcctgtccgtcacatgcactgtctca |
| | | ggggtctcattacccgactatggtgtaagctggattcgccagectccacgaaaggg |
| | | tctggagtggctgggagtaatatggggtagtgaaaccacatactataattcagctc |
| | | tcaaatccagactgaccatcatcaaggacaactccaagagccaagttttcttaaaa |
| | | atgaacagtctgcaaactgatgacacagccatttactactgtgccaaacattatta |
| | | ctacggtggtagctatgctatggactactggggccaaggaacctcagtcaccgtct |
| | | cctcaaccacgacgccagcgccgcgaccaccaacaccggcgcccaccatcgcgtcg |
| | | cagcccctgtccctgcgcccagaggcgtgccggccagcggcggggggcgcagtgca |
| | | cacgagggggctggacttcgcctgtgatatctacatctgggcgcccttggccggga |
| | | cttgtggggtccttctcctgtcactggttatcaccctttactgcaaacggggcaga |
| | | aagaaactcctgtatatattcaaacaaccatttatgagaccagtacaaactactca |
| | | agaggaagatggctgtagctgccgatttccagaagaagaagaaggaggatgtgaac |
| | | tgagagtgaagttcagcaggagcgcagacgcccccgcgtacaagcagggccagaac |
| | | cagctctataacgagctcaatctaggacgaagagaggagtacgatgttttggacaa |
| | | gagacgtggccgggaccctgagatggggggaaagccgagaaggaagaaccctcagg |
| | | aaggcctgtacaatgaactgcagaaagataagatggcggaggcctacagtgagatt |
| | | gggatgaaaggcgagcgccggaggggcaaggggcacgatggcctttaccagggtct |
| | | cagtacagccaccaaggacacctacgacgcccttcacatgcaggccctgccccctc |
| | | gc |
| CTL019 | 58 | MALPVTALLLPLALLLHAARPdiqmtqttsslsaslgdrvtiscrasqdiskylnw |
| Full-aa | | yqqkpdgtvklliyhtsrlhsgvpsrfsgsgsgtdysltisnlegediatyfcqqg |
| | | ntlpytfgggtkleitggggsggggsggggsevklqesgpglvapsqslsvtctvs |
| | | gvslpdygvswirqpprkglewlgviwgsettyynsalksrltiikdnsksqvflk |
| | | mnslqtddtaiyycakhyyyggsyamdywgqgtsvtvsstttpaprpptpaptias |
| | | qplslrpeacrpaaggavhtrgldfacdiyiwaplagtcgvlllslvitlyckrgr |
| | | kkllyifkqpfmrpvqttqeedgescrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |
| CTL019 | 59 | Diqmtqttsslsaslgdrvtiscrasqdiskylnwyqqkpdgtvklliyhtsrlhs |
| scFv | | gvpsrfsgsgsgtdysltisnlegediatyfcqqgntlpytfgggtkleitggggs |
| domain | | ggggsggggsevklgesgpglvapsqslsvtctvsgvslpdygvswirqpprkgle |
| | | wlgviwgsettyynsalksrltiikdnsksqvflkmnslqtddtaiyycakhyyyg |
| | | gsyamdywgqgtsvtvss |
| mCAR1 | 109 | QVQLLESGAELVRPGSSVKISCKASGYAFSSYWMNWVKQRPGQGLEWIGQIYPGDG |
| scFv | | DTNYNGKFKGQATLEADKSSSTAYMQLSGLTSEDSAVYSCARKTISSVVDFYFDYW |
| | | GQGTTVTGGGSGGGSGGGSGGGSELVLTQSPKFMSTSVGDRVSVTCKASQNVGINV |
| | | AWYQQKPGQSPKPLTYSATYRNSGVPDRFTGSGSGTDFTLTITNVQSKDLADYFCQ |
| | | YNRYPYTSFFFTKLEIKRRS |
| mCAR1 | 110 | QVQLLESGAELVRPGSSVKISCKASGYAFSSYWMNWVKQRPGQGLEWIGQIYPGDG |
| Full-aa | | DTNYNGKFKGQATLEADKSSSTAYMQLSGLTSEDSAVYSCARKTISSVVDFYFDYW |
| | | GQGTTVTGGGSGGGSGGGSGGGSELVLTQSPKFMSTSVGDRVSVTCKASQNVGINV |
| | | AWYQQKPGQSPKPLIYSATYRNSGVPDRFTGSGSGTDFTLTITNVQSKDLADYFCQ |
| | | YNRYPYTSFFFTKLEIKRRSKIEVMYPPPYLDNEKSNGTIIHVKGKHLCPSPLFPG |
| | | PSKPFWVLVVVGGVLACYSLLVTVAFIIFWVRSKRSRLLHSDYMNMTPRRPGPTRK |
| | | HYQPYAPPRDFAAYRSRVKFSRSADAPAYQQGQNQLYNELNLGRREEYDVLDKRRG |
| | | RDPEMGGKPRRKNPQEGLYNELQKDKMAEAYSEIGMKGERRRGKGHDGLYQGLSTA |
| | | TKDTYDALHMQALPPR |
| mCAR2 | 111 | DIQMTQTTSSLSASLGDRVTISCRASQDISKYLNWYQQKPDGTVKLLIYHTSRLHS |
| scFv | | GVPSRFSGSGSGTDYSLTISNLEQEDIATYFCQQGNTLPYTFGGGTKLEITGSTSG |
| | | SGKPGSGEGSTKGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIYYCAKHY |
| | | YYGGSYAMDYWGQGISVTVSSE |
| mCAR2 | 112 | DIQMTQTTSSLSASLGDRVTISCRASQDISKYLNWYQQKPDGTVKLLIYHTSRLHS |
| CAR-aa | | GVPSRFSGSGSGTDYSLTISNLEQEDIATYFCQQGNTLPYTFGGGTKLEITGSTSG |
| | | SGKPGSGEGSTKGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIY |
| | | YCAKHYYYGGSYAMDYWGQGTSVTVSSESKYGPPCPPCPMFWVLVVVGGVLACYSL |
| | | LVTVAFIIFWVKRGRKKLLYIFKQPFMRPVQTTQEEDGCSCRFEEEEGGCELRVKF |
| | | SRSADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPRL |
| mCAR2 | 113 | DIQMTQTT SSLSASLGDR VTISCRASQD ISKYLNWYQQ KPDGTVKLLI |
| Full-aa | | YHTSRLHSGV PSRFSGSGSG TDYSLTISNL EQEDIATYFC QQGNTLPYTE |
| | | GGGTKLEITG STSGSGKPGS GEGSTKGEVK LQESGPGLVA PSQSLSVTCT |
| | | VSGVSLPDYG VSWIRQPPRK GLEWLGVIWG SETTYYNSAL KSRLTIIKDN |
| | | SKSQVFLKMN SLQTDDTAIY YCAKHYYYGG SYAMDYWGQG TSVTVSSESK |
| | | YGPPCPPCPM FWVLVVVGGV LACYSLLVTV |
| | | AFIIFWVKRG RKKLLYIFKQ PFMRPVQTTQ EEDGCCRFE EEEGGCELRV |
| | | KFSRSADAPA YQQGQNQLYN ELNLGRREEY DVLDKRRGRD PEMGGKPRRK |
| | | NPQEGLYNEL QKDKMAEAYS EIGMKGERRR GKGHDGLYQG LSTATKDTYD |
| | | ALHMQALPPR LEGGGEGRGS LLTCGDVEEN PGPRMLLLVT SLLLCELPHP |
| | | AFLLIPRKVC NGIGIGEFKD SLSINATNIK HFKNCTSISG DLHILPVAFR |
| | | GDSFTHTPPL DPQELDILKT VKEITGELLI QAWPENRTDL HAFENLEIIR |
| | | GRTKQHGQFS LAVVSLNITS LGLRSLKEIS DGDVIISGNK NLCYANTINW |
| | | KKLFGTSGQK TKIISNRGEN SCKATGQVCH ALCSPEGCWG PEPRDCVSCR |
| | | NVSRGRECVD KCNLLEGEPR EFVENSECIQ CHPECLPQAM NITCTGRGPD |
| | | NCIQCAHYID GPHCVKTCPA GVMGENNTLV WKYADAGHVC HLCHPNCTYG |
| | | CTGPGLEGCP INGPKIPSIA TGMVGALLLL LVVALGIGLF M |
| mCAR3 | 114 | DIQMTQTTSSLSASLGDRVTISCRASQDISKYLNWYQQKPDGTVKLLIYHTSRLHS |
| scFv | | GVPSRFSGSGSGTDYSLTISNLEQEDIATYFCQQGNTLPYTFGGGTKLEITGSTSG |
| | | SGKPGSGEGSTKGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIYYCAKHY |
| | | YYGGSYAMDYWGQGESVTVSS |
| mCAR3 | 115 | DIQMTQTTSSLSASLGDRVTISCRASQDISKYLNWYQQKPDGTVKLLIYHTSRLHS |
| Full-aa | | GVPSRFSGSGSGTDYSLTISNLEQEDIATYFCQQGNTLPYTFGGGTKLEITGSTSG |
| | | SGKPGSGEGSTKGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIYYCAKHY |
| | | YYGGSYAMDYWGQGISVTVSSAAAIEVMYPPPYLDNEKSNGTIIHVKGKHLCPSPL |
| | | FPGPSKPFWVLVVVGGVLACYSLLVTVAFIIFWVRSKRSRLLHSDYMNMTPRRPGP |
| | | TRKHYQPYAPPRDFAAYRSRVKFSRSADAPAYQQGQNQLYNELNLGRREEYDVLDK |
| | | RRGRDPEMGGKPRRKNPQEGLYNELQKDKMAEAYSEIGMKGERRRGKGHDGLYQGL |
| | | STATKDTYDALHMQALPPR |

In some embodiments, the CDRs are defined according to the Kabat numbering scheme, the Chothia numbering scheme, or a combination thereof.

The sequences of humanized CDR sequences of the scFv domains are shown in Table 4 for the heavy chain variable domains and in Table 5 for the light chain variable domains. “ID” stands for the respective SEQ ID NO for each CDR.

TABLE 4
Heavy Chain Variable Domain CDRs

| Candidate | FW | HCDR1 | ID | HCDR2 | ID | HCDR3 | ID |
|---|---|---|---|---|---|---|---|
| murine_ | | GVSLP | 19 | VIWGS | 20 | HYYYG | 24 |
| CART19 | | DYGVS | | ETTYY | | GSYAM | |
| | | | | NSALK | | DY | |
| | | | | S | | | |
| humanized_ | VH4 | GVSLP | 19 | VIWGS | 21 | HYYYG | 24 |
| CART19 a | | DYGVS | | ETTYY | | GSYAM | |
| | | | | SSSLK | | DY | |
| | | | | S | | | |
| humanized_ | VH4 | GVSLP | 19 | VIWGS | 22 | HYYYG | 24 |
| CART19 b | | DYGVS | | ETTYY | | GSYAM | |
| | | | | QSSLK | | DY | |
| | | | | S | | | |
| humanized_ | VH4 | GVSLP | 19 | VIWGS | 23 | HYYYG | 24 |
| CART19 c | | DYGVS | | ETTYY | | GSYAM | |
| | | | | NSSLK | | DY | |
| | | | | S | | | |

TABLE 5
Light Chain Variable Domain CDRs

| Candidate | FW | LCDR1 | ID | LCDR2 | ID | ILCDR3 | ID |
|---|---|---|---|---|---|---|---|
| murine_ | | RASQDI | 25 | HTSR | 26 | QQGNT | 27 |
| CART19 | | SKYLN | | LHS | | LPYT | |
| humanized_ | VK3 | RASQDI | 25 | HTSR | 26 | QQGNT | 27 |
| CART19 a | | SKYLN | | LHS | | LPYT | |
| humanized_ | VK3 | RASQDI | 25 | HTSR | 26 | QQGNT | 27 |
| CART19 b | | SKYLN | | LHS | | LPYT | |
| humanized_ | VK3 | RASQDI | 25 | HTSR | 26 | QQGNT | 27 |
| CART19 c | | SKYLN | | LHS | | LPYT | |

The CAR scFv fragments were then cloned into lentiviral vectors to create a full length CAR construct in a single coding frame, and using the EF1 alpha promoter for expression (SEQ ID NO: 100).

EF1 alpha promoter
(SEQ ID NO: 100)
CGTGAGGCTCCGGTGCCCGTCAGTGGGCAGAGCGCACATCGCCCACAGTCCCCGAGAAGTTGGGGGGAGGGGTCGG
CAATTGAACCGGTGCCTAGAGAAGGTGGCGCGGGGTAAACTGGGAAAGTGATGTCGTGTACTGGCTCCGCCTTTTT
CCCGAGGGTGGGGGAGAACCGTATATAAGTGCAGTAGTCGCCGTGAACGTTCTTTTTCGCAACGGGTTTGCCGCCA
GAACACAGGTAAGTGCCGTGTGTGGTTCCCGCGGGCCTGGCCTCTTTACGGGTTATGGCCCTTGCGTGCCTTGAAT
TACTTCCACCTGGCTGCAGTACGTGATTCTTGATCCCGAGCTTCGGGTTGGAAGTGGGTGGGAGAGTTCGAGGCCT
TGCGCTTAAGGAGCCCCTTCGCCTCGTGCTTGAGTTGAGGCCTGGCCTGGGCGCTGGGGCCGCCGCGTGCGAATCT
GGTGGCACCTTCGCGCCTGTCTCGCTGCTTTCGATAAGTCTCTAGCCATTTAAAATTTTTGATGACCTGCTGCGAC
GCTTTTTTTCTGGCAAGATAGTCTTGTAAATGCGGGCCAAGATCTGCACACTGGTATTTCGGTTTTTGGGGCCGCG
GGCGGCGACGGGGCCCGTGCGTCCCAGCGCACATGTTCGGCGAGGCGGGGCCTGCGAGCGCGGCCACCGAGAATCG
GACGGGGGTAGTCTCAAGCTGGCCGGCCTGCTCTGGTGCCTGGCCTCGCGCCGCCGTGTATCGCCCCGCCCTGGGC
GGCAAGGCTGGCCCGGTCGGCACCAGTTGCGTGAGCGGAAAGATGGCCGCTTCCCGGCCCTGCTGCAGGGAGCTCA
AAATGGAGGACGCGGCGCTCGGGAGAGCGGGCGGGTGAGECACCCACACAAAGGAAAAGGGCCTTTCCGTCCTCAG
CCGTCGCTTCATGTGACTCCACGGAGTACCGGGCGCCGTCCAGGCACCTCGATTAGTTCTCGAGCTTTTGGAGTAC
GTCGTCTTTAGGTTGGGGGGAGGGGTTTTATGCGATGGAGTTTCCCCACACTGAGEGGGTGGAGACTGAAGTTAGG
CCAGCTTGGCACTTGATGTAATTCTCCTTGGAATTTGCCCTTTTTGAGTTTGGATCTTGGTTCATTCTCAAGCCTC
AGACAGTGGTTCAAAGTTTTTTTCTTCCATTTCAGGTGTCGTGA.

### CD20 CAR

In some embodiments, the CAR-expressing cell described herein is a CD20 CAR-expressing cell (e.g., a cell expressing a CAR that binds to human CD20). In some embodiments, the CD20 CAR-expressing cell includes an antigen binding domain according to WO2016/164731 and PCT/US2017/055627, incorporated herein by reference. Exemplary CD20-binding sequences or CD20 CAR sequences are disclosed in, e.g., Tables 1-5 of PCT/US2017/055627. In some embodiments, the CD20 CAR comprises a CDR, variable region, scFv, or full-length sequence of a CD20 CAR disclosed in PCT/US2017/055627 or WO2016/164731.

### CD22 CAR

In some embodiments, the CAR-expressing cell described herein is a CD22 CAR-expressing cell (e.g., a cell expressing a CAR that binds to human CD22). In some embodiments, the CD22 CAR-expressing cell includes an antigen binding domain according to WO2016/164731 and PCT/US2017/055627, incorporated herein by reference. Exemplary CD22-binding sequences or CD22 CAR sequences are disclosed in, e.g., Tables 6A, 6B, 7A, 7B, 7C, 8A, 8B, 9A, 9B, 10A, and 10B of WO2016/164731 and Tables 6-10 of PCT/US2017/055627. In some embodiments, the CD22 CAR sequences comprise a CDR, variable region, scFv or full-length sequence of a CD22 CAR disclosed in PCT/US2017/055627 or WO2016/164731.

### CD123 CAR

In some embodiments, the CAR-expressing cells can specifically bind to CD123, e.g., can include a CAR molecule (e.g., any of the CAR1 to CAR8), or an antigen binding domain according to Tables 1-2 of WO 2014/130635, incorporated herein by reference. The amino acid and nucleotide sequences encoding the CD123 CAR molecules and antigen binding domains (e.g., including one, two, three VH CDRs; and one, two, three VL CDRs according to Kabat or Chothia), are specified in WO 2014/130635. In other embodiments, the CAR-expressing cells can specifically bind to CD123, e.g., can include a CAR molecule (e.g., any of the CAR123-1 to CAR123-4 and hzCAR123-1 to hzCAR123-32), or an antigen binding domain according to Tables 2, 6, and 9 of WO2016/028896, incorporated herein by reference. The amino acid and nucleotide sequences encoding the CD123 CAR molecules and antigen binding domains (e.g., including one, two, three VH CDRs; and one, two, three VL CDRs according to Kabat or Chothia), are specified in WO2016/028896.

### BCMA CAR

In some embodiments, the CAR-expressing cells can specifically bind to BCMA, e.g., can include a CAR molecule (e.g., any of BCMA-1 to BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1), or an antigen binding domain according to Tables 1 and 16 of WO 2016/014565, incorporated herein by reference. The amino acid and nucleotide sequences encoding the BCMA CAR molecules and antigen binding domains (e.g., including one, two, three VH CDRs; and one, two, three VL CDRs according to Kabat or Chothia), are specified in WO 2016/014565.

### Other Exemplary CARs

In some embodiments, the CAR-expressing cells can bind a B-cell antigen, e.g., a B-cell antigen described herein.

In some embodiments, the CAR-expressing cells can specifically bind to ROR1. In some embodiments, the ROR1 CAR-expressing cells comprise an antigen binding domain against ROR1, and the antigen binding domain is an antigen binding portion, e.g., CDRs, of an antibody described in, e.g., Hudecek et al., Clin Cancer Res 19(12):3153-3164 (2013); WO 2011159847; and US20130101607.

In some embodiment, the CAR-expressing cells can specifically bind to FLT3. In some embodiments, the FLT3 CAR-expressing cell comprise an antigen binding domain against FLT3, and the antigen binding domain is an antigen binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam).

In some embodiments, the CAR-expressing cells can specifically bind to CD79a. In some embodiments, the CD79a CAR-expressing cells comprise an antigen binding domain against CD79a, and the antigen binding domain is an antigen binding portion, e.g., CDRs, of the antibody Anti-CD79a antibody [HM47/A9] (ab3121), available from Abcam; antibody CD79A Antibody #3351 available from Cell Signalling Technology; or antibody HPA017748—Anti-CD79A antibody produced in rabbit, available from Sigma Aldrich.

In some embodiments, the CAR-expressing cells can specifically bind CD79b. In some embodiments, the CD79b CAR-expressing cells comprise an antigen binding domain against CD79b, and the antigen binding domain is an antigen binding portion, e.g., CDRs, of the antibody polatuzumab vedotin, anti-CD79b described in Dornan et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56^{th} ASH

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

Bispecific CARs

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

In certain embodiments, the antibody molecule is a multi-specific (e.g., a bispecific or a trispecific) antibody molecule. Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also encompassed creating for bispecifc, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus futher associated with VL domains to form a series of FVs (or scFys), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811A1, US2009234105A1, US2009263392A1, US2009274649A1, EP346087A2, WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO9409131A1, WO9412625A2, WO9509917A1, WO9637621A2, WO9964460A1. The contents of the above-referenced applications are incorporated herein by reference in their entireties.

Within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁-VH₁-VH₂-VL₂. Optionally, a linker is disposed between the two antibodies or antibody fragments (e.g., scFvs), e.g., between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₄-Ser)n linker, wherein n is 1, 2, 3, 4, 5, or 6, preferably 4 (SEQ ID NO: 1264). In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, a bispecific CAR comprises VLs, VHs, and optionally one or more linkers in an arrangement as described herein.

Stability and Mutations

The stability of an antigen binding domain to a cancer associated antigen as described herein, e.g., scFv molecules (e.g., soluble scFv), can be evaluated in reference to the biophysical properties (e.g., thermal stability) of a conventional control scFv molecule or a full length antibody. In one embodiment, the humanized scFv has a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a control binding molecule (e.g. a conventional scFv molecule) in the described assays.

The improved thermal stability of the antigen binding domain to a cancer associated antigen described herein, e.g., scFv is subsequently conferred to the entire CAR construct, leading to improved therapeutic properties of the CAR construct. The thermal stability of the antigen binding domain of—a cancer associated antigen described herein, e.g., scFv, can be improved by at least about 2° C. or 3° C. as compared to a conventional antibody. In one embodiment, the antigen binding domain of—a cancer associated antigen described herein, e.g., scFv, has a 1° C. improved thermal stability as compared to a conventional antibody. In another embodiment, the antigen binding domain of a cancer associated antigen described herein, e.g., scFv, has a 2° C. improved thermal stability as compared to a conventional antibody. In another embodiment, the scFv has a 4, 5, 6, 7, 8, 9, 10, 11, 12, 13, 14, 15° C. improved thermal stability as compared to a conventional antibody. Comparisons can be made, for example, between the scFv molecules disclosed herein and scFv molecules or Fab fragments of an antibody from which the scFv VH and VL were derived. Thermal stability can be measured using methods known in the art. For example, in one embodiment, Tm can be measured. Methods for measuring Tm and other methods of determining protein stability are described in more detail below.

Mutations in scFv (arising through humanization or direct mutagenesis of the soluble scFv) can alter the stability of the scFv and improve the overall stability of the scFv and the CAR construct. Stability of the humanized scFv is compared against the murine scFv using measurements such as Tm, temperature denaturation and temperature aggregation.

The binding capacity of the mutant scFvs can be determined using assays know in the art and described herein.

In one embodiment, the antigen binding domain of—a cancer associated antigen described herein, e.g., scFv, comprises at least one mutation arising from the humanization process such that the mutated scFv confers improved stability to the CAR construct. In another embodiment, the antigen binding domain of—a cancer associated antigen described herein, e.g., scFv, comprises at least 1, 2, 3, 4, 5, 6, 7, 8, 9, 10 mutations arising from the humanization process such that the mutated scFv confers improved stability to the CAR construct.

Methods of Evaluating Protein Stability

The stability of an antigen binding domain may be assessed using, e.g., the methods described below. Such methods allow for the determination of multiple thermal unfolding transitions where the least stable domain either unfolds first or limits the overall stability threshold of a multidomain unit that unfolds cooperatively (e.g., a multidomain protein which exhibits a single unfolding transition). The least stable domain can be identified in a number of additional ways. Mutagenesis can be performed to probe which domain limits the overall stability. Additionally, protease resistance of a multidomain protein can be performed under conditions where the least stable domain is known to be intrinsically unfolded via DSC or other spectroscopic methods (Fontana, et al., (1997) Fold. Des., 2: R17-26; Dimasi et al. (2009) J. Mol. Biol. 393: 672-692). Once the least stable domain is identified, the sequence encoding this domain (or a portion thereof) may be employed as a test sequence in the methods. Exemplary methods for evaluating the stability of an antigen binding domain, e.g., thermal stability, propensity for aggregation (% aggregation), and binding affinity are described in International Publication No. WO2019/210153, the contents of which are hereby incorporated by reference.

In one aspect, the antigen binding domain of the CAR comprises an amino acid sequence that is homologous to an antigen binding domain amino acid sequence described herein, and the antigen binding domain retains the desired functional properties of the antigen binding domain described herein.

In one specific aspect, the CAR composition of the invention comprises an antibody fragment. In a further aspect, the antibody fragment comprises an scFv.

In various aspects, the antigen binding domain of the CAR is engineered by modifying one or more amino acids within one or both variable regions (e.g., VH and/or VL), for example within one or more CDR regions and/or within one or more framework regions. In one specific aspect, the CAR composition of the invention comprises an antibody fragment. In a further aspect, the antibody fragment comprises an scFv.

It will be understood by one of ordinary skill in the art that the antibody or antibody fragment of the invention may further be modified such that they vary in amino acid sequence (e.g., from wild-type), but not in desired activity. For example, additional nucleotide substitutions leading to amino acid substitutions at “non-essential” amino acid residues may be made to the protein For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

In one aspect, the present invention contemplates modifications of the starting antibody or fragment (e.g., scFv) amino acid sequence that generate functionally equivalent molecules. For example, the VH or VL of an antigen binding domain to—a cancer associated antigen described herein, e.g., scFv, comprised in the CAR can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting VH or VL framework region of the antigen binding domain to the cancer associated antigen described herein, e.g., scFv. The present invention contemplates modifications of the entire CAR construct, e.g., modifications in one or more amino acid sequences of the various domains of the CAR construct in order to generate functionally equivalent molecules. The CAR construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting CAR construct.

### Chimeric TCR

In one aspect, the antibodies and antibody fragments disclosed herein can be grafted to one or more constant domain of a T cell receptor (“TCR”) chain, for example, a TCR alpha or TCR beta chain, to create an chimeric TCR that binds specifically to a cancer associated antigen. Without being bound by theory, it is believed that chimeric TCRs will signal through the TCR complex upon antigen binding. For example, an scFv as disclosed herein, can be grafted to the constant domain, e.g., at least a portion of the extracellular constant domain, the transmembrane domain and the cytoplasmic domain, of a TCR chain, for example, the TCR alpha chain and/or the TCR beta chain. As another example, an antibody fragment, for example a VL domain as described herein, can be grafted to the constant domain of a TCR alpha chain, and an antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an antibody or antibody fragment, e.g., the CDRs of an antibody or antibody fragment as described in any of the Tables herein may be grafted into a TCR alpha and/or beta chain to create a chimeric TCR that binds specifically to a cancer associated antigen. For example, the LC CDRs disclosed herein may be grafted into the variable domain of a TCR alpha chain and the HC CDRs disclosed herein may be grafted to the variable domain of a TCR beta chain, or vice versa. Such chimeric TCRs may be produced by any appropriate method (For example, Willemsen R A et al, Gene Therapy 2000; 7: 1369-1377; Zhang T et al, Cancer Gene Ther 2004; 11: 487-496; Aggen et al, Gene Ther. 2012 April; 19(4):365-74).

### Linkers for Antigen Binding Domains

It was found that CAR molecules comprising a short or no linker between the variable domains (e.g., VH and VL) of the antigen binding domain showed equal to, or greater, activity than longer versions of the linker. For example, in some embodiments, CD22-65s (having (Gly4-Ser)n linker, wherein n is 1 (SEQ ID NO: 5037)) shows comparable or greater activity and/or efficacy in a tumor model, compared to CD22-65 (having (Gly4-Ser)n linker, wherein n is 3 (SEQ ID NO: 28)). Accordingly, any of the antigen binding domains or CAR molecules described herein can have a linker connecting the variable domains of the antigen binding domain of varying lengths, including for example, a short linker of about 3 to 6 amino acids, 4 to 5 amino acids, or about 5 amino acids. In some embodiments, a longer linker can be used, e.g., about 6 to 35 amino acids, e.g., 8 to 32 amino acids, 10 to 30 amino acids, 10 to 20 amino acids. For example, a (Gly4-Ser)n linker, wherein n is 0, 1, 2, 3, 4, 5, or 6 (SEQ ID NO: 5036) can be used. In one embodiment, the variable domains are not connected via a linker, e.g., (Gly4-Ser)n linker, n=0 (“Gly4-Ser” disclosed as SEQ ID NO: 18). In some embodiments, the variable domains are connected via a short linker, e.g., (Gly4-Ser)n linker, n=1 (SEQ ID NO: 5037). In some embodiments, the variable domains are connected via a (Gly4-Ser)n linker, n=2 (SEQ ID NO: 49). In some embodiments, the variable domains are connected via a (Gly4-Ser)n linker, n=3 (SEQ ID NO: 28). In some embodiments, the variable domains are connected via a (Gly4-Ser)n linker, n=4 (SEQ ID NO: 106). In some embodiments, the variable domains are connected via a (Gly4-Ser)n linker, n=5 (SEQ ID NO: 5034). In some embodiments, the variable domains are connected via a (Gly4-Ser)n linker, n=6 (SEQ ID NO: 5035). The order of the variable domain, e.g., in which the VL and VH domains appear in the antigen binding domain, e.g., scFv, can be varied (i.e., VL-VH, or VH-VL orientation). In one embodiment, the antigen binding domain binds to CD20, e.g., a CD20 antigen binding domain as described herein. In another embodiment, the antigen binding domain binds to CD22, e.g., a CD22 antigen binding domain as described herein. In another embodiment, the antigen binding domain binds to CD19, e.g., a CD19 antigen binding domain as described herein.

Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR, e.g., in one embodiment, the transmembrane domain may be from the same protein that the signaling domain, costimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect the amino acid sequence of the transmembrane domain may be modified or substituted so as to minimize interactions with the binding domains of the native binding partner present in the same CAR-expressing cell.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, or CD19.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, an IgD hinge, a GS linker (e.g., a GS linker described herein), a KIR2DS2 hinge, or a CD8a hinge. In one embodiment, the hinge or spacer comprises (e.g., consists of) the amino acid sequence of SEQ ID NO:14. In one aspect, the transmembrane domain comprises (e.g., consists of) a transmembrane domain of SEQ ID NO: 15.

In one aspect, the hinge or spacer comprises an IgG4 hinge. For example, in one embodiment, the hinge or spacer comprises a hinge of the amino acid sequence ESKYGPPCPPCPAPEFLGGPSVFLFPPKPKDTLMISRTPEVTCVVVDVSQEDPEVQFNW YVDGVEVHNAKTKPREEQFNSTYRVVSVLTVLHQDWLNGKEYKCKVSNKGLPSSIEK TISKAKGQPREPQVYTLPPSQEEMTKNQVSLTCLVKGFYPSDIAVEWESNGQPENNYK TTPPVLDSDGSFFLYSRLTVDKSRWQEGNVFSCSVMHEALHNHYTQKSLSLSLGKM (SEQ ID NO:45). In some embodiments, the hinge or spacer comprises a hinge encoded by a nucleotide sequence of

(SEQ ID NO: 46)
GAGAGCAAGTACGGCCCTCCCTGCCCCCCTTGCCCTGCCCCCGAGTTCC
TGGGCGGACCCAGCGTGTTCCTGTTCCCCCCCAAGCCCAAGGACACCCT
GATGATCAGCCGGACCCCCGAGGTGACCTGTGTGGTGGTGGACGTGTCC
CAGGAGGACCCCGAGGTCCAGTTCAACTGGTACGTGGACGGCGTGGAGG
TGCACAACGCCAAGACCAAGCCCCGGGAGGAGCAGTTCAATAGCACCTA
CCGGGTGGTGTCCGTGCTGACCGTGCTGCACCAGGACTGGCTGAACGGC
AAGGAATACAAGTGTAAGGTGTCCAACAAGGGCCTGCCCAGCAGCATCG
AGAAAACCATCAGCAAGGCCAAGGGCCAGCCTCGGGAGCCCCAGGTGTA
CACCCTGCCCCCTAGCCAAGAGGAGATGACCAAGAACCAGGTGTCCCTG
ACCTGCCTGGTGAAGGGCTTCTACCCCAGCGACATCGCCGTGGAGTGGG
AGAGCAACGGCCAGCCCGAGAACAACTACAAGACCACCCCCCCTGTGCT
GGACAGCGACGGCAGCTTCTTCCTGTACAGCCGGCTGACCGTGGACAAG
AGCCGGTGGCAGGAGGGCAACGTCTTTAGCTGCTCCGTGATGCACGAGG
CCCTGCACAACCACTACACCCAGAAGAGCCTGAGCCTGTCCCTGGGCAA
GATG.

In one aspect, the hinge or spacer comprises an IgD hinge. For example, in one embodiment, the hinge or spacer comprises a hinge of the amino acid sequence RWPESPKAQASSVPTAQPQAEGSLAKATTAPATTRNTGRGGEEKKKEKEKEEQEERET KTPECPSIHTQPLGVYLLTPAVQDLWLRDKATFTCFVVGSDLKDAHLTWEVAGKVPTG GVEEGLLERHSNGSQSQHSRLTLPRSLWNAGTSVTCTLNHPSLPPQRLMALREPAAQA PVKLSLNLLASSDPPEAASWLLCEVSGFSPPNILLMWLEDQREVNTSGFAPARPPPQPG STTFWAWSVLRVPAPPSPQPATYTCVVSHEDSRTLLNASRSLEVSYVTDH (SEQ ID NO:47). In some embodiments, the hinge or spacer comprises a hinge encoded by a nucleotide sequence of

(SEQ ID NO: 48)
AGGTGGCCCGAAAGTCCCAAGGCCCAGGCATCTAGTGTTCCTACTGCAC
AGCCCCAGGCAGAAGGCAGCCTAGCCAAAGCTACTACTGCACCTGCCAC
TACGCGCAATACTGGCCGTGGCGGGGAGGAGAAGAAAAAGGAGAAAGAG
AAAGAAGAACAGGAAGAGAGGGAGACCAAGACCCCTGAATGTCCATCCC
ATACCCAGCCGCTGGGCGTCTATCTCTTGACTCCCGCAGTACAGGACTT
GTGGCTTAGAGATAAGGCCACCTTTACATGTTTCGTCGTGGGCTCTGAC
CTGAAGGATGCCCATTTGACTTGGGAGGTTGCCGGAAAGGTACCCACAG
GGGGGGTTGAGGAAGGGTTGCTGGAGCGCCATTCCAATGGCTCTCAGAG
CCAGCACTCAAGACTCACCCTTCCGAGATCCCTGTGGAACGCCGGGACC
TCTGTCACATGTACTCTAAATCATCCTAGCCTGCCCCCACAGCGTCTGA
TGGCCCTTAGAGAGCCAGCCGCCCAGGCACCAGTTAAGCTTAGCCTGAA
TCTGCTCGCCAGTAGTGATCCCCCAGAGGCCGCCAGCTGGCTCTTATGC
GAAGTGTCCGGCTTTAGCCCGCCCAACATCTTGCTCATGTGGCTGGAGG
ACCAGCGAGAAGTGAACACCAGCGGCTTCGCTCCAGCCCGGCCCCCACC
CCAGCCGGGTTCTACCACATTCTGGGCCTGGAGTGTCTTAAGGGTCCCA
GCACCACCTAGCCCCCAGCCAGCCACATACACCTGTGTTGTGTCCCATG
AAGATAGCAGGACCCTGCTAAATGCTTCTAGGAGTCTGGAGGTTTCCTA
CGTGACTGACCATT.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of GGGGSGGGGS (SEQ ID NO:49). In some embodiments, the linker is encoded by a nucleotide sequence of

(SEQ ID NO: 50)
GGTGGCGGAGGTTCTGGAGGTGGAGGTTCC.

In one aspect, the hinge or spacer comprises a KIR2DS2 hinge.

Cytoplasmic Domain

The cytoplasmic domain or region of the CAR includes an intracellular signaling domain. An intracellular signaling domain is generally responsible for activation of at least one of the normal effector functions of the immune cell in which the CAR has been introduced.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of CD3 zeta, common FcR gamma (FCER1G), Fc gamma RIIa, FcR beta (Fc Epsilon Rib), CD3 gamma, CD3 delta, CD3 epsilon, CD79a, CD79b, CD278 (also known as “ICOS”), FcεRI, DAP10, DAP12, and CD66d. In one embodiment, a CAR of the invention comprises an intracellular signaling domain, e.g., a primary signaling domain of CD3-zeta.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

Further examples of molecules containing a primary intracellular signaling domain that are of particular use in the invention include those of DAP10, DAP12, and CD32.

### Costimulatory Signaling Domain

The intracellular signalling domain of the CAR can comprise the CD3-zeta signaling domain by itself or it can be combined with any other desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. In one embodiment, the intracellular domain is designed to comprise the signaling domain of CD3-zeta and the signaling domain of CD28. In one aspect, the intracellular domain is designed to comprise the signaling domain of CD3-zeta and the signaling domain of ICOS.

A costimulatory molecule can be a cell surface molecule other than an antigen receptor or its ligands that is required for an efficient response of lymphocytes to an antigen. Examples of such molecules include CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

The intracellular signaling sequences within the cytoplasmic portion of the CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB. In one aspect, the signaling domain of 4-1BB is a signaling domain of SEQ ID NO: 16. In one aspect, the signaling domain of CD3-zeta is a signaling domain of SEQ ID NO: 17.

In one aspect, the intracellular signaling domain is designed to comprise the signaling domain of CD3-zeta and the signaling domain of CD27. In one aspect, the signaling domain of CD27 comprises an amino acid sequence of

(SEQ ID NO: 51)
QRRKYRSNKGESPVEPAEPCRYSCPREEEGSTIPIQEDYRKPEPACSP.

In one aspect, the signalling domain of CD27 is encoded by a nucleic acid sequence of

(SEQ ID NO: 52)
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC.

### Natural Killer Cell Receptor (NKR) CARs

In an embodiment, a CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR molecules described herein may interact with an adaptor molecule or intracellular signaling domain, e.g., DAP12. Exemplary configurations and sequences of CAR molecules comprising NKR components are described in International Publication No. WO2014/145252, the contents of which are hereby incorporated by reference.

### Regulatable Chimeric Antigen Receptors

In some embodiments, a regulatable CAR (RCAR) where the CAR activity can be controlled is desirable to optimize the safety and efficacy of a CAR therapy. There are many ways CAR activities can be regulated. For example, inducing apoptosis using, e.g., a caspase fused to a dimerization domain (see, e.g., Di et al., N Engl. J. Med. 2011 Nov. 3; 365(18):1673-1683), can be used as a safety switch in the CAR therapy of the instant invention. In one embodiment, the cells (e.g., T cells or NK cells) expressing a CAR of the present invention further comprise an inducible apoptosis switch, wherein a human caspase (e.g., caspase 9) or a modified version is fused to a modification of the human FKB protein that allows conditional dimerization. In the presence of a small molecule, such as a rapalog (e.g., AP 1903, AP20187), the inducible caspase (e.g., caspase 9) is activated and leads to the rapid apoptosis and death of the cells (e.g., T cells or NK cells) expressing a CAR of the present invention. Examples of a caspase-based inducible apoptosis switch (or one or more aspects of such a switch) have been described in, e.g., US2004040047; US20110286980; US20140255360; WO1997031899; WO2014151960; WO2014164348; WO2014197638; WO2014197638; all of which are incorporated by reference herein.

In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; and Di Stasi et al. N. Engl. J. Med. 2011; 365:1673-83.

Alternative strategies for regulating the CAR therapy of the instant invention include utilizing small molecules or antibodies that deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or complement-induced cell death. For example, CAR expressing cells described herein may also express a receptor capable of being targeted by an antibody or antibody fragment. Examples of such receptors include EpCAM, VEGFR, integrins (e.g., integrins αvβ3, α4, αI¾β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11 a/LFA-1, CD15, CD18/ITGB2, CD19, CD20, CD22, CD23/lgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

For example, a CAR-expressing cell described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., Gene Ther. 2013; 20(8)853-860). Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., Blood. 2014; 124(8)1277-1287). Other methods for depleting CAR-expressing cells described herein include administration of CAMPATH, a monoclonal anti-CD52 antibody that selectively binds and targets mature lymphocytes, e.g., CAR-expressing cells, for destruction, e.g., by inducing ADCC. In other embodiments, the CAR-expressing cell can be selectively targeted using a CAR ligand, e.g., an anti-idiotypic antibody. In some embodiments, the anti-idiotypic antibody can cause effector cell activity, e.g., ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In other embodiments, a CAR-expressing cell described herein may also express a target protein recognized by the T cell depleting agent. In one embodiment, the target protein is CD20 and the T cell depleting agent is an anti-CD20 antibody, e.g., rituximab. In such embodiment, the T cell depleting agent is administered once it is desirable to reduce or eliminate the CAR-expressing cell, e.g., to mitigate the CAR induced toxicity. In other embodiments, the T cell depleting agent is an anti-CD52 antibody, e.g., alemtuzumab.

In an aspect, a RCAR comprises a set of polypeptides, typically two in the simplest embodiments, in which the components of a standard CAR described herein, e.g., an antigen binding domain and an intracellular signaling domain, are partitioned on separate polypeptides or members. In some embodiments, the set of polypeptides include a dimerization switch that, upon the presence of a dimerization molecule, can couple the polypeptides to one another, e.g., can couple an antigen binding domain to an intracellular signaling domain. In one embodiment, a CAR of the present invention utilizes a dimerization switch as those described in, e.g., WO2014127261, which is incorporated by reference herein. Additional description and exemplary configurations of such regulatable CARs are provided herein and in International Publication No. WO 2015/090229, hereby incorporated by reference in its entirety.

In some embodiments, an RCAR involves a switch domain, e.g., a FKBP switch domain, as set out SEQ ID NO: 122, or comprise a fragment of FKBP having the ability to bind with FRB, e.g., as set out in SEQ ID NO: 123. In some embodiments, the RCAR involves a switch domain comprising a FRB sequence, e.g., as set out in SEQ ID NO: 124, or a mutant FRB sequence, e.g., as set out in any of SEQ ID Nos. 125-130. D V P D Y A S L G G P S S P K K K R K V S R G V Q V E T I S P G D G R T F P K R G Q T C V V H Y T G M L E D G K K F D S S R D R N K P F K F M L G K Q E V I R G W E E G V A Q M S V G Q R A K L T I S P D Y A Y G A T G H P G I I P P H A T L V F D V E L L K L E T S Y (SEQ ID NO: 122)

(SEQ ID NO: 123)
VQVETISPGDGRTFPKRGQTCVVHYTGMLEDGKKFDSSR
DRNKPFKFMLGKQEVIRGWEEGVAQMSVGQRAKLTISPDYA
YGATGHPGIIPPHATLVFDVELLKLETS
(SEQ ID NO: 124)
ILWHEMWHEG LEEASRLYFG ERNVKGMFEV LEPLHAMMER
GPQTLKETSF NQAYGRDLME AQEWCRKYMK SGNVKDLTQA
WDLYYHVFRR ISK

TABLE 1
Exemplary mutant FRB having increased affinity for a dimerization molecule.

| | | SEQ |
|---|---|---|
| | | ID |
| FRB mutant | Amino Acid Sequence | NO: |
| E2032I mutant | ILWHEMWHEGLIEASRLYFGERNVKGMFEVLEPLHAMMERGPQTLKETSENQAYGR | 125 |
| | DLMEAQEWCRKYMKSGNVKDLTQAWDLYYHVFRRISKTS | |
| E2032L mutant | ILWHEMWHEGLLEASRLYFGERNVKGMFEVLEPLHAMMERGPQTLKETSFNQAYGR | 126 |
| | DLMEAQEWCRKYMKSGNVKDLTQAWDLYYHVFRRISKTS | |
| T2098L mutant | ILWHEMWHEGLEEASRLYFGERNVKGMFEVLEPLHAMMERGPQTLKETSENQAYGR | 127 |
| | DLMEAQEWCRKYMKSGNVKDLLQAWDLYYHVFRRISKTS | |
| E2032, T2098 | ILWHEMWHEGLXEASRLYFGERNVKGMFEVLEPLHAMMERGPQTLKETSFNQAYGR | 128 |
| mutant | DLMEAQEWCRKYMKSGNVKDLXQAWDLYYHVFRRISKTS | |
| E2032I, T2098L | ILWHEMWHEGLIEASRLYFGERNVKGMFEVLEPLHAMMERGPQTLKETSENQAYGR | 129 |
| mutant | DLMEAQEWCRKYMKSGNVKDLLQAWDLYYHVERRISKTS | |
| E2032L, T2098L | ILWHEMWHEGLLEASRLYFGERNVKGMFEVLEPLHAMMERGPQTLKETSENQAYGR | 130 |
| mutant | DLMEAQEWCRKYMKSGNVKDLLQAWDLYYHVFRRISKTS | |

### Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

### RNA Transfection

Disclosed herein are methods for producing an in vitro transcribed RNA CAR. The present invention also includes (among other things) a CAR encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length (SEQ ID NO:118). RNA so produced can efficiently transfect different kinds of cells. In one aspect, the template includes sequences for the CAR.

In one aspect the CAR is encoded by a messenger RNA (mRNA). In one aspect the mRNA encoding the CAR is introduced into an immune effector cell, e.g., a T cell or a NK cell, for production of a CAR-expressing cell, e.g., a CART cell or a CAR NK cell.

Methods of producing an in vitro transcribed RNA CAR are described on pages 192-196 of International Application WO 2016/164731 filed on 8 Apr. 2016, hereby incorporated by reference.

### Non-Viral Delivery Methods

In some aspects, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery systems and methods of using thereof are described on pages 196-198 of International Application WO 2016/164731 filed on 8 Apr. 2016, which is hereby incorporated by reference.

### Nucleic Acid Constructs Encoding a CAR

The present invention also provides nucleic acid molecules encoding one or more CAR constructs described herein, e.g., CD19 CAR. In one aspect, the nucleic acid molecule is provided as a messenger RNA transcript. In one aspect, the nucleic acid molecule is provided as a DNA construct.

Accordingly, in one aspect, the invention pertains to an isolated nucleic acid molecule encoding a chimeric antigen receptor (CAR), wherein the CAR comprises a binding domain (e.g., that binds a B-cell antigen, e.g., CD19, CD20, CD22, CD34, CD123, BCMA, FLT-3, ROR1, CD79b, CD179b, or CD79a) a transmembrane domain, and an intracellular signaling domain comprising a stimulatory domain, e.g., a costimulatory signaling domain and/or a primary signaling domain, e.g., zeta chain.

In one embodiment, the binding domain is an anti-CD19 binding domain described herein, e.g., an anti-CD19 binding domain which comprises a sequence selected from a group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12 and SEQ ID NO:59, or a sequence with 95-99% identity thereof.

In one embodiment, the nucleic acid comprises a CD20, CD22, CD34, CD123, BCMA, FLT-3, ROR1, CD79b, CD179b, or CD79a encoding nucleic acid.

In one embodiment, the transmembrane domain is transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment, the transmembrane domain comprises a sequence of SEQ ID NO: 15, or a sequence with 95-99% identity thereof. In one embodiment, the anti-CD19 binding domain is connected to the transmembrane domain by a hinge region, e.g., a hinge described herein. In one embodiment, the hinge region comprises SEQ ID NO:14 or SEQ ID NO:45 or SEQ ID NO:47 or SEQ ID NO:49, or a sequence with 95-99% identity thereof. In one embodiment, the isolated nucleic acid molecule further comprises a sequence encoding a costimulatory domain. In one embodiment, the costimulatory domain is a functional signaling domain of a protein selected from the group consisting of OX40, CD27, CD28, CDS, ICAM-1, LFA-1 (CD11a/CD18), ICOS (CD278), and 4-1BB (CD137). In one embodiment, the costimulatory domain is a functional signaling domain of a protein selected from the group consisting of MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. In one embodiment, the costimulatory domain comprises a sequence of SEQ ID NO:16, or a sequence with 95-99% identity thereof. In one embodiment, the intracellular signaling domain comprises a functional signaling domain of 4-1BB and a functional signaling domain of CD3 zeta. In one embodiment, the intracellular signaling domain comprises the sequence of SEQ ID NO: 16 or SEQ ID NO:51, or a sequence with 95-99% identity thereof, and the sequence of SEQ ID NO: 17 or SEQ ID NO:43, or a sequence with 95-99% identity thereof, wherein the sequences comprising the intracellular signaling domain are expressed in the same frame and as a single polypeptide chain.

In another aspect, the invention pertains to an isolated nucleic acid molecule encoding a CAR construct comprising a leader sequence of SEQ ID NO: 13, a scFv domain having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, and SEQ ID NO:59, (or a sequence with 95-99% identity thereof), a hinge region of SEQ ID NO:14 or SEQ ID NO:45 or SEQ ID NO:47 or SEQ ID NO:49 (or a sequence with 95-99% identity thereof), a transmembrane domain having a sequence of SEQ ID NO: 15 (or a sequence with 95-99% identity thereof), a 4-1BB costimulatory domain having a sequence of SEQ ID NO:16 or a CD27 costimulatory domain having a sequence of SEQ ID NO:51 (or a sequence with 95-99% identity thereof), and a CD3 zeta stimulatory domain having a sequence of SEQ ID NO:17 or SEQ ID NO:43 (or a sequence with 95-99% identity thereof).

In another aspect, the invention pertains to an isolated polypeptide molecule encoded by the nucleic acid molecule. In one embodiment, the isolated polypeptide molecule comprises a sequence selected from the group consisting of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:59 or a sequence with 95-99% identity thereof.

In another aspect, the invention pertains to a nucleic acid molecule encoding a chimeric antigen receptor (CAR) molecule that comprises an anti-CD19 binding domain, a transmembrane domain, and an intracellular signaling domain comprising a stimulatory domain, and wherein said anti-CD19 binding domain comprises a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:59, or a sequence with 95-99% identity thereof.

In one embodiment, the encoded CAR molecule (e.g., CD19 CAR, CD20 CAR, CD22 CAR, a CD34 CAR, a CD123 CAR, a BCMA CAR, a FLT-3 CAR, a ROR1 CAR, a CD79b CAR, a CD179b CAR, or a CD79a CAR) further comprises a sequence encoding a costimulatory domain. In one embodiment, the costimulatory domain is a functional signaling domain of a protein selected from the group consisting of OX40, CD27, CD28, CDS, ICAM-1, LFA-1 (CD11a/CD18) and 4-1BB (CD137). In one embodiment, the costimulatory domain comprises a sequence of SEQ ID NO:16. In one embodiment, the transmembrane domain is a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment, the transmembrane domain comprises a sequence of SEQ ID NO:15. In one embodiment, the intracellular signaling domain comprises a functional signaling domain of 4-1BB and a functional signaling domain of zeta. In one embodiment, the intracellular signaling domain comprises the sequence of SEQ ID NO: 16 and the sequence of SEQ ID NO: 17, wherein the sequences comprising the intracellular signaling domain are expressed in the same frame and as a single polypeptide chain. In one embodiment, the anti-CD19 binding domain is connected to the transmembrane domain by a hinge region. In one embodiment, the hinge region comprises SEQ ID NO:14. In one embodiment, the hinge region comprises SEQ ID NO:45 or SEQ ID NO:47 or SEQ ID NO:49.

In another aspect, the invention pertains to an encoded CAR molecule comprising a leader sequence of SEQ ID NO: 13, a scFv domain having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, and SEQ ID NO:59, or a sequence with 95-99% identity thereof, a hinge region of SEQ ID NO:14 or SEQ ID NO:45 or SEQ ID NO:47 or SEQ ID NO:49, a transmembrane domain having a sequence of SEQ ID NO: 15, a 4-1BB costimulatory domain having a sequence of SEQ ID NO:16 or a CD27 costimulatory domain having a sequence of SEQ ID NO:51, and a CD3 zeta stimulatory domain having a sequence of SEQ ID NO:17 or SEQ ID NO:43. In one embodiment, the encoded CAR molecule comprises a sequence selected from a group consisting of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, and SEQ ID NO:59, or a sequence with 95-99% identity thereof.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (V), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the invention is an adenoviral vector (A5/35). In another embodiment, the expression of nucleic acids encoding CARs can be accomplished using of transposons such as sleeping beauty, crispr, CAS9, and zinc finger nucleases. See below June et al. 2009 Nature Reviews Immunology 9.10: 704-716, is incorporated herein by reference.

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

In some aspects, the expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1α, ubiquitin C, or phosphoglycerokinase (PGK) promoters. In an embodiment, the promoter is a PGK promoter, e.g., a truncated PGK promoter as described herein.

An example of a promoter that is capable of expressing a CAR transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from transgenes cloned into a lentiviral vector. See, e.g., Milone et al., Mol. Ther. 17(8): 1453-1464 (2009). In one aspect, the EFla promoter comprises the sequence provided as SEQ ID NO:100.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired. The nucleotide sequences of exemplary PGK promoters are provided below.

WT PGK Promoter:

(SEQ ID NO: 1323)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTGTGGGGCGGAGGGCGTGGCGGGGAAGGGCC
GGCGACGAGAGCCGCGCGGGACGACTCGTCGGCGATAACCGGTGTCGGG
TAGCGCCAGCCGCGCGACGGTAACGAGGGACCGCGACAGGCAGACGCTC
CCATGATCACTCTGCACGCCGAAGGCAAATAGTGCAGGCCGTGCGGCGC
TTGGCGTTCCTTGGAAGGGCTGAATCCCCGCCTCGTCCTTCGCAGCGGC
CCCCCGGGTGTTCCCATCGCCGCTTCTAGGCCCACTGCGACGCTTGCCT
GCACTTCTTACACGCTCTGGGTCCCAGCCGCGGCGACGCAAAGGGCCTT
GGTGCGGGTCTCGTCGGCGCAGGGACGCGTTTGGGTCCCGACGGAACCT
TTTCCGCGTTGGGGTTGGGGCACCATAAGCT

Exemplary truncated PGK Promoters:

PGK100:

(SEQ ID NO: 1324)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTG

PGK200:

(SEQ ID NO: 1325)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTGTGGGGCGGAGGGCGTGGCGGGGAAGGGCC
GGCGACGAGAGCCGCGCGGGACGACTCGTCGGCGATAACCGGTGTCGGG
TAGCGCCAGCCGCGCGACGGTAACG

PGK300:

(SEQ ID NO: 1326)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTGTGGGGCGGAGGGCGTGGCGGGGAAGGGCC
GGCGACGAGAGCCGCGCGGGACGACTCGTCGGCGATAACCGGTGTCGGG
TAGCGCCAGCCGCGCGACGGTAACGAGGGACCGCGACAGGCAGACGCTC
CCATGATCACTCTGCACGCCGAAGGCAAATAGTGCAGGCCGTGCGGCGC
TTGGCGTTCCTTGGAAGGGCTGAATCCCCG

PGK400:

(SEQ ID NO: 1327)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTGTGGGGCGGAGGGCGTGGCGGGGAAGGGCC
GGCGACGAGAGCCGCGCGGGACGACTCGTCGGCGATAACCGGTGTCGGG
TAGCGCCAGCCGCGCGACGGTAACGAGGGACCGCGACAGGCAGACGCTC
CCATGATCACTCTGCACGCCGAAGGCAAATAGTGCAGGCCGTGCGGCGC
TTGGCGTTCCTTGGAAGGGCTGAATCCCCGCCTCGTCCTTCGCAGCGGC
CCCCCGGGTGTTCCCATCGCCGCTTCTAGGCCCACTGCGACGCTTGCCT
GCACTTCTTACACGCTCTGGGTCCCAGCCG

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In embodiments, the vector may comprise two or more nucleic acid sequences encoding a CAR, e.g., a first CAR that binds to CD19 and a second CAR, e.g., an inhibitory CAR or a CAR that specifically binds to a second antigen, e.g., CD20, CD22, CD34, CD123, BCMA, FLT-3, ROR1, CD79b, CD179b, or CD79a. In such embodiments, the two or more nucleic acid sequences encoding the CAR are encoded by a single nucleic molecule in the same frame and as a single polypeptide chain. In this aspect, the two or more CARs, can, e.g., be separated by one or more peptide cleavage sites. (e.g., an auto-cleavage site or a substrate for an intracellular protease). Examples of peptide cleavage sites include the following, wherein the GSG residues are optional:

T2A:
(SEQ ID NO: 1328)
(GSG)EGRGSLLTCGDVEENPGP
P2A:
(SEQ ID NO: 1329)
(GSG)ATNFSLLKQAGDVEENPGP
E2A:
(SEQ ID NO: 1330)
(GSG)QCTNYALLKLAGDVESNPGP
F2A:
(SEQ ID NO: 1331)
(GSG)VKQTLNFDLLKLAGDVESNPGP

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A suitable method for the introduction of a polynucleotide into a host cell is calcium phosphate transfection

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are described on page 209 of International Application WO 2016/164731 filed on 8 Apr. 2016, which is hereby incorporated by reference.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The present invention further provides a vector comprising a CAR encoding nucleic acid molecule. In one aspect, a CAR vector can be directly transduced into a cell, e.g., a T cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the CAR construct in mammalian T cells. In one aspect, the mammalian T cell is a human T cell.

### Sources of Cells

Prior to expansion and genetic modification or other modification, a source of cells, e.g., T cells or natural killer (NK) cells, can be obtained from a subject. The term “subject” is intended to include living organisms in which an immune response can be elicited (e.g., mammals). Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In certain aspects of the present disclosure, immune effector cells, e.g., T cells, can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one aspect, T cells are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is 1e7 cells to 20 uL, or 1e7 cells to 15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In an embodiment, a subject is pre-treated with one or more therapies that reduce T_{RE}G cells prior to collection of cells for CAR-expressing cell product manufacturing, thereby reducing the risk of subject relapse to CAR-expressing cell treatment. In an embodiment, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In an embodiment, a subject is pre-treated with cyclophosphamide prior to collection of cells for CAR-expressing cell product manufacturing, thereby reducing the risk of subject relapse to CAR-expressing cell treatment. In an embodiment, a subject is pre-treated with an anti-GITR antibody prior to collection of cells for CAR-expressing cell product manufacturing, thereby reducing the risk of subject relapse to CAR-expressing cell treatment.

In one embodiment, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, BCMA, CD20, CD14 or CD11b, to thereby provide a population of T regulatory depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary checkpoint inhibitors include B7-H1, B7-1, CD160, P1H, 2B4, PD1, TIM3, CEACAM (e.g., CEACAM-1, CEACAM-3 and/or CEACAM-5), LAG3, TIGIT, CTLA-4, BTLA and LAIR1. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

Methods described herein can include a positive selection step. For example, T cells can isolated by incubation with anti-CD3/anti-CD28 (e.g., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours, e.g., 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 10 billion cells/ml, 9 billion/ml, 8 billion/ml, 7 billion/ml, 6 billion/ml, or 5 billion/ml is used. In one aspect, a concentration of 1 billion cells/ml is used. In yet one aspect, a concentration of cells from 75, 80, 85, 90, 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR molecule, e.g., a CAR molecule described herein, are obtained from a subject that has received a low, immune enhancing dose of an mTOR inhibitor. In an embodiment, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In some embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

### Allogeneic CAR

In some embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II.

A T cell lacking a functional TCR can be, e.g., engineered such that it does not express any functional TCR on its surface, engineered such that it does not express one or more subunits that comprise a functional TCR or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class 1 and/or HLA class II, is downregulated.

In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not express or expresses at low levels an inhibitory molecule, e.g. by any method described herein. For example, the cell can be a cell that does not express or expresses at low levels an inhibitory molecule, e.g., that can decrease the ability of a CAR-expressing cell to mount an immune effector response. Examples of inhibitory molecules include PD1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4 and TGF beta. Inhibition of an inhibitory molecule, e.g., by inhibition at the DNA, RNA or protein level, can optimize a CAR-expressing cell performance. In some embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA in a T cell.

Expression of siRNA and shRNAs in T cells can be achieved using any conventional expression system, e.g., such as a lentiviral expression system.

Exemplary shRNAs that downregulate expression of components of the TCR are described, e.g., in US Publication No.: 2012/0321667. Exemplary siRNA and shRNA that downregulate expression of HLA class I and/or HLA class II genes are described, e.g., in U.S. publication No.: US 2007/0036773.

CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene.

Naturally-occurring CRISPR/Cas systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

The CRISPR/Cas system has been modified for use in gene editing (silencing, enhancing or changing specific genes) in eukaryotes such as mice or primates. Wiedenheft et al. (2012) Nature 482: 331-8. This is accomplished by introducing into the eukaryotic cell a plasmid containing a specifically designed CRISPR and one or more appropriate Cas.

The CRISPR sequence, sometimes called a CRISPR locus, comprises alternating repeats and spacers. In a naturally-occurring CRISPR, the spacers usually comprise sequences foreign to the bacterium such as a plasmid or phage sequence; in the TCR and/or HLA CRISPR/Cas system, the spacers are derived from the TCR or HLA gene sequence.

RNA from the CRISPR locus is constitutively expressed and processed by Cas proteins into small RNAs. These comprise a spacer flanked by a repeat sequence. The RNAs guide other Cas proteins to silence exogenous genetic elements at the RNA or DNA level. Horvath et al. (2010) Science 327: 167-170; Makarova et al. (2006) Biology Direct 1: 7. The spacers thus serve as templates for RNA molecules, analogously to siRNAs. Pennisi (2013) Science 341: 833-836.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stern et al. (2010) Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836.

The CRISPR/Cas system can thus be used to edit a TCR and/or HLA gene (adding or deleting a basepair), or introducing a premature stop which thus decreases expression of a TCR and/or HLA. The CRISPR/Cas system can alternatively be used like RNA interference, turning off TCR and/or HLA gene in a reversible fashion. In a mammalian cell, for example, the RNA can guide the Cas protein to a TCR and/or HLA promoter, sterically blocking RNA polymerases.

Artificial CRISPR/Cas systems can be generated which inhibit TCR and/or HLA, using technology known in the art, e.g., that described in U.S. Publication No. 20140068797, and Cong (2013) Science 339: 819-823. Other artificial CRISPR/Cas systems that are known in the art may also be generated which inhibit TCR and/or HLA, e.g., that described in Tsai (2014) Nature Biotechnol., 32:6 569-576, U.S. Pat. Nos. 8,871,445; 8,865,406; 8,795,965; 8,771,945; and 8,697,359.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene.

TALENs are produced artificially by fusing a TAL effector DNA binding domain to a DNA cleavage domain. Transcription activator-like effects (TALEs) can be engineered to bind any desired DNA sequence, including a portion of the HLA or TCR gene. By combining an engineered TALE with a DNA cleavage domain, a restriction enzyme can be produced which is specific to any desired DNA sequence, including a HLA or TCR sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

To produce a TALEN, a TALE protein is fused to a nuclease (N), which is a wild-type or mutated FokI endonuclease. Several mutations to FokI have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96.

The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. (2011) Nature Biotech. 29: 143-8.

A HLA or TCR TALEN can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN; depending on the sequences of the foreign DNA and chromosomal sequence, this process can be used to correct a defect in the HLA or TCR gene or introduce such a defect into a wt HLA or TCR gene, thus decreasing expression of HLA or TCR.

TALENs specific to sequences in HLA or TCR can be constructed using any method known in the art, including various schemes using modular components. Zhang et al. (2011) Nature Biotech. 29: 149-53; Geibler et al. (2011) PLoS ONE 6: e19509.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene.

Like a TALEN, a ZFN comprises a FokI nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160.

A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5.

Also like a TALEN, a ZFN can create a double-stranded break in the DNA, which can create a frame-shift mutation if improperly repaired, leading to a decrease in the expression and amount of HLA and/or TCR in a cell. ZFNs can also be used with homologous recombination to mutate in the HLA or TCR gene.

ZFNs specific to sequences in HLA AND/OR TCR can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; Guo et al. (2010) J. Mol. Biol. 400: 96; U.S. Patent Publication 2011/0158957; and U.S. Patent Publication 2012/0060230.

Telomerase Expression

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in an embodiment, an immune effector cell, e.g., a T cell, ectopically expresses a telomerase subunit, e.g., the catalytic subunit of telomerase, e.g., TERT, e.g., hTERT. In some aspects, this disclosure provides a method of producing a CAR-expressing cell, comprising contacting a cell with a nucleic acid encoding a telomerase subunit, e.g., the catalytic subunit of telomerase, e.g., TERT, e.g., hTERT. The cell may be contacted with the nucleic acid before, simultaneous with, or after being contacted with a construct encoding a CAR.

In one aspect, the disclosure features a method of making a population of immune effector cells (e.g., T cells, NK cells). In an embodiment, the method comprises: providing a population of immune effector cells (e.g., T cells or NK cells), contacting the population of immune effector cells with a nucleic acid encoding a CAR; and contacting the population of immune effector cells with a nucleic acid encoding a telomerase subunit, e.g., hTERT, under conditions that allow for CAR and telomerase expression.

In an embodiment, the nucleic acid encoding the telomerase subunit is DNA. In an embodiment, the nucleic acid encoding the telomerase subunit comprises a promoter capable of driving expression of the telomerase subunit.

In an embodiment, hTERT has the amino acid sequence of GenBank Protein ID AAC51724.1 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795) as follows:

(SEQ ID NO: 363)
MPRAPRCRAVRSLLRSHYREVLPLATFVRRLGPQGWRLVQRGDPAAFRA
LVAQCLVCVPWDARPPPAAPSFRQVSCLKELVARVLQRLCERGAKNVLA
FGFALLDGARGGPPEAFTTSVRSYLPNTVTDALRGSGAWGLLLRRVGDD
VLVHLLARCALFVLVAPSCAYQVCGPPLYQLGAATQARPPPHASGPRRR
LGCERAWNHSVREAGVPLGLPAPGARRRGGSASRSLPLPKRPRRGAAPE
PERTPVGQGSWAHPGRTRGPSDRGFCVVSPARPAEEATSLEGALSGTRH
SHPSVGRQHHAGPPSTSRPPRPWDTPCPPVYAETKHFLYSSGDKEQLRP
SFLLSSLRPSLTGARRLVETIFLGSRPWMPGTPRRLPRLPQRYWQMRPL
FLELLGNHAQCPYGVLLKTHCPLRAAVTPAAGVCAREKPQGSVAAPEEE
DTDPRRLVQLLRQHSSPWQVYGFVRACLRRLVPPGLWGSRHNERRFLRN
TKKFISLGKHAKLSLQELTWKMSVRGCAWLRRSPGVGCVPAAEHRLREE
ILAKFLHWLMSVYVVELLRSFFYVTETTFQKNRLFFYRKSVWSKLQSIG
IRQHLKRVQLRELSEAEVRQHREARPALLTSRLRFIPKPDGLRPIVNMD
YVVGARTFRREKRAERLTSRVKALFSVLNYERARRPGLLGASVLGLDDI
HRAWRTFVLRVRAQDPPPELYFVKVDVTGAYDTIPQDRLTEVIASIIKP
QNTYCVRRYAVVQKAAHGHVRKAFKSHVSTLTDLQPYMRQFVAHLQETS
PLRDAVVIEQSSSLNEASSGLFDVFLRFMCHHAVRIRGKSYVQCQGIPQ
GSILSTLLCSLCYGDMENKLFAGIRRDGLLLRLVDDFLLVTPHLTHAKT
FLRTLVRGVPEYGCVVNLRKTVVNFPVEDEALGGTAFVQMPAHGLFPWC
GLLLDTRTLEVQSDYSSYARTSIRASLTFNRGFKAGRNMRRKLFGVLRL
KCHSLFLDLQVNSLQTVCTNIYKILLLQAYRFHACVLQLPFHQQVWKNP
TFFLRVISDTASLCYSILKAKNAGMSLGAKGAAGPLPSEAVQWLCHQAF
LLKLTRHRVTYVPLLGSLRTAQTQLSRKLPGTTLTALEAAANPALPSDF
KTILD

In an embodiment, the hTERT has a sequence at least 80%, 85%, 90%. 95%, 96\hat{ }, 97%, 98%, or 99% identical to the sequence of SEQ ID NO: 363. In an embodiment, the hTERT has a sequence of SEQ ID NO: 363. In an embodiment, the hTERT comprises a deletion (e.g., of no more than 5, 10, 15, 20, or 30 amino acids) at the N-terminus, the C-terminus, or both. In an embodiment, the hTERT comprises a transgenic amino acid sequence (e.g., of no more than 5, 10, 15, 20, or 30 amino acids) at the N-terminus, the C-terminus, or both.

In an embodiment, the hTERT is encoded by the nucleic acid sequence of GenBank Accession No. AF018167 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795).

### Activation and Expansion of Immune Effector Cells (e.g., T Cells)

Immune effector cells such as T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells can comprise: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

The method of expansion of immune effector cells, and methods of introducing CAR nucleic acid molecules into immune effector cells, and method of detecting CAR expression is described on pages 236-246 of in International Application WO 2016/164731 filed on Apr. 8, 2016, which is incorporated by reference in its entirety.

Other assays, including those described in the Example section herein as well as those that are known in the art can also be used to evaluate the CARs described herein.

Alternatively, or in combination to the methods disclosed herein, methods and compositions for one or more of detection and/or quantification of CAR-expressing cells (e.g., in vitro or in vivo (e.g., clinical monitoring)), immune cell expansion and/or activation, and/or CAR-specific selection, that involve the use of a CAR ligand, are disclosed. In one exemplary embodiment, the CAR ligand is an antibody that binds to the CAR molecule, e.g., binds to the extracellular antigen binding domain of CAR (e.g., an antibody that binds to the antigen binding domain, e.g., an anti-idiotypic antibody; or an antibody that binds to a constant region of the extracellular binding domain). In other embodiments, the CAR ligand is a CAR antigen molecule (e.g., a CAR antigen molecule as described herein).

In yet other embodiments, a method for depleting (e.g., reducing and/or killing) a CAR expressing cell is provided. The method includes contacting the CAR expressing cell with a CAR ligand as described herein; and targeting the cell on the basis of binding of the CAR ligand thereby reducing the number, and/or killing, the CAR-expressing cell. In one embodiment, the CAR ligand is coupled to a toxic agent (e.g., a toxin or a cell ablative drug). In another embodiment, the anti-idiotypic antibody can cause effector cell activity, e.g., ADCC or ADC activities.

Exemplary anti-CAR antibodies that can be used in the methods disclosed herein are described, e.g., in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference. In some aspects and embodiments, the compositions and methods herein are optimized for a specific subset of T cells, e.g., as described in US Serial No. PCT/US2015/043219 filed Jul. 31, 2015, the contents of which are incorporated herein by reference in their entirety. In some embodiments, the optimized subsets of T cells display an enhanced persistence compared to a control T cell, e.g., a T cell of a different type (e.g., CD8+ or CD4+) expressing the same construct.

In some embodiments, a CD4+ T cell comprises a CAR described herein, which CAR comprises an intracellular signaling domain suitable for (e.g., optimized for, e.g., leading to enhanced persistence in) a CD4+ T cell, e.g., an ICOS domain. In some embodiments, a CD8+ T cell comprises a CAR described herein, which CAR comprises an intracellular signaling domain suitable for (e.g., optimized for, e.g., leading to enhanced persistence of) a CD8+ T cell, e.g., a 4-1BB domain, a CD28 domain, or another costimulatory domain other than an ICOS domain. In some embodiments, the CAR described herein comprises an antigen binding domain described herein, e.g., a CAR comprising an antigen binding domain.

In an aspect, described herein is a method of treating a subject, e.g., a subject having cancer. The method includes administering to said subject, an effective amount of:
1) a CD4+ T cell comprising a CAR (the CARCD4+) comprising:
an antigen binding domain, e.g., an antigen binding domain described herein;
a transmembrane domain; and
an intracellular signaling domain, e.g., a first costimulatory domain, e.g., an ICOS domain; and
2) a CD8+ T cell comprising a CAR (the CARCD8+) comprising:
an antigen binding domain, e.g., an antigen binding domain described herein;
a transmembrane domain; and
an intracellular signaling domain, e.g., a second costimulatory domain, e.g., a 4-1BB domain, a CD28 domain, or another costimulatory domain other than an ICOS domain;
wherein the CARCD4+ and the CARCD8+ differ from one another.

Optionally, the method further includes administering:
3) a second CD8+ T cell comprising a CAR (the second CARCD8+) comprising:
an antigen binding domain, e.g., an antigen binding domain described herein;
a transmembrane domain; and
an intracellular signaling domain, wherein the second CARCD8+ comprises an intracellular signaling domain, e.g., a costimulatory signaling domain, not present on the CARCD8+, and, optionally, does not comprise an ICOS signaling domain.

### Biopolymer Delivery Methods

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic. Exemplary biopolymers are described, e.g., in paragraphs 1004-1006 of International Application WO2015/142675, filed Mar. 13, 2015, which is herein incorporated by reference in its entirety.

### BCL2 Inhibitors

In some embodiments, the combination described herein includes a BCL2 inhibitor. In some embodiments, the BCL2 inhibitor is chosen from venetoclax, oblimersen (G3139), APG-2575, APG-1252, navitoclax (ABT-263), ABT-737, BP1002, SPC2996, obatoclax mesylate (GX15-070MS), or PNT2258. In some embodiments, the BCL2 inhibitor is administered in combination with a CAR therapy, e.g. a CD19 CAR therapy as described herein.

### Exemplary BCL2 Inhibitors

In some embodiments, the BCL2 inhibitor comprises venetoclax (CAS Registry Number:1257044-40-8), or a compound disclosed in U.S. Pat. Nos. 8,546,399, 9,174,982, and 9,539,251, which are incorporated by reference in their entirety. Venetoclax is also known as venclexta or ABT-0199 or 4-(4-{[2-(4-chlorophenyl)-4,4-dimethylcyclohex-1-en-1-yl]methyl}piperazin-1-yl)-N-(3-nitro-4-{[(oxan-4-yl)methyl]amino}benzenesulfonyl)-2-{1H-pyrrolo[2,3162yridinedin-5-yloxy}benzamide. In certain embodiments, the BCL2 inhibitor is venetoclax. In certain embodiments, the BCL2 inhibitor (e.g., venetoclax) has the following chemical structure:

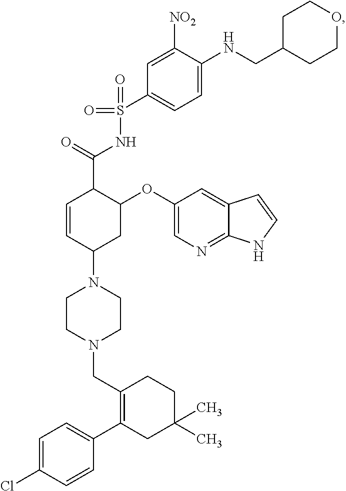 [SMILES: CC1(C)CCC(CN2CCN(C3C=CC(C(=O)NS(=O)(=O)c4ccc(NCC5CCOCC5)c([N+](=O)[O-])c4)C(Oc4cnc5[nH]ccc5c4)C3)CC2)=C(c2ccc(Cl)cc2)C1]

or a pharmaceutically acceptable salt thereof.

In some embodiments, the BCL2 inhibitor comprises a compound of Formula I:

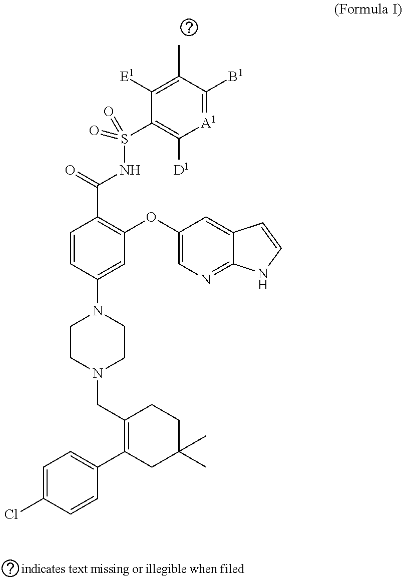

or a pharmaceutically acceptable salt thereof, wherein
A¹ is C(A²);
A² is H, F, Br, I, or Cl;
B¹ is R¹, OR¹, NHR¹, NHC(O)R¹, F, Br, I, or Cl;
D¹ is H, F, Br, I, or Cl;
E¹ is H; and
Y¹ is H, CN, NO₂, F, Cl, Br, I, CF₃, R¹⁷, OR¹⁷, SR¹⁷, SO₂R¹⁷, or C(O)NH₂;
R¹ is R⁴ or R⁵;
R⁴ is cycloalkyl or heterocycloalkyl;
R⁵ is alkyl or alkynyl, each of which is unsubstituted or substituted with one or two or three substituents independently selected from the group consisting of R⁷, OR⁷, NHR⁷, N(R⁷)₂, CN, OH, F, Cl, Br, and I;
R⁷ is R⁸, R⁹, R¹⁰, or R¹¹;
R⁸ is phenyl;
R⁹ is heteroaryl;
R¹⁰ is cycloalkyl, cycloalkenyl, or heterocycloalkyl; each of which is unfused or fused with R^{10A}; R^{10A} is heteroarene;
R¹¹ is alkyl, which is unsubstituted or substituted with one or two or three substituents independently selected from the group consisting of R¹², OR¹², and CF₃;
R¹² is R¹⁴ or R¹⁶.
R¹⁴ is heteroaryl;
R¹⁶ is alkyl;
R¹⁷ is alkyl or alkynyl, each of which is unsubstituted or substituted with one or two or three substituents independently selected from the group consisting of R²², F, Cl, Br and I;
R²² is heterocycloalkyl;
wherein the cyclic moieties represented by R⁴, R⁸, R¹⁰, and R²², are independently unsubstituted or substituted with one or two or three or four or five substituents independently selected from the group consisting of R^{57A}, R²⁷, OR⁵⁷, SO₂R⁵⁷, C(O)R⁵⁷, C(O)OR⁵⁷, C(O)N(R⁵⁷)₂, NH₂, NHR⁵⁷, N(R⁵⁷)₂, NHC(O)R⁵⁷, NHS(O)₂R⁵⁷, OH, CN, (O), F, Cl, Br and I;
R^{57A} is spiroalkyl or spiroheteroalkyl;
R⁵⁷ is R⁵⁸, R⁶⁰, or R⁶¹.
R⁵⁸ is phenyl;
R⁶⁰ is cycloalkyl or heterocycloalkyl;
R⁶¹ is alkyl, which is unsubstituted or substituted with one or two or three substituents independently selected from the group consisting of R⁶², OR⁶², N(R⁶²)₂, C(O)OH, CN, F, Cl, Br, and I;
R⁶² is R⁶⁵ or R⁶⁶.
R⁶⁵ is cycloalkyl or heterocycloalkyl;
R⁶⁶ is alkyl, which is unsubstituted or substituted with OR⁶⁷;
R⁶⁷ is alkyl;
wherein the cyclic moieties represented by R^{57A}, R⁵⁸, and R⁶⁰ are unsubstituted or substituted with one or two or three or four substituents independently selected from the group consisting of R⁶⁸, F, Cl, Br, and I;
R⁶⁸ is R⁷¹ or R⁷²;
R⁷¹ is heterocycloalkyl; and
R⁷² is alkyl, which is unsubstituted or substituted with one or two F.

In some embodiments, the BCL2 inhibitor comprises a compound of Formula II:

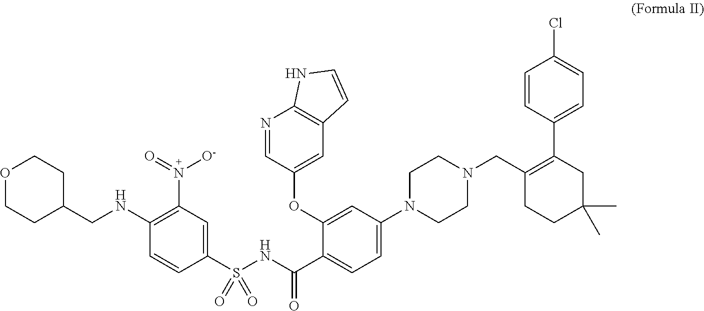

or a pharmaceutically acceptable salt thereof.

In some embodiments the BCL2 inhibitor comprises a compound chosen from:
4-(4-{[2-(4-chlorophenyl)-4,4-dimethylcyclohex-1-en-1-yl]methyl}piperazin-1-yl)-N-({3-nitro-4-[1-tetrahydro-2H-pyran-4-ylpiperidin-4-yl)amino]pheny-1}sulfonyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(1-methylpiperidin-4-yl)amino]-3-nitrophenyl}sulfonyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfony-1)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(4-methylpiperazin-1-yl)amino]-3-nitrophenyl}sulfonyl)-2-(1H-pyrrolo[2,3-b]pyridin-5-yloxy)benzamide;
Trans-4-(4-({[2-(4-chlorophenyl)-4,4-dimethylcyclohex-1-en-1-yl]methyl}pi-perazin-1-yl)-N-({4-[(4-morpholin-4-ylcyclohexyl)amino]-3-nitrophenyl}sulf-onyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(2-methoxyethyl)amino]-3-nitrophenyl}sulfonyl)-2-(1H-pyrrolo-[2,3-b]pyridin-5-yloxy)benzamide;
4-(4-{[2-(4-chlorophenyl)-4,4-dimethylcyclohex-1-en-1-yl]methyl}piperazin-1-yl)-N-[(3-nitro-4-1[(3S)-tetrahydro-2H-pyran-3-ylmethyl]amino}phenyl)su-lfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-{[4-(1,4-dioxan-2-ylmethoxy)-3-nitrophenyl]sulfonyl}-2-(1H-pyrrol-o(2,3-b)pyridin-5-yloxy)benzamide;
4-(4-{[2-(4-chlorophenyl)-4,4-dimethylcyclohex-1-en-1-yl]methyl}piperazin-1-yl)-N-[(3-nitro-4-{[(3R)-tetrahydro-2H-pyran-3-ylmethyl]amino}phenyl)su-lfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(2-methoxyethyl)amino]-3-[(trifluoromethyl)sulfonyl]phenyl}s-ulfonyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-2-(1H-pyrrolo[2,3-b]pyridin-5-yloxy)-N-({4-[(tetrahydro-2H-pyran-4-ylmethyl)amino]-3-[(trifluoromethyl)sulfonyl]phenyl}sulfonyl)benzamide;
4-(4-{[2-(4-chlorophenyl)-4,4-dimethylcyclohex-1-en-1-yl]methyl}piperazin-1-yl)-N-{[3-nitro-4-(tetrahydro-2H-pyran-4-ylmethoxy)phenyl]sulfonyl}-2-(-1H-pyrrolo[2,3-b]pyridin-5-yloxy)benzamide;
4-(4-{[(2-(4-chlorophenyl)-4,4-dimethylcyclohex-1-en-1-yl]methyl}piperazi-n-1-yl)-N-({4-[(1,4-dioxan-2-ylmethyl)amino]-3-nitrophenyl}sulfonyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({3-nitro-4-[(2,2,2-trifluoroethyl)amino]phenyl}sulfonyl)-2-(1H-p-yrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({3-nitro-4-[(3,3,3-trifluoropropyl)amino]phenyl}sulfonyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(2S)-1,4-dioxan-2-ylmethoxy]-3-nitrophenyl}sulfonyl)-2-(1H-p-yrrolo[2,3-b]pyridin-5-yloxy)benzamide; Cis-4-(4-{[2-(4-chlorophenyl)-4,4-dimethylcyclohex-1-en-1-yl]methyl}piper-azin-1-yl)-N-[{(4-1[(4-methoxycyclohexyl)methyl]amino}-3-nitrophenyl)sulfon-yl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(2R)-1,4-dioxan-2-ylmethoxy]-3-nitrophenyl}sulfonyl)-2-(1H-p-yrrolo[2,3-b]pyridin-5-yloxy)benzamide;
Trans-4-(4-{[2-(4-chlorophenyl)-4,4-dimethylcyclohex-1-en-1-yl]methyl}pip-erazin-1-yl)-N-[(4-{[(4-methoxycyclohexyl)methyl]amino}-3-nitrophenyl)sulf-onyl]-2-(1H-pyrrolo[2,3-b]pyridin-5-yloxy)benzamide;
4-(4-{[2-(4-chlorophenyl-4,4-dimethylcyclohex-1-en-1-yl]methyl}piperazin-1-yl)-N-({4-[(4-fluorotetrahydro-2H-pyran-4-yl)methoxy]-3-nitrophenyl}sulf-onyl)-2-(1H-pyrrolo[2,3-b]pyridin-5-yloxy)benzamide;
N-{[3-(aminocarbonyl)-4-(tetrahydro-2H-pyran-4-ylmethoxy)phenyl]sulfonyl}-4-(4-{[2-(4-chlorophenyl)-4,4-dimethylcyclohex-1-en-1-yl]methyl}piperazin-1-yl)-2-(1H-pyrrolo[2,3-b]pyridin-5-yloxy)benzamide;
Cis-4-(4-{[2-(4-chlorophenyl)-4,4-dimethylcyclohex-1-en-1-yl]methyl}piper-azin-1-yl)-N-({4-[(4-morpholin-4-ylcyclohexyl)amino]-3-nitrophenyl}sulfony-1)-2-(1H-pyrrolo(2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(1-methylpiperidin-4-yl)methoxy]-3-nitrophenyl}sulfonyl)-2-(-1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(2,2-dimethyltetrahydro-2H-pyran-4-yl)methoxy]-3-nitrophenyl-}sulfonyl)-2-(1H-pyrrolo[2,3-b]pyridin-5-yloxy)benzamide;
N-({3-chloro-5-cyano-4-[(tetrahydro-2H-pyran-4-ylmethyl)amino]phenyl}sulf-onyl)-4-(4-{(2-(4-chlorophenyl)-4,4-dimethylcyclohex-1-en-1-yl]methyl}pipe-razin-1-yl)-2-(1H-pyrrolo[2,3-b]pyridin-5-yloxy)benzamide;
N-({4-[(1-acetylpiperidin-4-yl)amino]-3-nitrophenyl}sulfonyl)-4-(4-{[2-(4-chlorophenyl)-4,4-dimethylcyclohex-1-en-1-yl]methyl}piperazin-1-yl)-2-(1H-pyrrolo[2,3-b]pyridin-5-yloxy)benzamide;
N-({2-chloro-5-fluoro-4-[(tetrahydro-2H-pyran-4-ylmethyl)amino]phenyl}sul-fonyl)-4-(4-{[2-(4-chlorophenyl)-4,4-dimethylcyclohex-1-en-1-yl]methyl}pip-erazin-1-yl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(3-morpholin-4-ylpropyl)amino]-3-nitrophenyl}sulfonyl)-2-(1H-pyrrolo[2,3-b]pyridin-5-yloxy)benzamide;
Trans-4-(4-{[2-(4-chlorophenyl)-4,4-dimethylcyclohex-1-en-1-yl]methyl}pip-erazin-1-yl)-N-({4-[(4-morpholin-4-ylcyclohexyl)oxy]-3-nitrophenyl}sulfony-1)-2-(1H-pyrrolo[2,3-h]pyridin-5-yloxy)benzamide;
4-(4-{[2-(4-chlorophenyl)-4,4-dimethylcyclohex-1-en-1-yl]methyl}piperazin-1-yl)-N-({4-[(2-cyanoethyl)amino]-3-nitrophenyl}sulfonyl)-2-(1H-pyrrolo[2-,3-b]pyridin-5-yloxy)benzamide;
Trans-N-{[4-({4-[bis(cyclopropylmethyl)amino]cyclohexyl}amino)-3-nitrophe-nyl]sulfonyl}-4-(4-{(2-(4-chlorophenyl)-4,4-dimethylcyclohex-1-en-1-yl]met-hyl}piperazin-1-yl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(4-{[(1-methylpiperidin-4-yl)methyl]amino}-3-nitrophenyl)sulfony-1]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(morpholin-3-ylmethyl)amino]-3-nitrophenyl}sulfonyl)-2-(1H-p-yrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(4-morpholin-4-ylbut-2-ynyl)oxy]-3-nitrophenyl}sulfonyl)-2-(-1H-pyrrolo[2,3-b]pyridin-5-yloxy)benzamide;
tert-butyl 3-{[4-({[4-(4-{[2-(4-chlorophenyl)-4,4-dimethylcyclohex-1-en-1-yl]methyl}-piperazin-1-yl)-2-(1H-pyrrolo[2,3-b]pyridin-5-yloxy)benzoyl]amino}sulfonyl-)-2-nitrophenoxy]methyl}morpholine-4-carboxylate;
4-(4-{[2-(4-chlorophenyl)-4,4-dimethylcyclohex-1-en-1-yl]methyl}piperazin-1-yl)-N-{[4-(morpholin-3-ylmethoxy)-3-nitrophenyl]sulfonyl}-2-(1H-pyrrolo-[2,3-b]pyridin-5-yloxy)benzamide;
4-(4-{[2-(4-chlorophenyl)-4,4-dimethylcyclohex-1-en-1-yl]methyl}piperazin-1-yl)-N-[(4-{[1H-(methylsulfonyl)piperidin-4-yl]amino}-3-nitrophenyl)sulfo-nyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(1,1-dioxidotetrahydro-2H-thiopyran-4-yl)amino]-3-nitropheny-1}sulfonyl)-2-(1H-pyrrolo[2,3-b]pyridin-5-yloxy)benzamide;
N-[(4-chloro-3-nitrophenyl)sulfonyl]-4-(4-{[2-(4-chlorophenyl)-4,4-dimeth-ylcyclohex-1-en-1-yl]methyl}piperazin-1-yl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(3-nitro-4-{[1-(2,2,2-trifluoroethyl)piperidin-4-yl]amino}phenyl-)sulfonyl]-2-(1H-pyrrolo[2,3-b]pyridin-5-yloxy)benzamide;
N-({3-chloro-5-fluoro-4-[(tetrahydro-2H-pyran-4-ylmethyl)amino]phenyl}sul-fonyl)-4-(4-{[2-(4-chlorophenyl)-4,4-dimethylcyclohex-1-en-1-yl]methyl}pip-erazin-1-yl-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-{[4-({1-[2-fluoro-1-(fluoromethyl)ethyl]piperidin-4-yl}amino)-3-n-itrophenyl]sulfonyl}-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(4-{[1-(2,2-difluoroethyl)piperidin-4-yl]amino}-3-nitrophenyl)su-lfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl]-N-({4-[(1-cyclopropylpiperidin-4-yl)amino]-3-nitrophenyl}sulfonyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(4-{1[(1-morpholin-4-ylcyclohexyl)methyl]amino}-3-nitrophenyl)sul-fonyl]-2-(1H-pyrrolo[2,3-b]pyridin-5-yloxy)benzamide;
Trans-4-(4-{[2-(4-chlorophenyl)-4,4-dimethylcyclohex-1-en-1-yl]methyl}pip-erazin-1-yl)-N-[(4-{[4-(dicyclopropylamino)cyclohexyl]amino}-3-nitrophenyl-)sulfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(4-ethylmorpholin-3-yl)methoxy]-3-nitrophenyl}sulfonyl)-2-(1-H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({3-nitro-4-[(4-tetrahydro-2H-pyran-4-ylmorpholin-3-yl)methoxy]ph-enyl}sulfonyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(3-nitro-4-{[(3S)-1-tetrahydro-2H-pyran-4-ylpiperidin-3-yl]amino-}phenyl)sulfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-(4-{[(1,1-dioxidothiomorpholin-4-yl)amino]-3-nitrophenyl}sulfonyl-)-2-(1H-pyrrolo[2,3-b]pyridin-5-yloxy)benzamide;
N-[(4-{[(4-aminotetrahydro-2H-pyran-4-yl)methyl]amino}-3-nitrophenyl)sulf-onyl]-4-(4-{[2-(4-chlorophenyl)-4,4-dimethylcyclohex-1-en-1-yl]methyl}pipe-razin-1-yl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({3-cyano-4-[(tetrahydro-2H-pyran-4-ylmethyl)amino]phenyl}sulfony-1)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(4-[(1S,3R)-3-morpholin-4-ylcyclopentyl]amino}-3-nitrophenyl)sul-fonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(4-{[(1R,3S)-3-morpholin-4-ylcyclopentyl]amino}-3-nitrophenyl)su-lfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({4-[(morpholin-2-ylmethyl)amino]-3-nitrophenyl}sulfonyl)-2-(1H-p-yrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({3-nitro-4-[(tetrahydrofuran-3-ylmethyl)amino]phenyl}sulfonyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-{[4-({1-[cis-3-fluorotetrahydro-2H-pyran-4-yl]piperidin-4-yl}amin-o)-3-nitrophenyl]sulfonyl}-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({3-nitro-4-[(1-tetrahydro-2H-pyran-4-ylazetidin-3-yl)amino]pheny-1}sulfonyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({3-nitro-4-[(1-tetrahydrofuran-3-ylazetidin-3-yl)amino]phenyl}su-lfonyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-{[3-nitro-4-({[(3R)-1-tetrahydro-2H-pyran-4-ylpyrrolidin-3-yl]met-hyl}amino)phenyl]sulfonyl}-2-(1H-pyrrolo[2,3-b]pyridin-5-yloxy)benzamide;
2-(1H-pyrrolo[2,3-b]pyridin-5-yloxy)-4-(4-((2-(4-chlorophenyl)-4,4-dimeth-ylcyclohex-1-enyl)methyl)piperazin-1-yl)-N-(4-((trans-4-hydroxycyclohexyl)-methoxy)-3-nitrophenylsulfonyl)benzamide;
2-(1H-pyrrolo[2,3-b]pyridin-5-yloxy)-4-(4-((2-(4-chlorophenyl)-4,4-dimeth-ylcyclohex-1-enyl)methyl)piperazin-1-yl)-N-(4-((cis-4-methoxycyclohexyl)me-thoxy)-3-nitrophenylsulfonyl)benzamide;
Cis-4-(4-{[2-(4-chlorophenyl)-4,4-dimethylcyclohex-1-en-1-yl]methyl}piper-azin-1-yl)-N-[(4-{[4-(cyclopropylamino)cyclohexyl]amino}-3-nitrophenyl)sul-fonyl]-2-(1H-pyrrolo[2,3-b]pyridin-5-yloxy)benzamide;
Trans-4-(4-{[2-(4-chlorophenyl)-4,4-dimethylcyclohex-1-en-1-yl]methyl}pip-erazin-1-yl)-N-[(3-nitro-4-{[4-tetrahydro-2H-pyran-4-ylamino)cyclohexyl]am-ino}phenyl)sulfonyl]-2-(1H-pyrrolo[2,3-b]pyridin-5-yloxy)benzamide;
Trans-4-(4-{[2-(4-chlorophenyl)-4,4-dimethylcyclohex-1-en-1-yl]methyl}pip-erazin-1-yl)-N-({4-[(4-methoxycyclohexyl)methoxy]-3-nitrophenyl}sulfonyl)-2-(1H-pyrrolo[2,3-b]pyridin-5-yloxy)benzamide;
tert-butyl 4-{[4-({[4-(4-{[2-(4-chlorophenyl)-4,4-dimethylcyclohex-1-en-1-yl]methyl}-piperazin-1-yl)-2-(1H-pyrrolo[2,3-b]pyridin-5-yloxy)benzoyl]amino}sulfonyl-)-2-nitrophenoxy]methyl}-4-fluoropiperidine-1-carboxylate;
4-(4-{[2-(4-chlorophenyl)-4,4-dimethylcyclohex-1-en-1-yl]methyl}piperazin-1-yl)-N-({4-[(4-fluoropiperidin-4-yl)methoxy]-3-nitrophenyl}sulfonyl)-2-(-1H-pyrrolo[2,3-b]pyridin-5-yloxy)benzamide;
Trans-4-(4-{[2-(4-chlorophenyl)-4,4-dimethylcyclohex-1-en-1-yl]methyl}pip-erazin-1-yl)-N-[(3-nitro-4-{(4-(4-tetrahydro-2H-pyran-4-ylpiperazin-1-yl)c-yclohexyl]amino}phenyl)sulfonyl]-2-(1H-pyrrolo[2,3-b]pyridin-5-yloxy)benza-mide;
4-(4-{[2-(4-chlorophenyl)-4,4-dimethylcyclohex-1-en-1-yl]methyl}pipe-razin-1-yl)-N-{[4-({1-[2-fluoro-1-(fluoromethyl)ethyl]piperidin-4-yl}metho-xy)-3-nitrophenyl]sulfonyl}-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(3-nitro-4-{[(3R)-1-tetrahydro-2H-pyran-4-ylpyrrolidin-3-yl]amin-o}phenyl)sulfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(4-[(3R)-1-(2,2-dimethyltetrahydro-2H-pyran-4-yl)pyrrolidin-3-yl-]amino}-3-nitrophenyl)sulfonyl]-2-(1H-pyrrolo[2,3-b]pyridin-5-yloxy)benzam-ide;
4-(4-{[2-(4-chlorophenyl-4,4-dimethylcyclohex-1-en-1-yl]methyl}pipera-zin-1-yl)-N-[(3-nitro-4-55 [(3S)-1-tetrahydro-2H-pyran-4-ylpyrrolidin-3-yl]a-mino}phenyl)sulfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(4-{[(3S)-1-(2,2-dimethyltetrahydro-2H-pyran-4-yl)pyrrolidin-3-y-1]amino}-3-nitrophenyl)sulfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}pipe-razin-1-yl)-N-[(4-{[(4-methylmorpholin-2-yl)methyl]amino}-3-nitrophenyl)su-lfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-{[4-({[4-(2-methoxyethyl)morpholin-2-yl]methyl}amino)-3-nitrophen-yl]sulfonyl}-2-(1H-pyrrolo[2,3-b]pyridin-5-yloxy)benzamide;
N-[(4-{[(4-acetylmorpholin-2-yl)methyl]amino}-3-nitrophenyl)sulfonyl]-4-(-4-{[2-(4-chlorophenyl)-4,4-dimethylcyclohex-1-en-1-yl]methyl}piperazin-1-y-1)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(4-([trans-4-(fluoromethyl)-1-oxetan-3-ylpyrrolidin-3-yl]methoxy-}-3-nitrophenyl)sulfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(4-{[(4-fluorotetrahydro-2H-pyran-4-yl)methyl]amino}-3-nitrophen-yl)sulfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({3-nitro-4-[(1-oxetan-3-ylpiperidin-4-yl)amino]phenyl}sulfonyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-(14-[(1-cyclobutylpiperidin-4-yl)amino]-3-nitrophenyl}sulfonyl)-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[{4-([1-(2,2-dimethyltetrahydro-2H-pyran-4-yl)piperidin-4-yl]amin-o}-3-nitrophenyl)sulfonyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-[(4-{[(3S)-1-cyclopropylpyrrolidin-3-yl]amino}-3-nitrophenyl)sulf-onyl]-2-(1H-pyrrolo[2,3-b]pyridin-5-yloxy)benzamide;
4-(4-{[2-(4-chlorophenyl)-4,4-dimethylcyclohex-1-en-1-yl]methyl}piperazin-1-yl)-N-({3-nitro-4-[(1-tetrahydrofuran-3-ylpiperidin-4-yl)amino]phenyl}s-ulfonyl)-2-(1H-pyrrolo[2,3-b]pyridin-5-yloxy)benzamide; or
   a pharmaceutically acceptable salt thereof.

In some embodiments, the BCL2 inhibitor is administered at dose of about 10 mg to about 500 mg, e.g., about 20 mg to about 400 mg, about 50 mg to about 350 mg, about 100 mg to about 300 mg, about 150 mg to about 250 mg, 50 mg to about 500 mg, about 100 mg to about 500 mg, about 150 mg to about 500 mg, about 200 mg to about 500 mg, about 250 mg to about 500 mg, about 300 mg to about 500 mg, about 350 mg to about 500 mg, about 400 mg to about 500 mg, about 450 mg to about 500 mg, about 10 mg to about 400 mg, about 10 mg to about 350 mg, about 10 mg to 300 mg, about 10 mg to about 250 mg, about 10 mg to about 200 mg, about 10 mg to about 150 mg, about 10 mg to about 100 mg, about 10 mg to about 50 mg, about 50 mg to about 150 mg, about 150 mg to about 250 mg, about 250 mg to about 350 mg, or about 350 mg to about 400 mg. In some embodiments, the BCL2 inhibitor is administered at a dose of about 20 mg, 50 mg, 100 mg, 150 mg, 200 mg, 250 mg, 300 mg, 350 mg, 400 mg, 450 mg, or 500 mg. In some embodiments, the BCL2 inhibitor is administered daily. In some embodiments, the BCL2 inhibitor is administered at least once a day. In some embodiments, the BCL2 inhibitor is administered for at least 5-10 consecutive days. In some embodiments, the BCL2 inhibitor is administered orally. In some embodiments, the BCL2 inhibitor is administered at a fixed dose. In some embodiments, the BCL2 inhibitor is administered in a ramp-up cycle. In some embodiments, the BCL2 inhibitor is administered in a ramp-up cycle followed by a fixed dose.

In some embodiments, the BCL2 inhibitor is administered in a ramp-up cycle for e.g. about 5 weeks, followed by fixed dose for e.g., at least about 24 months. In some embodiments, the BCL2 inhibitor is administered at a dose of about 10 mg to about 30 mg (e.g., about 20 mg) once a day for e.g., about 1 week, followed by about 40 mg to about 60 mg (e.g., about 50 mg) once a day for e.g., about 1 week, followed by about 80 mg to about 120 mg (e.g., about 100 mg) once a day for e.g., about 1 week, followed by about 150 mg to about 250 mg (e.g., about 200 mg) once a day for e.g., about 1 week, followed by about 350 mg to about 450 mg (e.g., about 400 mg) once a day for e.g., about 1 week, and followed by a fixed dose, e.g., about 350 mg to about 450 mg (e.g., about 400 mg), once a day, for e.g., at least about 24 months.

### Other Exemplary BCL2 Inhibitors

In some embodiments, the BCL2 inhibitor comprises oblimersen, e.g., oblimersen sodium (CAS Registry Number: 190977-41-4). Oblimersen or oblimersen sodium is also known as Genasense, Augmerosen, BCL2 antisense oligodeoxynucleotide G3139, or heptadecasodium; 1-[(2R,4S,5R)-5-[[[(2R,3S,5R)-2-[[[(2R,3S,5R)-2-[[[(2R,3S,5R)-2-[[[(2R,3S,5R)-5-(2-amino-6-oxo-1H-purin-9-yl)-2-[[[(2R,3S,5R)-2-[[[(2R,3S,5R)-5-(2-amino-6-oxo-1H-purin-9-yl)-2-[[[(2R,3S,5R)-2-[[[(2R,3S,5R)-5-(2-amino-6-oxo-1H-purin-9-yl)-2-[[[(2R,3S,5R)-2-[[[(2R,3S,5R)-5-(2-amino-6-oxo-1H-purin-9-yl)-2-[[[(2R,3S,5R)-2-[[[(2R,3S,5R)-5-(4-amino-2-oxopyrimidin-1-yl)-2-[[[(2R,3S,5R)-5-(4-amino-2-oxopyrimidin-1-yl)-2-[[[(2R,3S,5R)-5-(4-amino-2-oxopyrimidin-1-yl)-2-[[[(2R,3S,5R)-2-[[[(2R,3S,5R)-5-(4-amino-2-oxopyrimidin-1-yl)-2-[[[(2R,3S,5R)-2-(hydroxymethyl)-5-(5-methyl-2,4-dioxopyrimidin-1-yl)oxolan-3-yl]oxy-oxidophosphinothioyl]oxymethyl]oxolan-3-yl]oxy-oxidophosphinothioyl]oxymethyl]-5-(5-methyl-2,4-dioxopyrimidin-1-yl)oxolan-3-yl]oxy-oxidophosphinothioyl]oxymethyl]oxolan-3-yl]oxy-oxidophosphinothioyl]oxymethyl]oxolan-3-yl]oxy-oxidophosphinothioyl]oxymethyl]oxolan-3-yl]oxy-oxidophosphinothioyl]oxymethyl]-5-(6-aminopurin-9-yl)oxolan-3-yl]oxy-oxidophosphinothioyl]oxymethyl]oxolan-3-yl]oxy-oxidophosphinothioyl]oxymethyl]-5-(4-amino-2-oxopyrimidin-1-yl)oxolan-3-yl]oxy-oxidophosphinothioyl]oxymethyl]oxolan-3-yl]oxy-oxidophosphinothioyl]oxymethyl]-5-(5-methyl-2,4-dioxopyrimidin-1-yl)oxolan-3-yl]oxy-oxidophosphinothioyl]oxymethyl]oxolan-3-yl]oxy-oxidophosphinothioyl]oxymethyl]-5-(4-amino-2-oxopyrimidin-1-yl)oxolan-3-yl]oxy-oxidophosphinothioyl]oxymethyl]oxolan-3-yl]oxy-oxidophosphinothioyl]oxymethyl]-5-(4-amino-2-oxopyrimidin-1-yl)oxolan-3-yl]oxy-oxidophosphinothioyl]oxymethyl]-5-(4-amino-2-oxopyrimidin-1-yl)oxolan-3-yl]oxy-oxidophosphinothioyl]oxymethyl]-5-(6-aminopurin-9-yl)oxolan-3-yl]oxy-oxidophosphinothioyl]oxymethyl]-4-hydroxyoxolan-2-yl]-5-methylpyrimidine-2,4-dione. Oblimersen has the molecular formula of C172H221N62091P17S17. Oblimersen sodium is a sodium salt of a phosphorothioate antisense oligonucleotide that is targeted to the initiation codon region of the BCL2 mRNA where it inhibits BCL2 mRNA translation, and is disclosed, e.g., in Banerjee Curr Opin Mol Ther. 1999; 1(3):404-408.

In some embodiments, the BCL2 inhibitor comprises APG-2575. APG-2575 is also known as BCL2 inhibitor APG 2575, APG 2575, or APG2575. APG-2575 is an inhibitor selective for BCL2 with potential pro-apoptotic and antineoplastic activities. Upon oral administration, BCL2 inhibitor APG 2575 targets, binds to and inhibits the activity of BCL2. APG-2575 is disclosed, e.g., in Fang et al. Cancer Res. 2019 (79) (13 Supplement) 2058. In some embodiments, APG-2575 is administered at a dose of about 20 mg to about 800 mg (e.g., about 20 mg, 50 mg, 100 mg, 200 mg, 400 mg, 600 mg, or 800 mg). In some embodiments, APG-2575 is administered once a day. In some embodiments, APG-2575 is administered orally.

In some embodiments, the BCL2 inhibitor comprises APG-1252. APG-1252 is also known as BCL2/Bcl-XL inhibitor APG-1252 or APG 1252. APG-1252 is a BCL2 homology (BH)-3 mimetic and selective inhibitor of BCL2 and Bcl-XL, with potential pro-apoptotic and antineoplastic activities. Upon administration, APG-1252 specifically binds to and inhibits the activity of the pro-survival proteins BCL2 and Bcl-XL, which restores apoptotic processes and inhibits cell proliferation in BCL2/Bcl-XL-dependent tumor cells. APG-1252 is disclosed, e.g., in Lakhani et al. Journal of Clinical Oncology 2018 36:15_suppl, 2594-2594. In some embodiments, APG-1252 is administered at a dose of about 10 mg to about 400 mg (e.g., about 10 mg, about 40 mg, about 160 mg, or about 400 mg). In some embodiments, APG-1252 is administered twice a week. In some embodiments, APG-1252 is administered intravenously.

In some embodiments, the BCL2 inhibitor comprises navitoclax. Navitoclax is also known as ABT-263 or 4-[4-[[2-(4-chlorophenyl)-5,5-dimethylcyclohexen-1-yl]methyl]piperazin-1-yl]-N-[4-[[(2R)-4-morpholin-4-yl-1-phenylsulfanylbutan-2-yl]amino]-3-(trifluoromethylsulfonyl)phenyl]sulfonylbenzamide. Navitoclax is a synthetic small molecule and an antagonist of the BCL2 proteins. It selectively binds to apopotosis suppressor proteins BCL2, Bcl-XL, and Bcl-w, which are frequently overexpressed in cancerous cells. Inhibition of these protein prevents their binding to the apoptotic effector proteins, Bax and Bak, which triggers apoptotic processes. Navitoclax is disclosed, e.g., in Gandhi et al. J Clin Oncol. 2011 29(7):909-916. In some embodiments, navitoclax is administered orally.

In some embodiments, the BCL2 inhibitor comprises ABT-737. ABT-737 is also known as 4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide. ABT-737 is a small molecule, BCL2 Homology 3 (BH3) mimetic with pro-apoptotic and antineoplastic activities. ABT-737 binds to the hydrophobic groove of multiple members of the anti-apoptotic BCL2 protein family, including BCL2, Bcl-xl and Bcl-w. This inhibits the activity of these pro-survival proteins and restores apoptotic processes in tumor cells, via activation of Bak/Bax-mediated apoptosis. ABT-737 is disclosed, e.g., in Howard et al. Cancer Chemotherapy and Pharmacology 2009 65(1):41-54. In some embodiments, ABT-737 is administered orally.

In some embodiments, the BCL2 inhibitor comprises BP1002. BP1002 is an antisense therapeutic that is comprised of an uncharged P-ethoxy antisense oligodeoxynucleotide targeted against BCL2 mRNA. BP1002 is disclosed, e.g., in Ashizawa et al. Cancer Research 2017 77(13). In some embodiments, BP1002 is incorporated into liposomes for administration. In some embodiments, BP1002 is administered intravenously.

In some embodiments, the BCL2 inhibitor comprises SPC2996. SPC2996 is locked nucleic acid phosphorothioate antisense molecule targeting the mRNA of the BCL2 oncoprotein SPC2996 is disclosed, e.g., in Durig et al. Leukemia 2011 25(4)638-47. In some embodiments, SPC2996 is administered intravenously.

In some embodiments, the BCL2 inhibitor comprises obatoclax, e.g., obatoclax mesylate (GX15-070MS). Obatoclax mesylate is also known as (2E)-2-[(5E)-5-[(3,5-dimethyl-1H-pyrrol-2-yl)methylidene]-4-methoxypyrrol-2-ylidene]indole; methanesulfonic acid. It is the mesylate salt of obatoclax, which is a synthetic small-molecule inhibitor of the BCL2 protein family and has pro-apoptotic and antineoplastic activities. Obatoclax binds to members of the BCL2 protein family, preventing their binding to the pro-apoptotic proteins Bax and Bak. This promotes activation of apopotosis in BCL2-overexpressing cells. Obatoclax mesylate is disclosed, e.g., in O'Brien et al. Blood 2009 113(2):299-305. In some embodiments, obatoclax mesylate is administered intravenously.

In some embodiments, the BCL2 inhibitor comprises PNT2258. PNT225 is phosphodiester DNA oligonucleotide that hybridizes to genomic sequences in the 5′ untranslated region of the BCL2 gene and inhibits its transcription through the process of DNA interference (DNAi). PNT2258 is disclosed, e.g., in Harb et al. Blood (2013) 122(21):88. In some embodiments, PNT2258 is administered intravenously.

### BCL6 Inhibitors

In some embodiments, the combination described herein includes a BCL6 inhibitor. In some embodiments, the BCL6 inhibitor is chosen from compound 79-6, BI-3812, or FX1.

In some embodiments, the BCL6 inhibitor is compound 79-6. Compound 79-6 is disclosed, e.g., in Cerchietti et al. Cancer Cell 2010; 17(4):400-411. Compound 79-6 is a small molecule inhibitor that binds the BTB domain of BCL6 and induce expression of BCL6 target genes.

In some embodiments, the BCL6 inhibitor is BI-3812. BI-3812 is disclosed, e.g., in Kerres et al. Cell Reports 2017; 20:2860-2875. BI-3812 binds and inhibits the BTB domain of BCL6.

In some embodiments, the BCL6 inhibitor is FX1. FX1 is disclosed, e.g., in Cardenas et al. Journal of Clinical Investigation 2016; 126(9):3351-3362. FX1 binds an essential region of the BCL6 lateral groove and disrupts formation of the BCL6 repression complex, reactivating BCL6 target genes.

In some embodiments, the BCL6 inhibitor is administered in combination with a CAR therapy, e.g. a CD19 CAR therapy as described herein.

### MYC Inhibitors

In some embodiments, the combination described herein includes a MYC inhibitor. In some embodiments, the MYC inhibitor indirectly inhibits MYC, e.g., inhibits a gene target upstream and/or downstream of MYC. In some embodiments, the MYC inhibitor inhibits at least one of a myc-associated factor X (Max), ubiquitin proteasome, mammalian target of rapamycin (mTOR), glycogen synthase kinase-3 (GSK-33), histone deacetylase (HDAC), phosphoinositide 3 kinase (PI3K), BET bromodomain, or Aurora A and Aurora B kinases, polo-like kinase-1 (PLK-1).

In some embodiments, the MYC inhibitor is MLN0128. MLN0128 inhibits mTOR. In some embodiments, the MYC inhibitor is 9-ING-41. 9-ING-41 inhibits GSK-3p, a downstream target of mTOR and upstream target of MYC. In some embodiments, the MYC inhibitor is CUDC-907. CUDC-907 is an inhibitor of HDAC and P13K. CUDC-907 is also known as fimepinostat. MLN0128, 9-ING-41, and CUDC-907 are disclosed, e.g., in Li et al. Expert Review of Hematology 2019; 12(7):507-514.

In some embodiments, the MYC inhibitor is Omomyc. Omomyc is disclosed, e.g., in Demma et al ASM Molecular and Cellular Biol. 2019; 39(22):e00248-19. Oncomyx binds Max and inhibits the Max/MYC heterodimer formation.

In some embodiments, the MYC inhibitor is administered in combination with a CAR therapy, e.g. a CD19 CAR therapy as described herein.

### Combination Therapies

In some aspects, the disclosure provides a method of treating a subject, comprising administering a CAR therapy, e.g., a CAR that binds a B-cell antigen, produced as described herein, in combination with one or more other therapies. In some aspects, the disclosure provides a method of treating a subject, comprising administering a reaction mixture comprising a CAR therapy as as described herein, in combination with one or more other therapies. In some aspects, the disclosure provides a method of shipping or receiving a reaction mixture comprising CAR-expressing cells as described herein. In some aspects, the disclosure provides a method of treating a subject, comprising receiving a CAR-expressing cell that was produced as described herein, and further comprising administering the CAR-expressing cell to the subject, optionally in combination with one or more other therapies. In some aspects, the disclosure provides a method of treating a subject, comprising producing a CAR-expressing cell as described herein, and further comprising administering the CAR-expressing cell to the subject, in combination with one or more other therapies. In some embodiments, th CAR therapy, e.g., a CAR that binds a B-cell antigen is administered in combination with a BCL2 inhibitor, a BCL6 inhibitor, a MYC inhibitor, or a combination thereof. In some embodiments, the B-cell antigen is chosen from CD19, CD20, CD22, CD34, CD123, BCMA, FLT-3, or ROR1. In some embodiments, the B-cell antigen is CD19. In some embodiments, the B-cell antigen is CD20. In some embodiments, the B-cell antigen is CD22. In some embodiments, the B-cell antigen is CD34. In some embodiments, the B-cell antigen is CD123. In some embodiments, the B-cell antigen is BCMA. In some embodiments, the B-cell antigen is FLT-3. In some embodiments, the B-cell antigen is ROR1.

In some embodiments, the CAR therapy is a CD19 CAR therapy. In some embodiments, the CD19 CAR therapy, e.g., a CD19 CAR as described herein, is administered in combination with a BCL2 inhibitor, e.g., a BCL2 inhibitor described herein. In some embodiments, the BCL2 inhibitor is venetoclax.

In some embodiments, the CD123 CAR therapy, e.g., a CD123 CAR as described herein, is administered in combination with a BCL2 inhibitor, e.g., a BCL2 inhibitor described herein. In some embodiments, the BCL2 inhibitor is venetoclax.

In some embodiments, the CAR therapy is administered in a combination with one or more of a BCL2, a BLC6 inhibitor, or a MYC inhibitor optionally further comprising administration of one or more other therapies. In some embodiments, the other therapy may be, e.g., a B cell inhibitor (e.g., one or more inhibitors of CD19, CD20, CD22, CD34, CD123, BCMA, FLT-3, or ROR1, e.g., as described herein) or a cancer therapy such as, R-CHOP, DA-EPOC-R, R-CODOX-M/IVAC, R-Hyper-CVAD, and/or chemotherapy.

The combination of a CAR as described herein (e.g., or a CD19 CAR-expressing cell described herein and one or more of a BCL2 inhibitor, a BCL6 inhibitor, or a MYC inhibitor, e.g., as described herein) may be used in combination with other known agents and therapies.

B cell inhibitors, combination therapies comprising the same and uses thereof are described in International Application WO 2016/164731 filed on Apr. 8, 2016, which is incorporated by reference in its entirety.

In further aspects, the combination of the CAR therapy described herein (e.g., a CD19 CAR therapy, in combination with one or more of a BCL2 inhibitor, a BCL6 inhibitor, or a MYC inhibitor) may be used in a treatment regimen in combination with surgery, chemotherapy, radiation, an mTOR pathway inhibitor, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation, peptide vaccine, such as that described in Izumoto et al. 2008 J Neurosurg 108:963-971.

In one embodiment, the combination of a CAR therapy, e.g., a CD19 CAR therapy, described herein and one or more a BCL2 inhibitor, BCL6 inhibitor, or MYC inhibitor can be used in combination with a chemotherapeutic agent. Exemplary chemotherapeutic agents include an anthracycline (e.g., doxorubicin (e.g., liposomal doxorubicin)); a vinca alkaloid (e.g., vinblastine, vincristine, vindesine, vinorelbine); an alkylating agent (e.g., cyclophosphamide, decarbazine, melphalan, ifosfamide, temozolomide); an immune cell antibody (e.g., alemtuzamab, gemtuzumab, rituximab, tositumomab); an antimetabolite (including, e.g., folic acid antagonists, pyrimidine analogs, purine analogs and adenosine deaminase inhibitors (e.g., fludarabine)); a TNFR glucocorticoid induced TNFR related protein (GITR) agonist; a proteasome inhibitor (e.g., aclacinomycin A, gliotoxin or bortezomib); an immunomodulator such as thalidomide or a thalidomide derivative (e.g., lenalidomide).

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In an embodiment, the chemotherapeutic agent is administered prior to administration of the cell expressing a CAR molecule, e.g., a CAR molecule described herein. In chemotherapeutic regimens where more than one administration of the chemotherapeutic agent is desired, the chemotherapeutic regimen is initiated or completed prior to administration of a cell expressing a CAR molecule, e.g., a CAR molecule described herein. In embodiments, the chemotherapeutic agent is administered at least 1 day, 2 days, 3 days, 4 days, 5 days, 6 days, 7 days, 8 days, 9 days, 10 days, 11 days, 12 days, 13 days, 14 days, 15 days, 20 days, 25 days, or 30 days prior to administration of the cell expressing the CAR molecule. In embodiments, the chemotherapeutic regimen is initiated or completed at least 1 day, 2 days, 3 days, 4 days, 5 days, 6 days, 7 days, 8 days, 9 days, 10 days, 11 days, 12 days, 13 days, 14 days, 15 days, 20 days, 25 days, or 30 days prior to administration of the cell expressing the CAR molecule. In embodiments, the chemotherapeutic agent is a chemotherapeutic agent that increases expression of CD19, CD20, or CD22 on the cancer cells, e.g., the tumor cells, e.g., as compared to expression on normal or non-cancer cells. Expression can be determined, for example, by immunohistochemical staining or flow cytometry analysis. For example, the chemotherapeutic agent is cytarabine (Ara-C).

Anti-cancer agents of particular interest for combinations with the compounds of the present invention include: antimetabolites; drugs that inhibit either the calcium dependent phosphatase calcineurin or the p70S6 kinase FK506) or inhibit the p70S6 kinase; alkylating agents; mTOR inhibitors; immunomodulators; anthracyclines; vinca alkaloids; proteosome inhibitors; GITR agonists; protein tyrosine phosphatase inhibitors; a CDK4 kinase inhibitor; a BTK kinase inhibitor; a MKN kinase inhibitor; a DGK kinase inhibitor; or an oncolytic virus.

Exemplary antimetabolites include, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In embodiments, a CAR therapy combination described herein is further administered to a subject in combination with rituximab, cyclophosphamide, doxorubicine, vincristine, and/or a corticosteroid (e.g., prednisone). In embodiments, the CAR thereapy combination described herein is administered to a subject in combination with rituximab, cyclophosphamide, doxorubicine, vincristine, and prednisone (R-CHOP). In some embodiments a CD19 CAR therapy is administered in combination with a BCL2 inhibitor, optionally further comprising administration of R-CHOP. In embodiments, the subject has a high grade B-cell lymphoma (e.g., a double and/or triple hit lymphoma, or a non-specified NOS high-grade lymphoma) or a diffuse large B-cell lymphoma (DLBCL). In embodiments, the subject has a double hit lymphoma. In embodiments, the subject has a triple hit lymphoma. In embodiments, the subject has nonbulky limited-stage DLBCL (e.g., comprises a tumor having a size/diameter of less than 7 cm). In embodiments, the subject is treated with radiation in combination with the R-CHOP. For example, the subject is administered R-CHOP (e.g., 1-6 cycles, e.g., 1, 2, 3, 4, 5, or 6 cycles of R-CHOP), followed by radiation. In some cases, the subject is administered R-CHOP (e.g., 1-6 cycles, e.g., 1, 2, 3, 4, 5, or 6 cycles of R-CHOP) following radiation.

In embodiments, a CAR therapy combination described herein is further administered to a subject in combination with etoposide, prednisone, vincristine, cyclophosphamide, doxorubicin, and/or rituximab. In embodiments, a CAR therapy combination described herein is further administered to a subject in combination with etoposide, prednisone, vincristine, cyclophosphamide, doxorubicin, and rituximab (EPOCH-R). In embodiments, a CAR therapy combination described herein is further administered to a subject in combination with dose-adjusted EPOCH-R (DA-EPOCH-R). In some embodiments a CD19 CAR therapy is administered in combination with a BCL2 inhibitor, optionally further comprising administration of EPOCH-R or DA-EPOCH-R. In embodiments, the subject has a B cell lymphoma, e.g., a high grade B-cell lymphoma (e.g., a double and/or triple hit lymphoma or a non-specified NOS high-grade lymphoma), a DLBCL, or a FL.

In embodiments, a CAR therapy combination described herein is further administered to a subject in combination with cyclophosphamide, vincristine, adriamycin, dexamethasone (R-Hyper-CVAD). In some embodiments a CD19 CAR therapy is administered in combination with a BCL2 inhibitor, optionally further comprising administration of Hyper-CVAD. In embodiments, the subject has a B cell lymphoma, e.g., a high grade B-cell lymphoma (e.g., a double and/or triple hit lymphoma, or a non-specified NOS high-grade lymphoma), a DLBCL, or a FL.

In embodiments, a CAR therapy combination described herein is further administered to a subject in combination with cyclophosphamide, doxorubicin, vincristine, methotrexate, alternating with ifosfamide, etoposide and high-dose cytarabine (R-CODOX-M/IVAC). In some embodiments a CD19 CAR therapy is administered in combination with a BCL2 inhibitor, optionally further comprising administration of R-CODOX-M/IVAC. In embodiments, the subject has a B cell lymphoma, e.g., a high grade B-cell lymphoma (e.g., a double and/or triple hit lymphoma, or a non-specified NOS high-grade lymphoma), a DLBCL, or a FL.

In embodiments, a CAR-expressing cell described herein is administered to a subject in combination with rituximab and/or lenalidomide. Lenalidomide ((RS)-3-(4-Amino-1-oxo 1,3-dihydro-2H-isoindol-2-yl)piperidine-2,6-dione) is an immunomodulator. In embodiments, a CAR-expressing cell described herein is administered to a subject in combination with rituximab and lenalidomide. In embodiments, the subject has follicular lymphoma (FL) or mantle cell lymphoma (MCL). In embodiments, the subject has FL and has not previously been treated with a cancer therapy. In embodiments, lenalidomide is administered at a dosage of about 10-20 mg (e.g., 10-15 or 15-20 mg), e.g., daily. In embodiments, rituximab is administered at a dosage of about 350-550 mg/m² (e.g., 350-375, 375-400, 400-425, 425-450, 450-475, or 475-500 mg/m²), e.g., intravenously.

Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.0^{4,9}] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1) (SEQ ID NO: 1316), and XL765.

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

In embodiments, a CAR-expressing cell described herein is administered to a subject in combination with brentuximab. Brentuximab is an antibody-drug conjugate of anti-CD30 antibody and monomethyl auristatin E. In embodiments, the subject has Hodgkin's lymphoma (HL), e.g., relapsed or refractory HL. In embodiments, the subject comprises CD30+HL. In embodiments, the subject has undergone an autologous stem cell transplant (ASCT). In embodiments, the subject has not undergone an ASCT. In embodiments, brentuximab is administered at a dosage of about 1-3 mg/kg (e.g., about 1-1.5, 1.5-2, 2-2.5, or 2.5-3 mg/kg), e.g., intravenously, e.g., every 3 weeks.

In embodiments, a CAR-expressing cell described herein is administered to a subject in combination with brentuximab and dacarbazine or in combination with brentuximab and bendamustine. Dacarbazine is an alkylating agent with a chemical name of 5-(3,3-Dimethyl-1-triazenyl)imidazole-4-carboxamide. Bendamustine is an alkylating agent with a chemical name of 4-[5-[Bis(2-chloroethyl)amino]-1-methylbenzimidazol-2-yl]butanoic acid. In embodiments, the subject has Hodgkin's lymphoma (HL). In embodiments, the subject has not previously been treated with a cancer therapy. In embodiments, the subject is at least 60 years of age, e.g., 60, 65, 70, 75, 80, 85, or older. In embodiments, dacarbazine is administered at a dosage of about 300-450 mg/m² (e.g., about 300-325, 325-350, 350-375, 375-400, 400-425, or 425-450 mg/m²), e.g., intravenously. In embodiments, bendamustine is administered at a dosage of about 75-125 mg/m2 (e.g., 75-100 or 100-125 mg/m², e.g., about 90 mg/m2), e.g., intravenously. In embodiments, brentuximab is administered at a dosage of about 1-3 mg/kg (e.g., about 1-1.5, 1.5-2, 2-2.5, or 2.5-3 mg/kg), e.g., intravenously, e.g., every 3 weeks.

In some embodiments, a CAR-expressing cell described herein is administered to a subject in combination with a CD20 inhibitor, e.g., an anti-CD20 antibody (e.g., an anti-CD20 mono- or bispecific antibody) or a fragment thereof. Exemplary anti-CD20 antibodies include but are not limited to rituximab, ofatumumab, ocrelizumab, veltuzumab, obinutuzumab, TRU-015 (Trubion Pharmaceuticals), ocaratuzumab, and Pro131921 (Genentech). See, e.g., Lim et al. Haematologica. 95.1(2010):135-43.

Dosing regimens comprising CD20 inhibitors are described in International Application WO 2016/164731 filed on Apr. 8, 2016, which is incorporated by reference in its entirety.

In some embodiments, one or more CAR-expressing cells described herein is administered in combination with an oncolytic virus. In embodiments, oncolytic viruses are capable of selectively replicating in and triggering the death of or slowing the growth of a cancer cell. In some cases, oncolytic viruses have no effect or a minimal effect on non-cancer cells. An oncolytic virus includes but is not limited to an oncolytic adenovirus, oncolytic Herpes Simplex Viruses, oncolytic retrovirus, oncolytic parvovirus, oncolytic vaccinia virus, oncolytic Sinbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

In some embodiments, the oncolytic virus is a virus, e.g., recombinant oncolytic virus, described in US2010/0178684 A1, which is incorporated herein by reference in its entirety. In some embodiments, a recombinant oncolytic virus comprises a nucleic acid sequence (e.g., heterologous nucleic acid sequence) encoding an inhibitor of an immune or inflammatory response, e.g., as described in US2010/0178684 A1, incorporated herein by reference in its entirety. In embodiments, the recombinant oncolytic virus, e.g., oncolytic NDV, comprises a pro-apoptotic protein (e.g., apoptin), a cytokine (e.g., GM-CSF, interferon-gamma, interleukin-2 (IL-2), tumor necrosis factor-alpha), an immunoglobulin (e.g., an antibody against ED-B firbonectin), tumor associated antigen, a bispecific adapter protein (e.g., bispecific antibody or antibody fragment directed against NDV HN protein and a T cell co-stimulatory receptor, such as CD3 or CD28; or fusion protein between human IL-2 and single chain antibody directed against NDV HN protein). See, e.g., Zamarin et al. Future Microbiol. 7.3(2012):347-67, incorporated herein by reference in its entirety. In some embodiments, the oncolytic virus is a chimeric oncolytic NDV described in U.S. Pat. No. 8,591,881 B2, US 2012/0122185 A1, or US 2014/0271677 A1, each of which is incorporated herein by reference in their entireties.

In some embodiments, an oncolytic virus described herein is administering by injection, e.g., subcutaneous, intra-arterial, intravenous, intramuscular, intrathecal, or intraperitoneal injection. In embodiments, an oncolytic virus described herein is administered intratumorally, transdermally, transmucosally, orally, intranasally, or via pulmonary administration.

In an embodiment, cells expressing a CAR described herein are administered to a subject in combination with a molecule that decreases the Treg cell population. Methods that decrease the number of (e.g., deplete) Treg cells are known in the art and include, e.g., CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (e.g., Tregs) in the tumor microenvironment and reduces the subject's risk of relapse.

In an embodiment, a CAR-expressing cell described herein is administered to a subject in combination with a molecule that decreases the Treg cell population. Methods that decrease the number of (e.g., deplete) Treg cells are known in the art and include, e.g., CD25 depletion, cyclophosphamide administration, and modulating GITR function. Without wishing to be bound by theory, it is believed that reducing the number of Treg cells in a subject prior to apheresis or prior to administration of a CAR-expressing cell described herein reduces the number of unwanted immune cells (e.g., Tregs) in the tumor microenvironment and reduces the subject's risk of relapse. In one embodiment, CAR-expressing cells described herein are administered to a subject in combination with a molecule targeting GITR and/or modulating GITR functions, such as a GITR agonist and/or a GITR antibody that depletes regulatory T cells (Tregs). In one embodiment, CAR-expressing cells described herein are administered to a subject in combination with cyclophosphamide. In one embodiment, the GITR binding molecule and/or molecule modulating GITR function (e.g., GITR agonist and/or Treg depleting GITR antibodies) is administered prior to the CAR-expressing cells. For example, in one embodiment, the GITR agonist can be administered prior to apheresis of the cells. In embodiments, cyclophosphamide is administered to the subject prior to administration (e.g., infusion or re-infusion) of the CAR-expressing cell or prior to apheresis of the cells. In embodiments, cyclophosphamide and an anti-GITR antibody are administered to the subject prior to administration (e.g., infusion or re-infusion) of the CAR-expressing cell or prior to apheresis of the cells.

In one embodiment, a CAR expressing cell described herein is administered to a subject in combination with a GITR agonist, e.g., a GITR agonist described herein. In one embodiment, the GITR agonist is administered prior to the CAR-expressing cell. For example, in one embodiment, the GITR agonist can be administered prior to apheresis of the cells.

Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In one embodiment, a CAR-expressing cell described herein is administered in combination with a kinase inhibitor. Exemplary kinase inhibitors and uses thereof, are described in International Application WO 2016/164731 filed on Apr. 8, 2016, which is incorporated by reference in its entirety.

In embodiments, a CAR-expressing cell described herein is administered to a subject in combination with an indoleamine 2,3-dioxygenase (IDO) inhibitor. The IDO an emzyme and uses thereof are described on pages 292-293 of International Application WO 2016/164731 filed on 8 Apr. 2016, which is hereby incorporated by reference.

In embodiments, a CAR-expressing cell described herein is administered to a subject in combination with a modulator of myeloid-derived suppressor cells (MDSCs). MDSCs and compositions that can be used to modulate MDSCs are described on pages 293-294 of International Application WO 2016/164731 filed on 8 Apr. 2016, which is hereby incorporated by reference.

In embodiments, a CAR-expressing cell described herein is administered to a subject in combination with a CD19 CART cell (e.g., CTL019, e.g., as described in WO2012/079000, incorporated herein by reference). In embodiments, a CD19 CART is administered to the subject prior to, concurrently with, or after administration (e.g., infusion) of a non-CD19 CAR-expressing cell, e.g., a non-CD19 CAR-expressing cell described herein.

In embodiments, a CAR-expressing cell described herein also expresses a CAR targeting CD19, e.g., a CD19 CAR. In an embodiment, the cell expressing a CAR described herein and a CD19 CAR is administered to a subject for treatment of a cancer described herein. In an embodiment, the configurations of one or both of the CAR molecules comprise a primary intracellular signaling domain and a costimulatory signaling domain. In another embodiment, the configurations of one or both of the CAR molecules comprise a primary intracellular signaling domain and two or more, e.g., 2, 3, 4, or 5 or more, costimulatory signaling domains. In such embodiments, the CAR molecule described herein and the CD19 CAR may have the same or a different primary intracellular signaling domain, the same or different costimulatory signaling domains, or the same number or a different number of costimulatory signaling domains. Alternatively, the CAR described herein and the CD19 CAR are configured as a split CAR, in which one of the CAR molecules comprises an antigen binding domain and a costimulatory domain (e.g., 4-1BB), while the other CAR molecule comprises an antigen binding domain and a primary intracellular signaling domain (e.g., CD3 zeta).

In some embodiments, a CAR-expressing cell described herein is administered to a subject in combination with an interleukin-15 (IL-15) polypeptide, a interleukin-15 receptor alpha (IL-15Ra) polypeptide, or a combination of both a IL-15 polypeptide and a IL-15Ra polypeptide e.g., hetIL-15 (Admune Therapeutics, LLC). hetIL-15 is a heterodimeric non-covalent complex of IL-15 and IL-15Ra. hetIL-15 is described in, e.g., U.S. Pat. No. 8,124,084, U.S. 2012/0177598, U.S. 2009/0082299, U.S. 2012/0141413, and U.S. 2011/0081311, incorporated herein by reference. In embodiments, het-IL-15 is administered subcutaneously.

In embodiments, a subject having a disease described herein, e.g., a hematological disorder, e.g., a lymphoma, e.g., a B-cell lymphoma, is administered a CAR-expressing cell described herein in combination with an agent described on pages 296-297 of International Application WO 2016/164731 filed on 8 Apr. 2016, which is hereby incorporated by reference.

### Pharmaceutical Compositions and Treatments

Pharmaceutical compositions of the present invention may comprise, in some aspects, a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). In some embodiments, a pharmaceutical composition comprising the cells, e.g., T cells described herein may be administered at a dosage of 10⁴ to 10⁹ cells/kg body weight, in some instances 10⁵ to 10⁶ cells/kg body weight, including all integer values within those ranges. In some embodiments, the cells, e.g., T cells described herein may be administered at 3×10⁴, 1×10⁶, 3×10⁶, or 1×10⁷ cells/kg body weight. The cell compositions may also be administered multiple times at these dosages. The cells can be administered by using infusion techniques that are commonly known in immunotherapy (see, e.g., Rosenberg et al., New Eng. J. of Med. 319:1676, 1988).

In some embodiments, a dose of CAR cells (e.g., CD19 CAR cells) comprises about 1×10⁵, 2×10⁵, 5×10⁵, 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises at least about 1×10⁵, 2×10⁵, 5×10⁵, 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 cells) comprises up to about 1×10⁵, 2×10⁵, 5×10⁵, 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19, or CAR cells) comprises about 0.1×10⁶-1.8×10⁷ cells/kg, about 0.1×10⁶ to 3.0×10⁶ cells/kg, about 0.5×10⁶ to about 2.5×10⁶ cells/kg, about 8×10⁵-3.0×10⁶ cells/kg. In some embodiments, a dose of CAR cells (e.g., CD19 cells) comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5×10⁶ cells/kg. In some embodiments, a dose of CAR cells (e.g., CD19 CAR cells) comprises about 0.2×10⁶ cells/kg. In some embodiments, a dose of CAR cells (e.g., CD19 CAR cells) comprises about 0.6×10⁶ cells/kg. In some embodiments, a dose of CAR cells (e.g., CD19 CAR cells) comprises about 1.2×10⁶ cells/kg. In some embodiments, a dose of CAR cells (e.g., CD19 CAR cells) comprises about 2.0×10⁶ cells/kg. In some embodiments, a dose of CAR cells (e.g., CD19 CAR cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 CAR cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 CAR cells) comprises up to about 1×10⁷, 2×10⁷, 5×10⁷, 1×10⁸, 2×10⁸, 5×10⁸, 1×10⁹, 2×10⁹, or 5×10⁹ cells.

In certain aspects, it may be desired to administer activated cells, e.g., T cells or NK cells, to a subject and then subsequently redraw blood (or have an apheresis performed), activate the cells therefrom according to the present invention, and reinfuse the patient with these activated and expanded cells. This process can be carried out multiple times every few weeks. In certain aspects, cells, e.g., T cells or NK cells, can be activated from blood draws of from 10 cc to 400 cc. In certain aspects, cells, e.g., T cells or NK cells, are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the cell compositions, e.g., T cell or NK cell compositions, of the present invention are administered to a patient by intradermal or subcutaneous injection. In one aspect, the cell compositions e.g., T cell or NK cell compositions, of the present invention are administered by i.v. injection. The compositions of cells e.g., T cell or NK cell compositions, may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., T cells. These cell isolates, e.g., T cell or NK cell isolates, may be expanded by methods known in the art and treated such that one or more CAR constructs of the invention may be introduced, thereby creating a CAR-expressing cell, e.g., CAR T cell of the invention. Subjects in need thereof may subsequently undergo standard treatment with high dose chemotherapy followed by peripheral blood stem cell transplantation. In certain aspects, following or concurrent with the transplant, subjects receive an infusion of the expanded CAR-expressing cells of the present invention. In an additional aspect, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for a therapeutic, e.g., an antibody, e.g., CAMPATH, for example, may be, e.g., in the range 1 to about 100 mg for an adult patient, e.g., administered daily for a period between 1 and 30 days. A suitable daily dose is 1 to 10 mg per day although in some instances larger doses of up to 40 mg per day may be used (described in U.S. Pat. No. 6,120,766).

In one embodiment, the CAR is introduced into cells, e.g., T cells or NK cells, e.g., using in vitro transcription, and the subject (e.g., human) receives an initial administration of CAR-expressing cells, e.g., CAR T cells of the invention, and one or more subsequent administrations of the CAR-expressing cells, e.g., CAR T cells of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR-expressing cells, e.g., CAR T cells of the invention are administered to the subject (e.g., human) per week, e.g., 2, 3, or 4 administrations of the CAR-expressing cells, e.g., CAR T cells of the invention are administered per week. In one embodiment, the subject (e.g., human subject) receives more than one administration of the CAR-expressing cells, e.g., CAR T cells per week (e.g., 2, 3 or 4 administrations per week) (also referred to herein as a cycle), followed by a week of no CAR-expressing cells, e.g., CAR T cells administrations, and then one or more additional administration of the CAR-expressing cells, e.g., CAR T cells (e.g., more than one administration of the CAR-expressing cells, e.g., CAR T cells per week) is administered to the subject. In another embodiment, the subject (e.g., human subject) receives more than one cycle of CAR-expressing cells, e.g., CAR T cells, and the time between each cycle is less than 10, 9, 8, 7, 6, 5, 4, or 3 days. In one embodiment, the CAR-expressing cells, e.g., CAR T cells are administered every other day for 3 administrations per week. In one embodiment, the CAR-expressing cells, e.g., CAR T cells of the invention are administered for at least two, three, four, five, six, seven, eight or more weeks.

In some embodiments, subjects may be adult subjects (i.e., 18 years of age and older). In certain embodiments, subjects may be between 1 and 30 years of age. In some embodiments, the subjects are 16 years of age or older. In certain embodiments, the subjects are between 16 and 30 years of age. In some embodiments, the subjects are child subjects (i.e., between 1 and 18 years of age).

In one aspect, CAR-expressing cells, e.g., CARTs are generated using lentiviral viral vectors, such as lentivirus. CAR-expressing cells, e.g., CARTs generated that way will have stable CAR expression.

In one aspect, CAR-expressing cells, e.g., CARTs, are generated using a viral vector such as a gammaretroviral vector, e.g., a gammaretroviral vector described herein. CARTs generated using these vectors can have stable CAR expression.

In one aspect, CAR-expressing cells, e.g., CARTs transiently express CAR vectors for 4, 5, 6, 7, 8, 9, 10, 11, 12, 13, 14, 15 days after transduction. Transient expression of CARs can be effected by RNA CAR vector delivery. In one aspect, the CAR RNA is transduced into the cell, e.g., NK cell or T cell, by electroporation.

### Indications

In some embodiments, the disclosure provides a method of trating a subject having a hematological cancer, e.g., a lymphoma. In some aspects, the disclosure provides a method of treating a subject having, or at risk of having a lymphoma (e.g., a B-cell lymphoma disclosed herein, e.g. a high grade B-cell lymphoma, a DLBCL, a multiple myeloma, or a FL), comprising administering immune effector cells that express a chimeric antigen receptor (CAR) that binds a B-cell antigen, e.g., a B-cell antigen described herein, in combination with one or more of an apoptosis inhibitor (e.g., a BCL2 inhibitor, a BCL6 inhibitor, or a combination thereof), or a MYC inhibitor. In some aspects, the disclosure provides a method of treating a subject having, or at risk of having a leukemia (e.g., an acute leukemia disclosed herein, e.g. an acute myeloid leukemia (AML)), comprising administering immune effector cells that express a chimeric antigen receptor (CAR) that binds a B-cell antigen, e.g., a B-cell antigen described herein, in combination with one or more of an apoptosis inhibitor (e.g., a BCL2 inhibitor, a BCL6 inhibitor, or a combination thereof), or a MYC inhibitor.

In other aspects, the disclosure provides a method for treating a subject having a lymphoma, e.g., a lymphoma described herein, having an increased level and/or activity of a MYC gene or gene product and/or an anti-apoptotic gene or gene product (e.g., a high grade B-cell lymphoma). The method comprises administering to the subject one or more of a BCL-2 inhibitor, a BCL-6 inhibitor, or a MYC inhibitor, wherein the subject is, or is identified as being, a non-responder, a partial responder, or a relapser to a CAR therapy that binds to a B-cell antigen.

In another aspect, the disclosure provides a method for treating or preventing a relapse to a population of immune effector cells that expresses a Chimeric Antigen Receptor (CAR) that binds a B cell antigen in a subject with a lymphoma, e.g., a lymphoma described herein, having increased level and/or activity of a MYC gene or gene product and/or an anti-apoptotic gene or gene product, comprising administering a BCL-2 inhibitor, a BCL-6 inhibitor, or a MYC inhibitor, or a combination thereof, to a subject who has undergone, is undergoing, or will receive, the CAR therapy, thereby treating or preventing the relapse to the CAR therapy.

In some aspects, the subject has or has been identified as having a leukemia, e.g., an acute leukemia including but not limited to acute myeloid leukemia (AML), B-cell acute lymphoid leukemia (BALL), small lymphocytic leukemia (SLL), acute lymphoid leukemia (ALL); or a chronic leukemias including but not limited to chronic myeloid leukemia (CML), chronic lymphocytic leukemia (CLL). In some embodiments, leukemia is an acute myeloid leukemia (AML).

In some aspects, the subject has or has been identified as having a lymphoma, e.g., a relapsed and/or refractory lymphoma. In some embodiment, the lymphoma is chosen from: a high grade B-cell lymhphoma, DLBCL, follicular lymphoma (FL), mantle cell lymphoma (MCL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, Marginal zone lymphoma, multiple myeloma, myelodysplasia and myelodysplastic syndrome, non-Hodgkin lymphoma, Hodgkin lymphoma, or plasmablastic lymphoma. In some embodiments, the lymphoma is a high grade B-cell lymphoma (e.g., a double and/or triple hit lymphoma or a non-specified NOS high-grade lymphoma). In some embodiments, the lymphoma is a double hit lymphoma. In some embodiments, the lymphoma is a triple hit lymphoma. In some embodiments, the lymphoma is DLBCL (e.g., relapsed and/or refractory DLBCL). In some embodiments, the lymphoma is follicular lymphoma (FL).

In some embodiments, the B-cell lymphoma described herein is a relapsed or refractory lymphoma, wherein the subject comprises one or more criteria in Table 8 for partial metabolic response and partial radiological response, or progressive metabolic disease or progressive disease. In some embodiments, a subject having a relapsed or refractory B-cell lymphoma, may comprise, e.g., a new bone marrow involvement, a new malignant effusion, a new nodal lesion>1.5 cm in any axis (e.g., a previously normal lymph node becoming >1.5 cm in any axis) on CT scan or MRI after baseline, or any discrete extranodal lesion (including liver or spleen) on CT scan or MRI after baseline, or ≥50% increase in long axis from baseline of any residual lymph node or mass.

TABLE 8
Overall Response Assessment for B-cell lymphomas, e.g., DLBCL and FL

| | | PET-based response | CT-based response |
|---|---|---|---|
| | | Complete Metabolic | Complete Radiologic |
| | | Response (CMR) | Response (CR) |
| | | (All of the following) | (All of the following) |
| Complete | Index | Score 1, 2, or 3 with or | Nodal Disease: ≤1.5 cm |
| Response | nodal/extranodal | without residual mass on | in longest transverse |
| | (including splenic and | PET Five Point Scale (5- | diameter of a lesion (LDi) |
| | hepatic lesions) | PS) | Extranodal Disease: |
| | | | Absent |
| | Non-index | | Absent |
| | (including splenic and | | |
| | hepatic lesions) | | |
| | Spleen | | Regress to normal (≤13 cm) |
| | New lesions | None | None |
| | Bone Marrow | No evidence of | Normal by bone marrow |
| | | Fluorodeoxyglucose | assessment |
| | | (FDG)-avid | |
| | | disease in marrow. | |
| | | Normal by bone marrow | |
| | | assessment. | |
| | | Partial Metabolic | |
| | | Response (PMR) | Partial Remission (PR) |
| | | (all of the following) | (all of the following) |
| Partial Response | Index | Score of 3*, 4 or 5 with | ≥50% decrease from |
| | nodal/extranodal | reduced uptake | baseline in sum of the |
| | (including splenic and | compared to baseline | produc diameters (SPD) |
| | hepatic lesions) | with respect to SUV | of all index lesions |
| | Non-index | intensity or extent. This | No Increase |
| | (including splenic and | may apply to the specific | |
| | hepatic lesions) | hot spot and/or overall | |
| | Spleen | the subject. It is expected | >50% decrease from |
| | | that there will be residual | baseline in enlarged |
| | | mass(es) present. | portion of spleen (value |
| | | | >13 cm) |
| | | | Example: |
| | | | Spleen at baseline 16 cm |
| | | | (Enlarged portion is 3 cm). |
| | | | Thus a 2 cm decrease |
| | | | to a spleen that is 14 cm |
| | | | at follow-up is partial |
| | | | remission. |
| | New lesions | None | None |
| | Bone Marrow | Residual uptake higher | Not applicable |
| | | than uptake in normal | |
| | | marrow but reduced | |
| | | compared with baseline | |
| | | Persistent focal | |
| | | changes in the marrow | |
| | | with nodal response | |
| | | No Metabolic Response | |
| | | (NMR) | Stable Disease (SD) |
| | | (all of the following) | (all of the following) |
| Stable Disease | Index | Score of 3, 4 or 5 with no | <50% decrease from |
| | nodal/extranodal | significant change in FDG | baseline in SPD of all |
| | (including splenic and | uptake from baseline | index lesions |
| | hepatic lesions) | | No criteria for |
| | | | progressive disease (PD) |
| | | | are met |
| | Non-index | | No progression |
| | (including splenic and | | |
| | hepatic lesions) | | |
| | Spleen | | No progression |
| | New lesions | None | None |
| | Bone Marrow | No change in metabolic | Not applicable |
| | | activity from baseline | |
| | | Progressive Metabolic | |
| | | Disease (PMD) | Progressive disease |
| | | (At least one of the | (At least one of the |
| | | following) | following) |
| Progressive Disease | Index | Score of 3, 4 or 5 with | Product of Perpendicular |
| | nodal/extranodal | increased uptake | Diameters (PPD) |
| | (including splenic and | compared to the visually | Progression: |
| | hepatic lesions) | determined nadir with | An individual node/lesion |
| | | respect to standard | must be abnormal with: |
| | | uptake value (SUV) | LDi > 1.5 cm AND |
| | | intensity or extent. This | Increase by >=50% |
| | | may apply to the specific | from PPD nadir AND |
| | | hot spot and/or overall | An increase in LDi or |
| | | the subject. and/or | shortest axis |
| | | New FDG-avid foci | perpendicular to LDi (SDi) |
| | | consistent with lymphoma | from nadir |
| | | | ≥0.5 cm for lesions |
| | | | ≤2.0 cm |
| | | | ≥1.0 cm for lesions |
| | | | >2.0 cm |
| | Non-index | | Unequivocal Progression |
| | (including splenic and | | |
| | hepatic lesions) | | |
| | Spleen | | Progression of existing |
| | | | splenomegaly |
| | | | New or Recurrent |
| | | | splenomegaly |
| | New lesions | | Regrowth of previously |
| | | | resolved lesions |
| | | | New node > 1.5 cm in |
| | | | any axis |
| | | | New extranodal site |
| | | | >1.0 cm in any axis |
| | | | New extranodal site |
| | | | ≤1.0 cm in longest axis |
| | | | and unequivocal/ |
| | | | attributable to lymphoma. |
| | | | Any size assessable |
| | | | disease unequivocal/ |
| | | | attributable to lymphoma |
| | Bone Marrow | New/recurrent FDG-avid | New/recurrent |
| | | foci | involvement |

PET 5PS: 1, no uptake above background; 2, uptake mediastinum; 3, uptake > mediastinum but liver; 4, uptake moderately > liver; 5, uptake markedly higher than liver and/or new lesions; X, new areas of uptake unlikely to be related to lymphoma. GTD: Greatest transverse diameter; LDI: Longest transverse Diameter of a lesion; PPD: Product of perpendicular diameters.
Score 3 is considered PET negative in low risk disease and where no further treatment necessary (e.g. Follicular Lymphoma)
Score 3 is considered PET positive in high risk disease that is curable and aggressive (e.g. Hodgkin Lymphoma, Diffuse Large B Cell Lymphoma)
Score 4 should be applied to uptake greater than the maximum standard uptake value (SUV) in a large region of normal liver and score 5 to uptake 2X greater than the maximum SUV in the liver. The PET images should be converted to SUV maps to support comparison across time points and to standardize viewing conditions. (New) areas of uptake unlikely to be related to lymphoma will be marked as “X” (Barrington et al. 2014).

### High Grade B-Cell Lymphoma

In some embodiments, the subject has high-grade B-cell lymphoma, e.g., a double hit lymphoma, a triple hit lympoma, or a non-specified (NOS) high-grade lymphoma. In some embodiments, the subject has a double hit lymphoma. In some embodiments, the subject has a triple hit lymphoma. In some embodiments, the subject is less than 18 years of age. In some embodiments, the subject is an adult.

In some embodiments, the subject having the high grade lymphoma, e.g., the double or triple hit lymphoma, has an alteration in a MYC gene or gene product and an alteration in an anti-apoptotic gene product (e.g., BCL2 and/or BCL6), that results in increased expression and/or activity of the MYC gene or gene product and the anti-apoptotic gene or gene product (e.g., BCL2 and/or BCL6). In some embodiments, the alteration is a gene rearrangement, e.g., a translocation. In some embodiments, a double hit lymphoma is classified by an alteration in a MYC gene or gene product, a BCL2 gene or gene product and/or a BCL6 gene or gene product. In some embodiments, a triple hit lymphoma is classified by an alteration in a MYC gene or gene product, a BCL2 gene or gene product, and a BCL6 gene or gene product. In some embodiments, the high-grade lymphoma, e.g., a double hit lymphoma, comprises chromosomal breakpoints of 8q24/MYC and 18q21/BCL2 or 8q24/MYC and 3q27/BCL-6. In some embodiments, the high grade lymphoma, e.g., a triple hit lymphoma, comprises chromosomal breakpoints of 8q24/MYC, 18q21/BCL2, and 3q27/BCL-6.

In some embodiments, the subject having a high grade B-cell lymphoma, e.g., a double hit or triple hit lymphoma is diagnosed by a tumor biopsy, using a FISH assay and/or an immunohistochemistry assay.

### DLBCL and Relapsed/Refractory DLBCL

In some embodiments, the subject has DLBCL. In some embodiments the subject has relapsed or refractory DLBCL. In some embodiments, the subject is at least 18 years of age. In some embodiments, the DLBC arises from a cell population comprising a Germinal Center B-cell (GCB cell). In some embodiments, the DLBCL arises from a cell population comprising an activated B-cell (ABC cell). In some embodiments, the DLBCL arrises from an unclassified cell population. In some embodiments, the cell of origin for DLBCL is identified by an immunohistochemistry (IHC)-based algorithm (e.g., the Choi algorithm) or a microarray

In some embodiments, the subject having DLBCL, e.g., relapsed or refractory DLBCL has previously been administered one or more of: an anti-CD20 therapy, an anthracycline based chemotherapy or stem cell therapy, e.g., allogeneic or autologous SCT, e.g., as described herein, as a, e.g., first, second or third line therapy. In some embodiments, the subject has no response to, e.g., relapsed, refractory, has progressive disease, or has failed, the first, second or third line therapy.

In some embodiments, a subject having relapsed or refractory DLBCL is administered a combination therapy comprising a BCL2 inhibitor, a BCL6 inhibitor, a MYC inhibitor, or a combination thereof, and a CAR-expressing cell, e.g., according to a dosage regimen described herein. In some embodiments, the subject has previously been treated with a BCL2 inhibitor, a BCL6 inhibitor, a MYC inhibitor, or a combination thereof, e.g., for at least 4-6 weeks or 8-10 weeks.

In some embodiments, the subject is administered the a BCL2 inhibitor, a BCL6 inhibitor, a MYC inhibitor, or a combination thereof, e.g., daily, prior to apheresis, e.g., at least about 21 days, e.g., 21-30 days, e.g., 28 days prior to apheresis. In some embodiments, the subject is administered the BTK inhibitor for at least about 21 days, e.g., 10-100 days, after apheresis and prior to CAR therapy administration, e.g., infusion.

In some embodiments, the subject is administered a BCL2 inhibitor, a BCL6 inhibitor, a MYC inhibitor, or a combination thereof, concurrently with or after apheresis. In some embodiments, the subject is administered the a BCL2 inhibitor, a BCL6 inhibitor, a MYC inhibitor, or a combination thereof, for at least about 21 days, e.g., 10-100 days, after apheresis and prior to CAR therapy administration, e.g., infusion. In some embodiments, the subject is continuously administered with a BCL2 inhibitor, a BCL6 inhibitor, a MYC inhibitor, or a combination thereof, e.g., daily. In some embodiments, the subject is administered 0.6-6.0×10⁸ CAR expressing cells.

In some embodiments, the subject is administered lymphodepletion after initiation of the a BCL2 inhibitor, a BCL6 inhibitor, a MYC inhibitor, or a combination thereof, but prior to administration of the CAR therapy. In some embodiments, the lymphodepletion comprises administering cyclophosphamide and fludarabine. In some embodiments, the lymphodepletion comprises administering 500 mg/m2 cyclophosphamide daily for 2 days and 30 mg/m2 fludarabine daily for 3 days. In some embodiments, the lymphodepletion comprises administering 250 mg/m2 cyclophosphamide daily for 3 days, and 25 mg/m2 fludarabine daily for 3 days. In some embodiments, the lymphodepletion begins with the administration of the first dose of fludarabine. In some embodiments, cyclophosphamide and fludarabine are administered on the same day. In some embodiments, cyclophosphamide and fludarabine are not administered on the same day. In some embodiments, the daily dosages are administered on consecutive days. In embodiments, the lymphodepletion comprises administering bendamustine. In some embodiments, bendamustine is administered daily, e.g., twice daily, at a dosage of about 75-125 mg/m2 (e.g., 75-100 or 100-125 mg/m², e.g., about 90 mg/m²), e.g., intravenously. In some embodiments, bendamustine is administered at dosage of 90 mg/m2 daily, e.g., for 2 days. In some embodiments, the subject has a cancer, e.g., a hematological cancer as described herein.

In embodiments, the subject is administered a first lymphodepletion regimen and/or a second lymphodepletion regimen. In embodiments, the first lymphodepletion regimen is administered before the second lymphodepletion regimen. In embodiments, the second lymphodepletion regimen is administered before the first lymphodepletion regimen. In embodiments, the first lymphodepletion regimen comprises cyclophosphamide and fludarabine, e.g., 250 mg/m2 cyclophosphamide daily for 3 days, and 25 mg/m2 fludarabine daily for 3 days. In embodiments, the second lymphodepletion regimen comprises bendamustine, e.g., 90 mg/m² daily, e.g., for 2 days. In embodiments, the second lymphodepletion regimen is administered as an alternate lymphodepletion regimen, e.g., if a subject has experienced adverse effects, e.g., Grade 4 hemorrhagic cystitis, to a lymphodepletion regimen comprising cyclophosphamide. In some embodiments, the lymphoma is a DLBCL, e.g., a relapsed or refractory DLBCL (e.g., r/r DLBCL), e.g., a CD19+r/r DLBCL. In some embodiments, the subject is an adult and the lymphoma is an r/r DLBCL.

In some embodiments, a subject administered a therapy described herein, e.g., a therapy comprising a CAR-expressing therapy, e.g., a therapy comprising a CAR19-expressing therapy (e.g., a CAR19-expressing therapy in combination with a BCL2 inhibitor, a BCL6 inhibitor, a MYC inhibitor, or a combination thereof), has previously received, e.g., been administered, one or more lines of therapy, e.g., 2, 3, 4, or 5 or more lines of therapy (e.g., one or more therapies as described herein) and/or the subject was not eligible for or had failed stem cell therapy (SCT), e.g., autologous or allogeneic SCT. In some embodiments the subject has previously received 2 or more lines of therapy comprising rituximab and anthracycline. In some embodiments, the subject was not eligible for or had failed autologous SCT. In some embodiments, administration of a CAR19-expressing therapy (e.g., in combination with a BCL2 inhibitor, a BCL6 inhibitor, a MYC inhibitor, or a combination thereof) to the subject who has previously undergone 2 or more lines of therapy and/or was not eligible for or had failed autologous SCT results in a response, e.g., a high response rate and/or a durable response to the therapy, e.g., therapy comprising a CAR19-expressing therapy (e.g., in combination with a BCL2 inhibitor, a BCL6 inhibitor, a MYC inhibitor, or a combination thereof,). In some embodiments, the subject has a hematological cancer, e.g., DLBCL, e.g., relapsed and/or refractory DLBCL.

### Follicular Lymphoma

In some embodiments, the subject has follicular lymphoma (FL). In some embodiments, FL is also referred to as a Non-Hodgkin lymphoma. In some embodiments, the subject has relapsed or refractory FL. In some embodiments, the FL can be classified as a Stage I lymphoma, a Stage II lymphoma, a Stage III lymphoma or a Stage IV lymphoma. FL and standard of care for FL is described in Luminaari S et al. (2012); Rev Bras Hematol Hemoter. 34(1):54-59, the entire contents of which is hereby incorporated by reference.

In some embodiments, the subject having FL, e.g., relapsed or refractory FL, has previously been administered one or more of: a chemotherapy, immunotherapy, radiation therapy or radioimmunotherapy, e.g., as a first, second, or third line therapy. In some embodiments, the subject has been administered: an anti-CD20 therapy (e.g., rituximab); an anthracycline based chemotherapy; a stem cell therapy, e.g., allogeneic or autologous SCT; or a radioimmunotherapy. In some embodiments, the subject has no response to, e.g., relapsed, refractory, has progressive disease, or has failed, the first, second or third line therapy.

In some embodiments, FL progresses to a high grade lymphoma, e.g., a double hit lymphoma.

### Multiple Myeloma

In some embodiments, the subject has multiple myeloma. In some embodiments, the subject has an asyptomatic myeloma (e.g., a smoldering multiple myeloma or an indolent myeoloma). In some embodiments, the subject has relapsed or refractory multiple myeloma. In some embodiments, the multiple myeloma can be classified as Stage I, Stage II, or a Stage III multiple myeloma using the Revised International Staging System (RISS).

In some embodiments, the subject having multiple myeloma, e.g., relapsed or refractory multiple myeloma, has previously been administered one or more of: a chemotherapy, immunotherapy, radiation therapy or radioimmunotherapy, e.g., as a first, second, or third line therapy. In some embodiments, the subject has been administered: an anti-CD20 therapy (e.g., rituximab); an anthracycline based chemotherapy; a stem cell therapy, e.g., allogeneic or autologous SCT; or a radioimmunotherapy. In some embodiments, the subject has no response to, e.g., relapsed, refractory, has progressive disease, or has failed, the first, second or third line therapy. In some embodiments, the response of the subject to a multiple myeloma therapy is determined based on IMWG 2016 criteria, e.g., as disclosed in Kumar, et al., Lancet Oncol. 17, e328-346 (2016), hereby incorporated herein by reference in its entirety, e.g., as described in Table 16.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Correlation of Tumor Subtypes and Genomics with Efficacy Outcomes in a Tisagenlecleucel-Treated Relapsed/Refractory Diffuse Large B-Cell Lymphoma (r/r DLBCL) Patients in the JULIET Trial

Tisagenlecleucel (autologous anti-CD19 CAR-T cell therapy) has demonstrated durable responses and a manageable safety profile in adult patients with r/r DLBCL in the JULIET trial. This Example describes genomic analyses to further characterize efficacy outcomes across patient subgroups in tisagenlecleucel-treated patients with r/r DLBCL.

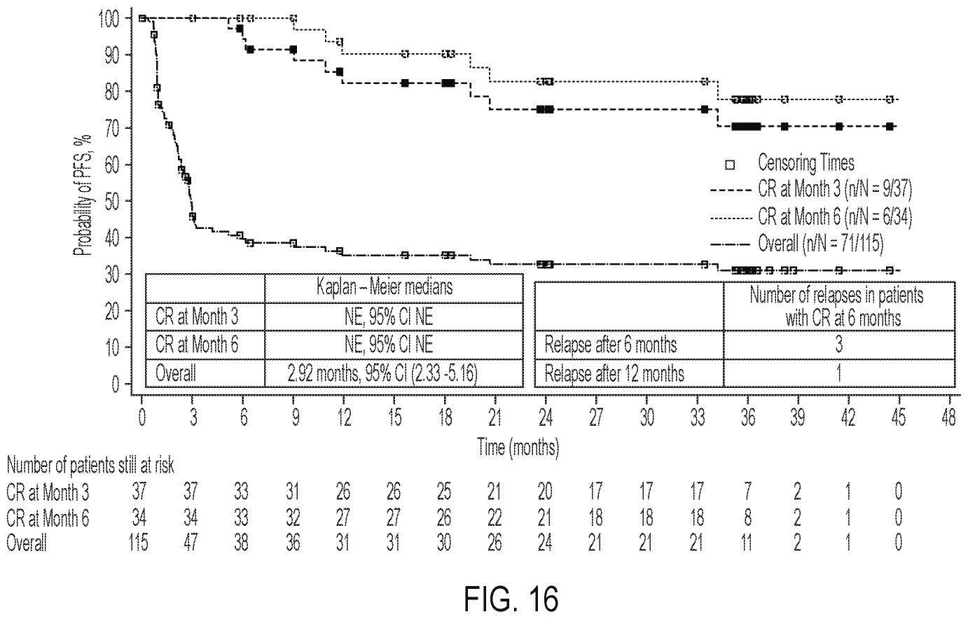

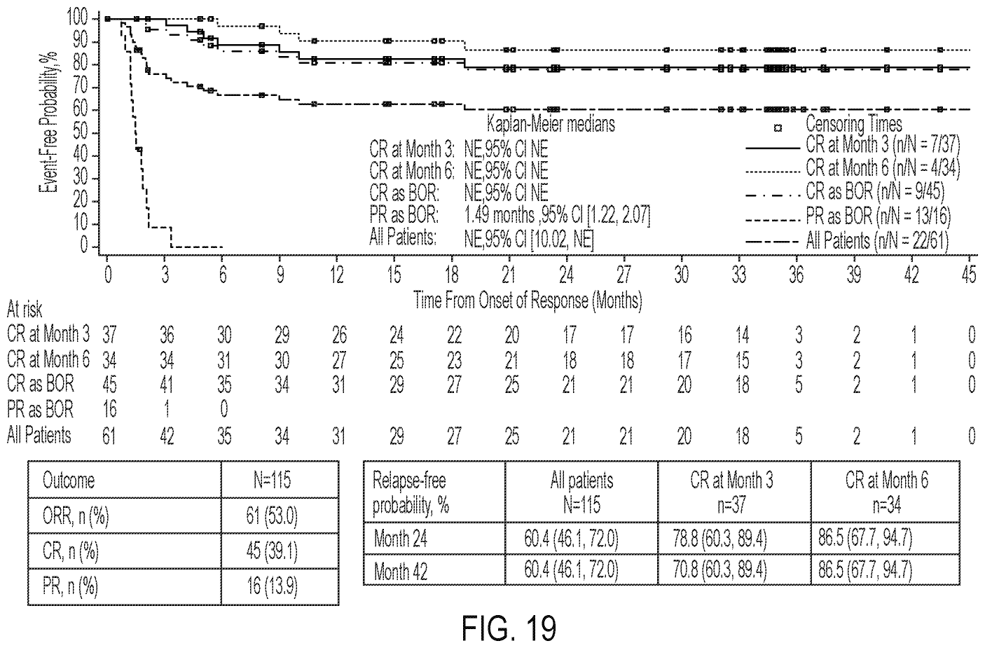

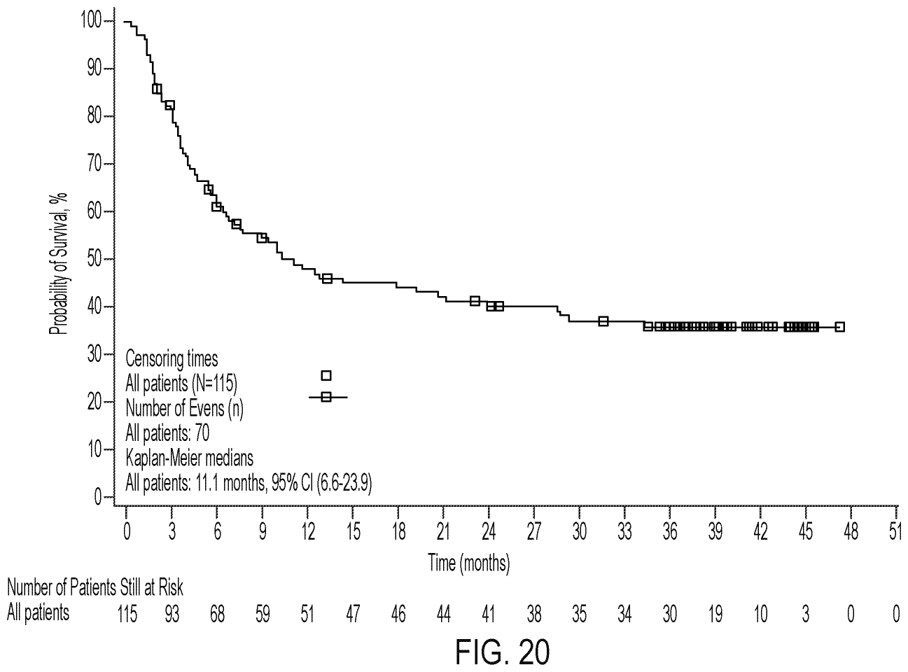

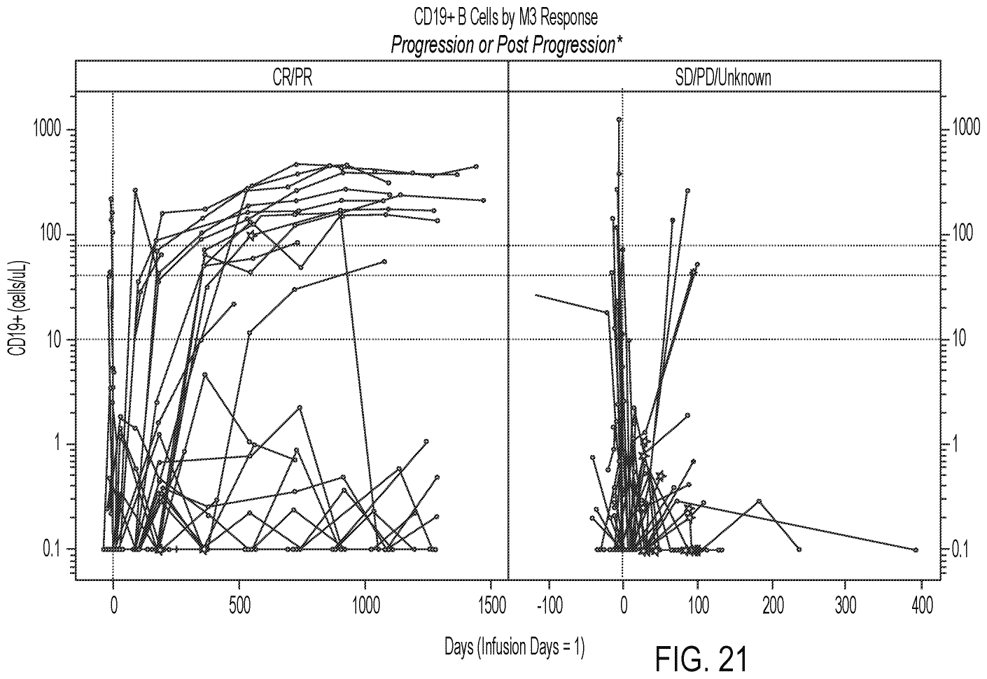

Methods: JULIET (NCT02445248) was a global, phase 2 trial of autologous anti-CD19 CAR-T cell therapy in adult patients with r/r DLBCL (relapsed or refractory to ≥2 prior lines of therapy). Median follow-up was 40.3 months. The relationship between MYC overexpression, tumor microenvironment (TME) characteristics (including CD3+ T-cell infiltration, myeloid-derived suppressor cells [MDSCs], and LAG3 expression by fluorescent immunohistochemistry [IHC]), activated B-cell/germinal center B-cell [ABC/GCB] subtype, double/triple-hit (DH/TH) status, and gene mutations including TP53 with efficacy outcomes (best overall response [BOR], response at Month 3 [M3], progression-free and overall survival [PFS/OS]) were assessed.
Results: 115 patients received autologous anti-CD19 CAR-T cell infusion. Among patients who were in CR at 6 months, 86% were estimated to maintain the response at 36 months. Additionally, among the 61 patients with a response, the relapse-free probability was 60.4% at 24 and 30 months; median duration of response (DOR) was not reached (95% CI, 10-not estimable [NE]) (FIG. 19). Median OS among all 115 infused patients was 11.1 months (95% CI, 6.6-23.9). Survival probability at 12, 24, and 36 months was 48.2%, 40.4%, and 36.2%, respectively (FIG. 20). Median OS of patients with CR (n=37) or PR (n=7) at M3 was not reached; 80% of patients with complete response (CR) had OS benefit≥20 months. No new safety signals were detected. Of the 23 patients with ongoing CR and B-cell count available, 11 had CD19+ B cells recovered back to normal (80 cells/μL) after 1 year, with similar patterns observed for CD20+ and CD22+ B cells (FIG. 21). The 24 month and 42 month PFS was 33% and 31% respectively (FIG. 16). Of patients with a CR at 6 months, 3 had relapses after 6 months and 1 had a relapse after 12 months (FIG. 16).

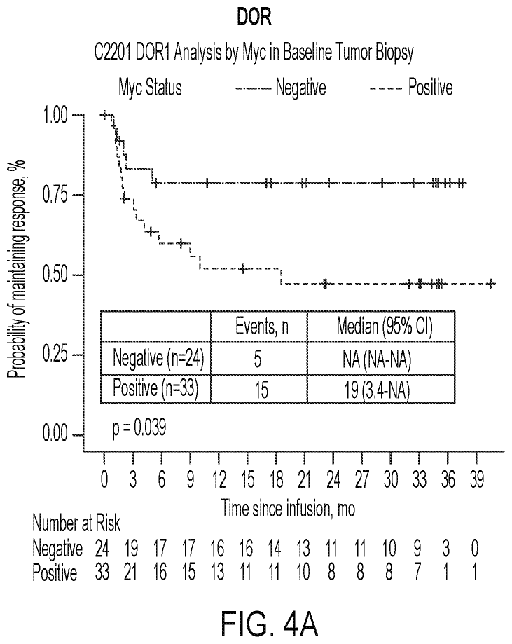

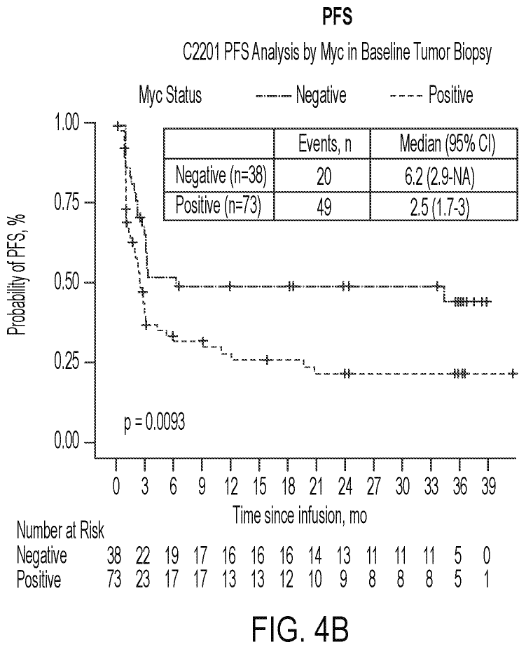

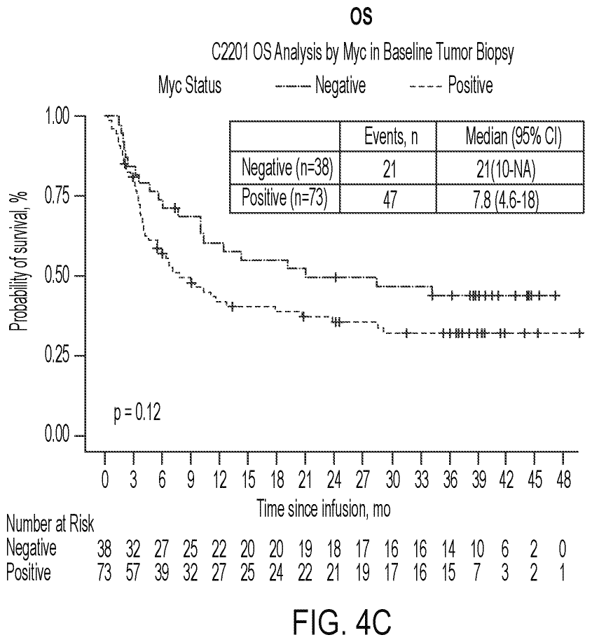

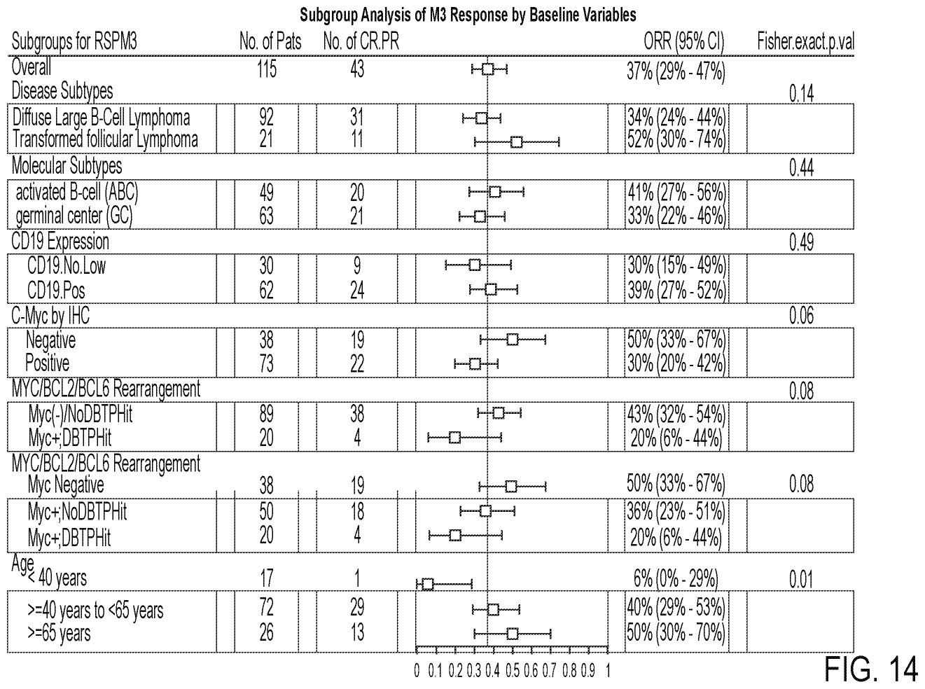

Of the 111 patients whose baseline tumor biopsies were tested for MYC expression by immunohistochemistry (IHC), 73 patients were positive for MYC expression (MYC(+) defined as >40% of cells expressing MYC, as measured by IHC) and 38 patients were negative for MYC expression (MYC(−) defined as <40% of cells expressing MYC, as measured by IHC). BOR rates for MYC(+) and MYC(−) patients were 44% (95% CI, 32-56) and 63% (95% CI, 46-78), respectively; M3 response rates were 30% (95% CI, 20-42) and 50% (95% CI, 33-67), respectively (Table 6, and FIG. 14). By month 6, 18/38 (47%) MYC(−) patients were CR/PR compared to 18/73 (25%) of MYC(+) patients. DOR was in a plateau phase at 79% starting in MYC(−) patients (n=24) at 6 months. Baseline MYC (−) status was associated with improved outcomes as compared to MYC(+) patients, including longer median DOR (not reached and 19 months [95% CI, 3.4-NE], respectively), PFS (6.2 months [95% CL, 2.9-NE] and 2.5 months [95% CI, 1.7-3.0], respectively), and OS (21 months [95% CL, 10-NE] and 7.8 months [95% CI, 4.6-18], respectively) (FIGS. 4A-AC). Thus, MYC(+) patients tended to have shorter PFS and OS compared with MYC(−) patients and a high-baseline of MYC expression in patients prior to treatment was associated with worse PFS and OS (FIGS. 4A-4C).

TABLE 6
Best overall response and response at 3 months as measured in
MYC positive and MYC negative patients

| | MYC(+) | MYC(−) |
|---|---|---|
| | (n = 73) | (n = 38) |
| Best Overall Response | 44 (32-56) | 63 (46-78) |
| (BOR), % (95% CI) | | |
| Response at Month 3 (M3), | 30 (20-42) | 50 (33-67) |
| % (95% CI) | | |

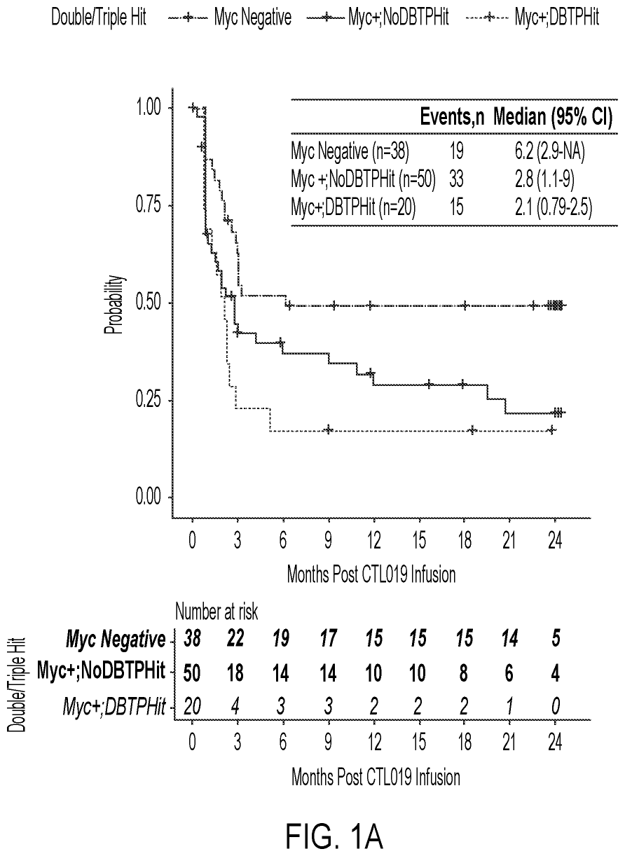

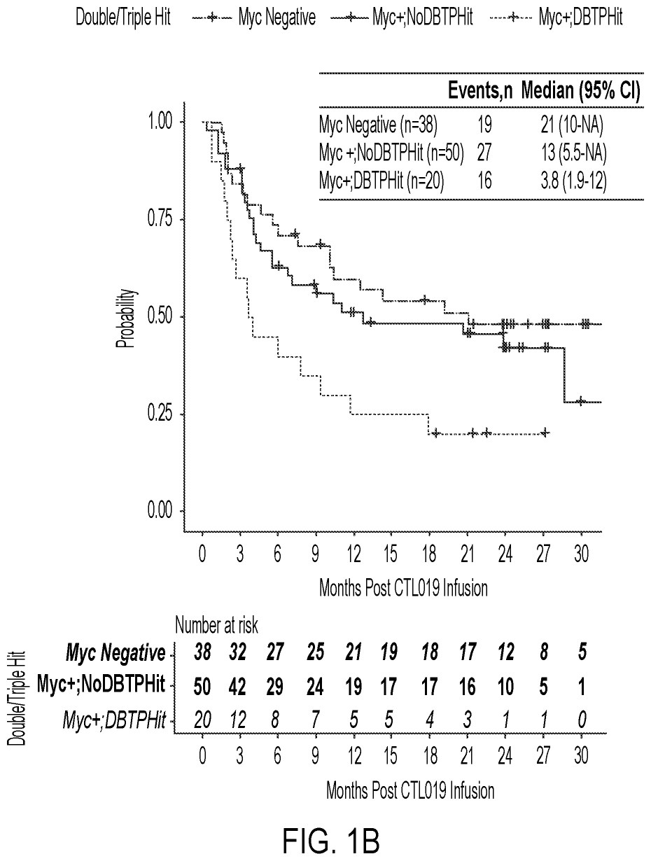

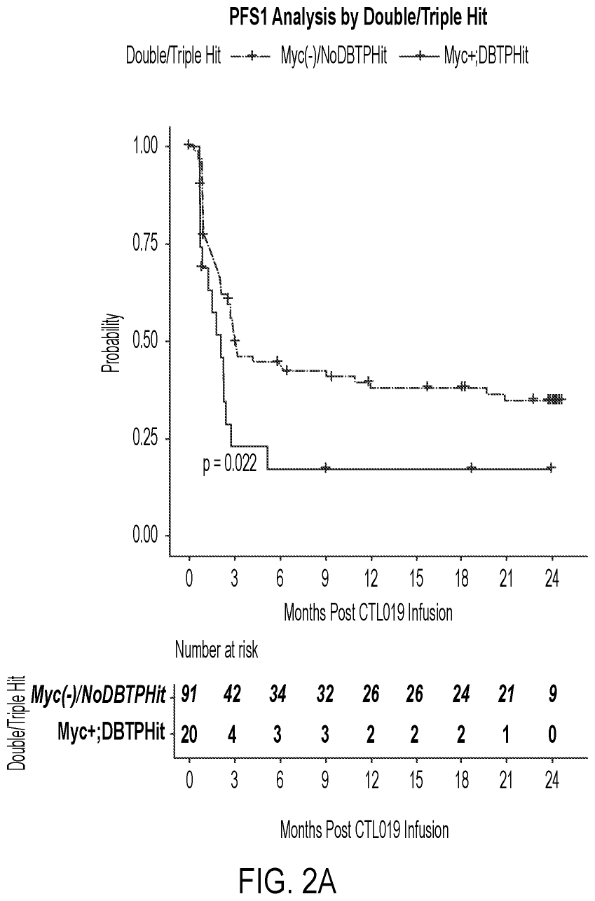

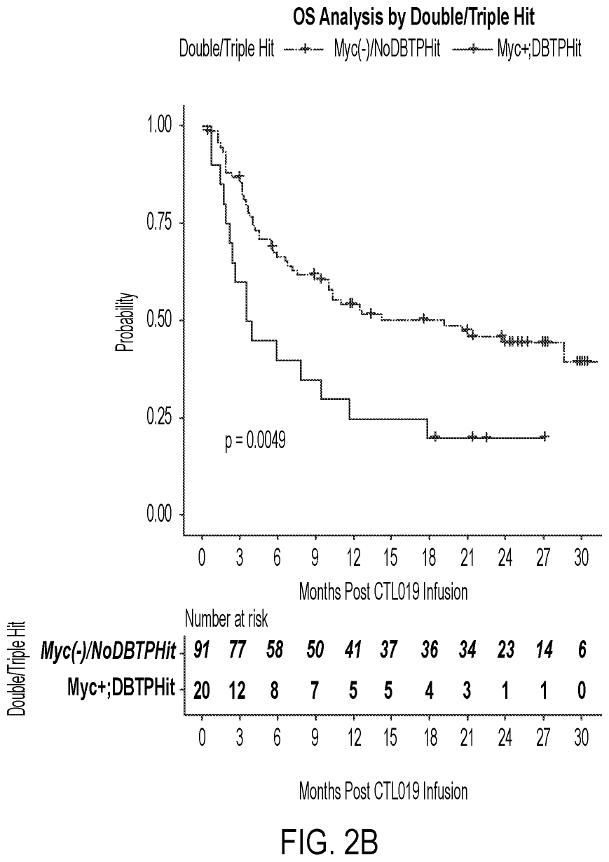

Of the 73 MYC(+) patients, 70 of them were also tested for rearrangements in MYC, BCL2, and BCL6 by FISH in order to identify double hit (DH) (MYC/BCL2 or MYC/BCL6) or triple hit (TH) (MYC/BCL2/BCL6) lymphomas. Of the 70 MYC(+) patients, 20 had DH/TH lymphoma and a BOR rate of 40% (95% CI, 19-64). BOR rate for patients that were MYC(+) but non-DH/TH lymphoma was 48% (95% CI, 34-63). Patients with DH/TH lymphoma had a response rate of 20% (95% CI, 6-44) at M3 compared with 36% (95% CI, 23-51) in patients with MYC(+) but non-DH/TH lymphoma (Table 7 and FIG. 14). Patients with DH/TH lymphoma also tended to have shorter progression free survival (PFS) and overall survival (OS) compared with other patients (FIG. 1A-B, comparing MYC negative patients, MYC positive and DH/TH negative patients, and MYC positive and double/triple hit positive patients; FIG. 2A-2B, comparing MYC negative and DH/TH negative patients, and MYC positive and DH/TH positive patients). ABC/GCB subtype, which was assessed by the Choi algorithm, did not appear to correlate with response.

TABLE 7
Best overall response and response at 3 months as measured in MYC positive
patients that were also positive or negative for double or triple hit (DH/TH) lymphoma;
as well as MYC negative patients

| | | MYC(+) Non- | |
|---|---|---|---|
| | MYC(+) DH/TH | DH/TH | MYC(−) |
| | (n = 20) | (n = 50) | (n = 38) |
| Best Overall Response (BOR), | 40 (19-64) | 48 (34-63) | 63 (46-78) |
| % (95% CI) | | | |
| Response at Month 3 | 20 (6-44) | 36 (23-51) | 50 (33-67) |
| (M3), % (95% CI) | | | |

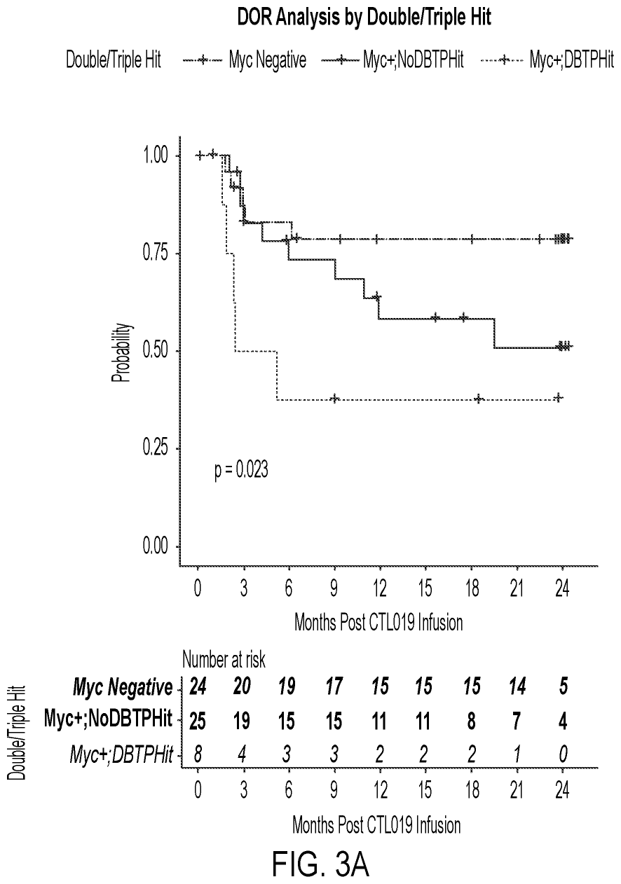

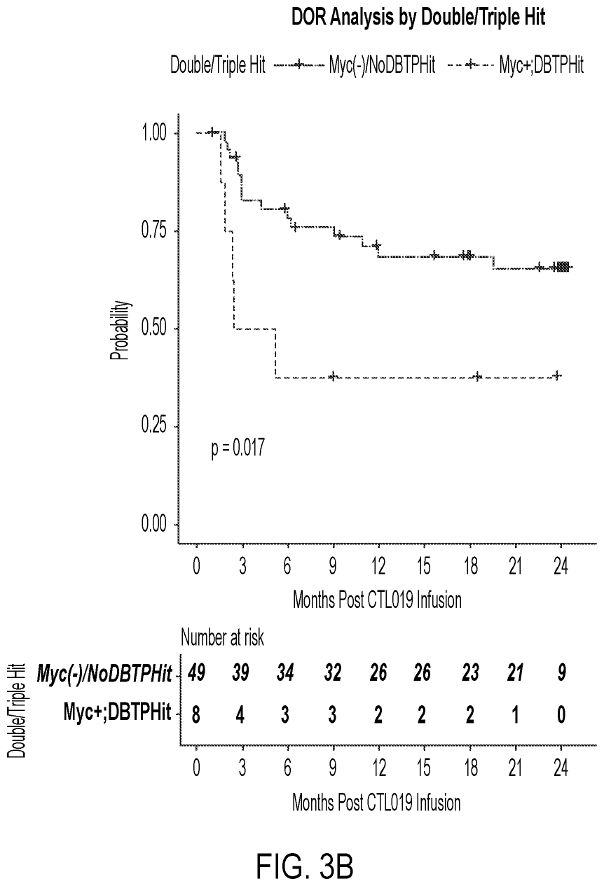

Additionally, the duration of response (DOR) to CART19 therapy was assessed in MYC(−) patients; MYC(−) patients and double/triple hit negative patients; MYC (+) and double/triple hit negative patients; and MYC(+) and double/triple hit positive patients, and less than 75% of patients identified as MYC positive by IHC, maintained longer than a 6 month response to CART19 therapy (FIGS. 3A-3B).

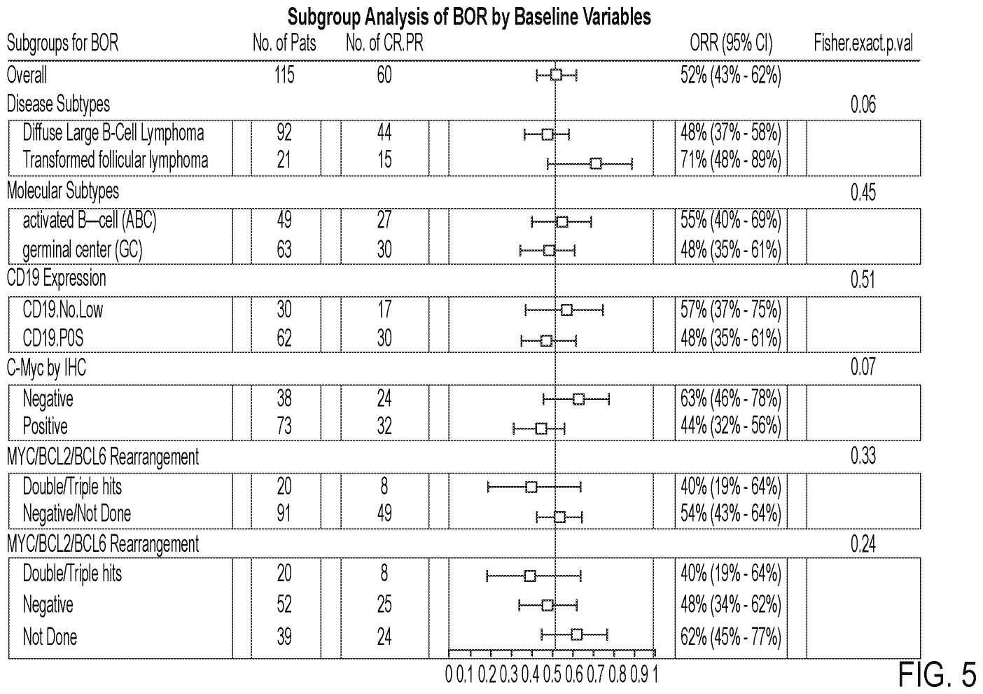

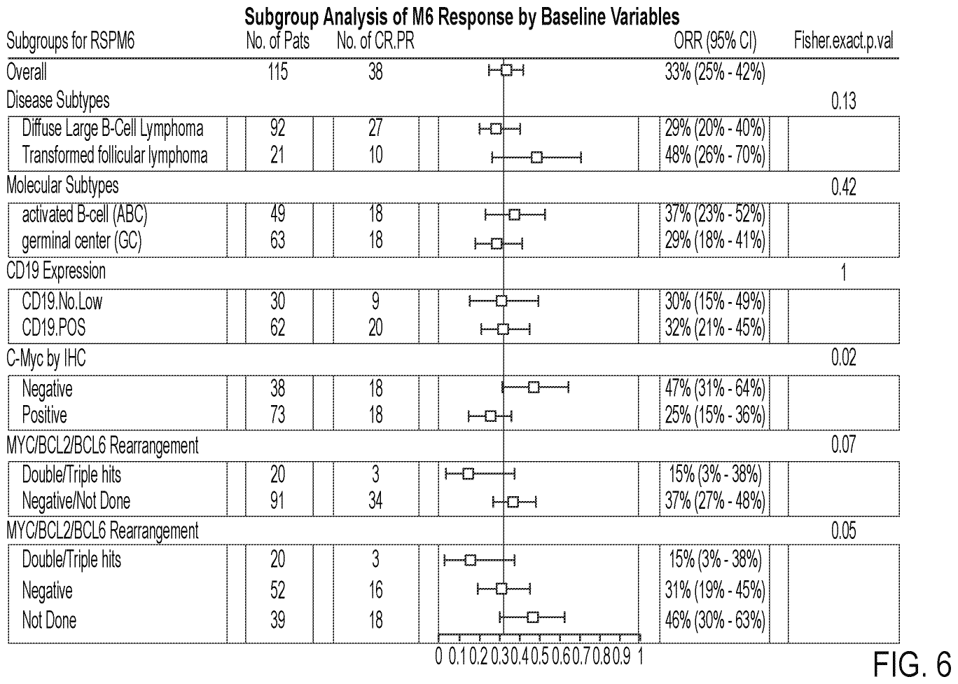

Responses to CART19 treatment in patients with relapsed or refractory DLBCL and additional B-cell lymphoma subsets, including those patients positive for MYC, patients negative for MYC, and/or patients positive for DH/TH lymphomas were comparable when assessed at 1 month post-treatment (FIG. 5). However, at both 3 months (FIG. 14) and 6 months (FIG. 6), patients with DH/TH lymphomas and high MYC expression appeared to relapse more frequently.

High levels of baseline lactate dehydrogenase (LDH) and grade 3-4 neurological events (NE) were previously reported to be associated with poor efficacy outcomes (Westin et al ASH 2019). 40% (8/20) of patients with DH/TH lymphoma had LDH levels>2 times upper limit of normal compared with 14% (12/88) in other patients. In multivariate Cox analyses, when adjusted for baseline LDH and NE grades, DH/TH was associated with shorter PFS and OS while MYC+ status was associated with shorter PFS, but not OS.

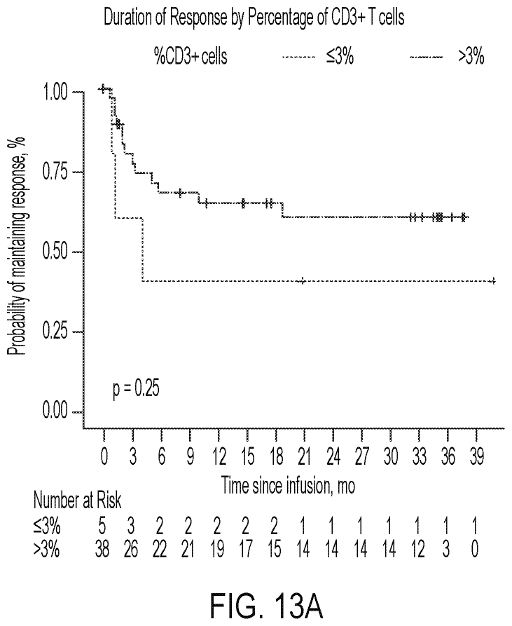

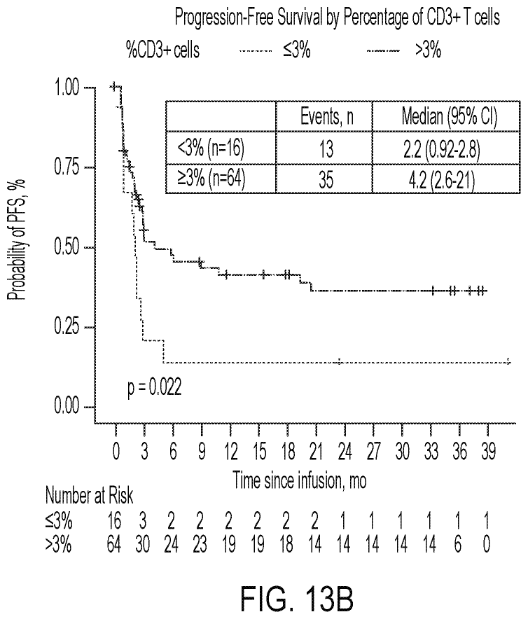

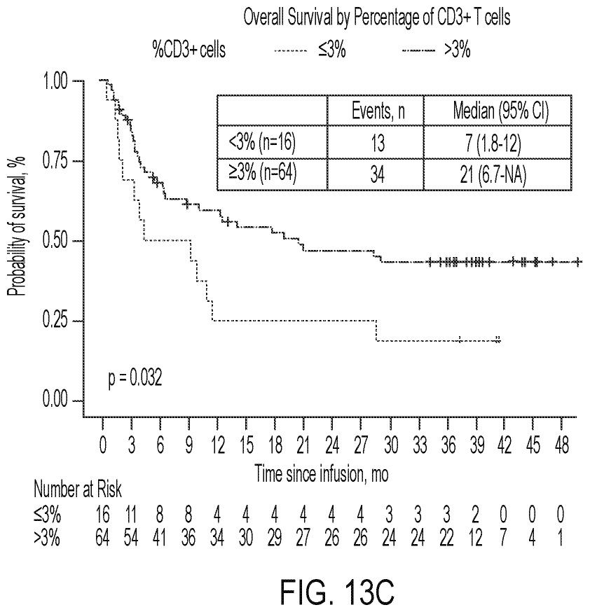

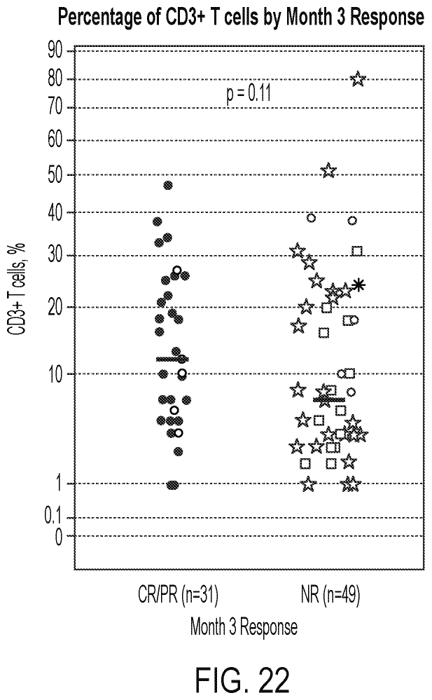

In the TME analysis of baseline biopsy, a low frequency of tumor infiltrating CD3+ T cells (cutoff of ≤3%; n=16) was associated with shorter median DOR, PFS (2.2 months [95% CI, 0.92-2.8] and 4.2 months [95% Cl, 2.6-21], respectively) and OS (7 months [95% CI, 1.8-12] and 21 months [95% CI, 6.7-NE], respectively) compared with patients with ≥3% CD3+ T cells (n=64) (FIGS. 13A-13C). At month 6, only 2/16 (13%) of patients with <3% tumor-infiltrating CD3+ T cells in baseline tumors were responders compared to 23/36 (64%) responders with >3% tumor infiltrating CD3+ T cells. Patients with low or no (≤3%) tumor-infiltrating CD3+ T cells (n=16) in baseline tumor biopsies were enriched with non-responders (FIG. 22).

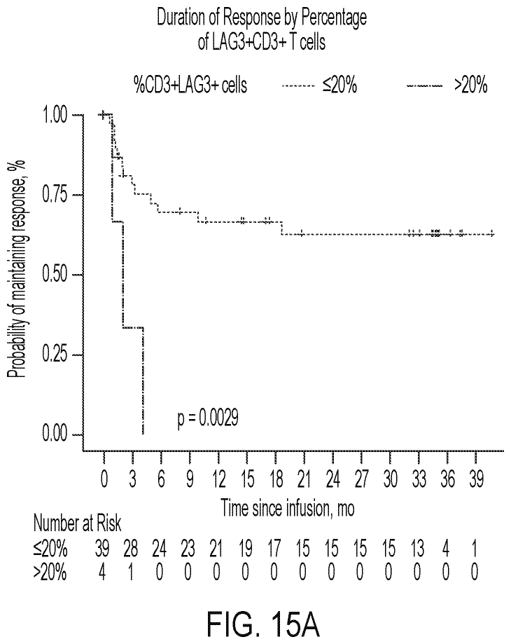

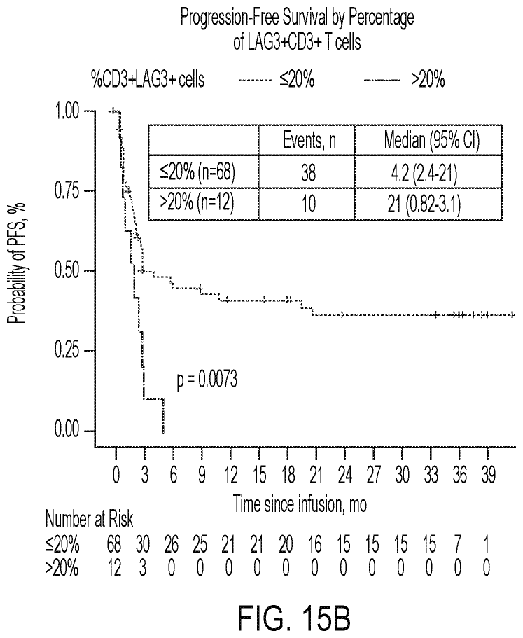

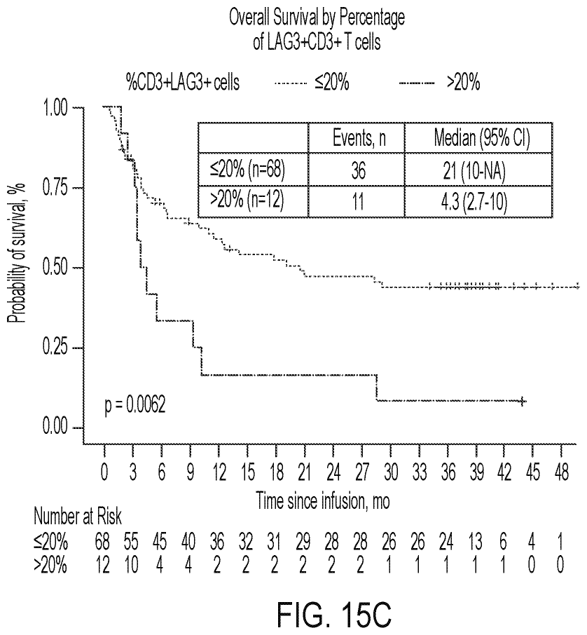

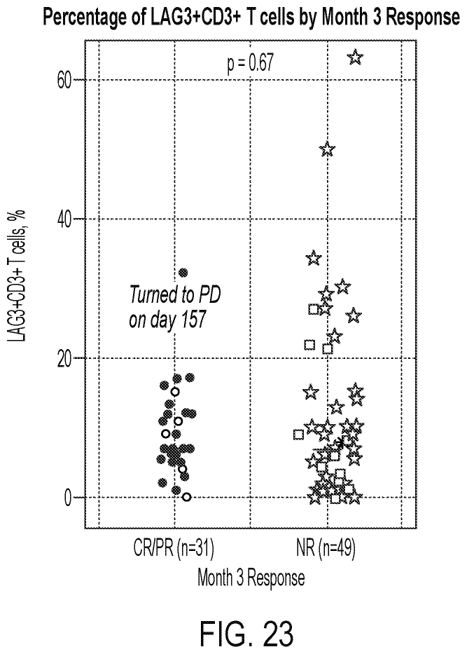

Interrogation of checkpoint molecule expression on tumor-infiltrating CD3+ cells revealed that patients with the highest frequency of LAG3+CD3+ out of CD3+ T cells (cutoff of ≥20%; n=12) at baseline had decreased median PFS (2.1 [95% CI, 0.82-3.1] and 4.2 months [95% CI, 2.4-21]) and OS (4.3 [95% CI, 2.7-10] and 21 months [95% CI, 10-NE]) compared with patients with <20% LAG3+CD3+ T cells (n=68; FIGS. 15A-15C). Patients with the highest frequency of LAG3+CD3+ cells out of the tumor infiltrating CD3+ T cell population (cutoff of >20%; n=12) at baseline were mostly non-responders at Month 3 (FIG. 23) and all 12 patients with >20% LAG3+CD3+ cells (out of the tumor infiltrating CD3+ T cells) were non-responders at Month 6.

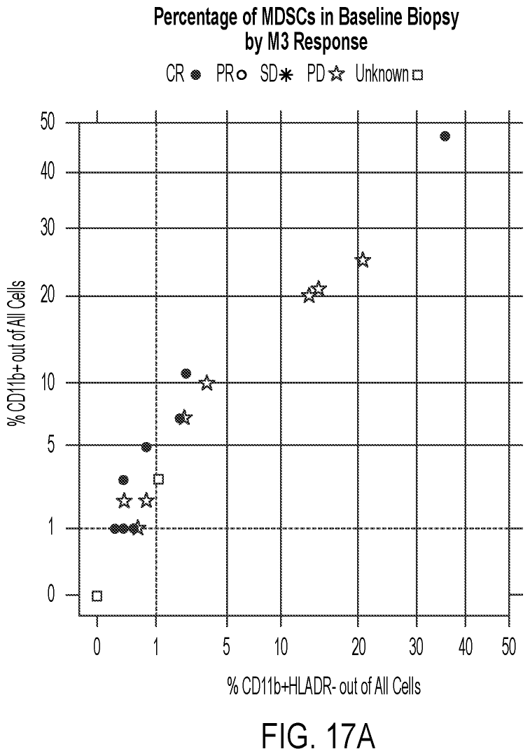

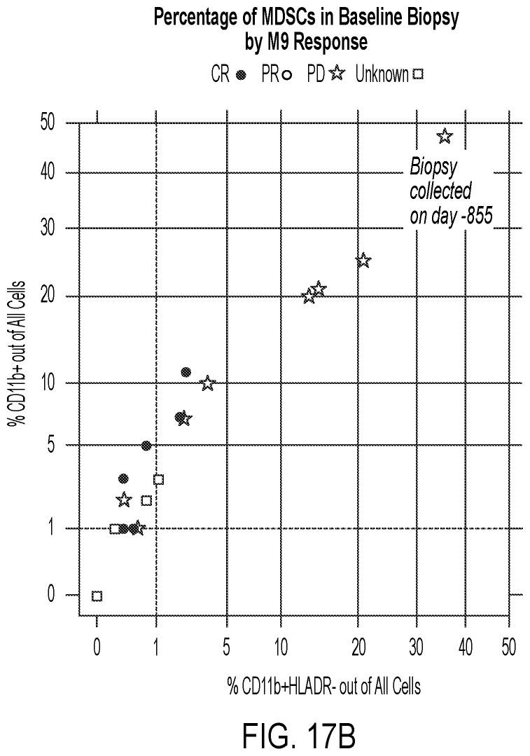

Additionally, in a small dataset, patients with the highest frequency of CD11b+HLA-DR− cells that represent myeloid-derived suppressor cell (MDSC) phenotype at baseline were enriched with non-responders. Approximately, 6/9 and 7/9 non-responders at month 3 and 9, respectively had a high level (≥1%) of CD11b+HLADR-MDSCs among all CD11b+myeloid-linage cells in the TME (FIG. 17A and FIG. 17B).

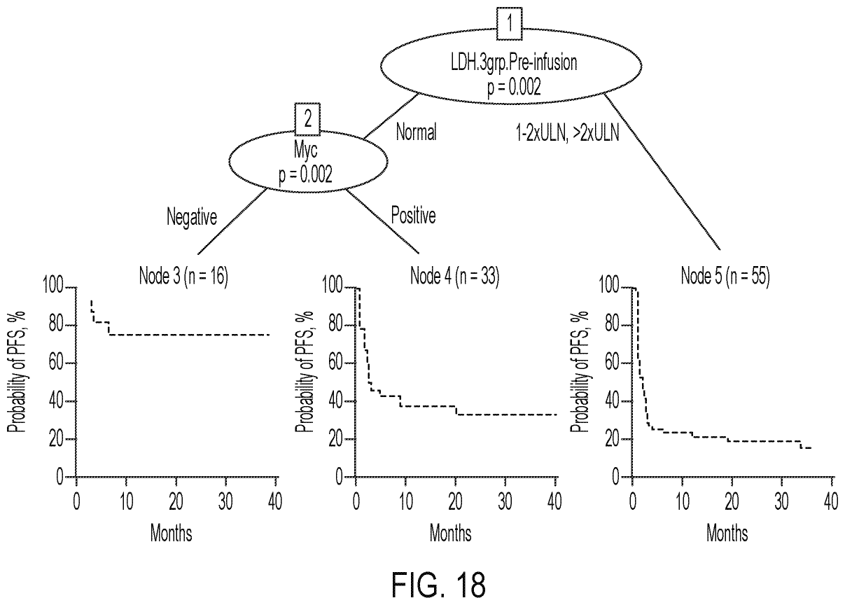

In a survival tree analysis including infiltrated T cells, CD3+LAG3+ T cells, MYC, and LDH patients with MYC(−) status and normal pre-infusion LDH levels (n=16) had superior PFS compared with normal LDH and MYC(+), and patients with LDH 1- to 2-fold or >2-fold above the upper limit of normal (ULN), with the latter group having poor PFS (FIG. 18 and Table 10). The probability of PFS in MYC(−) patients with normal LDH (n=16) was 81% at 6 months and stabilized at 75% starting at 12 months.

TABLE 10
Pre-infusion LDH levels

| | Pre-Infusion LDH Levels | | | |
|---|---|---|---|---|
| MYC Status | Normal | 1-2×ULN | >2×ULN | Sum |
| MYC(−) | 16 | 14 | 6 | 36 |
| MYC(+) | 33 | 21 | 14 | 68 |
| Sum | 49 | 35 | 20 | 104 |

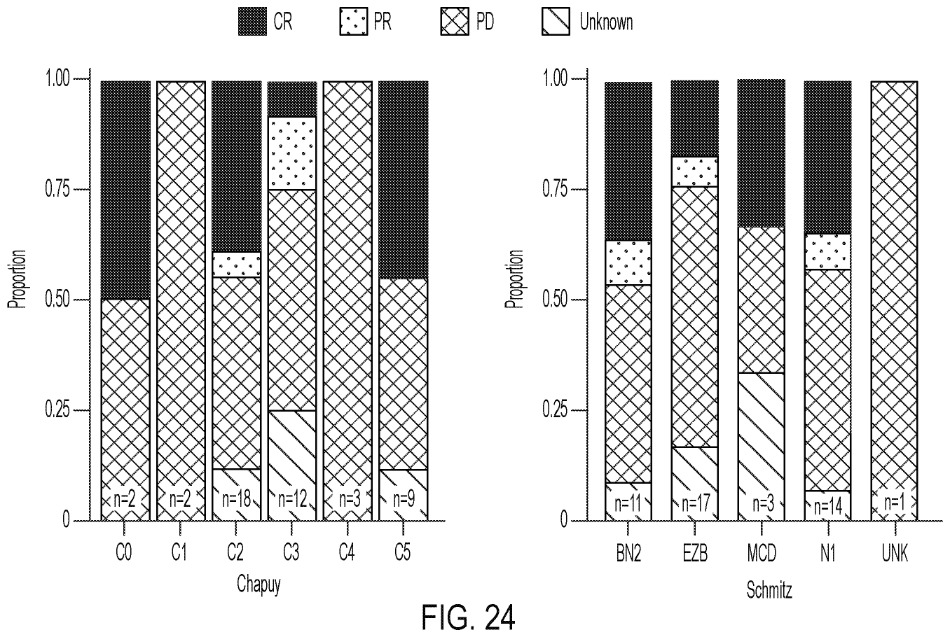

Whole exome sequencing of 46 baseline patient samples was performed to investigate the correlation between genetic subtypes and M3 response. Samples grouped into newly-identified DLBCL subsets (Chapuy et al Nat Med 2018; Schmitz et al N Engl J Med 2018) did not reveal an association with response (no significant association with response was observed in mutations at the single-gene level) (FIG. 24). Deletions/mutations in TP53 have been reported to be risk factors in DLBCL. 28% (13/46) of patients sequenced were found to have TP53 mutations, but no association with response was observed. No apparent difference in tumor mutational burden was observed in patients who achieved response compared with patients who did not.

Summary: Long-term data from the JULIET study demonstrated sustained benefit in responding patients, with 80% of patients with CR derived long-term OS benefit (≥20 months) from tisagenlecleucel. However, patients of the JULIET Trial exhibiting double or triple hit lymphoma, along with high MYC expression, tended to relapse faster to CART19 treatment when compared to patients from non-GCB subtypes of relapsed or refractory DLBCL. MYC overexpression or an immunosuppressive TME with restricted T cell response may impact CART cell efficacy in patients in DLBCL, as low to no CD3+ T cells and increase of CD3+LAG3+ T cells, or MDSCs within the TME were associated with worse outcomes. MYC(−) status and normal pre-infusion LDH levels demonstrated improved outcomes compared with MYC overexpression and high pre-infusion LDH levels. Taken together, the results from the biomarker analyses imply potential resistant or immunosuppressive mechanisms that may impact CAR-T cell efficacy in patients with DLBCL by restricting T-cell response and promoting an unfavorable TME.

### Example 2: Evaluation of an In Vitro Double-Hit Lymphoma Model for Sensitivity to CART19 Treatment

The Example describes the evaluation of CART19 killing of a double-hit lymphoma cell line in vitro.

SuDHL6 double-hit lymphoma cells were labeled with Cell Trace Far Red dye and plated in a 96-well plate at a concentration of 50,000 cells/well. These cells were grown in the presence of increasing amounts of dye-labeled CART19 cells or untransduced T cells containing no CAR (UTD) (negative control) and incubated for 44 hours. The CART19 cells or untransduced control T cells were added in concentrations that resulted in calculated ratios of effector T cells to SuDHL6 target cells (E:T) of 0.63, 1.25, 2.5, 5, and 10 (FIG. 1). As a control, SuDHL6 cells were plated and grown in the absence of any effector cells. Following incubation, the treated and untreated SuDHL6 cells were washed and stained with the Zombie Aqua viability dye and subsequently analyzed by flow cytometry. Cells were sorted and gated based on a live/dead cellular phenotype. The percentage (%) of cells that were killed by the added effector cells was calculated by subtracting the percentage of live cells remaining following treatment with CART19 cells or untransduced control T cells, from the percentage of live, untreated SuDHL6 cells.

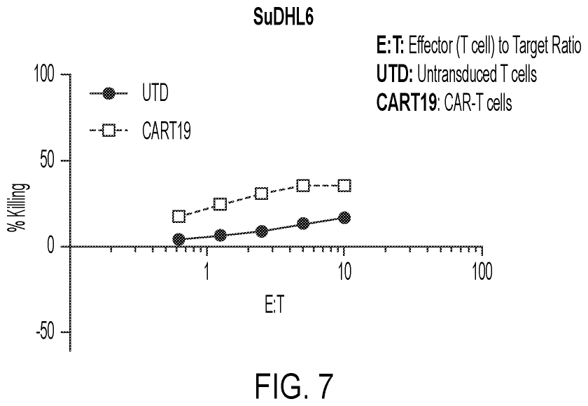

As depicted in FIG. 7, the SuDHL6 double-hit lymphoma cells appeared to be refractory to CART19 activity, as below 50% killing was observed for all ratios of effector cells to tumor cells investigated.

### Example 3: Evaluation of an In Vitro Double-Hit Lymphoma Model for Sensitivity to CART19 Treatment in Combination with a BCL2 Inhibitor

The Example describes the evaluation of CART19 cells in combination with a BCL2 inhibitor on the killing of double-hit lymphoma cells in vitro.

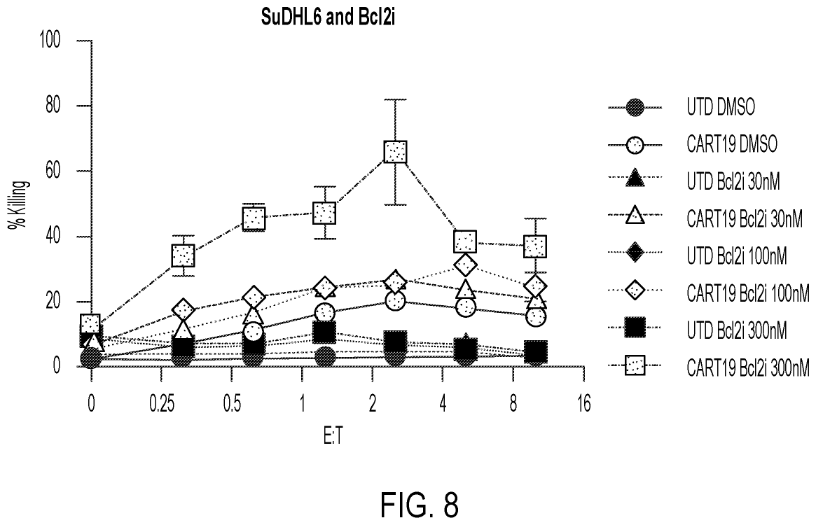

SuDHL6 double-hit lymphoma cells were labeled with Cell Trace Far Red dye and pre-incubated for four hours with increasing concentrations (30 nM, 100 nM, and 300 nM) of the BCL2 inhibitor (Bcl2i) venetoclax, or DSMO (negative control). The SuDHL6 cells were then plated as described in Example 1 and grown in the presence of increasing amounts of dye-labeled CART19 cells or untransduced T cells containing no CAR (UTD) (negative control) for at least 44 hours. The CART19 cells or untransduced control T cells were added in concentrations that resulted in calculated ratios of effector T cells to SuDHL6 target cells (E:T) of 0.31, 0.63, 1.25, 2.5, 5, and 10 (FIG. 8). As a control, SuDHL6 cells were plated and grown in the absence of any effector cells or BCL2 inhibitor. Following incubation, the treated and untreated SuDHL6 cells were washed and stained with the Zombia Aqua viability dye and subsequently analyzed by flow cytometry, as described in Example 1. The percentage (%) of cells that were killed by the added effector cells was calculated by subtracting the percentage of live cells remaining following treatment with CART19 cells or untransduced control T cells, from the percentage of live, untreated SuDHL6 cells.

As shown in FIG. 8, the addition of the BCL2 inhibitor in combination with the CART19 cells, led to a dose-dependent increase in killing of SuDHL6 cells, as compared to CART19 cells or the BCL2 inhibitor alone. This increase in killing was particularly observed when SuDHL6 cells were pre-incubated with 300 nM of the BCL2 inhibitor subsequently treated with CART19 cells at an effector cell to target cell ratio of 2, as this resulted in killing of 60% of double-hit lymphoma cells, compared to only a 20% of cells killed with CART19 cells alone at the same effector to target ratio. As such, the BCL2 inhibitor improved responses to CART19 cells in the SuDHL6, double-hit lymphoma cells in vitro.

### Example 4: Evaluation of In Vivo Model of Double-Hit Lymphoma

The Example describes the development of an in vivo, murine model of double-hit lymphoma.

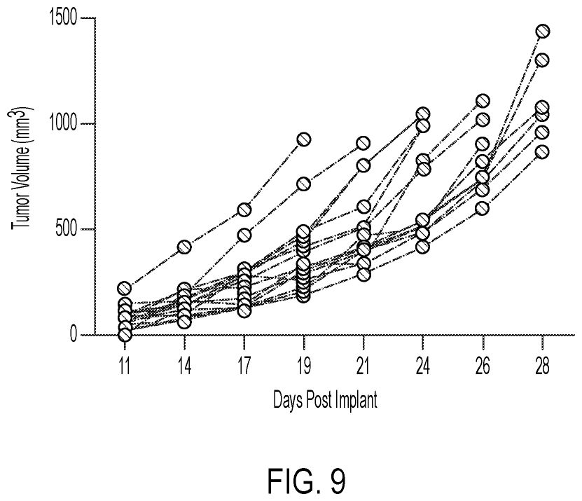

Ten NOD scid gamma (NSG) mice were implanted subcutaneously with a dose of 5e6 SuDHL6 cells/mouse. Tumor growth was monitored in these mice every three days starting on day 11 post-implantation, which was when tumors were first palpable. Tumor volume was quantified by caliper measurement at each timepoint sampled (FIG. 9). As shown in FIG. 9, all ten mice developed palpable tumors by day 11 after implant, and these tumors reached approximately 1000-15000 mm³ in volume by days 19-28 post-implantation. The Example demonstrates that SuDHL6 cells implanted in mice can be used as an in vivo double-hit lymphoma model for investigating responses to CART19 combination therapies, e.g., a CART19 combination therapy described herein.

### Example 5: Development of an In Vivo Double-Hit Lymphoma Model for Sensitivity to CART19 Treatment in Combination with a BCL2 Inhibitor

This Example describes the evaluation of CART19 cells in combination with a BCL2 inhibitor on the killing of double-hit lymphoma cells in vivo in a murine model.

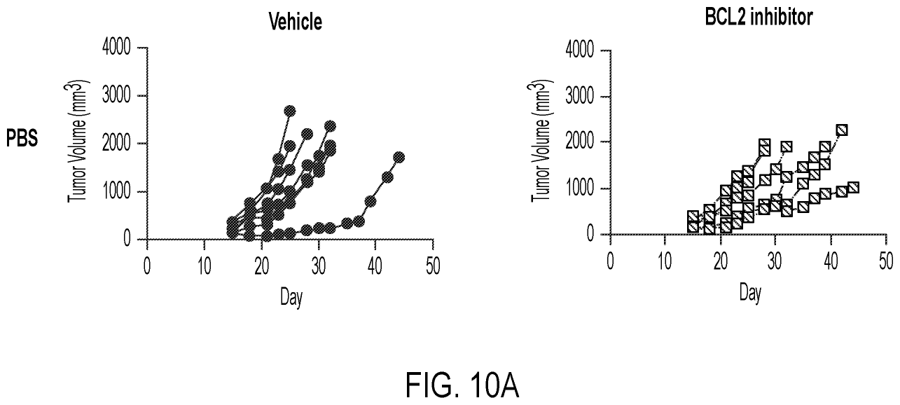

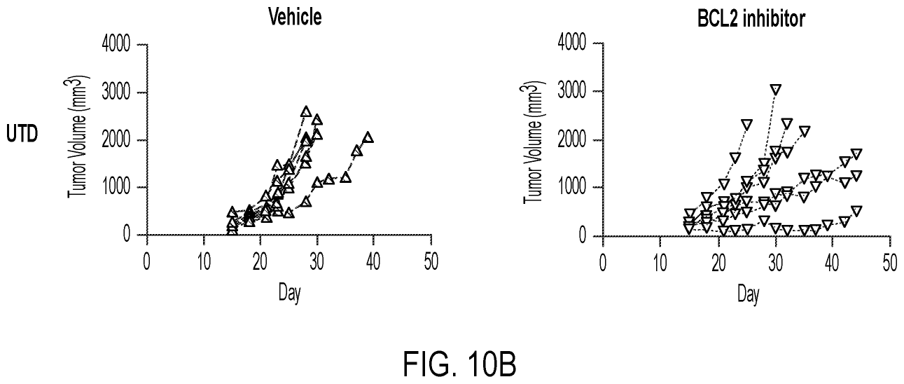

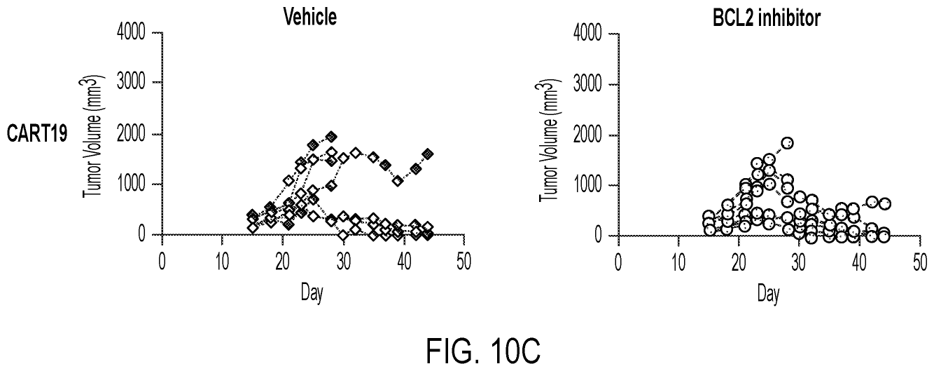

NOD scid gamma (NSG) mice were implanted subcutaneously with a dose of 5e6 SuDHL6 cells/mouse. At day 15 post-implant, mice were randomized into treatment groups, which included: PBS vehicle negative control (FIG. 10A); PBS vehicle and 100 mg/kg of the BCL2 inhibitor, venetoclax (FIG. 10A); 1.45e6 untransduced T cells containing no CAR (UTD) (FIG. 10B); 1.45e6 untransduced T cells containing no CAR (UTD) and 100 mg/kg of the BCL2 inhibitor, venetoclax (FIG. 10B); 1e6 CART19 cells (which were at 68% positivity, total cells injected were 1.45e6) (FIG. 10C); and 1e6 CART19 cells and 100 mg/kg of the BCL2 inhibitor, venetoclax (FIG. 10C). The BCL2 inhibitor was administered daily until sacrifice. Tumor growth was monitored in these mice every three days. Tumor volume was quantified by caliper measurement at each timepoint sampled (FIGS. 10A-C). Both the BCL2 inhibitor (FIG. 10A, right) and the CART19 cells (FIG. 10C, left), when administered as single agents, slowed tumor growth as compared to the PBS vehicle treated control (FIG. 10A, left) or untransduced CART control cells (FIG. 10B, left). However, administration of the BCL2 inhibitor in combination with the CART19 cells (FIG. 10C, right) resulted in superior tumor cell killing and more effective tumor clearance, as compared to CART19 cells administered alone (FIG. 10C, left).

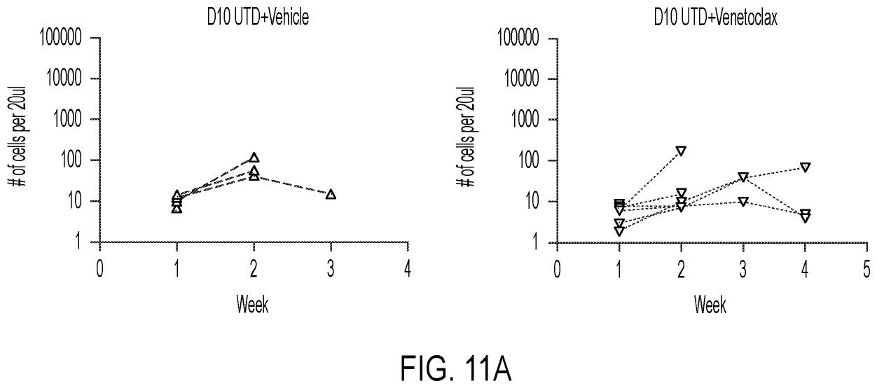

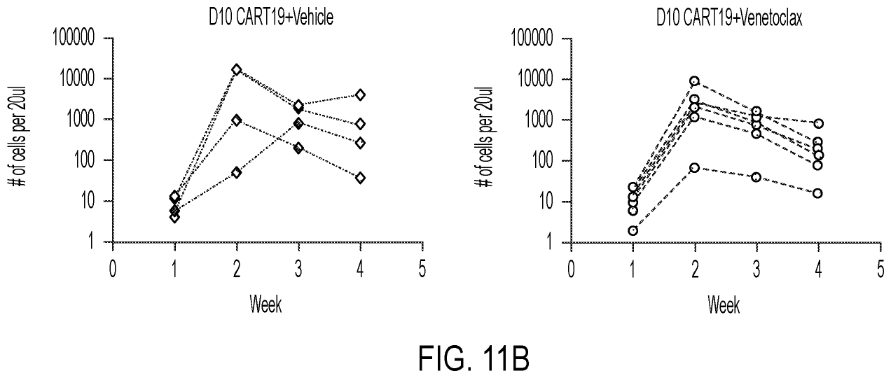

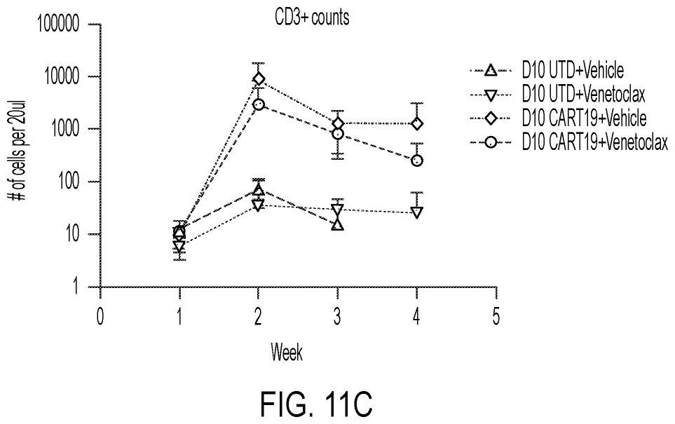

Additionally, blood was collected each week from week 1 to week 4 post-treatment. The blood was lysed to remove erythrocytes and the remaining white blood cells were stained for markers to quantify CD3+ T cells (FIGS. 11A-11C). Administration of the BCL2 inhibitor venetoclax, in combination with untransduced CART control cells (FIG. 11A) or CART19 cells (FIG. 11B) did not affect T cell growth or kinetics in the treated mice. FIG. 11C presents a summary, depicting the average number of T cells quantified per 20 μL of blood each week in the indicated treatment groups.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A method for treating a subject having, or identified as having, a B-cell lymphoma having an increased level and/or activity of a MYC gene or gene product and/or an anti-apoptotic gene or gene product, comprising:
administering to the subject a therapy comprising a population of immune effector cells that expresses a Chimeric Antigen Receptor (CAR) that binds to CD19 (“CD19 CAR therapy”), in combination with a BCL2 inhibitor, thereby treating the B-cell lymphoma in the subject.

2. A method of treating a subject having a B-cell lymphoma having an increased level and/or activity of a MYC gene or gene product and/or an anti-apoptotic gene or gene product (e.g., a high grade B-cell lymphoma), said method comprising:
administering to the subject a BCL2 inhibitor, wherein the subject is, or is identified as being, a non-responder, a partial responder, or a relapser to the CD19 CAR therapy.

3. The method of claim 2, wherein the subject has undergone, is undergoing, or will receive, the CD19 CAR therapy.

4. A method of treating or preventing a relapse to a population of immune effector cells that expresses a Chimeric Antigen Receptor (CAR) that binds to CD19 (“CD19 CAR therapy”) in a subject with a lymphoma having increased level and/or activity of a MYC gene or gene product and/or an anti-apoptotic gene or gene product (e.g., a high grade B-cell lymphoma), comprising:
administering a BCL2 inhibitor to a subject who has undergone, is undergoing, or will receive, the CD19 CAR therapy,
thereby treating or preventing the relapse to the CD19 CAR therapy.

5. The method of any of claims 1 to 4, wherein the subject has, or is identified as having, an alteration in a MYC gene or gene product, or an alteration in an anti-apoptotic gene or gene product, or a combination thereof.

6. The method of any of claims 1 to 5, wherein the subject has, or is identified as having an increased level of, e.g., increased number of cells positive for, a MYC gene or MYC gene product, e.g., as identified by detecting a rearrangement, e.g., translocation, using a FISH assay or an immunohistochemistry assay.

7. The method of claim 6, wherein the subject is identified as being MYC positive, e.g., by detecting greater than 40% of cells in a sample, e.g., a tumor biopsy or blood sample, from the subject as being positive for expression of a MYC gene product, e.g., by an immunohistochemistry assay.

8. The method of claim 7, wherein the MYC-positive subject, is further identified as having an increased level of a BCL2 gene or gene product and/or a BCL6 gene or gene product e.g., as identified by detecting a rearrangement, e.g., translocation, in a sample, e.g., a tumor biopsy or a blood sample, using a FISH assay or an immunohistochemistry assay.

9. The method of either of claim 7 or 8, wherein the MYC-positive subject having an increased level of the BCL2 gene or gene product or an increased level of the BCL6 gene or gene product is identified as having, a double hit (DH) lymphoma, e.g., a MYC and BCL2- or BCL6-positive lymphoma.

10. The method of any of claims 7 to 9, wherein the MYC-positive subject having an increased level of a BCL2 gene or gene product and a BCL6 gene or gene product is identified as having, a triple hit (TH) lymphoma, e.g., a MYC, BCL2, and BCL6-positive lymphoma.

11. The method of any of the preceding claims, wherein the lymphoma is chosen from a high grade B-cell lymphoma (e.g., a double hit lymphoma, a triple hit lymphoma, or a non-specified NOS high-grade lymphoma), a diffuse large B-cell lymphoma (DLBCL), or follicular lymphoma.

12. The method of claim 11, wherein the lymphoma is a high grade B-cell lymphoma.

13. The method of claim 12, wherein the high grade B-cell lymphoma is a double hit lymphoma.

14. The method of claim 12, wherein the high grade B-cell lymphoma is a triple hit lymphoma.

15. The method of claim 11, wherein lymphoma is DLBCL, e.g., a relapsed or refractory DLBCL.

16. The method of claim 11 or 15, wherein the DLBCL arises from a cell population comprising a Germinal Center B-Cell (GCB cell), an activated B-Cell (ABC cell), or an unclassified cell.

17. The method of claim 16, wherein the DLBCL arises from a cell population comprising a Germinal Center B-Cell (GCB cell).

18. The method of any of claims 11 or 15-17, wherein the DLBCL is relapsed or refractory DLBCL.

19. The method of claim 11, wherein the lymphoma is a follicular lymphoma (FL).

20. The method of claim 11 or 19, wherein the FL is a relapsed or refractory FL.

21. The method of any of claims 1-2, or 4-20, wherein the subject has undergone, is undergoing the CD19 CAR therapy, or will receive the CD19 CAR therapy.

22. The method of any of the preceding claims, wherein the subject has relapsed, or is identified as having relapsed, after treatment with the CD19 CAR therapy.

23. The method of any of the preceding claims, wherein the subject has relapsed or is identified as having relapsed based on one or more of:
(1) a reappearance of a bone marrow involvement, e.g., a lesion, after a complete response;
(2) a reappearance of a malignant effusion, after a complete response;
(3) a reappearance of a nodal lesion greater than 1.5 cm (e.g., a previously normal lymph node becoming greater than 1.5 cm) by CT scan or MRI, after a complete response;
(4) a reappearance of a discrete extranodal lesion (including liver or spleen) by CT scan or MRI after a complete response; or
(5) a ≥50% increase in the size of a residual lymph node or mass, e.g., the long axis from baseline of the lymph node or mass.

24. The method of any of the preceding claims, wherein the CD19 CAR therapy and the BCL2 inhibitor are administered concurrently or sequentially.

25. The method of any of the preceding claims, wherein the subject is treated with the BCL2 inhibitor before, concurrently, and/or after the CD19 CAR therapy.

26. The method of any of the preceding claims, wherein the subject is evaluated prior to, during, or after receiving the CD19 CAR therapy or the BCL2 inhibitor for the presence or absence of the alteration in the MYC gene or gene product, or the alteration in the anti-apoptotic gene or gene product, or a combination thereof.

27. The method of any one of the preceding claims, wherein the CD19 CAR therapy comprises a CD19 CAR comprising an anti-CD19 binding domain, a transmembrane domain, and an intracellular signaling domain comprising a stimulatory domain.

28. The method of claim 27, wherein the anti-CD19 binding domain of the CD19 CAR comprises one or more of light chain complementary determining region 1 (LC CDR1), light chain complementary determining region 2 (LC CDR2), and light chain complementary determining region 3 (LC CDR3) of any anti-CD19 light chain binding domain amino acid sequence listed in Tables 2 or 3, and one or more of heavy chain complementary determining region 1 (HC CDR1), heavy chain complementary determining region 2 (HC CDR2), and heavy chain complementary determining region 3 (HC CDR3) of any anti-CD19 heavy chain binding domain amino acid sequence listed in Tables 2 or 3.

29. The method of claim 27 or 28, wherein the anti-CD19 binding domain of the CD19 CAR comprises an amino acid sequence of SEQ ID NO: 1-12 or SEQ ID NO: 59, or a sequence at least 95% identical thereto.

30. The method of claim 27 or 28, wherein the anti-CD19 binding domain comprises a sequence of SEQ ID NO: 2, or SEQ ID NO:59, or a sequence at least 95% identical thereto.

31. The method of any of the preceding claims, wherein the CD19 CAR comprises an amino acid sequence of any of SEQ ID NO: 31-42 or SEQ ID NO: 58, or a sequence at least 95% identical thereto.

32. The method of any of the preceding claims, wherein the CD19 CAR comprises a polypeptide having the amino acid sequence of SEQ ID NO:32, or SEQ ID NO: 58, or a sequence at least 95% identical thereto.

33. The method of any of the preceding claims, wherein the CD19 CAR therapy is a therapy comprising CTL-019 or CTL-119 or both.

34. The method of any of the preceding claims, wherein the CD19 CAR therapy is administered intravenously.

35. The method of any of the preceding claims, wherein the BCL2 inhibitor is chosen from venetoclax (ABT-199), navitoclax (ABT-263), ABT-737, BP1002, SPC2996, APG-1252, obatoclax mesylate (GX15-070MS), PNT2258, or oblimersen (G3139), or a combination.

36. The method of any of the preceding claims, wherein the BCL2 inhibitor is venetoclax.

37. The method of any of the preceding claims, further comprising administering a standard of care for DLBCL, e.g., a CD20 inhibitor, a chemotherapeutic agent, and/or a corticosteroid.

38. A combination comprising a CD19 CAR therapy and a BCL2 inhibitor.


SEQUENCE LISTING (5037 sequences)

SEQ ID NO: 1 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 2 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 3 - protein, 242 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 4 - protein, 242 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 5 - protein, 247 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 6 - protein, 247 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 7 - protein, 247 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 8 - protein, 247 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 9 - protein, 247 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 10 - protein, 247 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 11 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 12 - protein, 242 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 13 - protein, 21 aa, Artificial Sequence
  source: /note=“Description of Artificial Sequence: Synthetic peptide”
MALPVTALLLPLALLLHAARP

SEQ ID NO: 14 - protein, 45 aa, Artificial Sequence
  source: /note=“Description of Artificial Sequence: Synthetic polypeptide”
TTTPAPRPPTPAPTIASQPLSLRPEACRPAAGGAVHTRGLDFACD

SEQ ID NO: 15 - protein, 24 aa, Artificial Sequence
  source: /note=“Description of Artificial Sequence: Synthetic peptide”
IYIWAPLAGTCGVLLLSLVITLYC

SEQ ID NO: 16 - protein, 42 aa, Artificial Sequence
  source: /note=“Description of Artificial Sequence: Synthetic polypeptide”
KRGRKKLLYIFKQPFMRPVQTTQEEDGCSCRFPEEEEGGCEL

SEQ ID NO: 17 - protein, 112 aa, Artificial Sequence
  source: /note=“Description of Artificial Sequence: Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 5 aa, Artificial Sequence
  source: /note=“Description of Artificial Sequence: Synthetic peptide”
GGGGS

SEQ ID NO: 19 - protein, 10 aa, Artificial Sequence
  source: /note=“Description of Artificial Sequence: Synthetic peptide”
GVSLPDYGVS

SEQ ID NO: 20 - protein, 16 aa, Artificial Sequence
  source: /note=“Description of Artificial Sequence: Synthetic peptide”
VIWGSETTYYNSALKS

SEQ ID NO: 21 - protein, 16 aa, Artificial Sequence
  source: /note=“Description of Artificial Sequence: Synthetic peptide”
VIWGSETTYYSSSLKS

SEQ ID NO: 22 - protein, 16 aa, Artificial Sequence
  source: /note=“Description of Artificial Sequence: Synthetic peptide”
VIWGSETTYYQSSLKS

SEQ ID NO: 23 - protein, 16 aa, Artificial Sequence
  source: /note=“Description of Artificial Sequence: Synthetic peptide”
VIWGSETTYYNSSLKS

SEQ ID NO: 24 - protein, 12 aa, Artificial Sequence
  source: /note=“Description of Artificial Sequence: Synthetic peptide”
HYYYGGSYAMDY

SEQ ID NO: 25 - protein, 11 aa, Artificial Sequence
  source: /note=“Description of Artificial Sequence: Synthetic peptide”
RASQDISKYLN

SEQ ID NO: 26 - protein, 7 aa, Artificial Sequence
  source: /note=“Description of Artificial Sequence: Synthetic peptide”
HTSRLHS

SEQ ID NO: 27 - protein, 9 aa, Artificial Sequence
  source: /note=“Description of Artificial Sequence: Synthetic peptide”
QQGNTLPYT

SEQ ID NO: 28 - protein, 15 aa, Artificial Sequence
  source: /note=“Description of Artificial Sequence: Synthetic peptide”
GGGGSGGGGSGGGGS

SEQ ID NO: 29 - protein, 4 aa, Artificial Sequence
  source: /note=“Description of Artificial Sequence: Synthetic peptide”
GGGS

SEQ ID NO: 30 - RNA, 5000 nt, Artificial Sequence
  source: /note=“Description of Artificial Sequence: Synthetic polynucleotide”
  misc_feature (1)..(5000): /note=“This sequence may encompass 50-5000 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 486 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 486 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 491 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 491 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 491 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 491 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 491 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 491 aa, Artificial Sequence; identical to SEQ ID NO: 39
  source: /note=“Description of Artificial Sequence: Synthetic polypeptide”

SEQ ID NO: 41 - protein, 491 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 45 - protein, 230 aa, Artificial Sequence
  source: /note=“Description of Artificial Sequence: Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 46 - DNA, 690 bp, Artificial Sequence
  source: /note=“Description of Artificial Sequence: Synthetic polynucleotide”
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 47 - protein, 282 aa, Artificial Sequence
  source: /note=“Description of Artificial Sequence: Synthetic polypeptide”
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 48 - DNA, 847 bp, Artificial Sequence
  source: /note=“Description of Artificial Sequence: Synthetic polynucleotide”
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 49 - protein, 10 aa, Artificial Sequence
  source: /note=“Description of Artificial Sequence: Synthetic peptide”
GGGGSGGGGS

SEQ ID NO: 50 - DNA, 30 bp, Artificial Sequence
  source: /note=“Description of Artificial Sequence: Synthetic oligonucleotide”
ggtggcggaggttctggaggtggaggttcc

SEQ ID NO: 51 - protein, 48 aa, Artificial Sequence
  source: /note=“Description of Artificial Sequence: Synthetic polypeptide”
QRRKYRSNKGESPVEPAEPCRYSCPREEEGSTIPIQEDYRKPEPACSP

SEQ ID NO: 52 - DNA, 123 bp, Artificial Sequence
  source: /note=“Description of Artificial Sequence: Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 53 - protein, 30 aa, Artificial Sequence
  source: /note=“Description of Artificial Sequence: Synthetic polypeptide”
  SITE (1)..(30): /note=“This sequence may encompass 1-6 ′Gly Gly Gly Gly Ser′ repeating units”
  source: /note=“See specification as filed for detailed description of substitutions and preferred embodiments”
GGGGSGGGGSGGGGSGGGGSGGGGSGGGGS

SEQ ID NO: 54 - DNA, 63 bp, Artificial Sequence
  source: /note=“Description of Artificial Sequence: Synthetic oligonucleotide”
atggccctgcctgtgacagccctgctgctgcctctggctctgctgctgcatgccgctagaccc

SEQ ID NO: 55 - DNA, 135 bp, Artificial Sequence
  source: /note=“Description of Artificial Sequence: Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 56 - DNA, 72 bp, Artificial Sequence
  source: /note=“Description of Artificial Sequence: Synthetic oligonucleotide”
atctacatctgggcgcccttggccgggacttgtggggtccttctcctgtcactggttatcaccctttactgc

SEQ ID NO: 57 - skipped

SEQ ID NO: 58 - protein, 486 aa, Artificial Sequence
  source: /note=“Description of Artificial Sequence: Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 242 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 60 - DNA, 126 bp, Artificial Sequence
  source: /note=“Description of Artificial Sequence: Synthetic polynucleotide”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 61 - DNA, 813 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 62 - DNA, 813 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 63 - DNA, 813 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactattcatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaacatcaccaccatcatcaccatcac

SEQ ID NO: 64 - DNA, 813 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactatcaatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaacatcaccaccatcatcaccatcac

SEQ ID NO: 65 - DNA, 828 bp, Artificial Sequence
  source: /note=“Description of Artificial Sequence: Synthetic polynucleotide”
atggccctcccagtgaccgctctgctgctgcctctcgcacttcttctccatgccgctcggcctgagatcgtcatgacccaaagccccgctaccctgtccctgtcacccggcgagagggcaaccctttcatgcagggccagccaggacatttctaagtacctcaactggtatcagcagaagccagggcaggctcctcgcctgctgatctaccacaccagccgcctccacagcggtatccccgccagattttccgggagcgggtctggaaccgactacaccctcaccatctcttctctgcagcccgaggatttcgccgtctatttctgccagcaggggaatactctgccgtacaccttcggtcaaggtaccaagctggaaatcaagggaggcggaggatcaggcggtggcggaagcggaggaggtggctccggaggaggaggttcccaagtgcagcttcaagaatcaggacccggacttgtgaagccatcagaaaccctctccctgacttgtaccgtgtccggtgtgagcctccccgactacggagtctcttggattcgccagcctccggggaagggtcttgaatggattggggtgatttggggatcagagactacttactactcttcatcacttaagtcacgggtcaccatcagcaaagataatagcaagaaccaagtgtcacttaagctgtcatctgtgaccgccgctgacaccgccgtgtactattgtgccaaacattactattacggagggtcttatgctatggactactggggacaggggaccctggtgactgtctctagccatcaccatcaccaccatcatcac

SEQ ID NO: 66 - DNA, 828 bp, Artificial Sequence
  source: /note=“Description of Artificial Sequence: Synthetic polynucleotide”
atggccctcccagtgaccgctctgctgctgcctctcgcacttcttctccatgccgctcggcctgagatcgtcatgacccaaagccccgctaccctgtccctgtcacccggcgagagggcaaccctttcatgcagggccagccaggacatttctaagtacctcaactggtatcagcagaagccagggcaggctcctcgcctgctgatctaccacaccagccgcctccacagcggtatccccgccagattttccgggagcgggtctggaaccgactacaccctcaccatctcttctctgcagcccgaggatttcgccgtctatttctgccagcaggggaatactctgccgtacaccttcggtcaaggtaccaagctggaaatcaagggaggcggaggatcaggcggtggcggaagcggaggaggtggctccggaggaggaggttcccaagtgcagcttcaagaatcaggacccggacttgtgaagccatcagaaaccctctccctgacttgtaccgtgtccggtgtgagcctccccgactacggagtctcttggattcgccagcctccggggaagggtcttgaatggattggggtgatttggggatcagagactacttactaccagtcatcacttaagtcacgggtcaccatcagcaaagataatagcaagaaccaagtgtcacttaagctgtcatctgtgaccgccgctgacaccgccgtgtactattgtgccaaacattactattacggagggtcttatgctatggactactggggacaggggaccctggtgactgtctctagccatcaccatcaccaccatcatcac

SEQ ID NO: 67 - DNA, 828 bp, Artificial Sequence
  source: /note=“Description of Artificial Sequence: Synthetic polynucleotide”
atggcactgcctgtcactgccctcctgctgcctctggccctccttctgcatgccgccaggccccaagtccagctgcaagagtcaggacccggactggtgaagccgtctgagactctctcactgacttgtaccgtcagcggcgtgtccctccccgactacggagtgtcatggatccgccaacctcccgggaaagggcttgaatggattggtgtcatctggggttctgaaaccacctactactcatcttccctgaagtccagggtgaccatcagcaaggataattccaagaaccaggtcagccttaagctgtcatctgtgaccgctgctgacaccgccgtgtattactgcgccaagcactactattacggaggaagctacgctatggactattggggacagggcactctcgtgactgtgagcagcggcggtggagggtctggaggtggaggatccggtggtggtgggtcaggcggaggagggagcgagattgtgatgactcagtcaccagccaccctttctctttcacccggcgagagagcaaccctgagctgtagagccagccaggacatttctaagtacctcaactggtatcagcaaaaaccggggcaggcccctcgcctcctgatctaccatacctcacgccttcactctggtatccccgctcggtttagcggatcaggatctggtaccgactacactctgaccatttccagcctgcagccagaagatttcgcagtgtatttctgccagcagggcaatacccttccttacaccttcggtcagggaaccaagctcgaaatcaagcaccatcaccatcatcaccaccat

SEQ ID NO: 68 - DNA, 828 bp, Artificial Sequence
  source: /note=“Description of Artificial Sequence: Synthetic polynucleotide”
atggcactgcctgtcactgccctcctgctgcctctggccctccttctgcatgccgccaggccccaagtccagctgcaagagtcaggacccggactggtgaagccgtctgagactctctcactgacttgtaccgtcagcggcgtgtccctccccgactacggagtgtcatggatccgccaacctcccgggaaagggcttgaatggattggtgtcatctggggttctgaaaccacctactaccagtcttccctgaagtccagggtgaccatcagcaaggataattccaagaaccaggtcagccttaagctgtcatctgtgaccgctgctgacaccgccgtgtattactgcgccaagcactactattacggaggaagctacgctatggactattggggacagggcactctcgtgactgtgagcagcggcggtggagggtctggaggtggaggatccggtggtggtgggtcaggcggaggagggagcgagattgtgatgactcagtcaccagccaccctttctctttcacccggcgagagagcaaccctgagctgtagagccagccaggacatttctaagtacctcaactggtatcagcaaaaaccggggcaggcccctcgcctcctgatctaccatacctcacgccttcactctggtatccccgctcggtttagcggatcaggatctggtaccgactacactctgaccatttccagcctgcagccagaagatttcgcagtgtatttctgccagcagggcaatacccttccttacaccttcggtcagggaaccaagctcgaaatcaagcaccatcaccatcatcatcaccac

SEQ ID NO: 69 - DNA, 828 bp, Artificial Sequence
  source: /note=“Description of Artificial Sequence: Synthetic polynucleotide”
atggccctcccagtgaccgctctgctgctgcctctcgcacttcttctccatgccgctcggcctgagatcgtcatgacccaaagccccgctaccctgtccctgtcacccggcgagagggcaaccctttcatgcagggccagccaggacatttctaagtacctcaactggtatcagcagaagccagggcaggctcctcgcctgctgatctaccacaccagccgcctccacagcggtatccccgccagattttccgggagcgggtctggaaccgactacaccctcaccatctcttctctgcagcccgaggatttcgccgtctatttctgccagcaggggaatactctgccgtacaccttcggtcaaggtaccaagctggaaatcaagggaggcggaggatcaggcggtggcggaagcggaggaggtggctccggaggaggaggttcccaagtgcagcttcaagaatcaggacccggacttgtgaagccatcagaaaccctctccctgacttgtaccgtgtccggtgtgagcctccccgactacggagtctcttggattcgccagcctccggggaagggtcttgaatggattggggtgatttggggatcagagactacttactacaattcatcacttaagtcacgggtcaccatcagcaaagataatagcaagaaccaagtgtcacttaagctgtcatctgtgaccgccgctgacaccgccgtgtactattgtgccaaacattactattacggagggtcttatgctatggactactggggacaggggaccctggtgactgtctctagccatcaccatcaccaccatcatcac

SEQ ID NO: 70 - DNA, 828 bp, Artificial Sequence
  source: /note=“Description of Artificial Sequence: Synthetic polynucleotide”
atggcactgcctgtcactgccctcctgctgcctctggccctccttctgcatgccgccaggccccaagtccagctgcaagagtcaggacccggactggtgaagccgtctgagactctctcactgacttgtaccgtcagcggcgtgtccctccccgactacggagtgtcatggatccgccaacctcccgggaaagggcttgaatggattggtgtcatctggggttctgaaaccacctactacaactcttccctgaagtccagggtgaccatcagcaaggataattccaagaaccaggtcagccttaagctgtcatctgtgaccgctgctgacaccgccgtgtattactgcgccaagcactactattacggaggaagctacgctatggactattggggacagggcactctcgtgactgtgagcagcggcggtggagggtctggaggtggaggatccggtggtggtgggtcaggcggaggagggagcgagattgtgatgactcagtcaccagccaccctttctctttcacccggcgagagagcaaccctgagctgtagagccagccaggacatttctaagtacctcaactggtatcagcaaaaaccggggcaggcccctcgcctcctgatctaccatacctcacgccttcactctggtatccccgctcggtttagcggatcaggatctggtaccgactacactctgaccatttccagcctgcagccagaagatttcgcagtgtatttctgccagcagggcaatacccttccttacaccttcggtcagggaaccaagctcgaaatcaagcaccatcaccatcatcaccaccat

SEQ ID NO: 71 - DNA, 813 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaattcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 72 - DNA, 813 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactataactcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaacatcaccaccatcatcaccatcac

SEQ ID NO: 73 - protein, 271 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSHHHHHHHH

SEQ ID NO: 74 - protein, 271 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSHHHHHHHH

SEQ ID NO: 75 - protein, 271 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 76 - protein, 271 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 77 - protein, 276 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSHHHHHHHH

SEQ ID NO: 78 - protein, 276 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSHHHHHHHH

SEQ ID NO: 79 - protein, 276 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 80 - protein, 276 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 81 - protein, 276 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSHHHHHHHH

SEQ ID NO: 82 - protein, 276 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 83 - protein, 271 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSHHHHHHHH

SEQ ID NO: 84 - protein, 271 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 85 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 86 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 87 - DNA, 1458 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactattcatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 88 - DNA, 1458 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactatcaatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 89 - DNA, 1473 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggcggaggcgggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 90 - DNA, 1473 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggaggcggagggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 91 - DNA, 1473 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactattcatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggtggcggaagcgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 92 - DNA, 1473 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactatcaatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggcggtgggtcagaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 93 - DNA, 1473 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggaggcggtgggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 94 - DNA, 1473 bp, Artificial Sequence; identical to SEQ ID NO: 93
  source: /note=“Description of Artificial Sequence: Synthetic polynucleotide”

SEQ ID NO: 95 - DNA, 1473 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactataactcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggtggcggaagcgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 96 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 97 - DNA, 813 bp, Artificial Sequence
  source: /note=“Description of Artificial Sequence: Synthetic polynucleotide”
atggccctgcccgtcaccgctctgctgctgccccttgctctgcttcttcatgcagcaaggccggacatccagatgacccaaaccacctcatccctctctgcctctcttggagacagggtgaccatttcttgtcgcgccagccaggacatcagcaagtatctgaactggtatcagcagaagccggacggaaccgtgaagctcctgatctaccatacctctcgcctgcatagcggcgtgccctcacgcttctctggaagcggatcaggaaccgattattctctcactatttcaaatcttgagcaggaagatattgccacctatttctgccagcagggtaataccctgccctacaccttcggaggagggaccaagctcgaaatcaccggtggaggaggcagcggcggtggagggtctggtggaggtggttctgaggtgaagctgcaagaatcaggccctggacttgtggccccttcacagtccctgagcgtgacttgcaccgtgtccggagtctccctgcccgactacggagtgtcatggatcagacaacctccacggaaaggactggaatggctcggtgtcatctggggtagcgaaactacttactacaattcagccctcaaaagcaggctgactattatcaaggacaacagcaagtcccaagtctttcttaagatgaactcactccagactgacgacaccgcaatctactattgtgctaagcactactactacggaggatcctacgctatggattactggggacaaggtacttccgtcactgtctcttcacaccatcatcaccatcaccatcac

SEQ ID NO: 98 - protein, 271 aa, Artificial Sequence
  source: /note=“Description of Artificial Sequence: Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSHHHHHHHH

SEQ ID NO: 99 - DNA, 1458 bp, Artificial Sequence
  source: /note=“Description of Artificial Sequence: Synthetic polynucleotide”
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 100 - DNA, 1184 bp, Artificial Sequence
  source: /note=“Description of Artificial Sequence: Synthetic polynucleotide”
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 101 - DNA, 336 bp, Artificial Sequence
  source: /note=“Description of Artificial Sequence: Synthetic polynucleotide”
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 102 - protein, 230 aa, Artificial Sequence; identical to SEQ ID NO: 45
  source: /note=“Description of Artificial Sequence: Synthetic polypeptide”

SEQ ID NO: 103 - DNA, 690 bp, Artificial Sequence; identical to SEQ ID NO: 46
  source: /note=“Description of Artificial Sequence: Synthetic polynucleotide”

SEQ ID NO: 104-105 - skipped (2 entries)

SEQ ID NO: 106 - protein, 20 aa, Artificial Sequence
  source: /note=“Description of Artificial Sequence: Synthetic peptide”
GGGGSGGGGSGGGGSGGGGS

SEQ ID NO: 107 - protein, 15 aa, Artificial Sequence
  source: /note=“Description of Artificial Sequence: Synthetic peptide”
GGGGSGGGGSGGGGS

SEQ ID NO: 108 - skipped

SEQ ID NO: 109 - protein, 244 aa, Artificial Sequence
  source: /note=“Description of Artificial Sequence: Synthetic polypeptide”
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGGGSGGGSGGGSGGGSELVLTQSPKFMSTSVGDRVSVTCKASQNVGTNVAWYQQKPGQSPKPLIYSATYRNSGVPDRFTGSGSGTDFTLTITNVQSKDLADYFCQYNRYPYTSFFFTKLEIKRRS

SEQ ID NO: 110 - protein, 464 aa, Artificial Sequence
  source: /note=“Description of Artificial Sequence: Synthetic polypeptide”
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGGGSGGGSGGGSGGGSELVLTQSPKFMSTSVGDRVSVTCKASQNVGTNVAWYQQKPGQSPKPLIYSATYRNSGVPDRFTGSGSGTDFTLTITNVQSKDLADYFCQYNRYPYTSFFFTKLEIKRRSKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 246 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSE

SEQ ID NO: 112 - protein, 439 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRL

SEQ ID NO: 113 - protein, 819 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 114 - protein, 245 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 115 - protein, 467 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116-117 - skipped (2 entries)

SEQ ID NO: 118 - RNA, 2000 nt, Artificial Sequence
  source: /note=“Description of Artificial Sequence: Synthetic polynucleotide”
  misc_feature (1)..(2000): /note=“This sequence may encompass 50-2000 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 119-121 - skipped (3 entries)

SEQ ID NO: 122 - protein, 132 aa, Artificial Sequence
  source: /note=“Description of Artificial Sequence: Synthetic polypeptide”
DVPDYASLGGPSSPKKKRKVSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSY

SEQ ID NO: 123 - protein, 108 aa, Artificial Sequence
  source: /note=“Description of Artificial Sequence: Synthetic polypeptide”
VQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETS

SEQ ID NO: 124 - protein, 93 aa, Artificial Sequence [sequence omitted]

SEQ ID NO: 125 - protein, 95 aa, Artificial Sequence
  source: /note=“Description of Artificial Sequence: Synthetic polypeptide”
ILWHEMWHEGLIEASRLYFGERNVKGMFEVLEPLHAMMERGPQTLKETSFNQAYGRDLMEAQEWCRKYMKSGNVKDLTQAWDLYYHVFRRISKTS

SEQ ID NO: 126 - protein, 95 aa, Artificial Sequence
  source: /note=“Description of Artificial Sequence: Synthetic polypeptide”
ILWHEMWHEGLLEASRLYFGERNVKGMFEVLEPLHAMMERGPQTLKETSFNQAYGRDLMEAQEWCRKYMKSGNVKDLTQAWDLYYHVFRRISKTS

SEQ ID NO: 127 - protein, 95 aa, Artificial Sequence
  source: /note=“Description of Artificial Sequence: Synthetic polypeptide”
ILWHEMWHEGLEEASRLYFGERNVKGMFEVLEPLHAMMERGPQTLKETSFNQAYGRDLMEAQEWCRKYMKSGNVKDLLQAWDLYYHVFRRISKTS

SEQ ID NO: 128 - protein, 95 aa, Artificial Sequence
  source: /note=“Description of Artificial Sequence: Synthetic polypeptide”
  MOD_RES (12)..(12): Any amino acid
  MOD_RES (78)..(78): Any amino acid
ILWHEMWHEGLXEASRLYFGERNVKGMFEVLEPLHAMMERGPQTLKETSFNQAYGRDLMEAQEWCRKYMKSGNVKDLXQAWDLYYHVFRRISKTS

SEQ ID NO: 129 - protein, 95 aa, Artificial Sequence
  source: /note=“Description of Artificial Sequence: Synthetic polypeptide”
ILWHEMWHEGLIEASRLYFGERNVKGMFEVLEPLHAMMERGPQTLKETSFNQAYGRDLMEAQEWCRKYMKSGNVKDLLQAWDLYYHVFRRISKTS

SEQ ID NO: 130 - protein, 95 aa, Artificial Sequence
  source: /note=“Description of Artificial Sequence: Synthetic polypeptide”
ILWHEMWHEGLLEASRLYFGERNVKGMFEVLEPLHAMMERGPQTLKETSFNQAYGRDLMEAQEWCRKYMKSGNVKDLLQAWDLYYHVFRRISKTS

SEQ ID NO: 131-362 - skipped (232 entries)

SEQ ID NO: 363 - protein, 1132 aa, Homo sapiens [sequence omitted]

SEQ ID NO: 364-1263 - skipped (900 entries)

SEQ ID NO: 1264 - protein, 30 aa, Artificial Sequence; identical to SEQ ID NO: 53
  source: /note=“Description of Artificial Sequence: Synthetic polypeptide”
  SITE (1)..(30): /note=“This sequence may encompass 1-6 ′Gly Gly Gly Gly Ser′ repeating units wherein some positions may be absent”
  source: /note=“See specification as filed for detailed description of substitutions and preferred embodiments”

SEQ ID NO: 1265-1315 - skipped (51 entries)

SEQ ID NO: 1316 - protein, 4 aa, Artificial Sequence
  source: /note=“Description of Artificial Sequence: Synthetic peptide”
RGDS

SEQ ID NO: 1317 - protein, 41 aa, Artificial Sequence
  source: /note=“Description of Artificial Sequence: Synthetic polypeptide”
RSKRSRLLHSDYMNMTPRRPGPTRKHYQPYAPPRDFAAYRS

SEQ ID NO: 1318 - DNA, 123 bp, Artificial Sequence; identical to SEQ ID NO: 52
  source: /note=“Description of Artificial Sequence: Synthetic polynucleotide”

SEQ ID NO: 1319 - protein, 35 aa, Unknown
  source: /note=“Description of Unknown: ICOS domain sequence”
TKKKYSSSVHDPNGEYMFMRAVNTAKKSRLTDVTL

SEQ ID NO: 1320 - DNA, 105 bp, Unknown
  source: /note=“Description of Unknown: ICOS domain sequence”
acaaaaaagaagtattcatccagtgtgcacgaccctaacggtgaatacatgttcatgagagcagtgaacacagccaaaaaatccagactcacagatgtgacccta

SEQ ID NO: 1321 - protein, 35 aa, Artificial Sequence
  source: /note=“Description of Artificial Sequence: Synthetic polypeptide”
TKKKYSSSVHDPNGEFMFMRAVNTAKKSRLTDVTL

SEQ ID NO: 1322 - skipped

SEQ ID NO: 1323 - DNA, 521 bp, Unknown
  source: /note=“Description of Unknown: PGK Promoter sequence”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccgcggcgacgcaaagggccttggtgcgggtctcgtcggcgcagggacgcgtttgggtcccgacggaaccttttccgcgttggggttggggcaccataagct

SEQ ID NO: 1324 - DNA, 118 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtg

SEQ ID NO: 1325 - DNA, 221 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacg

SEQ ID NO: 1326 - DNA, 324 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccg

SEQ ID NO: 1327 - DNA, 422 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccg

SEQ ID NO: 1328 - protein, 21 aa, Artificial Sequence
  source: /note=“Description of Artificial Sequence: Synthetic peptide”
  SITE (1)..(3): /note=“This region may or may not be present”
GSGEGRGSLLTCGDVEENPGP

SEQ ID NO: 1329 - protein, 22 aa, Artificial Sequence
  source: /note=“Description of Artificial Sequence: Synthetic peptide”
  SITE (1)..(3): /note=“This region may or may not be present”
GSGATNFSLLKQAGDVEENPGP

SEQ ID NO: 1330 - protein, 23 aa, Artificial Sequence
  source: /note=“Description of Artificial Sequence: Synthetic peptide”
  SITE (1)..(3): /note=“This region may or may not be present”
GSGQCTNYALLKLAGDVESNPGP

SEQ ID NO: 1331 - protein, 25 aa, Artificial Sequence
  source: /note=“Description of Artificial Sequence: Synthetic peptide”
  SITE (1)..(3): /note=“This region may or may not be present”
GSGVKQTLNFDLLKLAGDVESNPGP

SEQ ID NO: 1332-4999 - skipped (3668 entries)

SEQ ID NO: 5000 - DNA, 1467 bp, Artificial Sequence
  source: /note=“Description of Artificial Sequence: Synthetic polynucleotide”
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 5001 - protein, 489 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5002 - protein, 266 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 5003 - DNA, 1383 bp, Artificial Sequence
  source: /note=“Description of Artificial Sequence: Synthetic polynucleotide”
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 5004 - protein, 461 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5005 - protein, 267 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 5006 - DNA, 726 bp, Artificial Sequence
  source: /note=“Description of Artificial Sequence: Synthetic polynucleotide”
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagc

SEQ ID NO: 5007 - DNA, 1395 bp, Artificial Sequence
  source: /note=“Description of Artificial Sequence: Synthetic polynucleotide”
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 5008 - protein, 465 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5009 - DNA, 801 bp, Artificial Sequence
  source: /note=“Description of Artificial Sequence: Synthetic polynucleotide”
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 5010 - DNA, 1401 bp, Artificial Sequence
  source: /note=“Description of Artificial Sequence: Synthetic polynucleotide”
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 5011 - protein, 467 aa, Artificial Sequence; identical to SEQ ID NO: 115
  source: /note=“Description of Artificial Sequence: Synthetic polypeptide”

SEQ ID NO: 5012 - DNA, 735 bp, Artificial Sequence
  source: /note=“Description of Artificial Sequence: Synthetic polynucleotide”
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 5013 - protein, 245 aa, Artificial Sequence; identical to SEQ ID NO: 114
  source: /note=“Description of Artificial Sequence: Synthetic polypeptide”

SEQ ID NO: 5014 - DNA, 801 bp, Artificial Sequence
  source: /note=“Description of Artificial Sequence: Synthetic polynucleotide”
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 5015 - DNA, 1317 bp, Artificial Sequence
  source: /note=“Description of Artificial Sequence: Synthetic polynucleotide”
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 5016 - protein, 439 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5017 - DNA, 735 bp, Artificial Sequence
  source: /note=“Description of Artificial Sequence: Synthetic polynucleotide”
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 5018 - protein, 245 aa, Artificial Sequence; identical to SEQ ID NO: 114
  source: /note=“Description of Artificial Sequence: Synthetic polypeptide”

SEQ ID NO: 5019 - DNA, 63 bp, Artificial Sequence
  source: /note=“Description of Artificial Sequence: Synthetic oligonucleotide”
atggccctccctgtcaccgccctgctgcttccgctggctcttctgctccacgccgctcggccc

SEQ ID NO: 5020 - protein, 22 aa, Artificial Sequence
  source: /note=“Description of Artificial Sequence: Synthetic peptide”
MLLLVTSLLLCELPHPAFLLIP

SEQ ID NO: 5021 - DNA, 66 bp, Artificial Sequence
  source: /note=“Description of Artificial Sequence: Synthetic oligonucleotide”
atgcttctcctggtgacaagccttctgctctgtgagttaccacacccagcattcctcctgatccca

SEQ ID NO: 5022 - DNA, 66 bp, Artificial Sequence
  source: /note=“Description of Artificial Sequence: Synthetic oligonucleotide”
atgctgctgctggtgaccagcctgctgctgtgcgagctgccccaccccgcctttctgctgatcccc

SEQ ID NO: 5023 - protein, 4 aa, Artificial Sequence
  source: /note=“Description of Artificial Sequence: Synthetic peptide”
  source: /note=“See specification as filed for detailed description of substitutions and preferred embodiments”
GGGS

SEQ ID NO: 5024 - protein, 120 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 5025 - protein, 120 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 5026 - protein, 120 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 5027 - protein, 107 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 5028 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5029 - protein, 465 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5030 - DNA, 1461 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 5031 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 5032 - DNA, 1398 bp, Artificial Sequence
  source: /note=“Description of Artificial Sequence: Synthetic polynucleotide”
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 5033 - DNA, 1395 bp, Artificial Sequence
  source: /note=“Description of Artificial Sequence: Synthetic polynucleotide”
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 5034 - protein, 25 aa, Artificial Sequence
  source: /note=“Description of Artificial Sequence: Synthetic peptide”
GGGGSGGGGSGGGGSGGGGSGGGGS

SEQ ID NO: 5035 - protein, 30 aa, Artificial Sequence; identical to SEQ ID NO: 53
  source: /note=“Description of Artificial Sequence: Synthetic polypeptide”

SEQ ID NO: 5036 - protein, 30 aa, Artificial Sequence; identical to SEQ ID NO: 53
  source: /note=“Description of Artificial Sequence: Synthetic polypeptide”
  SITE (1)..(30): /note=“This sequence may encompass 0-6 ′Gly Gly Gly Gly Ser′ repeating units wherein some positions may be absent”

SEQ ID NO: 5037 - protein, 5 aa, Artificial Sequence
  source: /note=“Description of Artificial Sequence: Synthetic peptide”
GGGGS